(19) United States
(12) Patent Application Publication
(10) Pub. No.: US 2007/0082880 A1
(43) Pub. Date: Apr. 12, 2007

(54) Thieno [2,3-B] pyridine-5-carbonitriles as protein kinase inhibitors

(76) Inventors: Diane H. Boschelli, New City, NY (US); Derek C. Cole, New City, NY (US); Magda Asselin, Mahwah, NJ (US); Ana C. Barrios Sosa, Olanta, SC (US); Biqi Wu, Nanuet, NY (US); Lawrence N. Tumey, New Windsor, NY (US)

Correspondence Address: KIRKPATRICK & LOCKHART PRESTON GATES ELLIS LLP, STATE STREET FINANCIAL CENTER, ONE LINCOLN STREET, BOSTON, MA, 02111-2950, US

(21) Appl. No.: 11/527,996

(22) Filed: Sep. 27, 2006

Related U.S. Application Data
(60) Provisional application No. 60/720,821, filed on Sep. 27, 2005.

(51) Int. Cl. A61K 31/55; A61K 31/4743; C07D 498/02

(52) U.S. Cl. 514/183; 514/301; 514/217.07; 540/598; 514/481; 546/114

(57) ABSTRACT

Disclosed are compounds of Formula I: 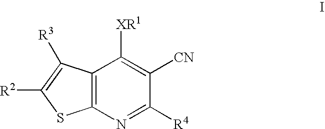
wherein R¹, R², R³, R⁴, and X, are defined hereinbefore in the specification, which can be useful in the treatment of autoimmune and inflammatory diseases, and processes for producing said compounds.

### CROSS-REFERENCE TO RELATED APPLICATION

This application claims priority to and the benefit of U.S. Provisional Patent Application Ser. No. 60/720,821, filed Sep. 27, 2005, the disclosure of which is incorporated herein by reference.

### INTRODUCTION

The present teachings relate to substituted thieno[2,3-b]pyridine-5-carbonitriles that are capable of inhibiting protein kinases and to methods for the preparation of the substituted thieno[2,3-b]pyridine-5-carbonitriles. The thienopyridines of the present teachings can be useful for the treatment of autoimmune and inflammatory diseases such as asthma, arthritis, multiple sclerosis, and diabetes.

Protein kinases are enzymes that catalyze the transfer of phosphate group from adenosine triphosphate (ATP) to an amino acid residue, such as tyrosine, serine, threonine, or histidine, on a protein. Regulation of these protein kinases is essential for the control of a wide variety of cellular events including proliferation and migration. A large number of diseases are associated with these kinase-mediated abnormal cellular events including various inflammatory diseases and autoimmune diseases such as asthma, psoriasis, arthritis, rheumatoid arthritis, osteoarthritis, joint inflammation, multiple sclerosis, diabetes including type II diabetes, and inflammatory bowel diseases such as Crohn's disease and colitis (Kim, J. et al. (2004), J. Clin. Invest., 114: 823-827; Schmitz-Peiffer, C. et al. (2005), Drug Discov Today, 2(2): 105-110; Salek-Ardakani, S. et al. (2005), J. Immunol., 175: 7635-7641; Healy. A. et al. (2006), J. Immunol., 177: 1886-1893; and Tan, S-L. (2006), J. Immunol., 176: 2872-2879).

One class of serine/threonine kinases is the protein kinase C (PKC) family. This group of kinases consists of 10 members that share sequence and structural homology. The PKCs are divided into 3 groups and include the classic, the novel, and the atypical isoforms. The theta isoform (PKCθ) is a member of the novel calcium-independent class of PKCs (Baier, G. et al. (1993), FEBS Lett., 326: 51-5), with some expression reported in mast cells (Liu, Y. et al. (2001), J. Leukoc. Biol., 69: 831-40), endothelial cells (Mattila, P. et al. (1994), Life Sci., 55: 1253-60), and skeletal muscle (Baier, G. et al. (1994), Eur. J. Biochem., 225: 195-203). It has been shown that PKCθ plays an essential role in T cell receptor (TCR)-mediated signaling (Tan, S. L. et al. (2003), Biochem. J., 376: 545-52). Specifically, it has been observed that inhibiting PKCθ signal transduction, as demonstrated with two independent PKCθ knockout mouse lines, will result in defects in T cell activation and interleukin-2 (IL-2) production (Sun, Z. et al. (2000), Nature, 404: 402-7; Pfeifhofer, C. et al. (2003), J. Exp. Med., 197: 1525-35). It also has been shown that PKCθ-deficient mice show impaired pulmonary inflammation and airway hyperresponsiveness (AHR) in a Th2-dependent murine asthma model, with no defects in viral clearance and Th1-dependent cytotoxic T cell function (Berg-Brown, N. N. et al. (2004), J. Exp. Med., 199: 743-52; Marsland, B. J. et al. (2004), J. Exp. Med., 200: 181-9). The impaired Th2 cell responses result in reduced levels of interleukin-4 (IL-4) and immunoglobulin E (IgE), contributing to the AHR and inflammatory pathophysiology.

Evidence also exists that PKCθ participates in the IgE receptor (FceRI)-mediated response of mast cells (Liu, Y. et al. (2001), J. Leukoc. Biol., 69: 831-840). In human-cultured mast cells (HCMC), it has been demonstrated that PKC kinase activity rapidly localizes (in less than five minutes) to the membrane following FceRI cross-linking (Kimata, M. et al. (1999), Biochem. Biophys. Res. Commun., 257(3): 895-900). A recent study examining in vitro activation of bone marrow mast cells (BMMCs) derived from wild-type and PKCθ-deficient mice shows that upon FceRI cross-linking, BMMCs from PKCθ-deficient mice produced reduced levels of interleukin-6 (IL-6), tumor necrosis factor-alpha (TNFα), and interleukin-13 (IL-13) in comparision with BMMCs from wild-type mice, suggesting a potential role for PKCθ in mast cell cytokine production in addition to T cell activation (Ciarletta, A. B. et al. (2005), poster presentation at the 2005 American Thorasic Society International Conference).

Other serine/threonine kinases include those of the mitogen-activated protein kinase (MAPK) pathway which consists of the MAP kinase kinases (MAPKK) (e.g., mek and their substrates) and the MAP kinases (MAPK) (e.g., erk). Members of the raf family of kinases phosphorylate residues on mek. The cyclin-dependent kinases (cdks), including cdc2/cyclin B, cdk2/cyclin A, cdk2/cyclin E and cdk4/cyclin D, and others, are serine/threonine kinases that regulate mammalian cell division. Additional serine/threonine kinases include the protein kinases A and B. These kinases, known as PKA or cyclic AMP-dependent protein kinase and PKB (Akt), play key roles in signal transduction pathways.

Tyrosine kinases (TKs) are divided into two classes: the non-transmembrane TKs and transmembrane growth factor receptor TKs (RTKs). Growth factors, such as epidermal growth factor (EGF), bind to the extracellular domain of their partner RTK on the cell surface which activates the RTK, initiating a signal transduction cascade that controls a wide variety of cellular responses. In addition to EGF, there are several other RTKs including FGFr (the receptor for fibroblast growth factor (FGF)); flk-1 (also known as KDR, and flt-1, the receptors for vascular endothelial growth factor (VEGF)); and PDGFr (the receptor for platelet derived growth factor (PDGF)). Other RTKs include tie-1 and tie-2, colony stimulating factor receptor, the nerve growth factor receptor, and the insulin-like growth factor receptor. In addition to the RTKs there is another family of TKs termed the cytoplasmic protein or non-receptor TKs. The cytoplasmic protein TKs have intrinsic kinase activity, are present in the cytoplasm and nucleus, and participate in diverse signaling pathways. There is a large number of non-receptor TKs including Abl, Jak, Fak, Syk, Zap-70 and Csk and also the Src family of kinases (SFKs) which includes Src, Lck, Lyn, Fyn, Yes and others.

Thieno[2,3-b]pyridines and certain pyridine and pyrimidine derivatives have been noted as kinase inhibitors. These compounds differ both in nature and placement of substituents at various positions when compared to the compounds disclosed herein.

### SUMMARY

The present teachings relate to thieno[2,3-b]pyridine-5-carbonitrile compounds of formula I: 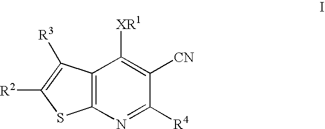
and pharmaceutically acceptable salts, hydrates, or esters thereof, wherein R¹, R², R³, R⁴, and X are defined as described herein. The present teachings also provide methods of making the compounds of formula I, and methods of treating autoimmune and inflammatory diseases, such as asthma and arthritis, comprising administering a therapeutically effective amount of a compound of formula I to a patient in need thereof.

### DETAILED DESCRIPTION

The present teachings provide compounds of formula I or a pharmaceutically acceptable salt, hydrate or ester thereof: 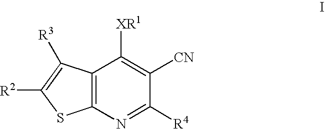
wherein:
X is a) —NR⁵—Y—, b) —O—Y—, c) —S(O)ₘ—Y—, d) —S(O)ₘNR⁵—Y—, e) —NR⁵S(O)ₘ—Y—, f) —C(O)NR⁵—Y—, g) —NR⁵C(O)—Y—, h) —C(S)NR⁵—Y—, i) —NR⁵C(S)—Y—, j) —C(O)O—Y—, k) —OC(O)—Y—, l) —C(O)—Y—, or m) a covalent bond;
Y, at each occurrence, independently is a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, or e) a covalent bond;
R¹ is a) a C₁₋₁₀ alkyl group, b) a C₃₋₁₀ cycloalkyl group, c) a 3-12 membered cycloheteroalkyl group, d) a C₆₋₁₄ aryl group, or e) a 5-13 membered heteroaryl group, wherein each of a)-e) optionally is substituted with 1-4 R⁶ groups, and provided that R¹ is not a phenyl group;
R² is a) H, b) halogen, c) —C(O)R⁸, d) —C(O)OR⁸, e) —C(O)NR⁹R¹⁰, f) —C(S)R⁸, g) —C(S)OR⁸, h) —C(S)NR⁹R¹⁰, i) a C₁₋₁₀ alkyl group, j) a C₂₋₁₀ alkenyl group, k) a C₂₋₁₀ alkynyl group, l) a C₃₋₁₀ cycloalkyl group, m) a C₆₋₁₄aryl group, n) a 3-12 membered cycloheteroalkyl group, or o) a 5-13 membered heteroaryl group, wherein each of i)-o) optionally is substituted with 1-4 R⁶ groups;
R³ is a) H, b) halogen, c) —OR⁸, d) —NR⁹R¹⁰, e) —N(O)R⁹R¹⁰, f) S(O)ₘR⁸, g) S(O)ₘOR⁸, h) —C(O)R⁸, i) —C(O)OR⁸, j) —C(O)NR⁹R¹⁰, k) —C(S)R⁸, l) —C(S)OR⁸, m) —C(S)NR⁹R¹⁰, n) —Si(C₁₋₁₀ alkyl group)₃, o) a C₁₋₁₀ alkyl group, p) a C₂₋₁₀ alkenyl group, q) a C₂₋₁₀ alkynyl group, r) a C₃₋₁₀ cycloalkyl group, s) a C₆₋₁₄ aryl group, t) a 3-12 membered cycloheteroalkyl group, or u) a 5-13 membered heteroaryl group, wherein each of o)-u) optionally is substituted with 1-4 R⁶ groups;
R⁴ is a) H, b) halogen, c) a C₁₋₁₀ alkyl group, d) a C₂₋₁₀ alkenyl group, e) a C₂₋₁₀ alkynyl group, f) a C₁₋₁₀ haloalkyl group, g) a C₃₋₁₀ cycloalkyl group, h) a C₆₋₁₄ aryl group, i) a 3-12 membered cycloheteroalkyl group, or j) a 5-13 membered heteroaryl group, wherein each of c)-j) optionally is substituted with 1-4 R⁶ groups;
R⁵ is a) H, b) a C₁₋₁₀ alkyl group, c) a C₂₋₁₀ alkenyl group, d) a C₂₋₁₀ alkynyl group, or e) a C₁₋₁₀ haloalkyl group;
R⁶, at each occurrence, independently is a) R⁷ or b) —Y—R⁷;
R⁷, at each occurrence, independently is a) halogen, b) —CN, c) —NO₂, d) oxo, e) —OR⁸, f) —NR⁹R¹⁰, g) —N(O)R⁹R¹⁰, h) —S(O)ₘR⁸, i) —S(O)ₘOR⁸, j) —SO₂NR⁹R¹⁰, k) —C(O)R⁸, l) —C(O)OR⁸, m) —C(O)NR⁹R¹⁰, n) —C(S)R⁸, o) —C(S)OR⁸, p) —C(S)NR⁹R¹⁰, q) —Si(C₁₋₁₀ alkyl)₃, r) a C₁₋₁₀ alkyl group, s) a C₂₋₁₀ alkenyl group, t) a C₂₋₁₀ alkynyl group, u) a C₁₋₁₀ haloalkyl group, v) a C₃₋₁₀ cycloalkyl group, w) a C₆₋₁₄ aryl group, x) a 3-12 membered cycloheteroalkyl group, or y) a 5-13 membered heteroaryl group, wherein each of r)-y) optionally is substituted with 1-4 R¹¹ groups;
R⁸, at each occurrence, independently is a) H, b) —C(O)R¹⁴, c) —C(O)OR¹⁴, d) a C₁₋₁₀ alkyl group, e) a C₂₋₁₀ alkenyl group, f) a C₂₋₁₀ alkynyl group, g) a C₁₋₁₀ haloalkyl group, h) a C₃₋₁₀ cycloalkyl group, i) a C₆₋₁₄ aryl group, j) a 3-12 membered cycloheteroalkyl group, or k) a 5-13 membered heteroaryl group, wherein each of d)-k) optionally is substituted with 1-4 R¹¹ groups;
R⁹ and R¹⁰, at each occurrence, independently are a) H, b) —OR¹³, c) —NR¹⁴R¹⁵, d) —S(O)ₘR¹⁴, e) —S(O)ₘOR¹⁴, f) —S(O)₂NR¹⁴R¹⁵, g) —C(O)R¹⁴, h) —(O)OR¹⁴, i) —C(O)NR¹⁴R¹⁵, j) —C(S)R¹⁴, k) —C(S)OR¹⁴, l) —C(S)NR¹⁴R¹⁵, m) a C₁₋₁₀ alkyl group, n) a C₂₋₁₀ alkenyl group, o) a C₂₋₁₀ alkynyl group, p) a C₁₋₁₀ haloalkyl group, q) a C₃₋₁₀ cycloalkyl group, r) a C₆₋₁₄ aryl group, s) a 3-12 membered cycloheteroalkyl group, or t) a 5-13 membered heteroaryl group; wherein each of m)-t) optionally is substituted with 1-4 R¹¹ groups;
R¹¹, at each occurrence, independently is a) R¹², orb) —Y—R¹²;
R¹², at each occurrence, independently is a) halogen, b) —CN, c) —NO₂, d) oxo, e) —R¹³, f) —NR¹⁴R¹⁵, g) —N(O)R¹⁴R¹⁵, h) —S(O)ₘR¹³ i) —S(O)ₘOR¹³, j) —SO₂NR¹⁴R¹⁵, k) —C(O)R¹³, l) —C(O)OR¹³, m) —C(O)NR⁴R¹⁵, n) —C(S)R¹³, o) —C(S)OR¹³, p) —C(S)NR¹⁴R¹⁵, q) —Si(C₁₋₁₀ alkyl)₃, r) a C₁₋₁₀ alkyl group, s) a C₂₋₁₀ alkenyl group, t) a C₂₋₁₀ alkynyl group, u) a C₁₋₁₀ haloalkyl group, v) a C₃₋₁₀ cycloalkyl group, w) a C₆₋₁₄ aryl group, x) a 3-12 membered cycloheteroalkyl group, or y) a 5-13 membered heteroaryl group, wherein each of r)-y) optionally is substituted with 1-4 R¹⁶ groups;
R¹³ is selected from a) H, b) —C(O)R¹⁴, c) —C(O)OR¹⁴, d) a C₁₋₁₀ alkyl group, e) a C₂₋₁₀ alkenyl group, f) a C₂₋₁₀ alkynyl group, g) a C₁₋₁₀ haloalkyl group, h) a C₃₋₁₀ cycloalkyl group, i) a C₆₋₁₄ aryl group, j) a 3-12 membered cycloheteroalkyl group, or k) a 5-13 membered heteroaryl group, wherein each of d)-k) optionally is substituted with 1-4 R¹⁶ groups;
R¹⁴ and R¹⁵, at each occurrence, independently are a) H, b) a C₁₋₁₀ alkyl group, c) a C₂₋₁₀ alkenyl group, d) a C₂₋₁₀ alkynyl group, e) a C₁₋₁₀ haloalkyl group, f) a C₃₋₁₀ cycloalkyl group, g) a C₆₋₁₄ aryl group, h) a 3-12 membered cycloheteroalkyl group, or i) a 5-13 membered heteroaryl group; wherein each of b)-i) optionally is substituted with 1-4 R¹⁶ groups;
R¹⁶, at each occurrence, independently is a) halogen, b) —CN, c) —NO₂, d) —OH, e) —NH₂, f) —NH(C₁₋₁₀ alkyl), g) oxo, h) —N(C₁₋₁₀ alkyl)₂, i) —SH, j) —S(O)ₘ—C₁₋₁₀ alkyl, k) —S(O)₂OH, l) —S(O)ₘ—OC₁₋₁₀ alkyl, m) —C(O)C₁₋₁₀ alkyl, n) —C(O)OH, o) —C(O)—OC₁₋₁₀ alkyl, p) —C(O)NH₂, q) —C(O)NH—C₁₋₁₀ alkyl, r) —C(O)N(C₁₋₁₀ alkyl)₂, s) —C(S)NH₂, t) —C(S)NH—C₁₋₁₀ alkyl, u) —C(S)N(C₁₋₁₀ alkyl)₂, v) a C₁₋₁₀ alkyl group, w) a C₂₋₁₀ alkenyl group, x) a C₂₋₁₀ alkynyl group, y) a C₁₋₁₀ alkoxy group, z) a C₁₋₁₀ alkylthio group, aa) a C₁₋₁₀ haloalkyl group, ab) a C₃₋₁₀ cycloalkyl group, ac) a C₆₋₁₄ aryl group, ad) a 3-12 membered cycloheteroalkyl group, or ae) a 5-13 membered heteroaryl group; and

m is 0, 1, or 2.

In some embodiments, the thieno[2,3-b]pyridine ring can be oxidized on the nitrogen atom to provide the corresponding N-oxide having the formula I′: 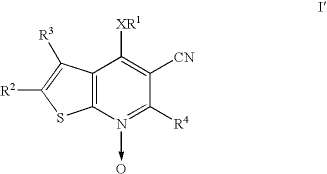
wherein R¹, R², R³, R⁴, and X are as defined hereinabove.

In other embodiments, the thieno[2,3-b]pyridine ring can be oxidized on the sulfur atom to provide the corresponding S-oxide or S,S-dioxide having the formula I″: 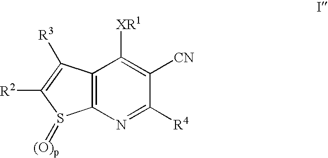
wherein p is 1 or 2, and R¹, R², R³, R⁴, and X are as defined hereinabove.

Formulae I, I′, and I″ can be collectively illustrated as: 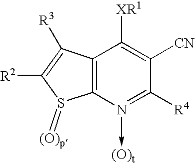
wherein p′ is 0, 1, or 2, t is 0 or 1, and R¹, R², R³, R⁴, and X are as defined hereinabove. As illustrated, the thieno[2,3-b]pyridine ring of compounds of formula I can undergo mono- or di-oxidation at the sulfur atom and/or mono-oxidation at the nitrogen atom to provide the corresponding thieno[2,3-b]pyridine-1-oxides, thieno[2,3-b]pyridine-1,1-dioxides, thieno[2,3-b]pyridine-1,1,7-trioxides, thieno[2,3-b]pyridine-1,7-dioxides, and thieno[2,3-b]pyridine-7-oxides.

In some embodiments, X can be —NR⁵—Y—, —O—, —NR⁵C(O)—, or a covalent bond, where R⁵ and Y are as defined hereinabove. For example, R⁵ can be H or a C₁₋₆ alkyl group, and Y can be a covalent bond or a divalent C₁₋₆ alkyl group. In particular, X can be —NH—, —N(CH₃)—, —NH—CH₂—, —NH—(CH₂)₂—, —N(CH₃)—CH₂—, —O—, —NHC(O)—, —N(CH₃)C(O)—, or a covalent bond.

In some embodiments, R¹ can be a 5-13 membered heteroaryl group optionally substituted with 1-4 R⁶ groups. Examples of 5-13 membered heteroaryl groups can include, but are not limited to, an indolyl group, a benzimidazolyl group, a pyrrolo[2,3-b]pyridinyl group, a pyridinyl group, and an imidazolyl group, each of which can be optionally substituted with 1-4 R⁶ groups.

In particular, R¹ can be an indolyl group optionally substituted with 1-4 R⁶ groups and connected to X or the thienopyridine ring at any of the available carbon ring atoms. For example, R¹ can be a 1H-indol-5-yl group, a 1H-indol-4-yl group, a 1H-indol-7-yl group, a 1H-indol-6-yl group, a 4-methyl-1H-indol-5-yl group, a 2-methyl-1H-indol-5-yl group, a 7-methyl-1H-indol-5-yl group, a 3-methyl-1H-indol-5-yl group, a 1-methyl-1H-indol-5-yl group, a 6-methyl-1H-indol-5-yl group, or a 4-ethyl-1H-indol-5-yl group.

In other embodiments, R¹ can be a 1H-benzimidazol-5-yl group, a 1H-benzimidazol-4-yl group, a 1H-pyrrolo[2,3-b]pyridin-5-yl group, a 1H-pyrrolo[2,3-b]pyridin-4-yl group, a pyridin-3-yl group, or a pyridin-4-yl group, each of which can be optionally substituted with 1-4 R⁶ groups. For example, R¹ can be a 4-chloro-1H-pyrrolo[2,3-b]pyridin-5-yl group or a 4-chloro-1-[(4-methylphenyl)sulfonyl]-1H-pyrrolo[2,3-b]pyridin-5-yl group.

Compounds of formula I where each of R², R³ and R⁴ is H are within the scope of the present teachings. However, the present teachings generally relate to those compounds of formula I where at least one of C2 and C3 of the thienopyridine ring is substituted, that is, at least one of R² and R³ is not H. In some embodiments, both C2 and C3 of the thienopyridine ring are substituted, that is, neither R² nor R³ is H. Exemplary substitution groups at C2 and/or C3 can include, but are not limited to, those described hereinbelow.

In some embodiments, R² can be H, a halogen, —C(O)R⁸, —C(O)OR⁸, or —C(O)NR⁹R¹⁰. In particular, R² can be H, I, Cl, Br, —C(O)R⁸, —C(O)OR⁸, or —C(O)NR⁹R¹⁰, where R⁸, R⁹ and R¹⁰ are as defined hereinabove. For example, R⁸, R⁹, and R¹⁰ independently can be H, a C₁₋₁₀ alkyl group, a 3-12 membered cycloheteroalkyl group, a 5-13 membered heteroaryl group, or a phenyl group, where each of the C₁₋₁₀ alkyl group, the 3-12 membered cycloheteroalkyl group, the 5-13 membered heteroaryl group, and the phenyl group can be optionally substituted with 1-4 R¹¹ groups as described hereinabove.

In other embodiments, R² can be a C₁₋₁₀ alkyl group, a C₂₋₁₀ alkenyl group, a C₂₋₁₀ alkynyl group, a C₃₋₁₀ cycloalkyl group, a 3-12 membered cycloheteroalkyl group, a C₆₋₁₄ aryl group, or a 5-13 membered heteroaryl group, each of which can be optionally substituted with 1-4 R⁶ groups as described hereinabove. For example, R⁶ can be a halogen, an oxo group, —OR⁸, —NR⁹R¹⁰, —S(O)₂R⁸, —S(O)₂R⁸, —SO₂NR⁹R¹⁰, —C(O)R⁸, —C(O)OR⁸, —C(O)NR⁹R¹⁰, —Si(CH₃)₃, a —C₁₋₄ alkyl-OR⁸, a —C₁₋₄ alkyl-NR⁹R¹⁰ group, a —C₁₋₄ alkyl-C₆₋₁₄ aryl group, a —C₁₋₄ alkyl-3-12 membered cycloheteroalkyl group, a —C₁₋₄ alkyl-5-13 membered heteroaryl group, a C₁₋₁₀ alkyl group, a C₂₋₁₀ alkenyl group, a C₂₋₁₀ alkynyl group, a C₁₋₁₀ haloalkyl group, a C₃₋₁₀ cycloalkyl group, a C₆₋₁₄ aryl group, a 3-12 membered cycloheteroalkyl group, or a 5-13 membered heteroaryl group, where R⁸, R⁹ and R¹⁰ are as defined hereinabove and each of the C₁₋₁₀ alkyl group, the C₂₋₁₀ alkenyl group, the C₂₋₁₀ alkynyl group, the C₃₋₁₀ cycloalkyl group, the C₆₋₁₄ aryl group, the 3-12 membered cycloheteroalkyl group, and the 5-13 membered heteroaryl group immediately above can be optionally substituted with 1-4 R¹¹ groups.

In particular embodiments, R² can be a C₁₋₆ alkyl group, a C₂₋₆ alkenyl group, or a C₂₋₆ alkynyl group, each of which can be optionally substituted 1-4 R⁶ groups, where R⁶, at each occurrence, independently can be a halogen, —OR⁸, —NR⁹R¹⁰, —C(O)R⁸, —C(O)OR⁸, —C(O)NR⁹R¹⁰, —Si(CH₃)₃, a phenyl group, a 5-6 membered cycloheteroalkyl group, or a 5-6 membered heteroaryl group, R⁸, R⁹ and R¹⁰ are as defined hereinabove, and each of the phenyl group, the 5-6 membered cycloheteroalkyl group, and the 5-6 membered heteroaryl group can be optionally substituted with 1-4 R¹¹ groups as described hereinabove.

For example, R⁸, at each occurrence, independently can be H, a C₁₋₆ alkyl group, a phenyl group, a 5-6 membered cycloheteroalkyl group, a 5-6 membered heteroaryl group, wherein the C₁₋₆ alkyl group, the phenyl group, the 5-6 membered cycloheteroalkyl group, and the 5-6 membered heteroaryl group can be optionally substituted with 1-4 R¹¹ groups. R⁹ and R¹⁰, at each occurrence, independently can be H, —N(C₁₋₆ alkyl)₂ group, a C₁₋₆ alkyl group, a phenyl group, a 5-6 membered cycloheteroalkyl group, or a 5-6 membered heteroaryl group, wherein the C₁₋₆ alkyl group, the phenyl group, the 5-6 membered cycloheteroalkyl group, and the 5-6 membered heteroaryl group can be optionally substituted with 1-4 R¹¹ groups. The 5-6 membered cycloheteroalkyl group and the 5-6 membered heteroaryl group, for example, can be a piperazinyl group, a piperidinyl group, pyrrolidinyl group, a morpholinyl group, a pyrazolyl group, a pyrimidinyl group, or a pyridinyl group, each of which can be optionally substituted with 1-4 R¹¹ groups. At each occurrence, R¹¹ independently can be a halogen, OR¹³, —NR¹⁴R¹⁵, —C(O)NR¹⁴R¹⁵, a C₁₋₆ alkyl group, a C₁₋₆ alkoxyl group, a C₁₋₆ haloalkyl group, a —C₁₋₄ alkyl-NR¹⁴R¹⁵ group, a —C₁₋₄ alkyl-phenyl group, a —C₁₋₄ alkyl-5-6 membered cycloheteroalkyl group, or a —C₁₋₄ alkyl-5-6 membered heteroaryl group, where R¹³, R¹⁴ and R¹⁵ are as defined hereinabove.

In other embodiments, R² can be a C₃₋₆ cycloalkyl group, a 3-10 membered cycloheteroalkyl group, a C₆₋₁₀ aryl group, or a 5-10 membered heteroaryl group, each of which can be optionally substituted with 1-4 R⁶ groups as described hereinabove. For example, the C₃₋₆ cycloalkyl group, the 3-10 membered cycloheteroalkyl group, the C₆₋₁₀ aryl group, and the 5-10 membered heteroaryl group can be a cyclohexanyl group, a cyclohexenyl group, a piperazinyl group, a piperidinyl group, a morpholinyl group, a pyrrolidinyl group, a tetrahydropyridinyl group, a dihydropyridinyl group, a phenyl group, a naphthyl group, a pyridinyl group, a pyrazolyl group, a pyridazinyl group, an indolyl group, a pyrazinyl group, a pyrimidinyl group, a thienyl group, a furyl group, a thiazolyl group, a quinolinyl group, a benzothienyl group, or an imidazolyl group, each of which can be optionally substituted with 1-4 R⁶ groups.

For example, R⁶, at each occurrence, independently can be a halogen, an oxo group, —OR⁸, —NR⁹R¹⁰, —S(O)₂R⁸, —S(O)₂OR⁸, —SO₂NR⁹R¹⁰, —C(O)R⁸, —C(O)OR⁸, —C(O)NR⁹R¹⁰, a C₁₋₁₀ alkyl group, a C₃₋₁₀ cycloalkyl group, a C₆₋₁₄ aryl group, a 3-12 membered cycloheteroalkyl group, or a 5-13 membered heteroaryl group, where R⁸, R⁹ and R¹⁰ are as defined hereinabove and each of the C₁₋₁₀ alkyl group, the C₃₋₁₀ cycloalkyl group, the C₆₋₁₄ aryl group, the 3-12 membered cycloheteroalkyl group, and the 5-13 membered heteroaryl group can be optionally substituted with 1-4 R¹¹ groups.

In particular embodiments, R² can be a phenyl group optionally substituted with 1-4 R⁶ groups, where R⁶, at each occurrence, independently can be a halogen, —OR⁸, —NR⁹R¹⁰, —S(O)₂R⁸, —SO₂NR⁹R¹⁰, —C(O)R⁸, —C(O)OR⁸, —C(O)NR⁹R¹⁰, a C₁₋₆ alkyl group, a C₃₋₆ cycloalkyl group, a C₆₋₁₀ aryl group, a 3-10 membered cycloheteroalkyl group, and a 5-10 membered heteroaryl group, where R⁸, R⁹ and R¹⁰ are as defined hereinabove and each of the C₁₋₆ alkyl group, the C₃₋₆ cycloalkyl group, the C₆₋₁₀ aryl group, the 3-10 membered cycloheteroalkyl group, and the 5-10 membered heteroaryl group can be optionally substituted with 1-4 R¹¹ groups. The C₃₋₁₀ cycloalkyl group, the C₆₋₁₀ aryl group, the 3-10 membered cycloheteroalkyl group, and the 5-10 membered heteroaryl group, for example, can be a cyclohexanyl group, a cyclohexenyl group, a piperazinyl group, a piperidinyl group, a morpholinyl group, a pyrrolidinyl group, a tetrahydropyridinyl group, a dihydropyridinyl group, a phenyl group, a naphthyl group, a pyridinyl group, a pyrazolyl group, a pyridazinyl group, an indolyl group, a pyrazinyl group, a pyrimidinyl group, a thienyl group, a furyl group, a thiazolyl group, a quinolinyl group, a benzothienyl group, or an imidazolyl group, each of which can be optionally substituted with 1-4 R¹¹ groups.

For example, R⁸, at each occurrence, independently can be H, a C₁₋₆ alkyl group, a phenyl group, a 5-6 membered cycloheteroalkyl group, or a 5-6 membered heteroaryl group, wherein the C₁₋₆ alkyl group, the phenyl group, the 5-6 membered cycloheteroalkyl group, and the 5-6 membered heteroaryl group can be optionally substituted with 1-4 R¹¹ groups. R⁹ and R¹⁰, at each occurrence, independently can be H, —C(O)OR¹⁴, —C(O)NR¹⁴R¹⁵, —S(O)₂R¹⁴, —S(O)₂NR¹⁴R¹⁵, —NR¹⁴R¹⁵, a C₁₋₆ alkyl group, a phenyl group, a 5-6 membered cycloheteroalkyl group, or a 5-6 membered heteroaryl group, wherein R¹⁴ and R¹⁵ are as defined hereinabove and each of the C₁₋₆ alkyl group, the phenyl group, the 5-6 membered cycloheteroalkyl group, and the 5-6 membered heteroaryl group can be optionally substituted with 1-4 R¹¹ groups. The 5-6 membered cycloheteroalkyl group and the 5-6 membered heteroaryl group, for example, can be a piperazinyl group, a piperidinyl group, pyrrolidinyl group, a morpholinyl group, a pyrazolyl group, a pyrimidinyl group, or a pyridinyl group, each of which can be optionally substituted with 1-4 R¹¹ groups. At each occurrence, R¹¹ independently can be a halogen, OR¹³, —NR¹⁴R¹⁵, —C(O)NR¹⁴R¹⁵, a C₁₋₆ alkyl group, a C₁₋₆ alkoxyl group, a C₁₋₆ haloalkyl group, a —C₁₋₂ alkyl-NR¹⁴R¹⁵ group, a —C₁₋₂ alkyl-phenyl group, a —C₁₋₂ alkyl-5-6 membered cycloheteroalkyl group, or a —C₁₋₂ alkyl-5-6 membered heteroaryl group, where R¹³, R¹⁴ and R¹⁵ are as defined hereinabove.

In certain embodiments, R² can have the formula -A-J-G, wherein A is a divalent C₂₋₁₀ alkenyl group, a divalent C₂₋₁₀ alkynyl group, a divalent C₃₋₁₀ cycloalkyl group, a divalent 3-12 membered cycloheteroalkyl group, a divalent C₆₋₁₄ aryl group, or a divalent 5-13 membered heteroaryl group; J is a divalent C₁₋₁₀ alkyl group or a covalent bond; and G is selected from H, —S(O)ₘR⁸, —S(O)ₘOR⁸, —SO₂NR⁹R¹⁰, —C(O)R⁸, —C(O)OR⁸, —C(O)NR⁹R¹⁰, NR⁹R¹⁰, a 3-12 membered cycloheteroalkyl group, a C₆₋₁₄ aryl group, and a 5-13 membered heteroaryl group, where each of the 3-12 membered cycloheteroalkyl group, the C₆₋₁₄ aryl group, and the 5-13 membered heteroaryl group optionally can be substituted with 1-4 R¹¹ groups. A can be optionally substituted with 1-3 R⁶ groups in addition to the -J-G group.

Certain compounds of these embodiments include those wherein A is a phenyl group, J is a divalent C₁₋₂ alkyl group, and G is a 3-12 membered cycloheteroalkyl group optionally substituted with 1-4 R¹¹ groups. Examples of 3-12 membered cycloheteroalkyl groups can include, but are not limited to, a pyrrolidinyl group, a piperidinyl group, a piperazinyl group, and a morpholinyl group. In particular, G can be an N-substituted piperazinyl group, wherein the substitution group has the formula —(CH₂)ₙ-D, wherein n is 1, 2, or 3, and D is selected from H, —OR¹³, —NR¹⁴R¹⁵, —C(O)R¹³, a 3-12 membered cycloheteroalkyl group, a C₆₋₁₄ aryl group, or a 5-13 membered heteroaryl group.

In other embodiments, G can be —NR⁹R¹⁰. For example, R⁹ can be H or a C₁₋₁₀ alkyl group, wherein the C₁₋₁₀ alkyl group optionally can be substituted with —OR¹¹, and R¹⁰ can be H or a C₁₋₁₀ alkyl group, wherein the C₁₋₁₀ alkyl group optionally can be substituted with 1-4 moieties selected from —OR¹³, —NR¹⁴R¹⁵, and a 3-10 membered cycloheteroalkyl group.

Other embodiments wherein R² has the formula -A-J-G include those wherein A is selected from a thienyl group, a furanyl group, an imidazolyl group, a 1-methyl-imidazolyl group, a thiazolyl group, and a pyridinyl group, where J and G are as defined hereinabove.

Further embodiments include those wherein A is a divalent C₂₋₁₀ alkenyl group or a divalent C₂₋₁₀ alkynyl group; J is a covalent bond; and G is selected from —NR⁹R¹⁰, —Si(C₁₋₆ alkyl)₃, a 3-12 membered cycloheteroalkyl group, a C₆₋₁₄ aryl group, and a 5-13 membered heteroaryl group, wherein each of the 3-12 membered cycloheteroalkyl group, the C₆₋₁₄ aryl group, and the 5-13 membered heteroaryl group can be optionally substituted with 1-4 R¹¹ groups. For example, R¹¹ can be selected from —NR¹⁴R¹⁵, a —C₁₋₂ alkyl-NR¹⁴R¹⁵ group, and a —C₁₋₂ alkyl-3-12 membered cycloheteroalkyl group, wherein the 3-12 membered cycloheteroalkyl group optionally can be substituted with 1-4 R¹⁶ groups.

In some embodiments, R³ can be H, a halogen, a C₁₋₆ alkyl group, a C₂₋₆ alkynyl group, or a phenyl group, wherein the C₁₋₆ alkyl group, the C₂₋₆ alkynyl group, and the phenyl group can be optionally substituted with 1-4 R⁶ groups. For example, R⁶, at each occurrence, independently can be —NR⁹R¹⁰, a C₁₋₆ alkyl group, a phenyl group, or a 5-10 cycloheteroalkyl group, wherein the C₁₋₆ alkyl group, the phenyl group, and the 5-10 cycloheteroalkyl group can be optionally substituted with 1-4 R¹¹ groups.

In some embodiments, R⁴ can be H.

It should be understood that the present teachings can exclude certain embodiments of compounds within the genus of compounds identified by formula I. For example, when R⁴ is an optionally substituted 3-12 membered cycloheteroalkyl group or an optionally substituted 5-13 membered heteroaryl group, the optionally substituted 3-12 membered cycloheteroalkyl group and the optionally substituted 5-13 membered heteroaryl group are not a 5-6 membered or 11-12 membered nitrogen-containing monocyclic or bicyclic group connected to the thienopyridine ring via a nitrogen atom.

Compounds of the present teachings include, but are not limited to, the compounds presented in Table 1 below.

TABLE 1

| Compound | |
|---|---|
| number | Compound Name |
| 101 | 4-(1H-indol-5-ylamino)-2-[(4-morpholin-4-ylmethyl)phenyl]thieno[2,3-b]pyridine-5- |
| | carbonitrile |
| 102 | 4-(1H-indol-5-ylamino)-2-iodothieno[2,3-b] pyridine-5-carbonitrile hydrochloride |
| 103 | 2-(4-formylphenyl)-4-(1H-indol-5-ylamino)thieno[2,3-b]pyridine-5-carbonitrile |
| 104 | 4-(1H-indol-5-ylamino)-2-{4-[(4-methylpiperazin-1-yl)methyl]phenyl}thieno[2,3-b]pyridine-5- |
| | carbonitrile |
| 105 | 2-{4-[(dimethylamino)methyl]phenyl}-4-(1H-indol-5-ylamino)thieno[2,3-b]pyridine-5- |
| | carbonitrile |
| 106 | 2-(4-{[(2-hydroxyethyl)amino]methyl}phenyl)-4-(1H-indol-5-ylamino)thieno[2,3-b]pyridine- |
| | 5-carbonitrile |
| 107 | 4-(1H-indol-5-ylamino)-2-phenylthieno[2,3-b]pyridine-5-carbonitrile |
| 108 | 4-(1H-indol-7-ylamino)-2-iodothieno[2,3-b]pyridine-5-carbonitrile |
| 109 | 4-(1H-indol-7-ylamino)-2-phenylthieno[2,3-b]pyridine-5-carbonitrile |
| 110 | 2-(5-formyl-3-furyl)-4-(1H-indol-5-ylamino)thieno[2,3-b]pyridine-5-carbonitrile |
| 111 | 2-[4-(dimethylamino)phenyl]-4-(1H-indol-5-ylamino)thieno[2,3-b]pyridine-5-carbonitrile |
| 112 | 2-{3-[2-(dimethylamino)ethyl]phenyl}-4-(1H-indol-5-ylamino)thieno[2,3-b]pyridine-5- |
| | carbonitrile |
| 113 | 2-{4-[(dimethylamino)methyl]phenyl}-4-[(4-methyl-1H-indol-5-yl)amino]thieno[2,3- |
| | b]pyridine-5-carbonitrile |
| 114 | 2-{2-[(dimethylamino)methyl]phenyl}-4-(1H-indol-5-ylamino)thieno[2,3-b]pyridine-5- |
| | carbonitrile |
| 115 | 4-(1H-indol-4-ylamino)-2-phenylthieno[2,3-b] pyridine-5-carbonitrile |
| 116 | 4-(1H-indol-6-ylamino)-2-phenylthieno[2,3-b] pyridine-5-carbonitrile hydrochloride |
| 117 | 4-[(4-methyl-1H-indol-5-yl)amino]-2-phenylthieno[2,3-b]pyridine-5-carbonitrile |
| 118 | 4-[(2-methyl-1H-indol-5-yl)amino]-2-phenylthieno[2,3-b]pyridine-5-carbonitrile |
| 119 | 4-(1H-benzimidazol-5-ylamino)-2-phenylthieno[2,3-b]pyridine-5-carbonitrile |
| 120 | 4-[(7-methyl-1H-indol-5-yl)amino]-2-phenylthieno[2,3-b]pyridine-5-carbonitrile |
| 121 | 4-[(4-methyl-1H-indol-5-yl)amino]-2-{4-[(4-methylpiperazin-1-yl)methyl]phenyl}thieno[2,3- |
| | b]pyridine-5-carbonitrile |
| 122 | 4-[1H-indol-5-yl(methyl)amino]-2-phenylthieno[2,3-b]pyridine-5-carbonitrile |
| 123 | 2-iodo-4-[(4-methyl-1H-indol-5-yl)amino]thieno[2,3-b]pyridine-5-carbonitrile |
| 124 | 4-(1H-indol-5-ylmethylamino)-2-phenylthieno[2,3-b]pyridine-5-carbonitrile |
| 125 | 4-(1H-indol-4-ylmethylamino)-2-phenylthieno[2,3-b]pyridine-5-carbonitrile |
| 126 | 4-[(4-ethyl-1H-indol-5-yl)amino]-2-phenylthieno[2,3-b]pyridine-5-carbonitrile |
| 127 | 4-{[2-(1H-imidazol-4-yl)ethyl]amino}-2-phenylthieno[2,3-b]pyridine-5-carbonitrile |
| 128 | 4-{[2-(1H-imidazol-4-yl)ethyl]amino}-2-{4-[(4-methylpiperazin-1- |
| | yl)methyl]phenyl}thieno[2,3-b]pyridine-5-carbonitrile |
| 129 | N-(5-cyano-2-phenylthieno[2,3-b]pyridin-4-yl)-1H-indole-5-carboxamide |
| 130 | 4-(1H-indol-5-yloxy)-2-phenylthieno[2,3-b]pyridine-5-carbonitrile |
| 131 | 4-(1H-indol-5-yl)-2-phenylthieno[2,3-b]pyridine-5-carbonitrile |
| 133 | 4-[(3-methyl-1H-indol-5-yl)amino]-2-phenylthieno[2,3-b]pyridine-5-carbonitrile |
| 134 | 4-[(1-methyl-1H-indol-5-yl)amino]-2-phenylthieno[2,3-b]pyridine-5-carbonitrile |
| 135 | 4-(1H-indol-4-ylamino)-2-iodothieno[2,3-b]pyridine-5-carbonitrile hydrochloride |
| 136 | 2-(4-formylphenyl)-4-(1H-indol-4-ylamino)thieno[2,3-b]pyridine-5-carbonitrile |
| 137 | 4-(1H-indol-4-ylamino)-2-{4-[(4-methylpiperazin-1-yl)methyl]phenyl}thieno[2,3-b]pyridine-5- |
| | carbonitrile |
| 138 | 4-(1H-indol-4-ylamino)-2-[4-(morpholin-4-ylmethyl)phenyl]thieno[2,3-b]pyridine-5- |
| | carbonitrile |
| 139 | 2-(3-formylphenyl)-4-(1H-indol-4-ylamino)thieno[2,3-b]pyridine-5-carbonitrile |
| 140 | 4-(1H-indol-4-ylamino)-2-{3-[(4-methyl piperazin-1-yl)methyl]phenyl}thieno[2,3-b]pyridine- |
| | 5-carbonitrile |
| 141 | 2-{3-[(dimethylamino)methyl]phenyl}-4-(1H-indol-4-ylamino)thieno[2,3-b]pyridine-5- |
| | carbonitrile |
| 142 | 2-(2-formylphenyl)-4-(1H-indol-4-ylamino)thieno[2,3-b]pyridine-5-carbonitrile |
| 143 | 4-(1H-indol-4-ylamino)-2-{2-[(4-methylpiperazin-1-yl)methyl]phenyl}thieno[2,3-b]pyridine-5- |
| | carbonitrile |
| 144 | 2-(3-formylphenyl)-4-(1H-indol-5-ylamino)thieno[2,3-b]pyridine-5-carbonitrile |
| 145 | 4-(1H-indol-5-ylamino)-2-{3-[(4-methylpiperazin-1-yl)methyl]phenyl}thieno[2,3-b]pyridine-5- |
| | carbonitrile |
| 146 | 4-(1H-indol-5-ylamino)-2-[3-(morpholin-4-ylmethyl)phenyl]thieno[2,3-b]pyridine-5- |
| | carbonitrile |
| 147 | 2-{3-[(dimethylamino)methyl]phenyl}-4-(1H-indol-5-ylamino)thieno[2,3-b]pyridine-5- |
| | carbonitrile |
| 148 | 2-(3-{[4-(2-hydroxyethyl)piperazin-1-yl]methyl}phenyl)-4-(1H-indol-5-ylamino)thieno[2,3- |
| | b]pyridine-5-carbonitrile |
| 149 | 2-(3-{[(2-hydroxyethyl)amino]methyl}phenyl)-4-(1H-indol-5-ylamino)thieno[2,3-b]pyridine- |
| | 5-carbonitrile |
| 150 | 4-(1H-indol-5-ylamino)-2-(3-{[4-(2-morpholin-4-ylethyl)piperazin-1-yl]methyl} |
| | phenyl)thieno[2,3-b]pyridine-5-carbonitrile |
| 151 | 2-(3-{[bis(2-hydroxyethyl)amino]methyl}phenyl)-4-(1H-indol-5-ylamino)thieno[2,3- |
| | b]pyridine-5-carbonitrile |
| 152 | 4-(1H-indol-5-ylamino)-2-(3-{[4-(2-phenylethyl)piperazin-1-yl]methyl}phenyl)thieno[2,3- |
| | b]pyridine-5-carbonitrile |
| 153 | 2-{5-[(dimethylamino)methyl]-2-furyl}-4-(1H-indol-5-ylamino)thieno[2,3-b]pyridine-5- |
| | carbonitrile |
| 154 | 2-{5-[(dimethylamino)methyl]-3-furyl}-4-(1H-indol-5-ylamino)thieno[2,3-b]pyridine-5- |
| | carbonitrile |
| 155 | 2-{5-[(dimethylamino)methyl]-2-methoxyphenyl}-4-(1H-indol-5-ylamino)thieno[2,3- |
| | b]pyridine-5-carbonitrile |
| 156 | 2-{2-[(dimethylamino)methyl]phenyl}-4-[(4-methyl-1H-indol-5-yl)amino]thieno[2,3- |
| | b]pyridine-5-carbonitrile |
| 157 | 2-(5-{[(3S)-3-hydroxypyrrolidin-1-yl]methyl}-3-thienyl)-4-(1H-indol-5-ylamino)thieno[2,3- |
| | b]pyridine-5-carbonitrile |
| 158 | 2-(5-{[(2S)-2-(hydroxymethyl)pyrrolidin-1-yl]methyl}-3-thienyl)-4-(1H-indol-5-ylamino) |
| 159 | 4-(1H-indol-5-ylamino)-2-[3-(pyrrolidin-1-ylmethyl)phenyl]thieno[2,3-b]pyridine-5- |
| | carbonitrile |
| 160 | 4-(1H-indol-5-ylamino)-2-[3-(piperidin-1-ylmethyl)phenyl]thieno[2,3-b]pyridine-5-carbonitrile |
| 161 | 2-{3-[(diethylamino)methyl]phenyl}-4-(1H-indol-5-ylamino)thieno[2,3-b]pyridine-5- |
| | carbonitrile |
| 162 | 4-(1H-indol-5-ylamino)-2-(3-{[(2-methoxyethyl)(methyl)amino]methyl}phenyl)thieno[2,3- |
| | b]pyridine-5-carbonitrile |
| 163 | 2-[3-({4-[2-(dimethylamino)ethyl]piperazin-1-yl}methyl)phenyl]-4-(1H-indol-5- |
| | ylamino)thieno[2,3-b]pyridine-5-carbonitrile |
| 164 | 2-(3-{[(2-hydroxyethyl)(methyl)amino]methyl}phenyl)-4-(1H-indol-5-ylamino)thieno[2,3- |
| | b]pyridine-5-carbonitrile |
| 165 | 4-(1H-indol-5-ylamino)-2-(3-{[(2-methoxyethyl)amino]methyl}phenyl)thieno[2,3-b]pyridine- |
| | 5-carbonitrile |
| 166 | 2-[3-({[2-(dimethylamino)ethyl]amino}methyl)phenyl]-4-(1H-indol-5-ylamino)thieno[2,3- |
| | b]pyridine-5-carbonitrile |
| 167 | 2-(3-{[(3-hydroxypropyl)amino]methyl}phenyl)-4-(1H-indol-5-ylamino)thieno[2,3-b]pyridine- |
| | 5-carbonitrile |
| 168 | 4-(1H-indol-5-ylamino)-2-(3-{[4-(2-oxo-2-pyrrolidin-1-ylethyl)piperazin-1- |
| | yl]methyl}phenyl)thieno[2,3-b]pyridine-5-carbonitrile |
| 169 | 4-(1H-indol-5-ylamino)-2-(3-{[4-(pyridin-4-ylmethyl)piperazin-1- |
| | yl]methyl}phenyl)thieno[2,3-b]pyridine-5-carbonitrile |
| 170 | 4-(1H-indol-5-ylamino)-2-(3-{[(2-morpholin-4-ylethyl)amino]methyl}phenyl)thieno[2,3- |
| | b]pyridine-5-carbonitrile |
| 171 | 2-(3-{[(3S)-3-hydroxypyrrolidin-1-yl]methyl}phenyl)-4-(1H-indol-5-ylamino)thieno[2,3- |
| | b]pyridine-5-carbonitrile |
| 172 | 2-(3-{[(3R)-3-hydroxypyrrolidin-1-yl]methyl}phenyl)-4-(1H-indol-5-ylamino)thieno[2,3- |
| | b]pyridine-5-carbonitrile |
| 173 | 2-(3-{[3-(hydroxymethyl)piperidin-1-yl]methyl}phenyl)-4-(1H-indol-5-ylamino)thieno[2,3- |
| | b]pyridine-5-carbonitrile |
| 174 | 2-(3-{[4-(hydroxymethyl)piperidin-1-yl]methyl}phenyl)-4-(1H-indol-5-ylamino)thieno[2,3- |
| | b]pyridine-5-carbonitrile |
| 175 | 2-[3-({4-[2-(1H-imidazol-1-yl)ethyl]piperazin-1-yl}methyl)phenyl]-4-(1H-indol-5- |
| | ylamino)thieno[2,3-b]pyridine-5-carbonitrile |
| 176 | 2-{3-[(4-hydroxypiperidin-1-yl)methyl]phenyl}-4-(1H-indol-5-ylamino) thieno[2,3-b]pyridine- |
| | 5-carbonitrile |
| 177 | 2-(3-{[4-(2-hydroxyethyl)piperidin-1-yl]methyl}phenyl)-4-(1H-indol-5-ylamino)thieno[2,3- |
| | b]pyridine-5-carbonitrile |
| 178 | 4-(1H-indol-5-ylamino)-2-(3-{[4-(2-methoxyethyl)piperazin-1-yl]methyl} phenyl)thieno[2,3- |
| | b]pyridine-5-carbonitrile |
| 179 | 4-(1H-indol-5-ylamino)-2-(3-{[(tetrahydrofuran-2-ylmethyl)amino]methyl}phenyl) thieno[2,3- |
| | b] pyridine-5-carbonitrile |
| 180 | 4-(1H-indol-5-ylamino)-2-(3-{[(3-morpholin-4-ylpropyl)amino]methyl}phenyl)thieno[2,3- |
| | b]pyridine-5-carbonitrile |
| 181 | 2-[3-({4-[2-(2-hydroxyethoxy) ethyl]piperazin-1-yl}methyl)phenyl]-4-(1H-indol-5- |
| | ylamino)thieno[2,3-b]pyridine-5-carbonitrile |
| 182 | 2-(3-{[(2-(hydroxymethyl)piperidin-1-yl]methyl}phenyl)-4-(1H-indol-5-ylamino)thieno[2,3- |
| | b]pyridine-5-carbonitrile |
| 183 | 2-[3-({[2-(2-hydroxyethoxy)ethyl]amino}methyl)phenyl]-4-(1H-indol-5-ylamino)thieno[2,3- |
| | b]pyridine-5-carbonitrile |
| 184 | 2-(2-formylphenyl)-4-(1H-indol-5-ylamino)thieno[2,3-b]pyridine-5-carbonitrile |
| 185 | 4-(1H-indol-5-ylamino)-2-{2-[(4-methylpiperazin-1-yl)methyl]phenyl} thieno[2,3-b]pyridine- |
| | 5-carbonitrile |
| 186 | 2-{2-[(4-hydroxypiperidin-1-yl)methyl]phenyl}-4-(1H-indol-5-ylamino)thieno [2,3-b] |
| | pyridine-5-carbonitrile |
| 187 | 2-{3-[(dimethylamino)methyl]phenyl}-4-[(4-methyl-1H-indol-5-yl)amino]thieno[2,3- |
| | b]pyridine-5-carbonitrile |
| 188 | 2-[4-(aminomethyl)phenyl]-4-(1H-indol-5-ylamino)thieno[2,3-b]pyridine-5-carbonitrile |
| 189 | 4-(1H-indol-5-ylamino)-2-(4-morpholin-4-ylphenyl)thieno[2,3-b]pyridine-5-carbonitrile |
| 190 | 2-[(1E)-4-(4-ethylpiperazin-1-yl)but-1-en-1-yl]-4-(1H-indol-5-ylamino)thieno[2,3-b]pyridine- |
| | 5-carbonitrile |
| 191 | 2-(5-formyl-2-thienyl)-4-(1H-indol-5-ylamino)thieno[2,3-b]pyridine-5-carbonitrile |
| 192 | 2-{4-[2-(dimethylamino)ethyl]phenyl}-4-(1H-indol-5-ylamino)thieno[2,3-b] pyridine-5- |
| | carbonitrile |
| 193 | 2-[3-(hydroxymethyl)phenyl]-4-(1H-indol-4-ylamino)thieno[2,3-b]pyridine-5-carbonitrile |
| 194 | 3-[5-cyano-4-(1H-indol-4-ylamino)thieno[2,3-b]pyridine-2-yl]-N,N-dimethylbenzamide |
| 195 | 3-[5-cyano-4-(1H-indol-5-ylamino)thieno[2,3-b]pyridine-2-yl]-N,N-dimethylbenzamide |
| 196 | 2-[3-(aminomethyl)phenyl]-4-(1H-indol-5-ylamino)thieno[2,3-b]pyridine-5-carbonitrile |
| 197 | 2-[3-(dimethylamino)phenyl]-4-(1H-indol-5-ylamino)thieno[2,3-b]pyridine-5-carbonitrile |
| 198 | 4-[5-cyano-4-(1H-indol-5-ylamino)thieno[2,3-b]pyridin-2-yl]-N-methylbenzenesulfonamide |
| 199 | 2-(5-formyl-3-thienyl)-4-(1H-indol-5-ylamino)thieno[2,3-b]pyridine-5-carbonitrile |
| 200 | 2-(5-formyl-2-furyl)-4-(1H-indol-5-ylamino)thieno[2,3-b]pyridine-5-carbonitrile |
| 201 | 2-(3-formylphenyl)-4-[(4-methyl-1H-indol-5-yl)amino]thieno[2,3-b]pyridine-5-carbonitrile |
| 202 | 2-(5-formyl-2-methoxyphenyl)-4-(1H-indol-5-ylamino)thieno[2,3-b]pyridine-5-carbonitrile |
| 203 | 4-(1H-indol-5-ylamino)-2-{5-[(4-methylpiperazin-1-yl)methyl]pyridine-2-yl}thieno[2,3- |
| | b]pyridine-5-carbonitrile |
| 204 | 2-{5-[(dimethylamino)methyl]pyridin-2-yl}-4-(1H-indol-5-ylamino)thieno[2,3-b] pyridine-5- |
| | carbonitrile |
| 205 | 4-(1H-indol-5-ylamino)-2-(1-methyl-1H-imidazol-5-yl)thieno[2,3-b]pyridine-5-carbonitrile |
| 206 | 2-(2-formyl-1-methyl-1H-imidazol-5-yl)-4-(1H-indol-5-ylamino)thieno[2,3-b]pyridine-5- |
| | carbonitrile |
| 207 | 2-[5-(1,3-dioxolan-2-yl)-2-thienyl]-4-(1H-indol-5-ylamino)thieno[2,3-b]pyridine-5-carbonitrile |
| 208 | 2-{2-[(dimethylamino)methyl]-1,3-thiazol-4-yl}-4-(1H-indol-5-ylamino)thieno[2,3-b]pyridine- |
| | 5-carbonitrile |
| 209 | 6-[5-cyano-4-(1H-indol-5-ylamino)thieno[2,3-b]pyridin-2-yl]-N,N-dimethylpyridine-2- |
| | carboxamide |
| 210 | 4-(1H-indol-5-ylamino)-2-{1-[(4-methylpiperazin-1-yl)methyl]-1H-imidazol-5-yl}thieno[2,3- |
| | b]pyridine-5-carbonitrile |
| 211 | 2-{5-[(dimethylamino)methyl]-2-thienyl}-4-(1H-indol-5-ylamino)thieno[2,3-b]pyridine-5- |
| | carbonitrile |
| 212 | 2-{5-[(dimethylamino)methyl]-3-thienyl}-4-(1H-indol-5-ylamino)thieno[2,3-b]pyridine-5- |
| | carbonitrile |
| 213 | 4-(1H-indol-5-ylamino)-2-(pyridine-2-ylethynyl)thieno[2,3-b]pyridine-5-carbonitrile |
| 214 | 4-(1H-indol-5-ylamino)-2-(pyridin-3-ylethynyl)thieno[2,3-b]pyridine-5-carbonitrile |
| 215 | 4-(1H-indol-5-ylamino)-2-(phenylethynyl)thieno[2,3-b]pyridine-5-carbonitrile |
| 216 | 4-(1H-indol-5-ylamino)-2-({6-[(4-methylpiperazin-1-yl)methyl]pyridin-2- |
| | yl}ethynyl)thieno[2,3-b]pyridine-5-carbonitrile |
| 217 | 2-({6-[(dimethylamino)methyl]pyridin-2-yl}ethynyl)-4-(1H-indol-5-ylamino)thieno[2,3- |
| | b]pyridine-5-carbonitrile |
| 218 | 4-(1H-indol-4-ylamino)-2-(pyridin-3-ylethynyl)thieno[2,3-b]pyridine-5-carbonitrile |
| 219 | 2-({6-[(dimethylamino)methyl]pyridin-2-yl}ethynyl)-4-(1H-indol-4-ylamino)thieno[2,3- |
| | b]pyridine-5-carbonitrile |
| 220 | 2-({6-[(dimethylamino)methyl]pyridin-2-yl}ethynyl)-4-(4-methyl-1H-indol-5- |
| | ylamino)thieno[2,3-b]pyridine-5-carbonitrile |
| 221 | 4-(1H-indol-5-ylamino)-2-[4-(4-methylpiperazin-1-yl)but-1-yn-1-yl]thieno[2,3-b]pyridine-5- |
| | carbonitrile |
| 222 | 4-(1H-indol-4-ylamino)-2-[4-(4-methylpiperazin-1-yl)but-1-yn-1-yl]thieno[2,3-b]pyridine-5- |
| | carbonitrile |
| 223 | 2-[3-(dimethylamino)prop-1-yn-1-yl]-4-(1H-indol-5-ylamino)thieno[2,3-b]pyridine-5- |
| | carbonitrile |
| 224 | 4-(1H-indol-5-ylamino)-2-[(trimethylsilyl)ethynyl]thieno[2,3-b]pyridine-5-carbonitrile |
| 225 | 2-[3-(diethylamino)prop-1-yn-1-yl]-4-(1H-indol-5-ylamino)thieno[2,3-b]pyridine-5- |
| | carbonitrile |
| 226 | 4-(1H-indol-5-ylamino)-2-(pyridin-4-ylethynyl)thieno[2,3-b]pyridine-5-carbonitrile |
| 227 | 4-(1H-indol-5-ylamino)-2-(1H-pyrazol-4-ylethynyl)thieno[2,3-b]pyridine-5-carbonitrile |
| 228 | 2-[(2-aminopyrimidin-5-yl)ethynyl]-4-(1H-indol-5-ylamino)thieno[2,3-b]pyridine-5- |
| | carbonitrile |
| 229 | 2-({5-[(dimethylamino)methyl]pyridin-2-yl}ethynyl)-4-(1H-indol-5-ylamino) thieno[2,3- |
| | b]pyridine-5-carbonitrile |
| 230 | 4-{5-cyano-4-[(4-methyl-1H-indol-5-yl)amino]thieno[2,3-b]pyridin-2-yl}-N- |
| | methylbenzenesulfonamide |
| 231 | 4-[5-cyano-4-(1H-indol-5-ylamino)thieno[2,3-b]pyridin-2-yl]-N,N- |
| | dimethylbenzenesulfonamide |
| 232 | 4-[5-cyano-4-(1H-indol-5-ylamino)thieno[2,3-b]pyridin-2-yl]-N-(2- |
| | hydroxyethyl)benzenesulfonamide |
| 233 | 4-[5-cyano-4-(1H-indol-5-ylamino)thieno[2,3-b]pyridin-2-yl]-N- |
| | cyclohexylbenzenesulfonamide |
| 234 | 4-(1H-indol-5-ylamino)-2-[4-(methylsulfonyl)phenyl]thieno[2,3-b]pyridine-5-carbonitrile |
| 235 | N-{4-[5-cyano-4-(1H-indol-5-ylamino)thieno[2,3-b]pyridin-2-yl]phenyl}methanesulfonamide |
| 236 | 4-{5-cyano-4-[(4-methyl-1H-indol-5-yl)amino]thieno[2,3-b]pyridin-2-yl}-N,N- |
| | dimethylbenzenesulfonamide |
| 237 | 3-{5-cyano-4-[(4-methyl-1H-indol-5-yl)amino]thieno[2,3-b]pyridin-2-yl}-N,N- |
| | dimethylbenzenesulfonamide |
| 238 | 2-{5-cyano-4-[(4-methyl-1H-indol-5-yl)amino]thieno[2,3-b]pyridin-2-yl}-N,N- |
| | dimethylbenzenesulfonamide |
| 239 | 4-(1H-indol-5-ylamino)-2-[3-(methylsulfonyl)phenyl]thieno[2,3-b]pyridine-5-carbonitrile |
| 240 | 3-{5-cyano-4-[(4-methyl-1H-indol-5-yl)amino]thieno[2,3-b]pyridin-2-yl}benzenesulfonamide |
| 241 | 4-{5-cyano-4-[(4-methyl-1H-indol-5-yl)amino]thieno[2,3-b]pyridin-2-yl}benzenesulfonamide |
| 242 | 4-[5-cyano-4-(1H-indol-5-ylamino)thieno[2,3-b]pyridin-2-yl]benzenesulfonamide |
| 243 | 3-bromo-4-(1H-indol-5-ylamino)thieno[2,3-b]pyridine-5-carbonitrile hydrobromide |
| 244 | 4-(1H-indol-5-ylamino)-3-[4-(4-methyl piperazin-1-yl)but-1-yn-1-yl]thieno[2,3-b]pyridine-5- |
| | carbonitrile |
| 245 | methyl 5-cyano-4-[(4-methyl-1H-indol-5-yl)amino]thieno[2,3-b]pyridine-2-carboxylate |
| 246 | methyl 5-cyano-4-(1H-indol-5-ylamino)thieno[2,3-b]pyridine-2-carboxylate |
| 247 | 5-cyano-4-[(4-methyl-1H-indol-5-yl)amino]thieno[2,3-b]pyridine-2-carboxylic acid |
| 248 | 5-cyano-4-(1H-indol-5-ylamino)thieno[2,3-b]pyridine-2-carboxylic acid |
| 249 | 4-[(4-methyl-1H-indol-5-yl)amino]-2-(pyrrolidin-1-ylcarbonyl)thieno[2,3-b]pyridine-5- |
| | carbonitrile |
| 250 | 4-(1H-indol-5-ylamino)-2-(pyrrolidin-1-ylcarbonyl)thieno[2,3-b]pyridine-5-carbonitrile |
| 251 | 5-cyano-4-(1H-indol-5-ylamino)-N-pyridin-3-ylthieno[2,3-b]pyridine-2-carboxamide |
| 252 | 5-cyano-4-(1H-indol-5-ylamino)-N-pyridin-4-ylthieno[2,3-b]pyridine-2-carboxamide |
| 253 | 4-(1H-indol-5-ylamino)-2-[(4-methylpiperazin-1-yl)carbonyl]thieno[2,3-b]pyridine-5- |
| | carbonitrile |
| 254 | 5-cyano-N-(2-hydroxyethyl)-4-(1H-indol-5-ylamino)thieno[2,3-b]pyridine-2-carboxamide |
| 255 | 4-[(4-methyl-1H-indol-5-yl)amino]-2-(morpholin-4-ylcarbonyl)thieno[2,3-b]pyridine-5- |
| | carbonitrile |
| 256 | 4-[(4-methyl-1H-indol-5-yl)amino]-2-[(4-methylpiperazin-1-yl)carbonyl]thieno[2,3-b]pyridine- |
| | 5-carbonitrile |
| 257 | 5-cyano-4-[(4-methyl-1H-indol-5-yl)amino]-N-pyridin-4-ylthieno[2,3-b]pyridine-2- |
| | carboxamide |
| 258 | 5-cyano-4-[(4-methyl-1H-indol-5-yl)amino]-N-phenylthieno[2,3-b]pyridine-2-carboxamide |
| 259 | N-benzyl-5-cyano-4-[(4-methyl-1H-indol-5-yl)amino]thieno[2,3-b]pyridine-2-carboxamide |
| 260 | 5-cyano-4-[(4-methyl-1H-indol-5-yl)amino]-N-(2-phenylethyl)thieno[2,3-b]pyridine-2- |
| | carboxamide |
| 261 | 5-cyano-N,N-dimethyl-4-[(4-methyl-1H-indol-5-yl)amino]thieno[2,3-b]pyridine-2- |
| | carboxamide |
| 262 | 5-cyano-N-(2-methoxyethyl)-4-[(4-methyl-1H-indol-5-yl)amino]thieno[2,3-b]pyridine-2- |
| | carboxamide |
| 263 | 5-cyano-4-[(4-methyl-1H-indol-5-yl)amino]-N-pyridin-3-ylthieno[2,3-b]pyridine-2- |
| | carboxamide |
| 264 | 4-(1H-indol-4-ylamino)-2-(pyrrolidin-1-ylcarbonyl)thieno[2,3-b]pyridine-5-carbonitrile |
| 265 | 4-[(4-methyl-1H-indol-5-yl)amino]-2-(piperazin-1-ylcarbonyl)thieno[2,3-b]pyridine-5- |
| | carbonitrile |
| 266 | 5-cyano-4-[(4-methyl-1H-indol-5-yl)amino]-N-piperidin-4-ylthieno[2,3-b]pyridine-2- |
| | carboxamide |
| 267 | 4-(1H-indol-5-ylamino)-2-(pyrrolidin-1-ylmethyl)thieno[2,3-b]pyridine-5-carbonitrile |
| 268 | 2-(3,4-dihydroisoquinolin-2(1H)-ylmethyl)-4-(1H-indol-5-ylamino)thieno[2,3-b]pyridine-5- |
| | carbonitrile |
| 269 | 4-(1H-indol-5-ylamino)-2-[(4-phenylpiperazin-1-yl)methyl]thieno[2,3-b]pyridine-5- |
| | carbonitrile |
| 270 | 2-[(1E)-buta-1,3-dien-1-yl]-4-(1H-indol-5-ylamino)thieno[2,3-b]pyridine-5-carbonitrile |
| 271 | 2-butyl-4-(1H-indol-5-ylamino)thieno[2,3-b]pyridine-5-carbonitrile |
| 272 | 4-[(4-methyl-1H-indol-5-yl)amino]-2-[(1E)-4-(4-methylpiperazin-1-yl)but-1-en-1- |
| | yl]thieno[2,3-b]pyridine-5-carbonitrile |
| 273 | 4-(1H-indol-5-ylamino)-2-[(1E)-4-(4-methylpiperazin-1-yl)but-1-en-1-yl]thieno[2,3- |
| | b]pyridine-5-carbonitrile |
| 274 | 4-(1H-indol-5-ylamino)-2-[4-(4-methylpiperazin-1-yl)butyl]thieno[2,3-b]pyridine-5- |
| | carbonitrile |
| 275 | 4-[(4-methyl-1H-indol-5-yl)amino]-2-[(1E)-3-morpholin-4-ylprop-1-en-1-yl]thieno[2,3- |
| | b]pyridine-5-carbonitrile |
| 276 | 4-[(4-methyl-1H-indol-5-yl)amino]-2-[(1E)-3-pyrrolidin-1-ylprop-1-en-1-yl]thieno[2,3- |
| | b]pyridine-5-carbonitrile |
| 277 | 4-(1H-indol-5-ylamino)-2-[(1E)-3-(4-methylpiperazin-1-yl)prop-1-en-1-yl]thieno[2,3- |
| | b]pyridine-5-carbonitrile |
| 278 | 4-[(4-methyl-1H-indol-5-yl)amino]-2-[(1E)-3-(4-methylpiperazin-1-yl)prop-1-en-1- |
| | yl]thieno[2,3-b]pyridine-5-carbonitrile |
| 279 | 3-methyl-4-[(4-methyl-1H-indol-5-yl)amino]-2-[(1E)-3-(4-methylpiperazin-1-yl)prop-1-en-1- |
| | yl]thieno[2,3-b]pyridine-5-carbonitrile |
| 280 | ethyl (2E)-3-[5-cyano-4-(1H-indol-5-ylamino)thieno[2,3-b]pyridin-2-yl]acrylate |
| 281 | (2E)-3-[5-cyano-4-(1H-indol-5-ylamino)thieno[2,3-b]pyridin-2-yl]acrylic acid |
| 282 | ethyl (2E)-3-{5-cyano-4-[(4-methyl-1H-indol-5-yl)amino]thieno[2,3-b]pyridin-2-yl}acrylate |
| 283 | (2E)-3-{5-cyano-4-[(4-methyl-1H-indol-5-yl)amino]thieno[2,3-b]pyridin-2-yl}acrylic acid |
| 284 | ethyl 3-[5-cyano-4-(1H-indol-5-ylamino)thieno[2,3-b]pyridin-2-yl]propanoate |
| 285 | 3-[5-cyano-4-(1H-indol-5-ylamino)thieno[2,3-b]pyridin-2-yl]propanoic acid |
| 286 | tert-butyl (2E)-3-{5-cyano-4-[(4-methyl-1H-indol-5-yl)amino]thieno[2,3-b]pyridin-2- |
| | yl}acrylate |
| 287 | (2E)-3-{5-Cyano-3-methyl-4-[(4-methyl-1H-indol-5-yl)amino]thieno[2,3-b]pyridin-2- |
| | yl}acrylic acid |
| 288 | 4-(1H-indol-5-ylamino)-2-[(1E)-3-oxo-3-pyrrolidin-1-ylprop-1-en-1-yl]thieno[2,3-b]pyridine- |
| | 5-carbonitrile |
| 289 | (2E)-3-[5-cyano-4-(1H-indol-5-ylamino)thieno[2,3-b]pyridin-2-yl]-N-(2- |
| | hydroxyethyl)acrylamide |
| 290 | 4-(1H-indol-5-ylamino)-2-[(1E)-3-(4-methylpiperazin-1-yl)-3-oxoprop-1-en-1-yl]thieno[2,3- |
| | b]pyridine-5-carbonitrile |
| 291 | 4-(1H-indol-5-ylamino)-2-[(1E)-3-(2-methylpyrrolidin-1-yl)-3-oxoprop-1-en-1-yl]thieno[2,3- |
| | b]pyridine-5-carbonitrile |
| 292 | (2E)-3-[5-cyano-4-(1H-indol-5-ylamino)thieno[2,3-b]pyridin-2-yl]acrylamide |
| 293 | (2E)-3-[5-cyano-4-(1H-indol-5-ylamino)thieno[2,3-b]pyridin-2-yl]-N-pyridin-3-ylacrylamide |
| 294 | 4-[(4-methyl-1H-indol-5-yl)amino]-2-[(1E)-3-oxo-3-pyrrolidin-1-ylprop-1-en-1-yl]thieno[2,3- |
| | b]pyridine-5-carbonitrile |
| 295 | 4-[(4-methyl-1H-indol-5-yl)amino]-2-[(1E)-3-(4-methylpiperazin-1-yl)-3-oxoprop-1-en-1- |
| | yl]thieno[2,3-b]pyridine-5-carbonitrile |
| 296 | (2E)-3-{5-cyano-4-[(4-methyl-1H-indol-5-yl)amino]thieno[2,3-b]pyridin-2-yl}-N-pyridin-3- |
| | ylacrylamide |
| 297 | (2E)-3-{5-cyano-4-[(4-methyl-1H-indol-5-yl)amino]thieno[2,3-b]pyridin-2-yl}acrylamide |
| 298 | 4-[(4-methyl-1H-indol-5-yl)amino]-2-[(1E)-3-oxo-3-piperidin-1-ylprop-1-en-1-yl]thieno[2,3- |
| | b]pyridine-5-carbonitrile |
| 299 | 4-[(4-methyl-1H-indol-5-yl)amino]-2-{(1E)-3-oxo-3-[(2S)-2-(pyrrolidin-1-ylmethyl)pyrrolidin- |
| | 1-yl]prop-1-en-1-yl}thieno[2,3-b]pyridine-5-carbonitrile |
| 300 | 2-{(1E)-3-[(3R)-3-(dimethylamino)pyrrolidin-1-yl]-3-oxoprop-1-en-1-yl}-4-[(4-methyl-1H- |
| | indol-5-yl)amino]thieno[2,3-b]pyridine-5-carbonitrile |
| 301 | 2-{(1E)-3-[(3S)-3-(dimethylamino)pyrrolidin-1-yl]-3-oxoprop-1-en-1-yl}-4-[(4-methyl-1H- |
| | indol-5-yl)amino]thieno[2,3-b]pyridine-5-carbonitrile |
| 302 | 3-methyl-4-[(4-methyl-1H-indol-5-yl)amino]-2-[(1E)-3-oxo-3-pyrrolidin-1-ylprop-1-en-1- |
| | yl]thieno[2,3-b]pyridine-5-carbonitrile |
| 303 | (2E)-3-{5-cyano-4-[(4-methyl-1H-indol-5-yl)amino]thieno[2,3-b]pyridin-2-yl}-N- |
| | phenylacrylamide |
| 304 | (2E)-3-{5-cyano-4-[(4-methyl-1H-indol-5-yl)amino]thieno[2,3-b]pyridin-2-yl}-N-pyridin-4- |
| | ylacrylamide |
| 305 | (2E)-3-{5-cyano-4-[(4-methyl-1H-indol-5-yl)amino]thieno[2,3-b]pyridin-2-yl}-N,N- |
| | dimethylacrylamide |
| 306 | (2E)-3-{5-cyano-4-[(4-methyl-1H-indol-5-yl)amino]thieno[2,3-b]pyridin-2-yl}-N,N- |
| | diethylacrylamide |
| 307 | (2E)-3-{5-cyano-4-[(4-methyl-1H-indol-5-yl)amino]thieno[2,3-b]pyridin-2-yl}-N- |
| | ethylacrylamide |
| 308 | (2E)-3-{5-cyano-4-[(4-methyl-1H-indol-5-yl)amino]thieno[2,3-b]pyridin-2-yl}-N-(2- |
| | methoxyethyl)acrylamide |
| 309 | 4-[(4-methyl-1H-indol-5-yl)amino]-2-[(1E)-3-morpholin-4-yl-3-oxoprop-1-en-1-yl]thieno[2,3- |
| | b]pyridine-5-carbonitrile |
| 310 | 2-[(1E)-3-(3-hydroxypyrrolidin-1-yl)-3-oxoprop-1-en-1-yl]-4-[(4-methyl-1H-indol-5- |
| | yl)amino]thieno[2,3-b]pyridine-5-carbonitrile |
| 311 | 4-[(4-methyl-1H-indol-5-yl)amino]-2-[(1E)-3-oxo-3-piperazin-1-ylprop-1-en-1-yl]thieno[2,3- |
| | b]pyridine-5-carbonitrile |
| 312 | (2E)-3-{5-cyano-4-[(4-methyl-1H-indol-5-yl)amino]thieno[2,3-b]pyridin-2-yl}-N-[2- |
| | (dimethylamino)ethyl]-N-methylacrylamide |
| 313 | (2E)-3-{5-cyano-4-[(4-methyl-1H-indol-5-yl)amino]thieno[2,3-b]pyridin-2-yl}-N′,N′- |
| | dimethylacrylohydrazide |
| 314 | (2E)-3-{5-cyano-4-[(4-methyl-1H-indol-5-yl)amino]thieno[2,3-b]pyridin-2-yl}-N-pyrrolidin-1- |
| | ylacrylamide |
| 315 | 4-[(4-methyl-1H-indol-5-yl)amino]-2-(3-oxo-3-pyrrolidin-1-ylpropyl)thieno[2,3-b]pyridine-5- |
| | carbonitrile |
| 316 | 4-(1H-indol-5-ylamino)-2-[(E)-2-phenylvinyl]thieno[2,3-b]pyridine-5-carbonitrile |
| 317 | 4-(1H-Indol-5-ylamino)-2-iodo-3-methylthieno[2,3-b]pyridine-5-carbonitrile |
| 318 | 2-Iodo-3-methyl-4-[(4-methyl-1H-indol-5-yl)amino]thieno[2,3-b]pyridine-5-carbonitrile |
| 319 | 4-[(5-methyl-1H-indol-4-yl)amino]-2-phenylthieno[2,3-b]pyridine-5-carbonitrilee |
| 320 | 2-{3-[(dimethylamino)methyl]phenyl}-4-(1H-pyrrolo[2,3-b]pyridin-5-ylamino)thieno[2,3- |
| | b]pyridine-5-carbonitrile |
| 321 | 4-(1H-indol-5-ylamino)-2-iodo-3-isopropylthieno[2,3-b]pyridine-5-carbonitrile |
| 322 | 2-phenyl-4-(1H-pyrrolo[2,3-b]pyridin-5-ylamino)thieno[2,3-b]pyridine-5-carbonitrile |
| 323 | 4-[(6-methyl-1H-indol-5-yl)amino]-2-phenylthieno[2,3-b]pyridine-5-carbonitrile |
| 324 | 2-iodo-3-isopropyl-4-[(4-methyl-1H-indol-5-yl)amino]thieno[2,3-b]pyridine-5-carbonitrile |
| 325 | 3-bromo-4-[(4-methyl-1H-indol-5-yl)amino]thieno[2,3-b]pyridine-5-carbonitrile |
| 326 | 2-{3-[(dimethylamino)methyl]phenyl}-4-(pyridin-3-ylamino)thieno[2,3-b]pyridine-5- |
| | carbonitrile |
| 327 | 2-{3-[(dimethylamino)methyl]phenyl}-4-(pyridin-4-ylamino)thieno[2,3-b]pyridine-5- |
| | carbonitrile |
| 328 | 4-(1H-indol-5-ylamino)-2-(2-naphthyl)thieno[2,3-b]pyridine-5-carbonitrile |
| 329 | 4-[(4-methyl-1H-indol-5-yl)amino]-2-(6-morpholin-4-ylpyridin-3-yl)thieno[2,3-b]pyridine-5- |
| | carbonitrile |
| 330 | 4-[(4-methyl-1H-indol-5-yl)amino]-2-(2-morpholin-4-ylpyrimidin-5-yl)thieno[2,3-b]pyridine- |
| | 5-carbonitrile |
| 331 | 2-[2-(dimethylamino)pyrimidin-5-yl]-4-[(4-methyl-1H-indol-5-yl)amino]thieno[2,3-b]pyridine- |
| | 5-carbonitrile |
| 332 | 2-(2-ethoxyphenyl)-4-[(4-methyl-1H-indol-5-yl)amino]thieno[2,3-b]pyridine-5-carbonitrile |
| 333 | methyl (4-{5-cyano-4-[(4-methyl-1H-indol-5-yl)amino]thieno[2,3-b]pyridin-2- |
| | yl}phenyl)carbamate |
| 334 | N-butyl-N′-{4-[5-cyano-4-(1H-indol-5-ylamino)thieno[2,3-b]pyridin-2-yl]phenyl}urea |
| 335 | 3-[5-cyano-4-(1H-indol-5-ylamino)thieno[2,3-b]pyridin-2-yl]-N-[2- |
| | (dimethylamino)ethyl]benzamide |
| 336 | 2-(4-formyl-3-thienyl)-4-(1H-indol-5-ylamino)thieno[2,3-b]pyridine-5-carbonitrile |
| 337 | 2-(3-formyl-4-methoxyphenyl)-4-(1H-indol-5-ylamino)thieno[2,3-b]pyridine-5-carbonitrile |
| 338 | 2-(5-formyl-2-methoxyphenyl)-4-[(4-methyl-1H-indol-5-yl)amino]thieno[2,3-b]pyridine-5- |
| | carbonitrile |
| 339 | 3-{5-cyano-4-[(4-methyl-1H-indol-5-yl)amino]thieno[2,3-b]pyridin-2-yl}-N-[2- |
| | (dimethylamino)ethyl]benzamide |
| 340 | 2-(3-acetylphenyl)-4-[(4-methyl-1H-indol-5-yl)amino]thieno[2,3-b]pyridine-5-carbonitrile |
| 341 | 2-(5-formyl-1-benzothien-2-yl)-4-(1H-indol-5-ylamino)thieno[2,3-b]pyridine-5-carbonitrile |
| 342 | methyl {4-[5-cyano-4-(1H-indol-5-ylamino)thieno[2,3-b]pyridin-2-yl]phenyl}carbamate |
| 343 | 3-{5-cyano-4-[(4-methyl-1H-indol-5-yl)amino]thieno[2,3-b]pyridin-2-yl}-N- |
| | methylbenzenesulfonamide |
| 344 | 3-[5-cyano-4-(1H-indol-5-ylamino)thieno[2,3-b]pyridin-2-yl]-N-methoxy-N-methylbenzamide |
| 345 | 4-{5-cyano-4-[(4-methyl-1H-indol-5-yl)amino]thieno[2,3-b]pyridin-2-yl}-N-[2- |
| | (dimethylamino)ethyl]benzenesulfonamide |
| 346 | 4-{5-cyano-4-[(4-methyl-1H-indol-5-yl)amino]thieno[2,3-b]pyridin-2-yl}-N-[2- |
| | (dimethylamino)ethyl]-N-methylbenzenesulfonamide |
| 347 | 2-{3-[1-(dimethylamino)ethyl]phenyl}-4-[(4-methyl-1H-indol-5-yl)amino]thieno[2,3- |
| | b]pyridine-5-carbonitrile |
| 348 | 3-{5-cyano-4-[(4-methyl-1H-indol-5-yl)amino]thieno[2,3-b]pyridin-2-yl}-N-[2- |
| | (dimethylamino)ethyl]-N-methylbenzamide |
| 349 | 4-{5-cyano-4-[(4-methyl-1H-indol-5-yl)amino]thieno[2,3-b]pyridin-2-yl}-N-[2- |
| | (dimethylamino)ethyl]benzamide |
| 350 | 2-{6-[3-(dimethylamino)propoxy]pyridin-3-yl}-4-[(4-methyl-1H-indol-5-yl)amino]thieno[2,3- |
| | b]pyridine-5-carbonitrile |
| 351 | 2-[4-(2-chloroethoxy)phenyl]-4-[(4-methyl-1H-indol-5-yl)amino]thieno[2,3-b]pyridine-5- |
| | carbonitrile |
| 352 | 4-[(4-methyl-1H-indol-5-yl)amino]-2-{6-[(2-morpholin-4-ylethyl)amino]pyridin-3- |
| | yl}thieno[2,3-b]pyridine-5-carbonitrile |
| 353 | 3-{5-cyano-4-[(4-methyl-1H-indol-5-yl)amino]thieno[2,3-b]pyridin-2-yl}-N,N- |
| | dimethylbenzamide |
| 354 | 3-{5-cyano-4-[(4-methyl-1H-indol-5-yl)amino]thieno[2,3-b]pyridin-2-yl}-N-methoxy-N- |
| | methylbenzamide |
| 355 | 3-{5-cyano-4-[(4-methyl-1H-indol-5-yl)amino]thieno[2,3-b]pyridin-2-yl}-N- |
| | methoxybenzamide |
| 356 | 4-{5-cyano-4-[(4-methyl-1H-indol-5-yl)amino]thieno[2,3-b]pyridin-2-yl}-N,N- |
| | dimethylbenzamide |
| 357 | 4-{5-cyano-4-[(4-methyl-1H-indol-5-yl)amino]thieno[2,3-b]pyridin-2-yl}-N-methoxy-N- |
| | methylbenzamide |
| 358 | 4-{5-cyano-4-[(4-methyl-1H-indol-5-yl)amino]thieno[2,3-b]pyridin-2-yl}-N- |
| | methoxybenzamide |
| 359 | N-{3-[5-cyano-4-(1H-indol-5-ylamino)thieno[2,3-b]pyridin-2-yl]phenyl}methanesulfonamide |
| 360 | N-(3-{5-cyano-4-[(4-methyl-1H-indol-5-yl)amino]thieno[2,3-b]pyridin-2- |
| | yl}phenyl)methanesulfonamide |
| 361 | N-(4-{5-cyano-4-[(4-methyl-1H-indol-5-yl)amino]thieno[2,3-b]pyridin-2- |
| | yl}phenyl)methanesulfonamide |
| 362 | 4-[(4-methyl-1H-indol-5-yl)amino]-2-(2-naphthyl)thieno[2,3-b]pyridine-5-carbonitrile |
| 363 | 4-[(4-methyl-1H-indol-5-yl)amino]-2-(1-naphthyl)thieno[2,3-b]pyridine-5-carbonitrile |
| 364 | 2-(2-methoxyphenyl)-4-[(4-methyl-1H-indol-5-yl)amino]thieno[2,3-b]pyridine-5-carbonitrile |
| 365 | 2-(3-formyl-5-isopropoxyphenyl)-4-[(4-methyl-1H-indol-5-yl)amino]thieno[2,3-b]pyridine-5- |
| | carbonitrile |
| 366 | 2-(2-methoxy-5-methylphenyl)-4-[(4-methyl-1H-indol-5-yl)amino]thieno[2,3-b]pyridine-5- |
| | carbonitrile |
| 367 | 2-{5-[(dimethylamino)methyl]-2-ethoxyphenyl}-4-[(4-methyl-1H-indol-5-yl)amino]thieno[2,3- |
| | b]pyridine-5-carbonitrile |
| 368 | 2-{5-[(dimethylamino)methyl]-2-methylphenyl}-4-[(4-methyl-1H-indol-5- |
| | yl)amino]thieno[2,3-b]pyridine-5-carbonitrile |
| 369 | 3-{5-cyano-4-[(4-methyl-1H-indol-5-yl)amino]thieno[2,3-b]pyridin-2-yl}-4-methoxy-N,N- |
| | dimethylbenzamide |
| 370 | N-{2-[5-cyano-4-(1H-indol-5-ylamino)thieno[2,3-b]pyridin-2-yl]phenyl}methanesulfonamide |
| 371 | N-(2-{5-cyano-4-[(4-methyl-1H-indol-5-yl)amino]thieno[2,3-b]pyridin-2- |
| | yl}phenyl)methanesulfonamide |
| 372 | N-(4-{5-cyano-3-methyl-4-[(4-methyl-1H-indol-5-yl)amino]thieno[2,3-b]pyridin-2- |
| | yl}phenyl)methanesulfonamide |
| 373 | 2-(1-benzothien-2-yl)-4-[(4-methyl-1H-indol-5-yl)amino]thieno[2,3-b]pyridine-5-carbonitrile |
| 374 | 2-(5-formyl-1-benzothien-2-yl)-4-[(4-methyl-1H-indol-5-yl)amino]thieno[2,3-b]pyridine-5- |
| | carbonitrile |
| 375 | 4-[(4-methyl-1H-indol-5-yl)amino]-2-[3-(methylsulfonyl)phenyl]thieno[2,3-b]pyridine-5- |
| | carbonitrile |
| 376 | 4-[(4-methyl-1H-indol-5-yl)amino]-2-[4-(methylsulfonyl)phenyl]thieno[2,3-b]pyridine-5- |
| | carbonitrile |
| 377 | 2-(3-bromophenyl)-4-[(4-methyl-1H-indol-5-yl)amino]thieno[2,3-b]pyridine-5-carbonitrile |
| 378 | 2-(3-formyl-5-methylphenyl)-4-[(4-methyl-1H-indol-5-yl)amino]thieno[2,3-b]pyridine-5- |
| | carbonitrile |
| 379 | 2-(3-formyl-5-methyl-2-propoxyphenyl)-4-[(4-methyl-1H-indol-5-yl)amino]thieno[2,3- |
| | b]pyridine-5-carbonitrile |
| 380 | 4-[(4-methyl-1H-indol-5-yl)amino]-2-quinolin-3-ylthieno[2,3-b]pyridine-5-carbonitrile |
| 381 | 2-(2-butoxyphenyl)-4-[(4-methyl-1H-indol-5-yl)amino]thieno[2,3-b]pyridine-5-carbonitrile |
| 382 | 4-[(4-methyl-1H-indol-5-yl)amino]-2-(2-propoxyphenyl)thieno[2,3-b]pyridine-5-carbonitrile |
| 383 | 2-{2-[3-(dimethylamino)propoxy]phenyl}-4-[(4-methyl-1H-indol-5-yl)amino]thieno[2,3- |
| | b]pyridine-5-carbonitrile |
| 384 | 2-{5-[(dimethylamino)methyl]-2-propoxyphenyl}-4-[(4-methyl-1H-indol-5- |
| | yl)amino]thieno[2,3-b]pyridine-5-carbonitrile |
| 385 | 2-(6-ethoxy-2-naphthyl)-4-[(4-methyl-1H-indol-5-yl)amino]thieno[2,3-b]pyridine-5- |
| | carbonitrile |
| 386 | 2-(2-formylphenyl)-4-[(4-methyl-1H-indol-5-yl)amino]thieno[2,3-b]pyridine-5-carbonitrile |
| 387 | 2-(5-formylpyridin-3-yl)-4-[(4-methyl-1H-indol-5-yl)amino]thieno[2,3-b]pyridin-5- |
| | carbonitrile |
| 388 | 2-(2-fluorophenyl)-4-[(4-methyl-1H-indol-5-yl)amino]thieno[2,3-b]pyridine-5-carbonitrile |
| 389 | 2-(2-fluoro-5-formylphenyl)-4-[(4-methyl-1H-indol-5-yl)amino]thieno[2,3-b]pyridine-5- |
| | carbonitrile |
| 390 | 2-(3-{[2-(dimethylamino)ethyl](methyl)amino}phenyl)-4-[(4-methyl-1H-indol-5- |
| | yl)amino]thieno[2,3-b]pyridine-5-carbonitrile |
| 391 | 2-[6-(dimethylamino)pyridin-3-yl]-4-[(4-methyl-1H-indol-5-yl)amino]thieno[2,3-b]pyridine-5- |
| | carbonitrile |
| 392 | 2-[2-(methoxymethyl)phenyl]-4-[(4-methyl-1H-indol-5-yl)amino]thieno[2,3-b]pyridine-5- |
| | carbonitrile |
| 393 | 2-{3-[(dimethylamino)methyl]phenyl}-3-isopropyl-4-[(4-methyl-1H-indol-5- |
| | yl)amino]thieno[2,3-b]pyridine-5-carbonitrile |
| 394 | 2-(3-{[2-(dimethylamino)ethyl]amino}phenyl)-4-[(4-methyl-1H-indol-5-yl)amino]thieno[2,3- |
| | b]pyridine-5-carbonitrile |
| 395 | 4-[(4-methyl-1H-indol-5-yl)amino]-2-(2-piperidin-1-ylpyrimidin-5-yl)thieno[2,3-b]pyridine-5- |
| | carbonitrile |
| 396 | 4-[(4-methyl-1H-indol-5-yl)amino]-2-(2-pyrrolidin-1-ylpyrimidin-5-yl)thieno[2,3-b]pyridine- |
| | 5-carbonitrile |
| 397 | 4-[(4-methyl-1H-indol-5-yl)amino]-2-(6-piperidin-1-ylpyridin-3-yl)thieno[2,3-b]pyridine-5- |
| | carbonitrile |
| 398 | 2-[2-(hydroxymethyl)phenyl]-4-[(4-methyl-1H-indol-5-yl)amino]thieno[2,3-b]pyridine-5- |
| | carbonitrile |
| 399 | 2-{2-[2-(dimethylamino)ethoxy]phenyl}-4-[(4-methyl-1H-indol-5-yl)amino]thieno[2,3- |
| | b]pyridine-5-carbonitrile |
| 400 | 4-(1H-indol-5-ylamino)-2-[(E)-2-phenylvinyl]thieno[2,3-b]pyridine-5-carbonitrile |
| 401 | 2-[(E)-2-(4-fluorophenyl)vinyl]-4-(1H-indol-5-ylamino)thieno[2,3-b]pyridine-5-carbonitrile |
| 402 | 2-[(E)-2-(3-fluorophenyl)vinyl]-4-(1H-indol-5-ylamino)thieno[2,3-b]pyridine-5-carbonitrile |
| 403 | 2-[(1E)-4-hydroxybut-1-en-1-yl]-4-(1H-indol-5-ylamino)thieno[2,3-b]pyridine-5-carbonitrile |
| 404 | 4-(1H-indol-5-ylamino)-2-[(E)-2-(3-methoxyphenyl)vinyl]thieno[2,3-b]pyridine-5-carbonitrile |
| 405 | 4-(1H-indol-5-ylamino)-2-[(E)-2-(4-methoxyphenyl)vinyl]thieno[2,3-b]pyridine-5-carbonitrile |
| 406 | 4-(1H-indol-5-ylamino)-2-[(E)-2-(4-methylphenyl)vinyl]thieno[2,3-b]pyridine-5-carbonitrile |
| 407 | 2-[(E)-2-(4-chlorophenyl)vinyl]-4-(1H-indol-5-ylamino)thieno[2,3-b]pyridine-5-carbonitrile |
| 408 | 4-(1H-indol-5-ylamino)-2-{(E)-2-[4-(trifluoromethyl)phenyl]vinyl}thieno[2,3-b]pyridine-5- |
| | carbonitrile |
| 409 | 4-(1H-indol-5-ylamino)-2-[(1E)-3-phenylprop-1-en-1-yl]thieno[2,3-b]pyridine-5-carbonitrile |
| 410 | 4-(1H-indol-5-ylamino)-2-(1-phenylvinyl)thieno[2,3-b]pyridine-5-carbonitrile |
| 411 | 2-[(1E)-hex-1-en-1-yl]-4-(1H-indol-5-ylamino)thieno[2,3-b]pyridine-5-carbonitrile |
| 412 | 4-(1H-indol-5-ylamino)-2-[(1E)-3-methoxyprop-1-en-1-yl]thieno[2,3-b]pyridine-5-carbonitrile |
| 413 | 4-{5-cyano-3-methyl-4-[(4-methyl-1H-indol-5-yl)amino]thieno[2,3-b]pyridin-2- |
| | yl}benzenesulfonamide |
| 414 | 4-{5-cyano-3-methyl-4-[(4-methyl-1H-indol-5-yl)amino]thieno[2,3-b]pyridin-2-yl}-N- |
| | methylbenzenesulfonamide |
| 415 | 4-[5-cyano-4-(1H-indol-5-ylamino)-3-methylthieno[2,3-b]pyridin-2-yl]-N- |
| | methylbenzenesulfonamide |
| 416 | 4-[(4-methyl-1H-indol-5-yl)amino]-2-[(E)-2-phenylvinyl]thieno[2,3-b]pyridine-5-carbonitrile |
| 417 | 4-[(4-methyl-1H-indol-5-yl)amino]-2-vinylthieno[2,3-b]pyridine-5-carbonitrile |
| 418 | tert-butyl 4-{5-cyano-4-[(4-methyl-1H-indol-5-yl)amino]thieno[2,3-b]pyridin-2-yl}-5,6- |
| | dihydropyridine-1(2H)-carboxylate |
| 419 | tert-butyl 4-[5-cyano-4-(1H-indol-5-ylamino)thieno[2,3-b]pyridin-2-yl]-3,6-dihydropyridine- |
| | 1(2H)-carboxylate |
| 420 | 2-[(1E)-4-hydroxybut-1-en-1-yl]-4-[(4-methyl-1H-indol-5-yl)amino]thieno[2,3-b]pyridine-5- |
| | carbonitrile |
| 421 | 2-cyclohex-1-en-1-yl-4-[(4-methyl-1H-indol-5-yl)amino]thieno[2,3-b]pyridine-5-carbonitrile |
| 422 | 2-[(1E)-3-methoxyprop-1-en-1-yl]-4-[(4-methyl-1H-indol-5-yl)amino]thieno[2,3-b]pyridine-5- |
| | carbonitrile |
| 423 | 4-[(4-methyl-1H-indol-5-yl)amino]-2-[3-(pyrrolidin-1-ylcarbonyl)phenyl]thieno[2,3- |
| | b]pyridine-5-carbonitrile |
| 424 | 3-methyl-4-[(4-methyl-1H-indol-5-yl)amino]-2-phenylthieno[2,3-b]pyridine-5-carbonitrile |
| 425 | 3-methyl-4-[(4-methyl-1H-indol-5-yl)amino]-2-[(E)-2-phenylvinyl]thieno[2,3-b]pyridine-5- |
| | carbonitrile |
| 426 | N-(3-{5-cyano-3-methyl-4-[(4-methyl-1H-indol-5-yl)amino]thieno[2,3-b]pyridin-2- |
| | yl}phenyl)methanesulfonamide |
| 427 | 3-{5-cyano-3-methyl-4-[(4-methyl-1H-indol-5-yl)amino]thieno[2,3-b]pyridin-2- |
| | yl}benzenesulfonamide |
| 428 | 3-{5-cyano-3-methyl-4-[(4-methyl-1H-indol-5-yl)amino]thieno[2,3-b]pyridin-2-yl}-N- |
| | methylbenzenesulfonamide |
| 429 | 3-{5-cyano-3-methyl-4-[(4-methyl-1H-indol-5-yl)amino]thieno[2,3-b]pyridin-2-yl}-N-[2- |
| | (dimethylamino)ethyl]benzamide |
| 430 | 4-{5-cyano-3-methyl-4-[(4-methyl-1H-indol-5-yl)amino]thieno[2,3-b]pyridin-2-yl}-N-[2- |
| | (dimethylamino)ethyl]benzamide |
| 431 | 4-{5-cyano-3-methyl-4-[(4-methyl-1H-indol-5-yl)amino]thieno[2,3-b]pyridin-2-yl}-N-(2- |
| | hydroxyethyl)benzenesulfonamide |
| 432 | 2-(1-methyl-1H-imidazol-2-yl)-4-[(4-methyl-1H-indol-5-yl)amino]thieno[2,3-b]pyridine-5- |
| | carbonitrile |
| 433 | 4-(1H-indol-5-ylamino)-2-(1-methyl-1H-imidazol-2-yl)thieno[2,3-b]pyridine-5-carbonitrile |
| 434 | 2-(1H-indol-2-yl)-4-[(4-methyl-1H-indol-5-yl)amino]thieno[2,3-b]pyridine-5-carbonitrile |
| 435 | N-(2-{5-cyano-4-[(4-methyl-1H-indol-5-yl)amino]thieno[2,3-b]pyridin-2-yl}phenyl)acetamide |
| 436 | 2-(2-aminophenyl)-4-[(4-methyl-1H-indol-5-yl)amino]thieno[2,3-b]pyridine-5-carbonitrile |
| 437 | 2-(3-hydroxyphenyl)-4-[(4-methyl-1H-indol-5-yl)amino]thieno[2,3-b]pyridine-5-carbonitrile |
| 438 | 4-[(4-methyl-1H-indol-5-yl)amino]-2-pyridin-3-ylthieno[2,3-b]pyridine-5-carbonitrile |
| 439 | 4-[(4-methyl-1H-indol-5-yl)amino]-2-pyridin-4-ylthieno[2,3-b]pyridine-5-carbonitrile |
| 440 | 4-[(4-methyl-1H-indol-5-yl)amino]-2-pyridin-2-ylthieno[2,3-b]pyridine-5-carbonitrile |
| 441 | 2-(4-hydroxyphenyl)-4-[(4-methyl-1H-indol-5-yl)amino]thieno[2,3-b]pyridine-5-carbonitrile |
| 442 | 2-(2-hydroxyphenyl)-4-[(4-methyl-1H-indol-5-yl)amino]thieno[2,3-b]pyridine-5-carbonitrile |
| 443 | 2-{6-[(dimethylamino)methyl]pyridin-2-yl}-4-(1H-indol-5-ylamino)thieno[2,3-b]pyridine-5- |
| | carbonitrile |
| 444 | 2-({4-[(dimethylamino)methyl]pyridin-2-yl}ethynyl)-4-[(4-methyl-1H-indol-5- |
| | yl)amino]thieno[2,3-b]pyridine-5-carbonitrile |
| 445 | 2-{[6-(dimethylamino)pyridin-3-yl]ethynyl}-4-[(4-methyl-1H-indol-5-yl)amino]thieno[2,3- |
| | b]pyridine-5-carbonitrile |
| 446 | 4-[(4-methyl-1H-indol-5-yl)amino]-2-[(trimethylsilyl)ethynyl]thieno[2,3-b]pyridine-5- |
| | carbonitrile |
| 447 | 4-[(4-methyl-1H-indol-5-yl)amino]-2-({5-[(4-methylpiperazin-1-yl)methyl]-3- |
| | furyl}ethynyl)thieno[2,3-b]pyridine-5-carbonitrile |
| 448 | 2-({5-[(dimethylamino)methyl]-2-thienyl}ethynyl)-4-[(4-methyl-1H-indol-5- |
| | yl)amino]thieno[2,3-b]pyridine-5-carbonitrile |
| 449 | 2-({5-[(dimethylamino)methyl]-2-furyl}ethynyl)-4-[(4-methyl-1H-indol-5- |
| | yl)amino]thieno[2,3-b]pyridine-5-carbonitrile |
| 450 | 4-[(4-methyl-1H-indol-5-yl)amino]-2-[4-(4-methylpiperazin-1-yl)but-1-yn-1-yl]thieno[2,3- |
| | b]pyridine-5-carbonitrile |
| 451 | 5-({5-cyano-4-[(4-methyl-1H-indol-5-yl)amino]thieno[2,3-b]pyridin-2-yl}ethynyl)-N,N- |
| | dimethylnicotinamide |
| 452 | 2-({5-[(dimethylamino)methyl]pyridin-3-yl}ethynyl)-4-[(4-methyl-1H-indol-5- |
| | yl)amino]thieno[2,3-b]pyridine-5-carbonitrile |
| 453 | 2-({6-[(dimethylamino)methyl]pyridin-3-yl}ethynyl)-4-[(4-methyl-1H-indol-5- |
| | yl)amino]thieno[2,3-b]pyridine-5-carbonitrile |
| 454 | 2-{3-[(dimethylamino)methyl]-5-methylphenyl}-4-[(4-methyl-1H-indol-5- |
| | yl)amino]thieno[2,3-b]pyridine-5-carbonitrile |
| 455 | 2-{3-[(dimethylamino)methyl]-5-methyl-2-propoxyphenyl}-4-[(4-methyl-1H-indol-5- |
| | yl)amino]thieno[2,3-b]pyridine-5-carbonitrile |
| 456 | 2-{3-[(dimethylamino)methyl]-4-methoxyphenyl}-4-(1H-indol-5-ylamino)thieno[2,3- |
| | b]pyridine-5-carbonitrile |
| 457 | 2-{5-[(dimethylamino)methyl]-2-methoxyphenyl}-4-[(4-methyl-1H-indol-5- |
| | yl)amino]thieno[2,3-b]pyridine-5-carbonitrile |
| 458 | 2-{3-[(dimethylamino)methyl]phenyl}-3-methyl-4-[(4-methyl-1H-indol-5- |
| | yl)amino]thieno[2,3-b]pyridine-5-carbonitrile |
| 459 | 2-{5-[(dimethylamino)methyl]-2-methoxyphenyl}-3-methyl-4-[(4-methyl-1H-indol-5- |
| | yl)amino]thieno[2,3-b]pyridine-5-carbonitrile |
| 460 | 2-{4-[(dimethylamino)methyl]phenyl}-3-methyl-4-[(4-methyl-1H-indol-5- |
| | yl)amino]thieno[2,3-b]pyridine-5-carbonitrile |
| 461 | 2-{2-methoxy-5-[(4-methylpiperazin-1-yl)methyl]phenyl}-4-[(4-methyl-1H-indol-5- |
| | yl)amino]thieno[2,3-b]pyridine-5-carbonitrile |
| 462 | 2-{5-[(dimethylamino)methyl]pyridin-3-yl}-4-[(4-methyl-1H-indol-5-yl)amino]thieno[2,3- |
| | b]pyridine-5-carbonitrile |
| 463 | 2-{5-[(dimethylamino)methyl]-2-fluorophenyl}-4-[(4-methyl-1H-indol-5-yl)amino]thieno[2,3- |
| | b]pyridine-5-carbonitrile |
| 464 | 2-{4-[(dimethylamino)methyl]-3-thienyl}-4-(1H-indol-5-ylamino)thieno[2,3-b]pyridine-5- |
| | carbonitrile |
| 465 | 2-{5-[(dimethylamino)methyl]-1-benzothien-2-yl}-4-(1H-indol-5-ylamino)thieno[2,3- |
| | b]pyridine-5-carbonitrile |
| 466 | 2-{3-[(dimethylamino)methyl]-5-isopropoxyphenyl}-4-[(4-methyl-1H-indol-5- |
| | yl)amino]thieno[2,3-b]pyridine-5-carbonitrile |
| 467 | 2-{5-[(dimethylamino)methyl]-1-benzothien-2-yl}-4-[(4-methyl-1H-indol-5- |
| | yl)amino]thieno[2,3-b]pyridine-5-carbonitrile |
| 468 | 2-{5-[(dimethylamino)methyl]-3-thienyl}-4-[(4-methyl-1H-indol-5-yl)amino]thieno[2,3- |
| | b]pyridine-5-carbonitrile |
| 469 | 2-(2-{[(3-hydroxypropyl)amino]methyl}phenyl)-4-[(4-methyl-1H-indol-5-yl)amino]thieno[2,3- |
| | b]pyridine-5-carbonitrile |
| 470 | 2-(2-{[(2-hydroxyethyl)amino]methyl}phenyl)-4-[(4-methyl-1H-indol-5-yl)amino]thieno[2,3- |
| | b]pyridine-5-carbonitrile |
| 471 | 2-(3-{[(2-hydroxyethyl)amino]methyl}phenyl)-4-[(4-methyl-1H-indol-5-yl)amino]thieno[2,3- |
| | b]pyridine-5-carbonitrile |
| 472 | 3-methyl-4-[(4-methyl-1H-indol-5-yl)amino]-2-(1,2,3,6-tetrahydropyridin-4-yl)thieno[2,3- |
| | b]pyridine-5-carbonitrile |
| 473 | 4-(1H-indol-4-ylamino)-2-(1,2,3,6-tetrahydropyridin-4-yl)thieno[2,3-b]pyridine-5-carbonitrile |
| 474 | 4-[(4-methyl-1H-indol-5-yl)amino]-2-(1,2,3,6-tetrahydropyridin-4-yl)thieno[2,3-b]pyridine-5- |
| | carbonitrile |
| 475 | 4-[(4-methyl-1H-indol-5-yl)amino]-2-[1-(methylsulfonyl)-1,2,3,6-tetrahydropyridin-4- |
| | yl]thieno[2,3-b]pyridine-5-carbonitrile |
| 476 | 2-(1-benzyl-1,2,3,6-tetrahydropyridin-4-yl)-4-[(4-methyl-1H-indol-5-yl)amino]thieno[2,3- |
| | b]pyridine-5-carbonitrile |
| 477 | 4-[(4-Methyl-1H-indol-5-yl)amino]-2-(1-methyl-1,2,3,6-tetrahydropyridin-4-yl)thieno[2,3- |
| | b]pyridine-5-carbonitrile |
| 478 | 4-[(4-methyl-1H-indol-5-yl)amino]-2-piperidin-4-ylthieno[2,3-b]pyridine-5-carbonitrile |
| 479 | 2-(1-benzylpyrrolidin-3-yl)-4-[(4-methyl-1H-indol-5-yl)amino]thieno[2,3-b]pyridine-5- |
| | carbonitrile |
| 480 | 4-[(4-methyl-1H-indol-5-yl)amino]thieno[2,3-b]pyridine-5-carbonitrile |
| 481 | 3-Methyl-4-[(4-methyl-1H-indol-5-yl)amino]thieno[2,3-b]pyridine-5-carbonitrile |
| 482 | 2-(3-{[(2R)-2-hydroxypropyl]-oxy}phenyl)-4-[(4-methyl-1H-indol-5-yl)amino]thieno[2,3- |
| | b]pyridine-5-carbonitrile |
| 483 | 2-(3-{[(2S)-2-hydroxypropyl]-oxy}phenyl)-4-[(4-methyl-1H-indol-5-yl)amino]thieno[2,3- |
| | b]pyridine-5-carbonitrile |
| 484 | 2-(3-{[(2R)-2,3-Dihydroxypropyl]-oxy}phenyl)-4-[(4-methyl-1H-indol-5-yl)amino]thieno[2,3- |
| | b]pyridine-5-carbonitrile |
| 485 | 2-(3-{[(2S)-2,3-Dihydroxypropyl]-oxy}phenyl)-4-[(4-methyl-1H-indol-5-yl)amino]thieno[2,3- |
| | b]pyridine-5-carbonitrile |
| 486 | 2-(4-{[(2R)-2,3-dihydroxypropyl]-oxy}phenyl)-4-[(4-methyl-1H-indol-5-yl)amino]thieno[2,3- |
| | b]pyridine-5-carbonitrile |
| 487 | 2-(4-{[(2S)-2,3-dihydroxypropyl]-oxy}phenyl)-4-[(4-methyl-1H-indol-5-yl)amino]thieno[2,3- |
| | b]pyridine-5-carbonitrile |
| 488 | 2-(4-{[(2S)-2-hydroxypropyl]-oxy}phenyl)-4-[(4-methyl-1H-indol-5-yl)amino]thieno[2,3- |
| | b]pyridine-5-carbonitrile |
| 489 | 2-(4-{[(2R)-2-hydroxypropyl]-oxy}phenyl)-4-[(4-methyl-1H-indol-5-yl)amino]thieno[2,3- |
| | b]pyridine-5-carbonitrile |
| 490 | 2-(2-{[(2R)-2-hydroxypropyl]-oxy}phenyl)-4-[(4-methyl-1H-indol-5-yl)amino]thieno[2,3- |
| | b]pyridine-5-carbonitrile |
| 491 | 2-(2-{[(2S)-2-hydroxypropyl]-oxy}phenyl)-4-[(4-methyl-1H-indol-5-yl)amino]thieno[2,3- |
| | b]pyridine-5-carbonitrile |
| 492 | 2-(2-{[(2S)-2,3-dihydroxypropyl]-oxy}phenyl)-4-[(4-methyl-1H-indol-5-yl)amino]thieno[2,3- |
| | b]pyridine-5-carbonitrile |
| 493 | 2-(2-{[(2R)-2,3-dihydroxypropyl]-oxy}phenyl)-4-[(4-methyl-1H-indol-5-yl)amino]thieno[2,3- |
| | b]pyridine-5-carbonitrile |
| 494 | 2-{4-[2-(dimethylamino)ethoxy]phenyl}-4-[(4-methyl-1H-indol-5-yl)amino]thieno[2,3- |
| | b]pyridine-5-carbonitrile |
| 495 | 2-chloro-4-(1H-indol-5-ylamino)thieno[2,3-b]pyridine-5-carbonitrile |
| 496 | 3-(Hydroxymethyl)-4-[(4-methyl-1H-indol-5-yl)amino]thieno[2,3-b]pyridine-5-carbonitrile |
| 497 | 4-(4-methyl-1H-indol-5-ylamino)-3-((4-methylpiperazin-1-yl)methyl)thieno[2,3-b]pyridine-5- |
| | carbonitrile |
| 498 | 4-(4-chloro-1H-pyrrolo[2,3-b]pyridin-5-ylamino)-2-(3- |
| | ((dimethylamino)methyl)phenyl)thieno[2,3-b]pyridine-5-carbonitrile |
| 499 | 4-(1H-indol-5-ylamino)thieno[2,3-b]pyridine-5-carbonitrile-7-oxide |
| 500 | 4-(1H-indol-5-ylamino)-2-phenylthieno[2,3-b]pyridine-5-carbonitrile 7-oxide |
| 501 | 4-(4-Chloro-1H-pyrrolo[2,3-b]pyridin-5-ylamino)-2-iodothieno[2,3-b]pyridine-5-carbonitrile |
| 502 | ethyl 5-cyano-4-(1H-indol-5-ylamino)thieno[2,3-b]pyridine-2-carboxylate |
| 503 | 3-isopropyl-4-[(4-methyl-1H-indol-5-yl)amino]thieno[2,3-b]pyridine-5-carbonitrile |
| 504 | 4-[(4-methyl-1H-indol-5-yl)amino]-2-pyrazin-2-ylthieno[2,3-b]pyridine-5-carbonitrile |
| 505 | 2-(1H-indol-4-yl)-4-[(4-methyl-1H-indol-5-yl)amino]thieno[2,3-b]pyridine-5-carbonitrile |
| 506 | 4-[(4-methyl-1H-indol-5-yl)amino]-2-pyrimidin-5-ylthieno[2,3-b]pyridine-5-carbonitrile |
| 507 | 4-[(4-chloro-1H-pyrrolo[2,3-b]pyridin-5-yl)amino]-2-iodothieno[2,3-b]pyridine-5-carbonitrile |
| 508 | 3-{3-[(dimethylamino)methyl]phenyl}-4-[(4-methyl-1H-indol-5-yl)amino]thieno[2,3- |
| | b]pyridine-5-carbonitrile |
| 509 | N′-(3-{5-cyano-4-[(4-methyl-1H-indol-5-yl)amino]thieno[2,3-b]pyridin-2-yl}phenyl)-N,N- |
| | dimethylsulfamide |
| 510 | 3-{5-cyano-4-[(4-methyl-1H-indol-5-yl)amino]thieno[2,3-b]pyridin-2-yl}-N-(2- |
| | hydroxyethyl)benzenesulfonamide |
| 511 | 3-{5-cyano-4-[(4-methyl-1H-indol-5-yl)amino]thieno[2,3-b]pyridin-2-yl}-4-fluoro-N,N- |
| | dimethylbenzamide |
| 512 | 3-{5-cyano-4-[(4-methyl-1H-indol-5-yl)amino]thieno[2,3-b]pyridin-2-yl}-5-fluoro-N,N- |
| | dimethylbenzamide |
| 513 | 2-[3,4-bis(2-methoxyethoxy)phenyl]-4-[(4-methyl-1H-indol-5-yl)amino]thieno[2,3-b]pyridine- |
| | 5-carbonitrile |
| 514 | 2-(2-formyl-5-methoxyphenyl)-4-[(4-methyl-1H-indol-5-yl)amino]thieno[2,3-b]pyridine-5- |
| | carbonitrile |
| 515 | 2-{2-[(dimethylamino)methyl]-5-methoxyphenyl}-4-[(4-methyl-1H-indol-5- |
| | yl)amino]thieno[2,3-b]pyridine-5-carbonitrile |
| 516 | 2-{3-[(dimethylamino)methyl]phenyl}-4-(1H-indol-5-ylamino)-3-isopropylthieno[2,3- |
| | b]pyridine-5-carbonitrile |
| 517 | 2-(5-{[(2-hydroxyethyl)amino]methyl}-2-methoxyphenyl)-4-[(4-methyl-1H-indol-5- |
| | yl)amino]thieno[2,3-b]pyridine-5-carbonitrile |
| 518 | 5-{5-cyano-4-[(4-methyl-1H-indol-5-yl)amino]thieno[2,3-b]pyridin-2-yl}-2-fluoro-N,N- |
| | dimethylbenzamide |
| 519 | 2-[3-(1,1-dioxidoisothiazolidin-2-yl)phenyl]-4-[(4-methyl-1H-indol-5-yl)amino]thieno[2,3- |
| | b]pyridine-5-carbonitrile |
| 520 | 2-(1H-indol-5-yl)-4-[(4-methyl-1H-indol-5-yl)amino]thieno[2,3-b]pyridine-5-carbonitrile |
| 521 | 2-(1H-indol-6-yl)-4-[(4-methyl-1H-indol-5-yl)amino]thieno[2,3-b]pyridine-5-carbonitrile |
| 522 | 4-({4-chloro-1-[(4-methylphenyl)sulfonyl]-1H-pyrrolo[2,3-b]pyridin-5-yl}amino)-2- |
| | iodothieno[2,3-b]pyridine-5-carbonitrile |
| 523 | 4-[(4-methyl-1H-indol-5-yl)amino]-2-pyridazin-3-ylthieno[2,3-b]pyridine-5-carbonitrile |
| 524 | 4-[(4-chloro-1H-pyrrolo[2,3-b]pyridin-5-yl)amino]-2-phenylthieno[2,3-b]pyridine-5- |
| | carbonitrile |
| 525 | 2-(3-formyl-2-methoxyphenyl)-4-[(4-methyl-1H-indol-5-yl)amino]thieno[2,3-b]pyridine-5- |
| | carbonitrile |
| 526 | 2-{3-[(dimethylamino)methyl]-2-methoxyphenyl}-4-[(4-methyl-1H-indol-5- |
| | yl)amino]thieno[2,3-b]pyridine-5-carbonitrile |
| 527 | 4-[(4-methyl-1H-indol-5-yl)amino]-2-(1-methyl-1H-pyrazol-5-yl)thieno[2,3-b]pyridine-5- |
| | carbonitrile |
| 528 | 2-(5-{[bis(2-hydroxyethyl)amino]methyl}-2-methoxyphenyl)-4-[(4-methyl-1H-indol-5- |
| | yl)amino]thieno[2,3-b]pyridine-5-carbonitrile |
| 529 | 2-{3-[2-(dimethylamino)ethoxy]phenyl}-4-[(4-methyl-1H-indol-5-yl)amino]thieno[2,3- |
| | b]pyridine-5-carbonitrile |
| 530 | 4-[(4-chloro-1H-pyrrolo[2,3-b]pyridin-5-yl)amino]-2-{2-[2- |
| | (dimethylamino)ethoxy]phenyl}thieno[2,3-b]pyridine-5-carbonitrile |

Also provided in accordance with the present teachings are prodrugs of the disclosed herein.

The compounds of the present teachings can be useful for the treatment or inhibition of a pathological condition or disorder in a mammal. The present teachings accordingly include a method of providing to a mammal a pharmaceutical composition that comprises a compound of the present teachings in combination or association with a pharmaceutically acceptable carrier. The compound of the present teachings can be administered alone or in combination with other therapeutically effective compounds or therapies for the treatment or inhibition of the pathological condition or disorder.

The present teachings further include use of the compounds disclosed herein as active therapeutic substances for the treatment or inhibition of the pathological condition or disorder, for example, a condition mediated by a protein kinase such as protein kinase C (PKC) and its theta isoform (PKCθ), and for the alleviation of symptoms thereof. The pathological condition or disorder can include, but is not limited to, inflammatory diseases and autoimmune diseases such as asthma, psoriasis, arthritis, rheumatoid arthritis, osteoarthritis, joint inflammation, multiple sclerosis, diabetes including type II diabetes, and inflammatory bowel diseases (IBD) such as Crohn's disease and colitis.

Accordingly, the present teachings further provide methods of treating these pathological conditions and disorders using the compounds described herein. In some embodiments, the methods include identifying a mammal having a pathological condition or disorder mediated by a protein kinase such as PKC and PKCθ, and administering to the mammal a therapeutically effective amount of a compound as described herein.

Pharmaceutically acceptable salts of the compounds of formula I, which can have an acidic moiety, can be formed using organic and inorganic bases. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Internal salts also may be formed. Similarly, when a compound disclosed herein contains a basic moiety, salts can be formed using organic and inorganic acids. For example, salts can be formed from the following acids: acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, and camphorsulfonic as well as other known pharmaceutically acceptable acids.

The present teachings also include prodrugs of the compounds described herein. As used herein, “prodrug” refers to a moiety that produces, generates or releases a compound of the present teachings when administered to a mammalian subject. Prodrugs can be prepared by modifying functional groups present in the compounds in such a way that the modifications are cleaved, either by routine manipulation or in vivo, from the parent compounds. Examples of prodrugs include compounds described herein that contain one or more molecular moieties appended to a hydroxyl, amino, sulfhydryl, or carboxyl group of the compound, and that when administered to a mammalian subject, is cleaved in vivo to form the free hydroxyl, amino, sulfhydryl, or carboxyl group, respectively. Examples of prodrugs can include, but are not limited to, acetate, formate and benzoate derivatives of alcohol and amine functional groups in the compounds of the present teachings. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, the entire disclosure of which is incorporated by reference herein for all purposes.

The present teachings provide pharmaceutical compositions comprising at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington 's Pharmaceutical Sciences, 17th edition, ed. Alfonoso R. Gennaro, Mack Publishing Company, Easton, Pa. (1985), the entire disclosure of which is incorporated by reference herein for all purposes. Pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known antiinflammatory agents. Oral formulations containing an active compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided active ingredient. In tablets, an active compound can be mixed with a carrier having the necessary compression properties in suitable proportions and compacted in the shape and size desired. The powders and tablets can contain up to 99% of the active ingredient.

Capsules can contain mixtures of the active compound(s) with inert filler(s) and/or diluent(s) such as the pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents can include nonionic and anionic surface modifying agents. Examples of surface modifying agents can include, but are not limited to, poloxamer 188, benzalkonium chloride, calcium stearate, cetostearl alcohol, cetomacrogol emulsifying wax, sorbitan esters, colliodol silicon dioxide, phosphates, sodium dodecylsulfate, magnesium aluminum silicate, and triethanolamine. Oral formulations herein can utilize standard delay or time-release formulations to alter the absorption of the active compound(s). The oral formulation can also consist of administering an active compound in water or fruit juice, containing appropriate solubilizers or emulisifiers as needed.

Liquid carriers can be used in preparing solutions, suspensions, emulsions, syrups, and elixirs. An active compound disclosed herein can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Examples of liquid carriers for oral and parenteral administration include, but are not limited to, water (particularly containing additives as described above, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the active compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of active ingredient to about 500 mg/kg of active ingredient, and can be given in a single dose or in two or more doses. Such doses can be administered in any manner useful in directing the active compound(s) herein to the recipient's bloodstream, including orally, via implants, parenterally (including intravenous, intraperitoneal and subcutaneous injections), rectally, vaginally, and transdermally. Such administrations can be carried out using compounds of the present teachings including pharmaceutically acceptable salts thereof, in lotions, creams, foams, patches, suspensions, solutions, and suppositories (rectal and vaginal).

When administered for the treatment or inhibition of a particular disease state or disorder, it is understood that the effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. An amount adequate to accomplish this result is defined as a “therapeutically effective amount.” The dosage to be used in the treatment of a specific individual typically must be subjectively determined by the attending physician. The variables involved include the specific condition and its state as well as the size, age and response pattern of the patient.

In some cases, it may be desirable to administer a compound directly to the airways of the patient in the form of an aerosol. For administration by intranasal or intrabronchial inhalation, the compounds of the present teachings can be formulated into an aqueous or partially aqueous solution.

Compounds described herein can be administered parenterally or intraperitoneally. Solutions or suspensions of these active compounds or pharmaceutically acceptable salts thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations contain a preservative to inhibit the growth of microorganisms.

The pharmaceutical forms suitable for injection can include sterile aqueous solutions or dispersions and sterile powders for the extemporaneous preparation of sterile injectable solutions or dispersions. In preferred embodiments, the form is sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using compounds of the present teachings including pharmaceutically acceptable salts thereof, in lotions, creams, foams, patches, suspensions, solutions, and suppositories (rectal and vaginal). Topical formulations that deliver active compound(s) through the epidermis can be useful for localized treatment of inflammation and arthritis.

Transdermal administration can be accomplished through the use of a transdermal patch containing an active compound and a carrier that can be inert to the active compound, can be non-toxic to the skin, and can allow delivery of the active compound for systemic absorption into the blood stream via the skin. The carrier can take any number of forms such as creams and ointments, pastes, gels and occlusive devices. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable. A variety of occlusive devices can be used to release the active ingredient into the blood stream, such as a semi-permeable membrane covering a reservoir containing the active ingredient with or without a carrier, or a matrix containing the active ingredient. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of the compounds of the present teachings, it can be desirable to combine the compositions with other agents effective in the treatment of the target disease. For inflammatory diseases, other active compounds (e.g., other active ingredient or agents) effective in their treatment, and particularly in the treatment of asthma and arthritis, can be administered with the active compounds of the present teachings. The other agents can be administered at the same time or at different times than the compounds disclosed herein.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

As used herein, “halo” or “halogen” includes fluoro, chloro, bromo, and iodo.

As used herein, “oxo” refers to a double-bonded oxygen (i.e., ═O).

As used herein, the term “alkyl” refers to a straight-chain or branched saturated hydrocarbon group. Examples of alkyl groups include methyl (Me), ethyl (Et), propyl (e.g., n-propyl and isopropyl), butyl (e.g., n-butyl, isobutyl, s-butyl, t-butyl), pentyl groups (e.g., n-pentyl, isopentyl, neopentyl) and the like. In some embodiments, alkyl groups can be substituted with up to four independently selected R⁶, R¹¹, or R¹⁶ groups, where R⁶, R¹¹ and R¹⁶ are as described herein but typically exclude alkyl groups, alkenyl groups, and alkynyl groups. A lower alkyl group typically has up to 6 carbon atoms. Examples of lower alkyl groups include methyl, ethyl, propyl (e.g., n-propyl and isopropyl), and butyl groups (e.g., n-butyl, isobutyl, s-butyl, t-butyl).

As used herein, “alkenyl” refers to a straight-chain or branched alkyl group having one or more double carbon-carbon bonds. Examples of alkenyl groups include, but are not limited to, ethenyl, propenyl, butenyl, pentenyl, hexenyl, butadienyl, pentadienyl, hexadienyl groups, and the like. The one or more double carbon-carbon bonds can be internal (such as in 2-butene) or terminal (such as in 1-butene). In some embodiments, alkenyl groups can be substituted with up to four independently selected R⁶, R¹¹, or R¹⁶ groups, where R⁶, R¹¹ and R¹⁶ are as described herein but typically exclude alkyl groups, alkenyl groups, and alkynyl groups.

As used herein, “alkynyl” refers to a straight-chain or branched alkyl group having one or more triple carbon-carbon bonds. Examples of alkynyl groups include, but are not limited to, ethynyl, propynyl, butynyl, pentynyl, and the like. The one or more triple carbon-carbon bonds can be internal (such as in 2-butyne) or terminal (such as in 1-butyne). In some embodiments, alkynyl groups can be substituted with up to four independently selected R⁶, R¹¹, or R¹⁶ groups, where R⁶, R¹¹ and R¹⁶ are as described herein but typically exclude alkyl groups, alkenyl groups, and alkynyl groups.

As used herein, “alkoxy” refers to an —O-alkyl group. Examples of alkoxy groups include, but are not limited to, methoxy, ethoxy, propoxy (e.g., n-propoxy and isopropoxy), t-butoxy groups, and the like.

As used herein, “alkylthio” refers to an —S-alkyl group. Examples of alkylthio groups include, but are not limited to, methylthio, ethylthio, propylthio (e.g., n-propylthio and isopropylthio), t-butylthio groups, and the like.

As used herein, “haloalkyl” refers to an alkyl group having one or more halogen substituents. Examples of haloalkyl groups include, but are not limited to, CF₃, C₂F₅, CHF₂, CH₂F, CCl₃, CHCl₂, CH₂Cl, C₂Cl₅, and the like. Perhaloalkyl groups, i.e., alkyl groups wherein all of the hydrogen atoms are replaced with halogen atoms (e.g., CF₃ and C₂F₅), are included within the definition of “haloalkyl.”

As used herein, “cycloalkyl” refers to a non-aromatic carbocyclic group including cyclized alkyl, alkenyl, and alkynyl groups. A cycloalkyl group can be monocyclic (e.g., cyclohexyl) or polycyclic (e.g. containing fused, bridged, or spiro ring systems), wherein the carbon atoms are located inside or outside of the ring system. Any suitable ring position of the cycloalkyl moiety can be covalently linked to the defined chemical structure. Examples of cycloalkyl groups include, but are not limited to, cyclopropyl, cyclopropylmethyl, cyclobutyl, cyclopentyl, cyclohexyl, cyclohexylmethyl, cyclohexylethyl, cycloheptyl, cyclopentenyl, cyclohexenyl, cyclohexadienyl, cycloheptatrienyl, norbornyl, norpinyl, norcanyl, adamantyl, spiro[4.5]decanyl groups, as well as homologs, isomers, and the like. Also included in the definition of cycloalkyl groups are moieties that have one or more aromatic rings fused (i.e., having a bond in common with) to the cycloalkyl ring, for example, benzo derivatives of cyclopentane (i.e., an indanyl group), cyclohexane (i.e., a tetrahydronaphthyl group), and the like. In some embodiments, cycloalkyl groups can be substituted with up to four independently selected R⁶, R¹¹, or R¹⁶ groups, where R⁶, R¹¹ and R¹⁶ are as described herein. For example, a cycloalkyl group can include substitution of one or more oxo groups.

As used herein, “aryl” refers to an aromatic monocyclic or polycyclic hydrocarbon ring system such as, for example, phenyl, 1-naphthyl, 2-naphthyl, anthracenyl, phenanthrenyl groups, and the like. In some embodiments, a monocyclic aryl group can have from 6 to 14 carbon atoms and a polycyclic aryl group can have from 8 to 14 carbon atoms. Any suitable ring position of the aryl group can be covalently linked to the defined chemical structure. In some embodiments, aryl groups optionally contain up to four independently selected R⁶, R¹¹, or R¹⁶ groups, where R⁶, R¹¹ and R¹⁶ are as described herein.

As used herein, “heteroatom” refers to an atom of any element other than carbon or hydrogen and includes, for example, nitrogen, oxygen, sulfur, phosphorus, and selenium.

As used herein, “heteroaryl” refers to a monocyclic or polycyclic aromatic ring system having 5 to 13 ring atoms and containing 1-3 ring heteroatoms selected from oxygen (O), nitrogen (N) and sulfur (S). Generally, heteroaryl groups do not contain O—O, S—S, or S—O bonds. Heteroaryl groups include monocyclic heteroaryl rings fused to a phenyl ring. The heteroaryl group can be attached to the defined chemical structure at any heteroatom or carbon atom that results in a stable structure. Examples of heteroaryl groups can include, for example: 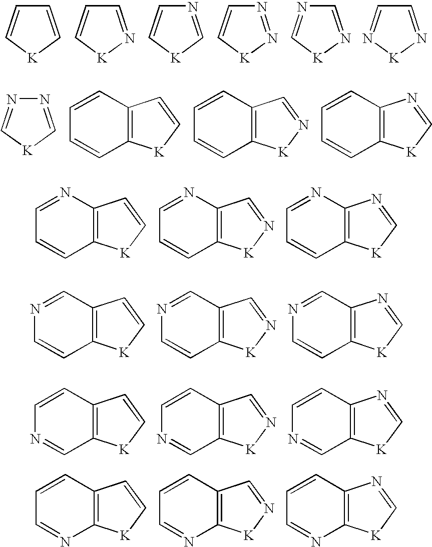
wherein K is defined as O, S, NH, NR , NR⁶, or NR¹¹, where R¹⁶, R¹¹, and R¹⁶ are described herein. One or more N or S atoms in a heteroaryl ring can be oxidized (e.g., pyridine N-oxide, thiophene S-oxide, thiophene S,S-dioxide). Examples of heteroaryl rings include, but are not limited to, pyrrole, furan, thiophene, pyridine, pyrimidine, pyridazine, pyrazine, triazole, pyrazole, imidazole, isothiazole, thiazole, thiadiazole, isoxazole, oxazole, oxadiazole, indole, isoindole, benzofuran, benzothiophene, quinoline, 2-methylquinoline, isoquinoline, quinoxaline, quinazoline, benzotriazole, benztetrazole, indazole, benzimidazole, benzothiazole, benzisothiazole, benzisoxazole, benzoxadiazole, benzoxazole, cinnoline, 1H-indazole, 2H-indazole, indolizin, isobenzofuran, naphthyridine, phthalazine, pteridine, purine, oxazolopyridine, thiazolopyridine, imidazopyridine, furopyridine, thienopyridine, pyridopyrimidine, pyridopyrazine, pyridopyridazine, thienothiazole, thienoxazole, and thienoimidazole. In some embodiments, heteroaryl groups can be substituted with up to four independently selected R⁶, R¹¹, or R¹⁶ groups, where R⁶, R¹¹, and R¹⁶ are as described herein.

As used herein, “cycloheteroalkyl” refers to a non-aromatic cycloalkyl group having 3 to 12 ring atoms, among which 1 to 3 ring atoms are heteroatoms selected from oxygen (O), nitrogen (N) and sulfur (S), and optionally containing one or more, e.g., two, double or triple bonds. One or more N or S atoms in a cycloheteroalkyl ring can be oxidized (e.g., morpholine N-oxide, thiomorpholine S-oxide, thiomorpholine S,S-dioxide). Examples of cycloheteroalkyl groups include, but are not limited to, morpholine, thiomorpholine, pyran, imidazolidine, imidazoline, oxazolidine, pyrazolidine, pyrazoline, pyrrolidine, pyrroline, tetrahydrofuran, tetrahydrothiophene, piperidine, piperazine, and the like. In some embodiments, cycloheteroalkyl groups can be optionally substituted with up to four independently selected R⁶, R¹¹, or R¹⁶ groups, where R⁶, R¹¹, and R¹⁶ are as described herein. In some embodiments, nitrogen atoms of cycloheteroalkyl groups can bear a substituent, for example an R⁶, R¹¹, or R¹⁶ group, where R⁶, R¹¹, and R¹⁶ are as described herein. Also included in the definition of cycloheteroalkyl are moieties that have one or more aromatic rings fused (i.e., have a bond in common with) to the cycloheteroalkyl group, for example, benzimidazoline, chromane, chromene, indolinetetrahydroquinoline, and the like. Cycloheteroalkyl groups can also contain one or more oxo groups, such as phthalimide, piperidone, oxazolidinone, pyrimidine-2,4(1H,3H)-dione, pyridin-2(1H)-one, and the like.

When one or more nitrogen atoms in a heteroaryl or cycloheteroalkyl group are oxidized, the bond between the nitrogen atom and the oxygen atom can be illustrated herein as a “dative” (or “coordinate covalence”) bond. In such depictions, the arrow represents a two-electron bond in which the two electrons are considered as belonging to the atom to which the arrow points, i.e., the oxygen atom. It is understood that the nitrogen atom will have the correct valence when oxidized. For example, when a trivalent nitrogen atom is oxidized, the resulting structure, in relevant part, can be alternatively illustrated as:

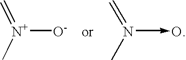

Compounds of the present teachings can include a “divalent group” defined herein as a linking group capable of forming a covalent bond with two other moieties. For example, compounds of the present teachings can include a divalent C₁₋₁₀ alkyl group, such as, for example, a methylene group.

At various places in the present specification substituents of compounds of the invention are disclosed in groups or in ranges. It is specifically intended that the description include each and every individual subcombination of the members of such groups and ranges. For example, the term “C₁₋₁₀ alkyl” is specifically intended to individually disclose C₁, C₂, C₃, C₄, C₅, C₆, C₇, C₈, C₉, C₁₀, C₁-C₁₀, C₁-C₉, C₁-C₈, C₁-C₇, C₁-C₆, C₁-C₅, C₁-C₄, C₁-C₃, C₁-C₂, C₂-C₁₀, C₂-C₉, C₂-C₈, C₂-C₇, C₂-C₆, C₂-C₅, C₂-C₄, C₂-C₃, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉, and C₉-C₁₀ alkyl. By way of another example, the term “5-13 membered heteroaryl group” is specifically intended to individually disclose a heteroaryl group having 5, 6, 7, 8, 9, 10, 11, 12, 13, 5-13, 5-12, 5-11, 5-10, 5-9, 5-8, 5-7, 5-6, 6-13, 6-12, 6-11, 6-10, 6-9, 6-8, 6-7, 7-13, 7-12, 7-11, 7-10, 7-9, 7-8, 8-13, 8-12, 8-11, 8-10, 8-9, 9-13, 9-12, 9-11, 9-10, 10-13, 10-12, 10-11, 11-13, 11-12, and 12-13 ring atoms.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such optical isomers (enantiomers) and diastereomers (geometric isomers), as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

The compounds of the present teachings can be conveniently prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatograpy (HPLC) or thin layer chromatography.

Preparation of compounds can involve the protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, the entire disclosure of which is incorporated by reference herein for all purposes.

The reactions of the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

Scheme 1 below depicts two exemplary synthetic routes for the preparation of compounds of formula I.

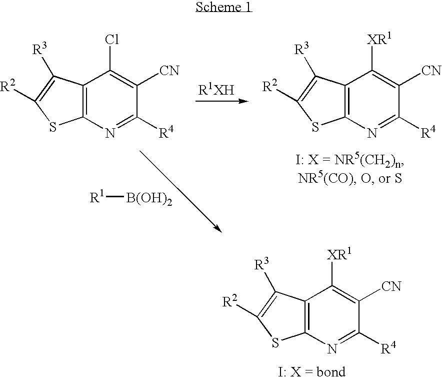

Generally, treatment of a 4-chlorothieno[2,3-b]pyridine-5-carbonitrile with a reagent of formula R¹XH, where X is an amine, amide, —O— or —S— linker group, provides compounds of formula I where R¹, R², R³, and R⁴ are as defined hereinabove.

There are several methods for adding an amine of formula R¹(CH₂)ₙNHR⁵ to a 4-chlorothieno[2,3-b]pyridine-5-carbonitrile. For instance, when n is 0, i.e., the amine has the formula R¹NHR⁵, one option is to add the amine to the 4-chlorothieno[2,3-b]pyridine-5-carbonitrile in a solvent such as ethanol, 2-propanol or 2-ethoxyethanol, optionally in the presence of pyridine hydrochloride, at elevated temperatures of 60-130° C. Other reaction conditions include the use of sodium hydride in a solvent such as tetrahydrofuran (THF) or dimethylformamide (DMF) at elevated temperatures of 60-70° C., or the use of a palladium catalyst such as tris(dibenzylideneacetone)dipalladium in the presence of potassium phosphate and a ligand such as 2-dicyclohexylphosphino-2′-(N, N-dimethylamino)biphenyl, in a solvent such as dimethoxyethane (DME). In other instances, such as when n is 1-4, i.e., the amine has the formula R¹(CH₂)₁₋₄NHR⁵, the addition reaction can be conducted in a solvent such as DMF in the presence of a base, such as sodium hydride, or in a solvent such as 2-ethoxyethanol in the presence of a base such as triethylamine or diisopropylethylamine, to provide compounds of formula I where X is NR⁵(CH₂)ₙ.

Addition of an amide of formula R¹(CO)NHR⁵ to a 4-chlorothieno[2,3-b]pyridine-5-carbonitrile in a solvent such as DMF in the presence of a base such as sodium hydride provides compounds of formula I where X is NR⁵(CO).

Addition of a compound of formula R¹OH to a 4-chlorothieno[2,3-b]pyridine-5-carbonitrile in a solvent such as acetonitrile at elevated temperature, preferably 80° C., in the presence of a base such as potassium carbonate, provides compounds of formula I where X is O.

Addition of a boronic acid of formula R¹—B(OH)₂ to a 4-chlorothieno[2,3-b]pyridine-5-carbonitrile in a solvent such as a mixture of DME and aqueous sodium bicarbonate in the presence of a palladium catalyst, such as (Ph₃P)₄Pd, provides compounds of formula I where X is a covalent bond.

A key intermediate for preparing compounds of formula I is a 4-chlorothieno[2,3-b]pyridine-5-carbonitrile where C2 or C3 is substituted with a leaving group such as a halide. Scheme 2 below depicts several possible routes for the preparation of this family of intermediates.

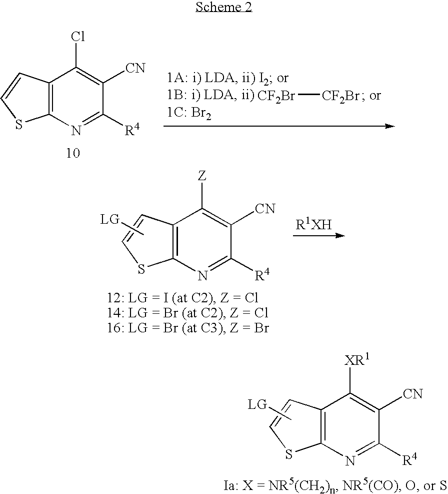

4-Chlorothieno[2,3-b]pyridine-5-carbonitrile 10 may be obtained according to any procedure known to those skilled in the art (see e.g., Khan, M. A. et al. (1977), J. Heterocyclic Chem., 14: 807-812; Boschelli, D. H. et al. (2004), J. Med. Chem., 47: 6666-6668). Treatment of 4-chlorothieno[2,3-b]pyridine-5-carbonitrile 10 with a base, preferentially lithium diisopropylamine (LDA), in an inert solvent such as THF at reduced temperature, preferably −78° C., followed by the addition of iodine provides the key intermediate 4-chloro-2-iodothieno[2,3-b]pyridine-5-carbonitrile 12. Alternatively, treatment of 4-chlorothieno[2,3-b]pyridine-5-carbonitrile 10 with a base, preferentially LDA, in an inert solvent such as THF at reduced temperature, preferably −78° C., followed by the addition of 1,2-dibromo-1,1,2,2,-tetrafluoroethane provides the key intermediate 2-bromo-4-chlorothieno[2,3-b]pyridine-5-carbonitrile 14. Furthermore, treatment of 4-chlorothieno[2,3-b]pyridine-5-carbonitrile 10 with bromine in acetic acid at elevated temperatures provides the key intermediate 3,4-dibromothieno[2,3-b]pyridine-5-carbonitrile 16. Addition of a compound of formula R¹XH to intermediates 12 and 14, under the conditions referred to earlier, provides compounds of formula Ia where R² is I or Br and R³ is H. Addition of a compound of formula R¹XH to intermediate 16, under the conditions referred to earlier, provides compounds of formula Ia where R² is H and R³ is Br.

Scheme 3 below depicts the preparation of additional compounds of the invention of formula I where R² (or R³) is an alkenyl, alkynyl, heteroaryl or aryl group beginning with compounds having the formula Ia described above. It should be understood that in Schemes 3-17 and the descriptions thereof, R² is in some cases used interchangeably with R³, to illustrate that various substituents can be added at either C2 or C3 of the thieno[2,3-b]pyridine-5-carbonitrile by using the same synthetic routes.

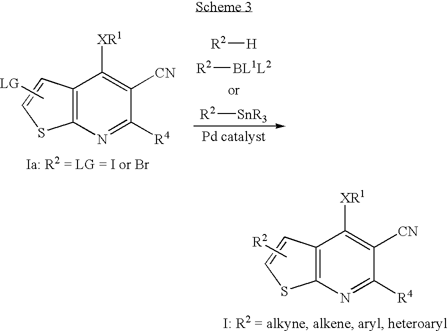

Treatment of compounds of formula Ia, where LG is either I or Br, with an alkene or alkyne of formula R²—H in the presence of a palladium catalyst provides compounds of formula I where R² (or R³) is either an alkenyl or alkynyl group. This alkenyl or alkynyl group can be substituted, for example, by aryl and heteroaryl groups and also by alkyl and alkyl amino groups among others. The aryl or heteroaryl group itself can also be substituted, for example, by alkoxy, alkylamino groups and others.

For the addition of alkenes of formula R²—H, the preferred palladium catalyst is palladium acetate in the presence of a ligand, preferably tri-o-tolylphosphine, in a solvent system that includes triethylamine or preferably a mixture of triethylamine and DMF.

For the addition of alkynes of formula R²—H, the preferred palladium catalyst is tetrakis(triphenylphosphine)palladium (0) along with a catalytic amount of copper(I)iodide in a solvent mixture that includes triethylamine and dioxane. If the alkynyl group is substituted by an alkyl amine, then the preferred palladium catalyst is dichlorobis(triphenylphosphine)palladium (II) and the reaction is performed in the presence of potassium carbonate along with catalytic amounts of both copper(I)iodide and triphenylphosphine in a solvent mixture that includes triethylamine and dioxane.

Treatment of compounds of formula Ia, where LG is either I or Br, with an aryl, heteroaryl or alkenyl organoboron compound of formula R²—BL¹L² in the presence of a palladium catalyst provides compounds of formula I where R² (or R³) is either an aryl, heteroaryl or alkenyl group. In compounds of formula R²—BL¹L², the L¹L² group represents ligands and includes such groups as lower alkoxy or preferably hydroxyl groups. The aryl, heteroaryl or alkenyl group of compound R²—BL¹L² can be substituted by groups including aryl, heteroaryl, formyl, carboxylate, carboxamide, alkyl, hydroxyalkyl and alkylamino groups among others. The aryl or heteroaryl group of compound R²—BL¹L² can also be fused to a second aryl or heteroaryl group.

For the addition of compounds of formula R²—BL¹L² the preferred palladium catalyst is tetrakis(triphenylphosphine)palladium (0) in a solvent mixture that includes saturated aqueous sodium bicarbonate and DME.

Compounds of formula I, where R² (or R³) is either an aryl group or a heteroaryl group, can also be prepared by reacting a compound of formula Ia, where LG is either I or Br, with an aryl or heteroaryl stannane compound of formula R²—SnR₃ in the presence of a palladium catalyst.

In compounds of formula R²—SnR₃, the R group is a lower alkyl group such as a butyl group or a methyl group. The aryl or heteroaryl group of compound R²—SnR₃ can be substituted, for example, by aryl, heteroaryl, formyl, acetal, carboxylate, carboxamide, alkyl and alkylamino groups among others. The aryl or heteroaryl group of compound R²—SnR₃ can also be fused to a second aryl or heteroaryl group. For the addition of compounds of formula R²—SnR₃, the preferred palladium catalyst is dichlorobis(triphenylphosphine)palladium (II) in a solvent such as dioxane.

Additional compounds of formula I, where R² (or R³) is an alkynyl group and X, R¹ and R⁴ are as defined hereinabove, can be prepared by the route shown in Scheme 4 below.

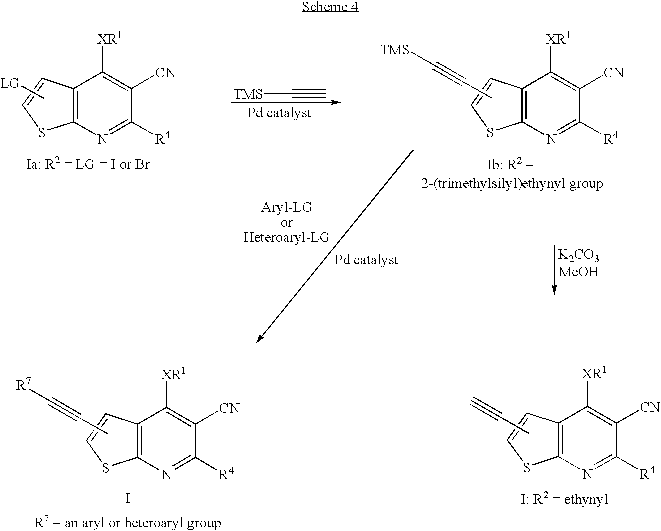

Treatment of a compound of formula Ia, where LG is either Br or I, with, for example, (trimethylsilyl)acetylene in the presence of a palladium catalyst, preferably tetrakis(triphenylphosphine)palladium(0), with a catalytic amount of copper(I) iodide in a solvent system such as triethylamine and dioxane, provides compounds of formula Ib where R² is a 2-(trimethylsilyl)ethynyl group. Reaction of compounds of formula Ib with aryliodides, arylbromides or heteroaryliodides or heteroarylbromides in the presence of a palladium catalyst, preferably dichlorobis(triphenylphosphine)palladium (II), in the presence of triphenylphosphine, potassium carbonate and copper(I) iodide, in a solvent mixture of THF and methanol (MeOH), provides compounds of formula I where R² is a 2-(aryl)ethynyl or a 2-(heteroaryl)ethynyl group. In addition, the 2-(trimethylsilyl)ethynyl group can be cleaved by treatment with potassium carbonate in MeOH to provide compounds of formula I, where R² is an ethynyl group.

Further compounds of formula I, where X, R¹ and R⁴ are as defined hereinabove and R² (or R³) is an alkyl, alkenyl, alkynyl, aryl, or heteroaryl group substituted by an amine or amide group, can be prepared by the exemplary routes shown in Scheme 5 below.

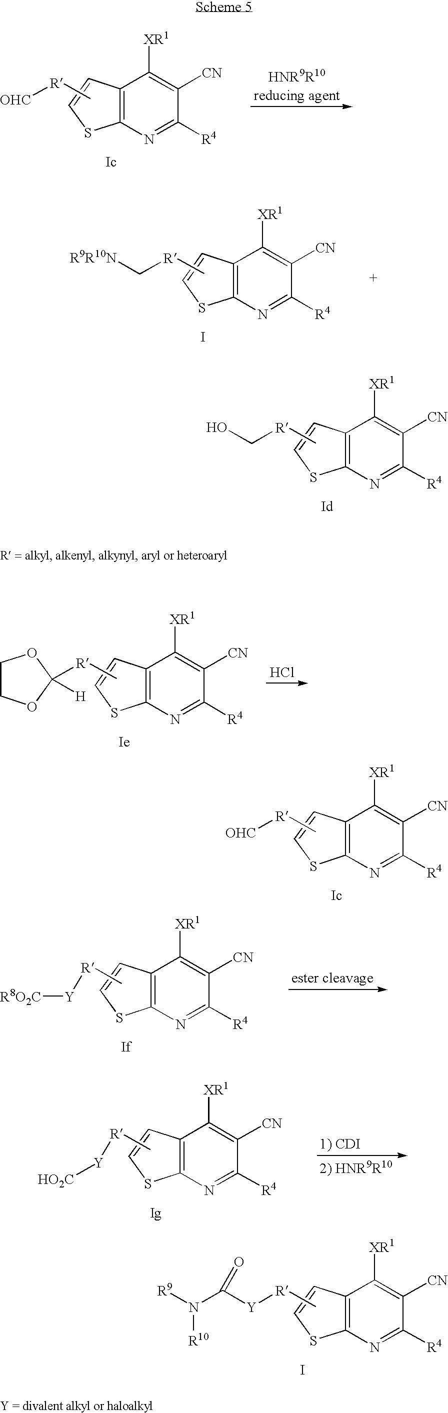

Aldehydes of formula Ic can be converted to compounds of formula I where R² (or R³) is R′—CH₂NR⁹R¹⁰ via reductive amination. The group R′ can be an alkyl, alkenyl, alkynyl, aryl, or heteraryl group. Specifically, treatment of compounds of formula Ic with an amine of formula HNR⁹R¹⁰ in the presence of a reducing agent, preferably sodium triacetoxyborohydride, in a solvent system that can include dichloromethane and either DMF or N-methyl-2-pyrrolidone (NMP), provides compounds of formula I where R² (or R³) is R′—CH₂NR⁹R¹⁰. Alcohols of formula Id can be obtained as a by-product of this reaction via reduction of the formyl group of compounds of formula Ic.

Compounds of formula Ic can be prepared by hydrolysis of the acetal group of compounds of formula Ie, preferably with aqueous hydrochloric acid in the presence of a co-solvent such as THF.

Scheme 5 also depicts the preparation of compounds of formula I, where R² (or R³) is R′ substituted by Y—C(O)NR⁹R¹⁰, from esters of formula If, where R⁸ is a lower alkyl group. Esters of formula If are converted to the corresponding acids of formula Ig by treatment with aqueous sodium hydroxide in a co-solvent such as ethanol at elevated temperatures. The corresponding amides of formula I, where R² (or R³) is R′ substituted by Y—C(O)NR⁹R¹⁰, are prepared by treatment of the acids with a coupling agent such as N,N-carbonyldiimidazole or alternatively thionyl chloride or the like, followed by the addition of an amine of formula HNR⁹R¹⁰.

Compounds of the invention having formula I can also be prepared by reversing the order of the previously mentioned steps. As depicted in Scheme 6 below, the I or Br group at C2 or C3 of the thieno[2,3-b]pyridine-5-carbonitrile is first replaced by the group R², followed by addition of the compound of formula R¹XH to give the compounds of formula I. The general reaction conditions are those referred to previously.

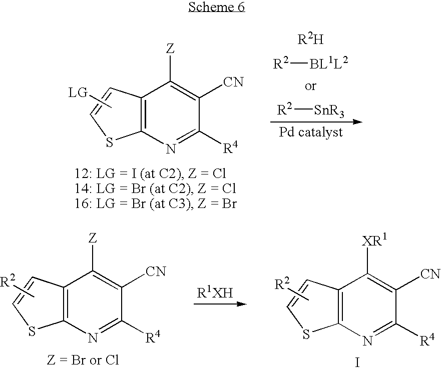

Compounds of the invention of formula I where the pyridine of the thieno[2,3-b]pyridine ring is oxidized can be prepared as shown in Scheme 7 below, where X, R¹, R² and R⁴ are as defined hereinabove.

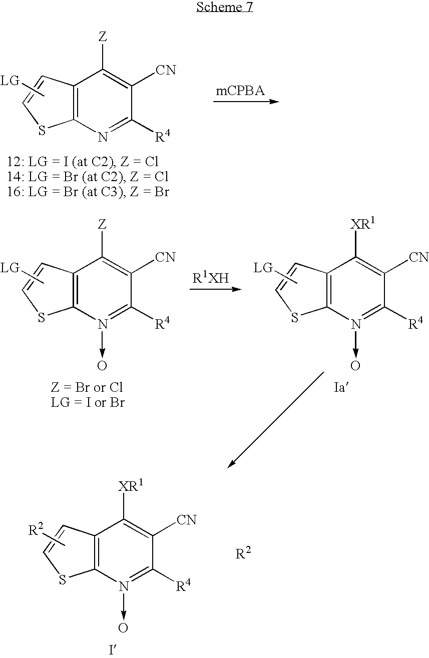

Treatment of a halide-substituted thienopyridine (e.g., intermediates 12, 14, or 16) with an oxidizing agent such as m-chloroperbenzoic acid (mCPBA) in a solvent such as chloroform provides an N-oxide of the thienopyridine. Addition of a compound of formula R¹XH, under the conditions previously noted provides an N-oxide of compounds of formula Ia. Displacement of the Br or Cl at C-2 or C-3, under the general reaction conditions referred to previously, yields compounds of of formula I where the nitrogen of the thienopyridine ring is oxidized and R⁴ is H.

Compounds of the invention of formula I where the sulfur of the thieno[2,3-b]pyridine ring is oxidized can be prepared as shown in Scheme 8 below.

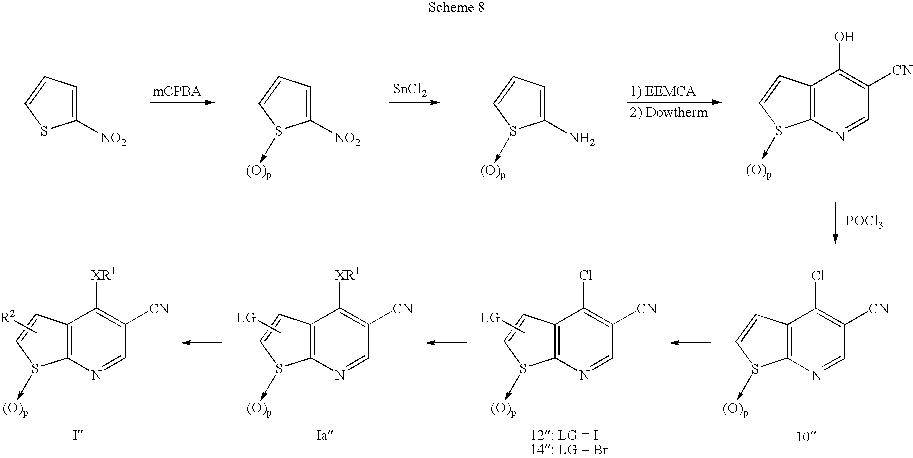

Treatment of 2-nitrothiophene with an oxidizing agent, such as mCPBA in a solvent such as chloroform, provides the sulfoxide (p=1) or sulfone (p=2), depending on the reaction conditions, including nature of the oxidant, equivalents of oxidant used and temperature. Reduction of the nitro group to an amine, followed by addition of ethyl (ethoxymethylene)cyanoacetate (EEMCA), thermal cyclization in a solvent such as Dowtherm®, and subsequent chlorination at C-4 provides the key intermediate 10″. This route corresponds to that used to prepare 4-chlorothieno[2,3-b]pyridine-5-carbonitrile (Khan, M. A. et al. (1977), J. Heterocyclic Chem., 14: 807). lodination or bromination at C-2 or C-3 followed by addition of a compound of formula R¹XH, and displacement of the leaving group at C-2 or C-3 under the general reaction conditions referred to previously, yields the compounds of the invention of formula I″ where the sulfur of the thienopyridine ring is oxidized and R⁴ is H.

Scheme 9 below depicts an alternate route for the preparation of 4-chlorothieno[2,3-b]pyridine-5-carbonitriles 10 and 4-chloro-2-iodothieno[2,3-b]pyridine-5-carbonitriles 12, where R³ can be H or other substituents as defined hereinabove.

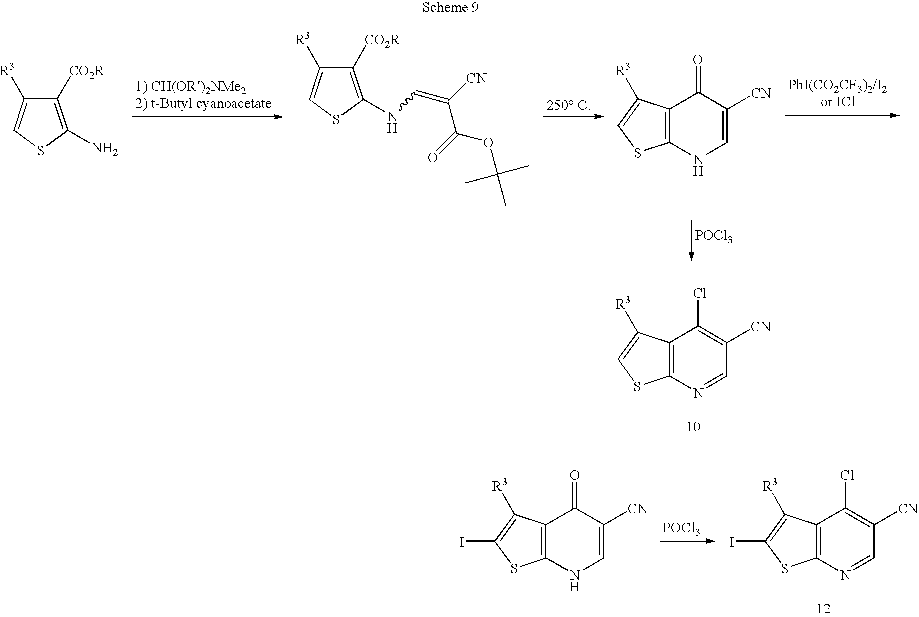

The starting 2-aminothiophene-3-carboxylic ester is treated with a dialkylacetal of DMF, preferably dimethylformamide dimethylacetal. The resultant amidine is reacted with t-butyl cyanoacetate to provide a (Z)-2-(1-amino-3-tert-butoxy-2-cyano-3-oxoprop-1-enyl)thiophene-3-carboxylic ester intermediate, which is heated, preferably to 250° C., in a solvent such as diphenyl ether to provide a 4-hydroxythieno[2,3-b]pyridine-5-carbonitrile. Reaction of the 4-hydroxythieno[2,3-b]pyridine-5-carbonitrile with either [bis(trifluoroacetoxy)iodo]benzene and iodine in a solvent such as chloroform, or alternatively reaction with iodine monochloride and sodium acetate in a solvent such as methanol gives a 4-hydroxy-2-iodothieno[2,3-b]pyridine-5-carbonitrile. Treatment of the 4-hydroxy-2-iodothieno[2,3-b]pyridine-5-carbonitrile with phosphorus oxychloride provides a 4-chloro-2-iodothieno[2,3-b]pyridine-5-carbonitrile of formula 12. Treatment of the 4-hydroxythieno[2,3-b]pyridine-5-carbonitrile with phosphorus oxychloride provides a 4-chlorothieno[2,3-b]pyridine-5-carbonitrile of formula 10.

Scheme 10 below shows the preparation of compounds of formula I where R² is C(O)OR⁸ or C(O)NR⁹R¹⁰, and X, R¹, R³, R⁴, R⁸, R⁹ and R¹⁰ are as defined hereinabove.

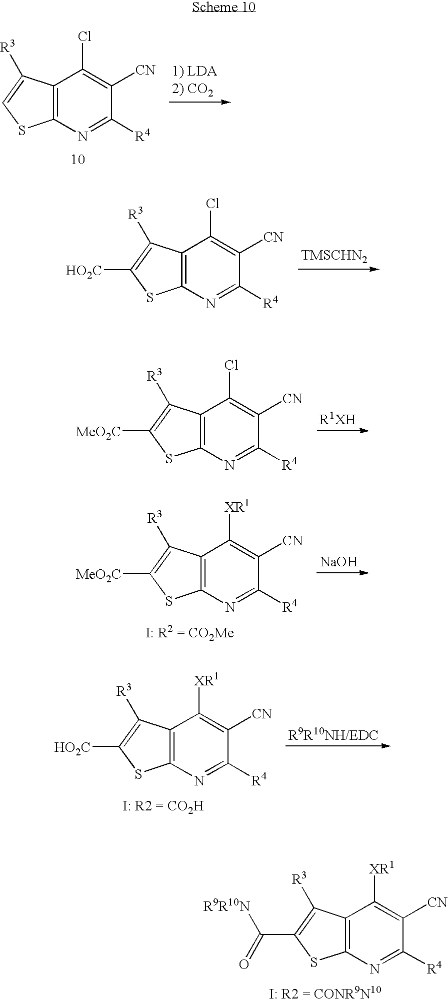

Treatment of compounds of formula 10, where R³ can be H or other substituents as defined hereinabove, with LDA at a low temperature followed by addition of carbon dioxide, preferably in the form of dry ice, provides an acid derivative of formula 10. Trimethylsilyl diazomethane converts the acid to the corresponding methyl ester. The C-4 chloro group can then be displaced by R¹XH, using the general conditions referred to previously, to provide compounds of formula I where R² is CO₂CH₃. Hydrolysis of the ester to the acid with base provides compounds of formula I where R² is CO₂H. Subsequent reaction of the acid with an amine of formula R⁹R¹⁰NH in the presence of a coupling reagent, preferably N-(3-dimethylaminopropyl)-N′-ethylcarbodiimide hydrochloride (EDC), provides compounds of formula I where R² is an C(O)NR⁹R¹⁰ group.

Additional compounds of formula I, where X, R¹, R³, R⁴, R⁹ and R¹⁰ are as defined hereinabove, can be prepared as shown in Scheme 11 below.

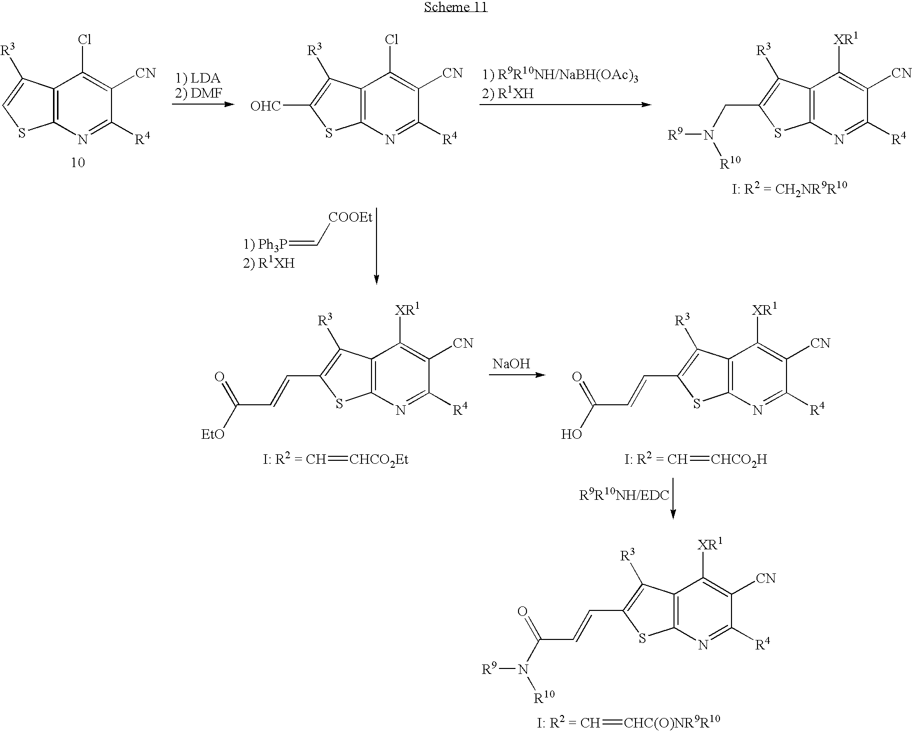

Treatment of compounds of formula 10 with LDA at reduced temperature followed by the addition of DMF provides an aldehyde analog of formula 10. Reductive amination via treatment of the aldehyde intermediate with an amine of formula R⁹R¹⁰NH in the presence of a reducing agent, preferably sodium triacetoxyborohydride, with subsequent displacement of the 4-chloro group with R¹XH, using the general conditions referred to previously, provides compounds of formula I where the R² group is CH₂NR⁹R¹⁰. Wittig reaction of the aldehyde intermediate, preferably with (carbethoxyrnethylene)triphenylphosphorane, in a solvent such as THF, provides the α,β-unsaturated ethyl ester. Subsequent displacement of the 4-chloro group with R¹XH provides compounds of formula I where the R² group is an α,β-unsaturated ethyl ester. Ester hydrolysis with a base, preferably aqueous NaOH, provides compounds of formula I where the R² group is an α,β-unsaturated carboxylic acid. The acid functionality is converted to an amide by reaction with an amine of formula R⁹R¹⁰NH in the presence of a coupling reagent, preferably EDC.

Scheme 12 depicts an alternative route to prepare compounds of formula I where R² is an α,β-unsaturated t-butyl ester or carboxylic acid, and X, R¹, R⁴ and R³ are as defined hereinabove.

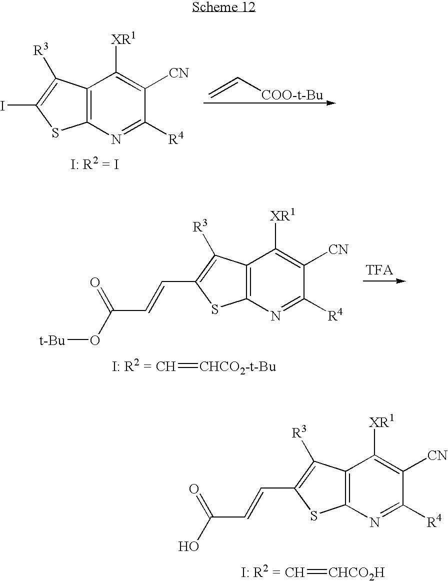

Coupling of a 2-iodo analog of formula I, where X, R¹, R³ and R⁴ are as defined hereinabove, with t-butyl acrylate in the presence of a palladium catalyst, preferably palladium acetate, in the presence of trimethyl phosphite and triethylamine in a solvent such as DMF, provides compounds of formula I where R² is an α,β-unsaturated t-butyl ester. Hydrolysis of the ester, preferably with trifluoroacetic acid, provides compounds of formula I where R² is an α,β-unsaturated carboxylic acid.

Scheme 13 depicts the preparation of additional compounds of formula I from a C-2 phenol analog of formula I, where X, R¹, R³ and R⁴ are as defined hereinabove.

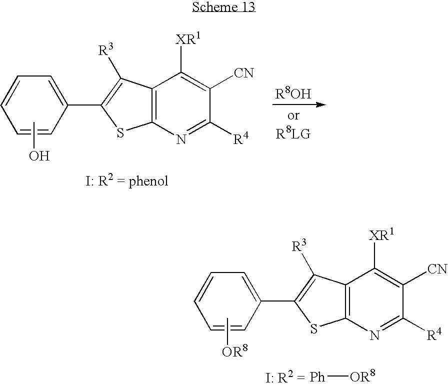

Treatment of the phenol with an alcohol of the formula R⁸OH under Mitsunobu conditions, preferably diethylazodicarboxylate (DEAD) or di-t-butyl-azodicarboxylate and triphenylphosphine, provides compounds of formula I where the R² group is a phenyl ring substituted by an —OR⁸ group, where R⁸ is as defined hereinabove. Treatment of the phenol with an alkyl halide or alkyl tosylate of the formula R⁸LG in the presence of a base, also provides compounds of formula I where the R² group is a phenyl ring substituted by an —OR⁸ group, where R⁸ is as defined hereinabove.

Scheme 14 depicts the preparation of compounds of formula I where R² is substituted by an aminoalkyl group of the formula —Y—NR⁹R¹⁰, where Y is a divalent C₁₋₁₀ alkyl group and X, R¹, R², R³, R⁴, R⁹ and R¹⁰ are as defined hereinabove.

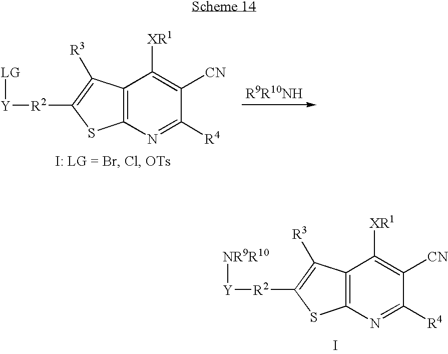

As shown, treatment of a haloalkyl-substituted analog (other leaving groups such as tosylate and mesylate can be used instead of the halide) with an amine of formula R⁹R¹⁰NH in a solvent such as dimethoxyethane (DME), optionally in the presence of Nal, at elevated temperature, provides compounds of formula I where R² is substituted by a group —Y—NR⁹R¹⁰.

Scheme 15 depicts the preparation of compounds of formula I where R³ is CH₂OH or a CH₂NR⁹R¹⁰ group, and X, R¹, R², R⁴, R⁹ and R¹⁰ are as defined hereinabove.

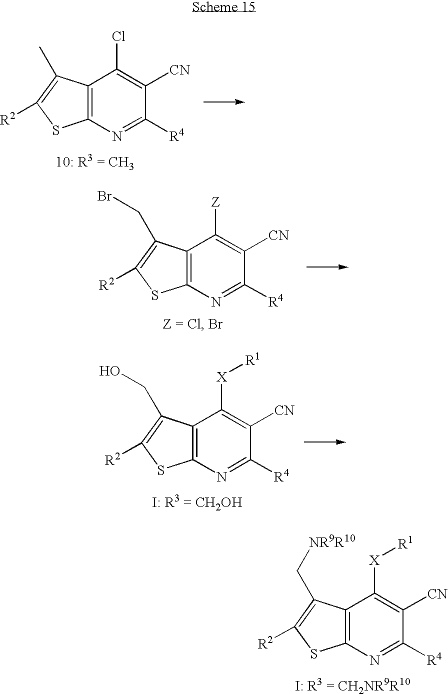

Reaction of the C-3 methyl group with a brominating agent, preferably N-bromosuccinimide (NBS) in the presence of a free radical source such as 2,2′-azobis(2-methylpropionitrile) (AIBN) in a solvent such as carbon tetrachloride, provides the C-3 CH₂Br derivative where Z is Cl or Br. Treatment with a base such as calcium carbonate in a solvent system such as dioxane and water provides the C-3 CH₂OH derivative. Subsequent displacement of the 4-chloro/bromo group on the C-3 CH₂OH derivative with R¹XH using the general conditions referred to previously, provides the compounds of formula I where R³ is CH₂OH. Conversion of the CH₂OH group to a CH₂OTs or a CH₂OMs group and subsequent reaction with an amine of formula R⁹R¹⁰NH provides compounds of formula I where R³ is a CH₂NR⁹R¹⁰ group.

Scheme 16 depicts an alternate route to that shown in Scheme 7 for the preparation of compounds of formula I where the pyridine ring of the core is oxidized, and X, R¹, R², R³ and R⁴ are as defined hereinbelow.

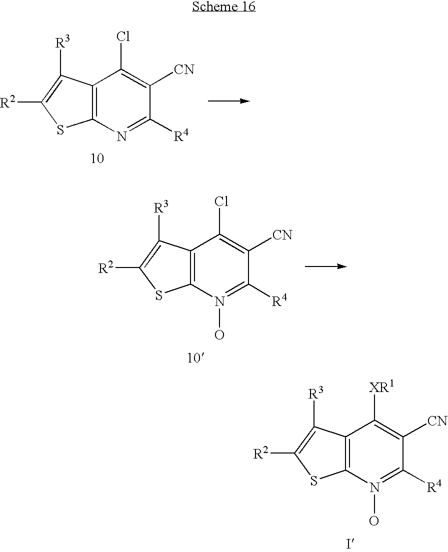

Treatment of compounds of formula 10 with an oxidizing agent, preferably m-CPBA or H₂O₂ in acetic acid, provides a 7-oxide analog of compounds of formula 10. Subsequent displacement of the 4-chloro group with R¹XH, using the general conditions referred to previously, provides compounds of formula I where the pyridine ring of the core is oxidized.

Scheme 17 depicts the synthesis of compounds of formula I from a 4-fluoro intermediate, where X, R¹, R², R³ and R⁴ are as defined hereinabove.

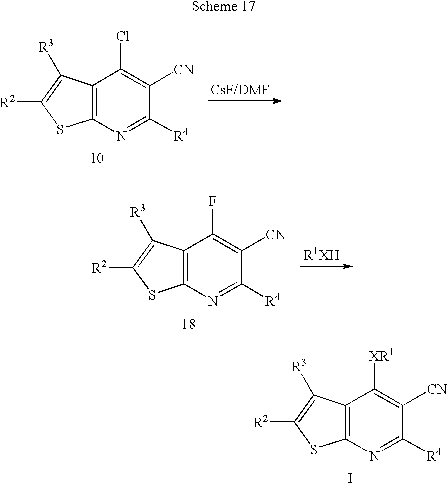

Treatment of compounds of formula 10 with CsF in a solvent such as DMF provides a 4-fluoro analog of compounds of formula 10. Subsequent displacement of the 4-fluoro group with R¹XH in a solvent such as DMF provides compounds of formula I.

The following examples illustrate various synthetic routes which can be used to prepare compounds of formula I.

Unless stated otherwise the analytical HPLC conditions were as follows. A Prodigy ODS3 (0.46×15 cm) column was used. The gradient was 10% acetonitrile to 90% acetonitrile with 0.01% TFA additive in water over 20 minutes. The flow rate was 1.0 mL/min, and the temperature was 40° C.

### EXAMPLE 1

### Preparation of 4-(1H-Indol-5-ylamino)-2-[(4-morpholin-4-ylmethyl)phenyl]thieno[2,3-b]pyridine-5-carbonitrile 101

4-Chloro-2-iodothieno[2,3-b]pyridine-5-carbonitrile 12, prepared according to, for example, the method depicted in Scheme 2 above or other methods known by those skilled in the art (see e.g., Boschelli, D. et al. (2004), J. Med. Chem., 47: 6666-6668), was used as the starting reagent. A solution of 4-chloro-2-iodothieno[2,3-b]pyridine-5-carbonitrile 12 (20 mg), tetrakis(triphenylphosphine)palladium (2 mg), 4-[4-(4,4,5,5-tetramethyl-[1,3,2]dioxaborolan-2-yl)-benzyl]-morpholine (30 mg), sodium carbonate (2.0 M solution, 1 mL) and dioxane (1 mL) was heated to reflux for 6 hours, then cooled to room temperature. A yellow solid formed and was filtered and washed with ether to give 4-chloro-2-[(4-morpholin-4-ylmethyl)phenyl]thieno [2,3-b]pyridine-5-carbonitrile, HPLC retention time 1.9 min, MS 370 (M+H).

A solution of 4-chloro-2-[(4-morpholin-4-ylmethyl)phenyl]thieno[2,3-b]pyridine-5-carbonitrile (30 mg), 5-aminoindole (20 mg) and pyridine HCl (10 mg) in ethoxyethanol was heated to 120° C. for 2 hours. After cooling to room temperature, the mixture was filtered and the filtrate purified by preparative HPLC to give 4-(1H-indol-5-ylamino)-2-[(4-morpholin-4-ylmethyl)phenyl]thieno[2,3-b]pyridine-5-carbonitrile 101 (6 mg), HPLC retention time 2.1 min, MS 466 (M+H).

### EXAMPLE 2

### Alternate Preparation of 4-(1H-indol-5-ylamino)-2-[(4-morpholin-4-ylmethyl)phenyl]thieno[2,3-b]pyridine-5-carbonitrile 101

4-(1H-indol-5-ylamino)-2-[(4-morpholin-4-ylmethyl)phenyl]thieno[2,3-b]pyridine-5-carbonitrile 101 was alternatively prepared as follows. A mixture of 4-chloro-2-iodothieno[2,3-b]pyridine-5-carbonitrile 12 (5.10 g, 15.91 mmol) and 5-aminoindole (2.21 g, 16.71 mmol) in ethanol was heated at reflux for 21 hours. An additional 310 mg of 5-aminoindole was added and the mixture was heated at reflux for 27 hours. The mixture was cooled to room temperature and the solid was collected by filtration, washed with ethanol and dried in vacuo to give 6.40 g of 4-(1H-indol-5-ylamino)-2-iodothieno[2,3-b]pyridine-5-carbonitrile hydrochloride 102 as a tan solid, mp 250-252° C., MS 417.0 (M+H)+.

A mixture of 4-(1H-indol-5-ylamino)-2-iodothieno[2,3-b]pyridine-5-carbonitrile hydrochloride 102 (3.00 g, 6.64 mmol), tetrakis(triphenylphosphine)palladium (381 mg, 0.33 mmol), 4-formylphenylboronic acid (1.30 g, 8.63 mmol) in 48 mL of saturated aqueous sodium bicarbonate and 55 mL of DME was heated at reflux for 6 hours. The reaction mixture was cooled to room temperature and concentrated in vacuo. The precipitate was collected by filtration, washed with water, dichloromethane, ethyl acetate and diethyl ether and dried in vacuo to give 1.84 g of 2-(4-formylphenyl)-4-(1H-indol-5-ylamino)thieno[2,3-b]pyridine-5-carbonitrile 103. Further purification of 360 mg of this material by flash column chromatography eluting with a gradient of 0 to 15% ethyl acetate in dichloromethane gave 175 mg of pure 2-(4-formylphenyl)-4-(1H-indol-5-ylamino)thieno[2,3-b]pyridine-5-carbonitrile 103, mp>260° C., MS 395.0 (M+H)+.

To a 0° C. mixture of 2-(4-formylphenyl)-4-(1H-indol-5-ylamino)thieno[2,3-b]pyridine-5-carbonitrile 103 (800 mg, 2.03 mmol), and morpholine (884 mg, 10.15 mmol) in 32 mL of dichloromethane and 1.5 mL of N-methylpyrrolidone (NMP) was added sodium triacetoxyborohydride (2.15 g, 10.15 mmol), followed by 10 drops of acetic acid. After stirring at room temperature overnight, the reaction mixture was partitioned between ethyl acetate and water. The aqueous layer was extracted with ethyl acetate, and the organic layers were combined, dried over magnesium sulfate, filtered and concentrated in vacuo. Two purifications by flash column chromatography gave 431 mg of 4-(1H-indol-5-ylamino)-2-[(4-morpholin-4-ylmethyl)phenyl]thieno[2,3-b]pyridine-5-carbonitrile 101 as a yellow solid, mp 251-253° C., MS 466.1 (M+H)⁺.

Following one the procedures for the preparation of compound 101 described above, 2-(4-formylphenyl)-4-(1H-indol-5-ylamino)thieno[2,3-b]pyridine-5-carbonitrile 103 was reacted with the appropriate amine to provide the following analogs listed in Table 2.

TABLE 2

| Compound | | | MP |
|---|---|---|---|
| number | Compound Name | MS | (° C.) |
| 104 | 4-(1H-indol-5-ylamino)-2-{4-[(4-methylpiperazin-1- | 479.1 (M + H)⁺ | 285-287 |
| | yl)methyl]phenyl}thieno[2,3-b]pyridine-5-carbonitrile | | |
| 105 | 2-{4-[(dimethylamino)methyl]phenyl}-4-(1H-indol-5- | 424.1 (M + H)⁺ | 220-222 |
| | ylamino)thieno[2,3-b]pyridine-5-carbonitrile | | |
| 106 | 2-(4-{[(2-hydroxyethyl)amino]methyl}phenyl)-4-(1H-indol-5- | 440.3 (M + H)⁺ | 160 dec. |
| | ylamino)thieno[2,3-b]pyridine-5-carbonitrile | | |

### EXAMPLE 3

### Preparation of 4-(1H-indol-5-ylamino)-2-phenylthieno[2,3-b]pyridine-5-carbonitrile 107

A mixture of 4-chloro-2-iodothieno[2,3-b]pyridine-5-carbonitrile 12 (1.27 g, 3.96 mmol), phenylboronic acid (531 mg, 4.36 mmol) and tetrakis(triphenylphosphine)palladium (279 mg, 0.20 mmol) in 50 mL of DME and 36 mL of saturated aqueous sodium bicarbonate was heated at reflux for 4 hours. The reaction mixture was cooled to room temperature and the precipitate was collected by filtration, washing with water and diethyl ether. Additional washing with ethyl acetate and dichloromethane gave 900 mg of 4-chloro-2-phenylthieno[2,3-b]pyridine-5-carbonitrile 20 as a cream-colored solid, mp 202-204° C., MS 271.1 (M+H)⁺.

A mixture of 4-chloro-2-phenylthieno[2,3-b]pyridine-5-carbonitrile 20 (120 mg, 0.44 mmol), 5-aminoindole (70 mg, 0.53 mmol) and pyridine hydrochloride (51 mg, 0.49 mmol) in 10 mL of 2-ethoxyethanol was heated at 120° C. for 5 hours then stirred at room temperature overnight. An additional 70 mg of 5-aminoindole and 52 mg of pyridine hydrochloride were added and the reaction was heated at 120° C. for 4.5 hours. The reaction mixture was cooled to room temperature and partitioned between ethyl acetate and saturated aqueous sodium bicarbonate. The organic layer was dried over magnesium sulfate, filtered and concentrated in vacuo. The solid was washed with hot methanol and dichloromethane to provide 94 mg of 4-(1H-indol-5-ylamino)-2-phenylthieno[2,3-b]pyridine-5-carbonitrile 107 as a tan solid, mp>245° C., MS 367.1 (M+H)⁺.

### EXAMPLE 4

### Preparation of 4-(1H-indol-7-ylamino)-2-phenylthieno[2,3-b]pyridine-5-carbonitrile 109

A mixture of 4-chloro-2-iodothieno[2,3-b]pyridine-5-carbonitrile 12 (300 mg, 0.94 mmol) and 7-aminoindole (280 mg, 2.06 mmol) in 12 mL of ethanol was heated at reflux for 2 days. The reaction mixture was cooled slightly and the precipitate was collected and washed with diethyl ether. The solids were stirred with saturated aqueous sodium bicarbonate for 1.5 hours then filtered and washed with water. The solids were treated with hot ethyl acetate and the mixture was filtered. Concentration of the filtrate and trituration with diethyl ether provided 89 mg of 4-(1H-indol-7-ylamino)-2-iodothieno[2,3-b]pyridine-5-carbonitrile 108 as a tan solid, mp>245° C., MS 416.9 (M+H).

A mixture of 4-(1H-indol-7-ylamino)-2-iodothieno[2,3-b]pyridine-5-carbonitrile 108 (153 mg, 0.37 mmol), tetrakis(triphenylphosphine)palladium (9 mg) and phenylboronic acid (90 mg, 0.74 mmol) in 3 mL of DME and 1.5 mL of saturated aqueous sodium bicarbonate was heated at reflux for 6 hours. The reaction mixture was partitioned between ethyl acetate and saturated aqueous sodium bicarbonate. The organic layer was washed with water, dried over magnesium sulfate, filtered and concentrated in vacuo. Trituration of the residue with diethyl ether gave 80 mg of 4-(1H-indol-7-ylamino)-2-phenylthieno[2,3-b]pyridine-5-carbonitrile 109 as a light brown solid, mp>245° C., MS 367.1 (M+H)+.

Following the procedure for the preparation of compound 109, 4-(1H-indol-5-ylamino)-2-iodothieno[2,3-b]pyridine-5-carbonitrile hydrochloride 102 was reacted with the appropriate boronic acid or boronic ester to provide the following analogs listed in Table 3.

TABLE 3

| Compound | | | MP |
|---|---|---|---|
| Number | Compound Name | MS | (° C.) |
| 110 | 2-(5-formyl-3-furyl)-4-(1H-indol-5-ylamino)thieno[2,3- | 383.1 (M − H)⁻ | 170-180 |
| | b]pyridine-5-carbonitrile | | |
| 111 | 2-[4-(dimethylamino)phenyl]-4-(1H-indol-5-ylamino)thieno[2,3- | 410.1 (M + H)⁺ | >255 |
| | b]pyridine-5-carbonitrile | | |
| 112 | 2-{3-[2-(dimethylamino)ethyl]phenyl}-4-(1H-indol-5- | 483.3 (M + H)⁺ | 254 |
| | ylamino)thieno[2,3-b]pyridine-5-carbonitrile | | |
| 114 | 2-{2-[(dimethylamino)methyl]phenyl}-4-(1H-indol-5- | 424.0 (M + H)⁺ | 162 |
| | ylamino)thieno[2,3-b]pyridine-5-carbonitrile | | |

### EXAMPLE 5

### Preparation of 4-(1H-indol-4-ylamino)-2-phenylthieno[2,3-b]pyridine-5-carbonitrile 115

A mixture of 4-chloro-2-phenylthieno[2,3-b]pyridine-5-carbonitrile 20 (150 mg, 0.55 mmol), 4-aminoindole (123 mg, 0.93 mmol), 2-dicyclohexylphosphino-2′-(N,N-dimethylamino)biphenyl (92 mg, 0.23 mmol), tris(dibenzylidineacetone)dipalladium (71 mg, 0.078 mmol) and potassium phosphate (245 mg, 1.15 mmol) in 4 mL of DME was heated at 120° C. for 4 hours. The reaction mixture was partitioned between ethyl acetate and water. The aqueous layer was extracted with ethyl acetate, and the organic layers were combined, dried over magnesium sulfate, filtered and concentrated in vacuo. The residue was purified by flash column chromatography eluting with a gradient of 2 to 5% methanol in dichloromethane. Trituration with methanol and dichloromethane provided 35 mg of 4-(1H-indol-4-ylamino)-2-phenylthieno[2,3-b]pyridine-5-carbonitrile 115 as a tan solid, mp>245° C., MS 367.1 (M+H)⁺.

### EXAMPLE 6

### Preparation of 4-(1-H-indol-6-ylamino)-2-phenylthieno[2,3-b]pyridine-5-carbonitrile hydrochloride 116

A mixture of 4-chloro-2-phenylthieno[2,3-b]pyridine-5-carbonitrile 20 (120 mg, 0.44 mmol) and 6-aminoindole (88 mg, 0.67 mmol) in 3 mL of ethanol was heated at reflux for 28 hours. The reaction mixture was cooled to room temperature and the precipitate was collected and washed with ethanol. Additional washing with warm ethanol gave 61 mg of 4-(1H-indol-6-ylamino)-2-phenylthieno[2,3-b]pyridine-5-carbonitrile hydrochloride 116 as a tan solid, mp>245° C., MS 416.9 (M+H).

Following the procedure for the preparation of compound 116, the appropriate 4-chlorothieno[2,3-b]pyridine-5-carbonitrile or 4-bromothieno[2,3-b]pyridine-5-carbonitrile was reacted with the appropriate amine to provide the following analogs listed in Table 4. In some cases, other solvents such as 2-propanol and 2-ethoxyethanol were used as the solvent.

TABLE 4

| Compound | | | MP |
|---|---|---|---|
| Number | Compound Name | MS | (° C.) |
| 117 | 4-[(4-methyl-1H-indol-5-yl)amino]-2-phenylthieno[2,3- | 381.1 (M + H)⁺ | 238-240 |
| | b]pyridine-5-carbonitrile | | |
| 118 | 4-[(2-methyl-1H-indol-5-yl)amino]-2-phenylthieno[2,3- | 381.1 (M + H)⁺ | >245 |
| | b]pyridine-5-carbonitrile | | |
| 119 | 4-(1H-benzimidazol-5-ylamino)-2-phenylthieno[2,3-b]pyridine- | 368.2 (M + H)⁺ | 220-222 |
| | 5-carbonitrile | | |
| 120 | 4-[(7-methyl-1H-indol-5-yl)amino]-2-phenylthieno[2,3- | 381.2 (M + H)⁺ | >250 |
| | b]pyridine-5-carbonitrile | | |
| 121 | 4-[(4-methyl-1H-indol-5-yl)amino]-2-{4-[(4-methylpiperazin- | 493.2 (M + H)⁺ | 240-242 |
| | 1-yl)methyl]phenyl}thieno[2,3-b]pyridine-5-carbonitrile | | |
| 122 | 4-[1H-indol-5-yl(methyl)amino]-2-phenylthieno[2,3- | 381.0 (M + H)⁺ | 245-247 |
| | b]pyridine-5-carbonitrile | | |
| 123 | 2-iodo-4-[(4-methyl-1H-indol-5-yl)amino]thieno[2,3- | 431.0 (M + H)⁺ | |
| | b]pyridine-5-carbonitrile | | |
| 126 | 4-[(4-ethyl-1H-indol-5-yl)amino]-2-phenylthieno[2,3- | 395.1 (M + H)⁺ | 225-226 |
| | b]pyridine-5-carbonitrile | | |
| 133 | 4-[(3-methyl-1H-indol-5-yl)amino]-2-phenyl | 381.0 (M + H)⁺ | 263-265 |
| | thieno[2,3-b]pyridine-5-carbonitrile | | |
| 134 | 4-[(1-methyl-1H-indol-5-yl)amino]-2-phenyl | 381.1 (M + H)⁺ | 225-226 |
| | thieno[2,3-b]pyridine-5-carbonitrile | | |

### EXAMPLE 7

### Preparation of 4-(1H-indol-5-ylmethylamino)-2-phenylthieno[2,3-b]pyridine-5-carbonitrile 124

A mixture of 4-chloro-2-phenylthieno[2,3-b]pyridine-5-carbonitrile 20 (114 mg, 0.42 mmol), 5-aminomethylindole (83 mg, 0.56 mmol) and N,N-diisopropylethyl amine (Hunig's base, 0.100 mL, 0.57 mmol) in 10 mL of 2-ethoxyethanol was heated at reflux overnight. The reaction mixture was partitioned between ethyl acetate and water. The organic layer was washed with brine, dried over magnesium sulfate, filtered and concentrated in vacuo. The residue was purified by flash column chromatography eluting with a gradient of 3:1 to 1:1Hexane:ethyl acetate to provide 84 mg of 4-(1H-indol-5-ylmethylamino)-2-phenylthieno[2,3-b]pyridine-5-carbonitrile 124 as an off-white solid, mp 219-221° C., MS 381.1 (M+H)⁺.

Following the procedure for the preparation of compound 124, the appropriate 4-chloro-2-phenylthieno[2,3-b]pyridine-5-carbonitrile or 4-bromo-2-phenylthieno[2,3-b]pyridine-5-carbonitrile was reacted with the appropriate amine to provide the following analog listed in Table 5.

TABLE 5

| Compound | | | MP |
|---|---|---|---|
| Number | Compound Name | MS | (° C.) |
| 125 | 4-(1H-indol-4-ylmethylamino)- | 381.1 (M + H)+ | >240 |
| | 2-phenylthieno[2,3-b]pyridine- | | |
| | 5-carbonitrile | | |

### EXAMPLE 8

### Preparation of 4-{[2-(1H-imidazol-4-yl)ethyl]amino}-2-phenylthieno[2,3-b]pyridine-5-carbonitrile 127

To a solution of histamine (123 mg, 1.11 mmol) in 5 mL of DMF at 65° C. was added NaH (44 mg of 60% in oil, 1.10 mmol) and the solution was heated at 65° C. for 30 minutes. 4-Chloro-2-phenylthieno[2,3-b]pyridine-5-carbonitrile 20 (120 mg, 0.44 mmol) was added and the mixture was heated at 65° C. for 1.5 hours. The reaction mixture was cooled to room temperature and poured into ethyl acetate, washed with water and brine. The organic layer was dried over magnesium sulfate, filtered and concentrated in vacuo. The solid was triturated with methanol and dichloromethane to give 99 mg of 4-{[2-(1H-imidazol-4-yl)ethyl]amino}-2-phenylthieno[2,3-b]pyridine-5-carbonitrile 127 as an off-white solid, mp>245° C., MS 346.2 (M+H)⁺.

Following the procedure for the preparation of compound 127, the appropriate 4-chloro-thieno[2,3-b]pyridine-5-carbonitrile or 4-bromo-thieno[2,3-b]pyridine-5-carbonitrile was reacted with the appropriate alkyl amine to provide the analog listed in Table 6.

TABLE 6

| Compound | | | MP |
|---|---|---|---|
| Number | Compound Name | MS | (° C.) |
| 128 | 4-{[2-(1H-imidazol-4- | 458.3 | 147-150 |
| | yl)ethyl]amino}-2- | (M + H)+ | |
| | {4-[(4-methylpiperazin-1- | | |
| | yl)methyl]phenyl}thieno[2,3- | | |
| | b]pyridine-5-carbonitrile | | |

### EXAMPLE 9

### Preparation of N-(5-cyano-2-phenylthieno[2,3-b]pyridin-4-yl)-1H-indole-5-carboxamide 129

A mixture of 1H-indole-5-carboxamide (142 mg, 0.88 mmol) and NaH (35 mg of 60% in oil, 0.88 mmol) in 8 mL of DMF was stirred at room temperature for 15 minutes. 4-Chloro-2-phenylthieno[2,3-b]pyridine-5-carbonitrile 20 (120 mg, 0.44 mmol) was added and the mixture was heated at 50° C. for 30 minutes. The reaction mixture was partitioned between ethyl acetate and aqueous sodium bicarbonate. The organic layer was dried over magnesium sulfate, filtered and concentrated in vacuo. The residue was purified by flash column chromatography eluting with a gradient of 3:1Hexane:ethyl acetate to all ethyl acetate. Trituration with diethyl ether provided 11 mg of N-(5-cyano-2-phenylthieno[2,3-b]pyridin-4-yl)-1H-indole-5-carboxamide 129 as a white solid, mp softens at 125° C., MS 395.1 (M+H)⁺.

### EXAMPLE 10

### Preparation of 4-(1H-indol-5-yloxy)-2-phenylthieno[2,3-b]pyridine-5-carbonitrile 130

A mixture of 4-chloro-2-phenylthieno[2,3-b]pyridine-5-carbonitrile 20 (120 mg, 0.44 mmol), 5-hydroxyindole (71 mg, 0.53 mmol) and potassium carbonate (91 mg, 0.66 mmol) in 4 mL of acetonitrile was heated at 80° C. for 5 hours. The reaction mixture was cooled and diluted with 10 mL of water. The precipitate was collected by filtration and washed with water followed by diethyl ether to give 127 mg (79%) of 4-(1H-indol-5-yloxy)-2-phenylthieno[2,3-b]pyridine-5-carbonitrile 130 as an off-white solid, mp 219-221° C., MS 368.1 (M+H)⁺.

### EXAMPLE 11

### Preparation of 4-(1H-indol-5-yl)-2-phenylthieno[2,3-b]pyridine-5-carbonitrile 131

A mixture of 4-chloro-2-phenylthieno[2,3-b]pyridine-5-carbonitrile 20 (151 mg, 0.56 mmol), 5-indolyl boronic acid (137 mg, 0.86 mmol) and tetrakis(triphenylphosphine)palladium (75 mg) in 10 mL of DME and 5 mL of saturated aqueous sodium bicarbonate was heated at reflux for 2 hours. The reaction mixture was partitioned between ethyl acetate and saturated aqueous sodium bicarbonate. The organic layer was dried over magnesium sulfate, filtered and concentrated in vacuo. The residue was triturated with diethyl ether to provide a solid. The filtrate was concentrated and purified by flash column chromatography eluting with a gradient of 3:1 to 1:1Hexane:ethyl acetate. The product was combined with the previously isolated solid and stirred with diethyl ether. Filtration provided 34 mg of 4-(1H-indol-5-yl)-2-phenylthieno[2,3-b]pyridine-5-carbonitrile 131 as a light tan solid, mp>245° C., MS 352.2 (M+H)⁺.

### EXAMPLE 12

### Preparation of 4-(1H-indol-4ylamino)-2-{4-[(4-methylpiperazin-1-yl)methyl]phenyl}thieno[2,3-b]pyridine-5-carbonitrile 137

A mixture of 4-chloro-2-iodothieno[2,3-b]pyridine-5-carbonitrile 12 (200 mg, 0.62 mmol) and 4-aminoindole (124 mg, 0.94 mrnol) in 4 mL of ethanol was heated at reflux for 28 hours. The reaction mixture was cooled to room temperature and the solid was washed with ethanol. Additional washing with warm ethanol and dichloromethane gave 113 mg of 4-(1H-indol-4-ylamino)-2-iodothieno[2,3-b]pyridine-5-carbonitrile hydrochloride 135 as a light brown solid, mp>245° C., MS 417.0 (M+H)⁺.

A mixture of 4-(1H-indol-4-ylamino)-2-iodothieno[2,3-b]pyridine-5-carbonitrile hydrochloride 135 (850 mg, 1.88 mmol), tetrakis(triphenylphosphine)palladium (118 mg, 0.10 mmol) and 4-formylphenylboronic acid (337 mg, 2.25 mmol) in 24 mL of saturated aqueous sodium bicarbonate and 30 mL of DME was heated at reflux for 4 hours. The reaction mixture was cooled to room temperature and ethyl acetate and water were added. The precipitated solid was collected by filtration and washed with water, ethyl acetate and methanol. The organic layers were combined, dried over magnesium sulfate, filtered and concentrated in vacuo. The residue was combined with the previously obtained solid and washed with diethyl ether, methanol and dichloromethane to provide 510 mg of 2-(4-formylphenyl)-4-(1H-indol-4-ylamino)thieno[2,3-b]pyridine-5-carbonitrile 136 as an orange solid, mp>245° C., MS 395.1 (M+H)⁺.

To a 0° C. solution of 2-(4-formylphenyl)-4-(1H-indol-4-ylamino)thieno[2,3-b]pyridine-5-carbonitrile 136 (120 mg, 0.30 mmol) in 5.5 mL of dichloromethane was added 0.6 mL of NMP followed by 1-methylpiperazine (0.068 mL, 0.61 mmol) then sodium triacetoxyborohydride (322 mg, 1.52 mmol) and 2 drops of acetic acid. After stirring at room temperature for 5 hours, water was added followed by ethyl acetate. The organic layer was washed with saturated aqueous sodium bicarbonate, dried over magnesium sulfate, filtered and concentrated in vacuo. The residue was purified by preparative thin layer chromatography developing with 1% concentrated aqueous ammonium hydroxide in 9% methanol in dichloromethane to provide 47 mg of 4-(1H-indol-4-ylamino)-2-{4-[(4-methylpiperazin-1-yl)methyl]phenyl}thieno[2,3-b]pyridine-5-carbonitrile 137 as a yellow solid, mp>245° C., MS 479.1 (M+H)⁺.

Following the procedure for the preparation of compound 137, 2-(4-formylphenyl)-4-(1H-indol-4-ylamino)thieno[2,3-b]pyridine-5-carbonitrile 136 was reacted with the appropriate amine to provide the analog listed in Table 7.

TABLE 7

| Compound | | | MP |
|---|---|---|---|
| Number | Compound Name | MS | (° C.) |
| 138 | 4-(1H-indol-4-ylamino)-2-[4- | 466.1 (M + H)+ | >245 |
| | (morpholin-4- | | |
| | ylmethyl)phenyl]thieno[2,3- | | |
| | b]pyridine-5-carbonitrile | | |

### EXAMPLE 13

### Preparation of 4-(1H-indol-4-ylamino)-2-{3-[(4-methylpiperazin-1-yl)methyl]phenyl}thieno[2,3-bpyridine-5-carbonitrile 140

A mixture of 4-(1H-indol-4-ylamino)-2-iodothieno[2,3-b]pyridine-5-carbonitrile hydrochloride 135 (1.20 g, 2.65 mmol), tetrakis(triphenylphosphine)palladium (167 mg, 0.145 mmol) and 3-formylphenylboronic acid (475 mg, 3.17 mmol) in 36 mL of saturated aqueous sodium bicarbonate and 45 mL of DME was heated at reflux for 3.5 hours. Ethyl acetate and water were added to the reaction mixture and the solid was collected by filtration. Washing with ethyl acetate, dichloromethane and methanol gave 429 mg of 2-(3-formylphenyl)-4-(1H-indol-4-ylamino)thieno[2,3-b]pyridine-5-carbonitrile 139. The filtrate layers were separated. The organic phase was washed with saturated aqueous sodium bicarbonate followed by brine, then dried over magnesium sulfate, filtered and concentrated in vacuo. The solid was washed with acetone and methanol to provide an additional 253 mg of 2-(3-formylphenyl)-4-(1H-indol-4-ylamino)thieno[2,3-b]pyridine-5-carbonitrile 139. The filtrate was concentrated in vacuo and dichloromethane was added. The mixture was filtered and the filtrate was concentrated and purified by flash column chromatography eluting with a gradient of 0 to 10% methanol in dichloromethane to provide 84 mg of 2-(3-formylphenyl)-4-(1H-indol-4-ylamino)thieno[2,3-b]pyridine-5-carbonitrile 139 as a yellow solid, mp>245° C., MS 395.1 (M+H)⁺.

A solution of 2-(3-formylphenyl)-4-(1H-indol-4-ylamino)thieno[2,3-b]pyridine-5-carbonitrile 139 (120 mg, 0.30 mmol) in 5 mL of dichloromethane and 0.5 mL of NMP was cooled to 0° C. and sodium triacetoxyborohydride (322 mg, 1.52 mmol) was added followed by 1-methylpiperazine (0.169 mL, 1.52 mmol). After stirring at room temperature overnight, the reaction mixture was partitioned between saturated aqueous sodium bicarbonate and ethyl acetate. The organic layer was dried over magnesium sulfate, filtered and concentrated in vacuo. The residue was purified by preparative thin layer chromatography developing with 10% methanol in dichloromethane to provide 68 mg of 4-(1H-indol-4-ylamino)-2-{3-[(4-methylpiperazin-1-yl)methyl]phenyl}thieno[2,3-b]pyridine-5-carbonitrile 140 as a yellow solid, mp>245° C., MS 479.1 (M+H)⁺.

Following the procedure for the preparation of compound 140, 2-(3-formylphenyl)-4-(1H-indol-4-ylamino)thieno[2,3-b]pyridine-5-carbonitrile 139 was reacted with the appropriate amine to provide the analog listed in Table 8.

TABLE 8

| Compound | | | MP |
|---|---|---|---|
| Number | Compound Name | MS | (° C.) |
| 141 | 2-{3- | 424.1 | 216-218 |
| | [(dimethylamino)methyl]phenyl}- | (M + H)+ | |
| | 4-(1H-indol-4- | | |
| | ylamino)thieno[2,3- | | |
| | b]pyridine-5-carbonitrile | | |

### EXAMPLE 14

### Preparation of 4-(1H-indol-4-ylamino)-2-{2-1(4-methylpiperazin-1-yl)methyl]phenyl}thieno[2,3-b]pyridine-5-carbonitrile 143

A mixture of 4-(1H-indol-4-ylamino)-2-iodothieno[2,3-b]pyridine-5-carbonitrile hydrochloride 135 (400 mg, 0.88 mmol), tetrakis(triphenylphosphine)palladium (56 mg, 0.048 mmol) and 2-formylphenylboronic acid (159 mg, 1.06 mmol) in 12 mL of saturated aqueous sodium bicarbonate and 15 mL of DME was heated at reflux for 5 hours. Ethyl acetate and water were added to the reaction mixture and the layers were separated. The organic phase was washed with saturated aqueous sodium bicarbonate followed by brine, then dried over magnesium sulfate, filtered and concentrated in vacuo. The residue was purified by preparative thin layer chromatography developing with 2% methanol in dichloromethane containing a trace of concentrated aqueous ammonium hydroxide to provide 127 mg of 2-(2-formylphenyl)-4-(1H-indol-4-ylamino)thieno[2,3-b]pyridine-5-carbonitrile 142 as a yellow solid, mp 137-139° C., MS 395.1 (M+H)⁺.

A solution of 2-(2-formylphenyl)-4-(1H-indol-4-ylamino)thieno[2,3-b]pyridine-5-carbonitrile 142 (90 mg, 0.23 mmol) in 4 mL of dichloromethane and 0.5 mL of NMP was cooled to 0° C. and sodium triacetoxyborohydride (242 mg, 1.14 mmol) was added followed by 1-methylpiperazine (0.127 mL, 1.14 mmol). After stirring at room temperature overnight, the reaction mixture was partitioned between saturated aqueous sodium bicarbonate and ethyl acetate. The organic layer was dried over magnesium sulfate, filtered and concentrated in vacuo. The residue was purified by preparative thin layer chromatography developing with 10% methanol in dichloromethane to provide 27 mg of 4-(1H-indol-4-ylamino)-2-{2-[(4-methylpiperazin-1-yl)methyl]phenyl}thieno[2,3-b]pyridine-5-carbonitrile 143 as a glassy solid, MS 479.1 (M+H)⁺.

### EXAMPLE 15

### Preparation of 4-(1H-indol-5-ylamino)-2-{3-[(4-methylpiperazin-1-yl)methyl]phenyl}thieno[2,3-b]pyridine-5-carbonitrile 145

A mixture of 4-(1H-indol-5-ylamino)-2-iodothieno[2,3-b]pyridine-5-carbonitrile hydrochloride 102 (700 mg, 1.55 mmol), tetrakis(triphenylphosphine)palladium (117 mg, 0.10 mmol) and 3-formylphenylboronic acid (378 mg, 2.52 mmol) in 20 mL of saturated aqueous sodium bicarbonate and 35 mL of DME was heated at reflux for 6 hours. The reaction mixture was partitioned between ethyl acetate and water. The aqueous layer was extracted with ethyl acetate, and the organic layers were combined, dried over magnesium sulfate, filtered and concentrated in vacuo. The residue was triturated with diethyl ether and ethyl acetate to provide 200 mg of 2-(3-formylphenyl)-4-(1H-indol-5-ylamino)thieno[2,3-b]pyridine-5-carbonitrile 144. The filtrate was concentrated in vacuo and triturated with methanol to provide an additional amount of 2-(3-formylphenyl)-4-(1H-indol-5-ylamino)thieno[2,3-b]pyridine-5-carbonitrile 144 as a yellow solid, mp 221-223° C., MS 395.1 (M+H).

To a 0° C. mixture of 2-(3-formylphenyl)-4-(1H-indol-5-ylamino)thieno[2,3-b]pyridine-5-carbonitrile 144 (150 mg, 0.38 mmol), and 1-methylpiperazine (0.127 mL, 1.14 mmol) in 4 mL of dichloromethane and 2 mL of NMP was added sodium triacetoxyborohydride (402 mg, 1.89 mmol) followed by 3 drops of acetic acid. After stirring at room temperature overnight, the reaction mixture was partitioned between ethyl acetate and water. The aqueous layer was extracted with ethyl acetate, and the organic layers were combined, dried over magnesium sulfate, filtered and concentrated in vacuo. The residue was purified by flash column chromatography eluting with a gradient of 0 to 15% methanol in dichloromethane to 1% concentrated aqueous ammonium hydroxide in 15% methanol in dichloromethane. The product was triturated with hot diethyl ether to give 58 mg of 4-(1H-indol-5-ylamino)-2-{3-[(4-methylpiperaz 1-yl)methyl]phenyl}thieno[2,3-b]pyridine-5-carbonitrile 145 as a light yellow solid, mp 128-130° C., MS 479.1 (M+H)⁺.

Following the procedure for the preparation of compound 145, 2-(3-formylphenyl)-4-(1-indol-5-ylamino)thieno[2,3-b]pyridine-5-carbonitrile 144 (or another aldehyde such as 2-(5-formyl-3-furyl)-4-(1H-indol-5-ylamino)thieno[2,3-b]pyridine-5-carbonitrile 110 and 2-(5-formyl-3-thienyl)-4-(1H-indol-5-ylamino)thieno[2,3-b]pyridine-5-carbonitrile 199) was reacted with the appropriate amine to provide the following analogs listed in Table 9.

TABLE 9

| Compound | | | MP |
|---|---|---|---|
| Number | Compound Name | MS | (° C.) |
| 146 | 4-(1H-indol-5-ylamino)-2-[3-(morpholin-4- | 466.2 (M + H)+ | 206 |
| | ylmethyl)phenyl]thieno[2,3-b]pyridine-5-carbonitrile | | |
| 147 | 2-{3-[(dimethylamino)methyl]phenyl}-4-(1H-indol-5- | 424.1 (M + H)+ | >245 |
| | ylamino)thieno[2,3-b]pyridine-5-carbonitrile | | |
| 148 | 2-(3-{[4-(2-hydroxyethyl)piperazin-1-yl]methyl}phenyl)-4-(1H- | 509.2 (M + H)+ | 115-118 |
| | indol-5-ylamino)thieno[2,3-b]pyridine-5-carbonitrile | | |
| 149 | 2-(3-{[(2-hydroxyethyl)amino]methyl}phenyl)-4-(1H-indol-5- | 440.1 (M + H)+ | 190-194 |
| | ylamino)thieno[2,3-b]pyridine-5-carbonitrile | | |
| 150 | 4-(1H-indol-5-ylamino)-2-(3-{[4-(2-morpholin-4- | 578.2 (M + H)+ | 131-134 |
| | ylethyl)piperazin-1-yl]methyl}phenyl)thieno[2,3-b]pyridine-5- | | |
| | carbonitrile | | |
| 151 | 2-(3-{[bis(2-hydroxyethyl)amino]methyl}phenyl)-4-(1H-indol-5- | 484.2 (M + H)+ | 192-194 |
| | ylamino)thieno[2,3-b]pyridine-5-carbonitrile | | |
| 152 | 4-(1H-indol-5-ylamino)-2-(3-{[4-(2-phenylethyl)piperazin-1- | 569.3 (M + H)+ | 198-200 |
| | yl]methyl}phenyl)thieno[2,3-b]pyridine-5-carbonitrile | | |
| 153 | 2-{5-[(dimethylamino)methyl]-2-furyl}-4-(1H-indol-5- | 414.2 (M + H)+ | 222-225 |
| | ylamino)thieno[2,3-b]pyridine-5-carbonitrile | | |
| 154 | 2-{5-[(dimethylamino)methyl]-2-furyl}-4-(1H-indol-5- | 414.1 (M + H)+ | 217-220 |
| | ylamino)thieno[2,3-b]pyridine-5-carbonitrile | | |
| 155 | 2-{5-[(dimethylamino)methyl]-2-methoxyphenyl}-4-(1H-indol-5- | 454.2 (M + H)+ | 194-195 |
| | ylamino)thieno[2,3-b]pyridine-5-carbonitrile | | |
| 157 | 2-(5-{[(3S)-3-hydroxypyrrolidin-1-yl]methyl}-3-thienyl)-4-(1H- | 472.2 (M + H)+ | 212-216 |
| | indol-5-ylamino)thieno[2,3-b]pyridine-5-carbonitrile | | |
| 158 | 2-(5-{[(2S)-2-(hydroxymethyl)pyrrolidin-1-yl]methyl}-3-thienyl)- | 486.2 (M + H)+ | 165 softens |
| | 4-(1H-indol-5-ylamino)thieno[2,3-b]pyridine-5-carbonitrile | | |

Following the procedure for the preparation of compound 145, 2-(3-formylphenyl)-4-(1H-indol-5-ylamino)thieno[2,3-b]pyridine-5-carbonitrile 144 was reacted with the appropriate amine to provide the analogs listed in Table 10, which were isolated as the corresponding trifluoroacetic (TFA) salts. The resulting compounds were analyzed by HPLC using the following parameters. An HPLC system from Gilson, Inc. (Middleton, Wis.) with a phenomenex Luna 5 u C18(2) column of dimensions 60×21.20 mm was used. The mobile phase was 20 minutes, and the gradient solvents were 0.02% TFA/H₂O (solvent A) and 0.02% TFA/CH₃CN (solvent B). Compounds were dissolved in either methanol or dimethylsulfoxide. The flow rate was 12.5 mL/min, and detection was carried out at 254 nm and 215 nm.

TABLE 10

| | | | HPLC |
|---|---|---|---|
| Compound | | MS | retention time |
| Number | Compound Name | (M + H) | (Min) |
| 159 | 4-(1H-indol-5-ylamino)-2-[3-(pyrrolidin-1-ylmethyl) | 450.1 | 2.429 |
| | phenyl]thieno[2,3-b]pyridine-5-carbonitrile | | |
| 160 | 4-(1H-indol-5-ylamino)-2-[3-(piperidin-1-ylmethyl) phenyl]thieno[2,3- | 464.1 | 2.475 |
| | b]pyridine-5-carbonitrile | | |
| 161 | 2-{3-[(diethylamino)methyl]phenyl}-4-(1H-indol-5- | 452.1 | 2.45 |
| | ylamino)thieno[2,3-b]pyridine-5-carbonitrile | | |
| 162 | 4-(1H-indol-5-ylamino)-2-(3-{[(2-methoxyethyl)(methyl) | 468.1 | 2.445 |
| | amino]methyl}phenyl)-thieno[2,3-b]pyridine-5-carbonitrile | | |
| 163 | 2-[3-({4-[2-(dimethylamino)ethyl]piperazin-1-yl}methyl) phenyl]-4- | 536.2 | 2.082 |
| | (1H-indol-5-ylamino)thieno[2,3-b]pyridine-5-carbonitrile | | |
| 164 | 2-(3-{[(2-hydroxyethyl)(methyl)amino]methyl} phenyl)-4-(1H-indol- | 454.2 | 2.354 |
| | 5-ylamino)thieno[2,3-b]pyridine-5-carbonitrile | | |
| 165 | 4-(1H-indol-5-ylamino)-2-(3-{[(2-methoxyethyl)amino] | 454.2 | 2.412 |
| | methyl}phenyl)thieno[2,3-b]pyridine-5-carbonitrile | | |
| 166 | 2-[3-({[2-(dimethylamino)ethyl]amino}methyl) phenyl]-4-(1H-indol- | 467.2 | 2.09 |
| | 5-ylamino)thieno[2,3-b]pyridine-5-carbonitrile | | |
| 167 | 2-(3-{[(3-hydroxypropyl)amino]methyl}phenyl)-4-(1H-indol-5- | 454.2 | 2.348 |
| | ylamino)thieno[2,3-b]pyridine-5-carbonitrile | | |
| 168 | 4-(1H-indol-5-ylamino)-2-(3-{[4-(2-oxo-2-pyrrolidin-1- | 576.2 | 2.482 |
| | ylethyl)piperazin-1-yl]methyl}phenyl)thieno[2,3-b] pyridine-5- | | |
| | carbonitrile | | |
| 169 | 4-(1H-indol-5-ylamino)-2-(3-{[4-(pyridin-4-ylmethyl) piperazin-1- | 556.2 | 2.247 |
| | yl]methyl}phenyl)thieno[2,3-b]pyridine-5-carbonitrile | | |
| 170 | 4-(1H-indol-5-ylamino)-2-(3-{[(2-morpholin-4-ylethyl) | 509.1 | 2.318 |
| | amino]methyl}phenyl)thieno[2,3-b]pyridine-5-carbonitrile | | |
| 171 | 2-(3-{[(3S)-3-hydroxypyrrolidin-1-yl]methyl}phenyl)-4-(1H-indol-5- | 466.1 | 2.365 |
| | ylamino)thieno[2,3-b]pyridine-5-carbonitrile | | |
| 172 | 2-(3-{[(3R)-3-hydroxypyrrolidin-1-yl]methyl}phenyl)-4-(1H-indol-5- | 466.1 | 2.363 |
| | ylamino)thieno[2,3-b]pyridine-5-carbonitrile | | |
| 173 | 2-(3-{[3-(hydroxymethyl)piperidin-1-yl]methyl}phenyl)-4-(1H-indol- | 494.1 | 2.397 |
| | 5-ylamino)thieno[2,3-b]pyridine-5-carbonitrile | | |
| 174 | 2-(3-{[4-(hydroxymethyl)piperidin-1-yl]methyl}phenyl)-4-(1H-indol- | 494.1 | 2.381 |
| | 5-ylamino)thieno[2,3-b]pyridine-5-carbonitrile | | |
| 175 | 2-[3-({4-[2-(1H-imidazol-1-yl)ethyl]piperazin-1-yl}methyl)phenyl]-4- | 559.1 | 2.081 |
| | (1H-indol-5-ylamino)thieno[2,3-b]pyridine-5-carbonitrile | | |
| 176 | 2-{3-[(4-hydroxypiperidin-1-yl)methyl]phenyl}-4-(1H-indol-5- | 480.1 | 2.365 |
| | ylamino)thieno[2,3-b]pyridine-5-carbonitrile | | |
| 177 | 2-(3-{[4-(2-hydroxyethyl)piperidin-1-yl]methyl}phenyl)-4-(1H-indol- | 508.1 | 2.408 |
| | 5-ylamino)thieno[2,3-b]pyridine-5-carbonitrile | | |
| 178 | 4-(1H-indol-5-ylamino)2-(3-{[4-(2-methoxyethyl)piperazin-1- | 523.1 | 2.493 |
| | yl]methyl}phenyl)-thieno[2,3-b]pyridine-5-carbonitrile | | |
| 179 | 4-(1H-indol-5-ylamino)2-(3-{[(tetrahydrofuran-2- | 480.1 | 2.455 |
| | ylmethyl)amino]methyl}phenyl)thieno[2,3-b]pyridine-5-carbonitrile | | |
| 180 | 4-(1H-indol-5-ylamino)2-(3-{[(3-morpholin-4- | 523.2 | 2.062 |
| | ylpropyl)amino]methyl}phenyl)thieno[2,3-b]pyridine-5-carbonitrile | | |
| 181 | 2-[3-({4-[2-(2-hydroxyethoxy)ethyl]piperazin-1-yl}methyl)phenyl]-4- | 553.2 | 2.437 |
| | (1H-indol-5-ylamino)thieno[2,3-b]pyridine-5-carbonitrile | | |
| 182 | 2-(3-{[(2-(hydroxymethyl)piperidin-1-yl]methyl}phenyl)-4-(1H-indol- | 494.1 | 2.43 |
| | 5-ylamino)thieno[2,3-b]pyridine-5-carbonitrile | | |
| 183 | 2-[3-({[2-(2-hydroxyethoxy)ethyl]amino}methyl) phenyl]-4-(1H- | 484.1 | 2.357 |
| | indol-5-ylamino)thieno[2,3-b]pyridine-5-carbonitrile | | |

### EXAMPLE 16

### Preparation of 4-(1H-indol-5-ylamino)-2-{2-[(4-methylpiperazin-1-yl)methyl]phenyl}thieno[2,3-b]pyridine-5-carbonitrile 185

A mixture of 4-(1H-indol-5-ylamino)-2-iodothieno[2,3-b]pyridine-5-carbonitrile hydrochloride 102 (400 mg, 0.88 mmol), tetrakis(triphenylphosphine)palladium (78 mg, 0.067 mmol) and 2-formylphenylboronic acid (159 mg, 1.06 mmol) in 12 mL of saturated aqueous sodium bicarbonate and 15 mL of DME was heated at reflux for 4 hours. The reaction mixture was cooled to room temperature and ethyl acetate and water were added. The layers were separated and the organic layer was washed with saturated aqueous sodium bicarbonate followed by water. The organic layer was dried over magnesium sulfate, filtered and concentrated in vacuo. Purification by preparative thin layer chromatography developing with 0.5% concentrated aqueous ammonium hydroxide and 2% methanol in dichloromethane provided 219 mg of 2-(2-formylphenyl)-4-(1H-indol-5-ylamino)thieno[2,3-b]pyridine-5-carbonitrile 184 as a yellow solid, mp 222-224° C., MS 395.1 (M+H)⁺.

To a 0° C. solution of 2-(2-formylphenyl)-4-(1H-indol-5-ylamino)thieno[2,3-b]pyridine-5-carbonitrile 184 (100 mg, 0.25 mmol) in 5 mL of dichloromethane was added 0.5 mL of NMP followed by I-methylpiperazine (0.14 mL, 1.27 mmol) then sodium triacetoxyborohydride (322 mg, 1.52 mmol). After stirring at room temperature overnight, water and ethyl acetate were added. The organic layer was washed with saturated aqueous sodium bicarbonate, dried over magnesium sulfate, filtered and concentrated in vacuo. The residue was purified by preparative thin layer chromatography developing with 1% concentrated aqueous ammonium hydroxide and 10% methanol in dichloromethane to provide 93 mg of 4-(1H-indol-5-ylamino)-2-{2-[(4-methylpiperazin-1-yl)methyl]phenyl}thieno[2,3-b]pyridine-5-carbonitrile 185 as an off-white solid, mp 226-228° C., MS 479.2 (M+H)⁺.

Following the procedure for the preparation of compound 185, 2-(2-formylphenyl)-4-(1H-indol-5-ylamino)thieno[2,3-b]pyridine-5-carbonitrile 184 was reacted with the appropriate amine to provide the analog listed in Table 11.

TABLE 11

| Compound | | | MP |
|---|---|---|---|
| Number | Compound Name | MS | (° C.) |
| 186 | 2-{2-[(4-hydroxypiperidin- | 480.2 | 235-237 |
| | 1-yl)methyl]phenyl}- | (M + H)+ | |
| | 4-(1H-indol-5-ylamino)thieno[2,3- | | |
| | b]pyridine-5-carbonitrile | | |

### EXAMPLE 17

### Preparation of 2-{3-[(dimethylamino)methyl]phenyl}-4-[(4-methyl-1H-indol-5-yl)amino]thieno[2,3-b]pyridine-5-carbonitrile 187

A mixture of 4-chloro-2-iodothieno[2,3-b]pyridine-5-carbonitrile 12 (3.00 g, 9.36 mmol), tetrakis(triphenylphosphine)palladium (541 mg, 0.31 mmol), and 3-formylphenyl boronic acid (1.54 g, 10.30 mmol) in 100 mL of DME and 85 mL of saturated aqueous sodium bicarbonate was heated at reflux for 3 hours. The reaction mixture was cooled to room temperature and the solids were filtered, washing with water, ethyl acetate and diethyl ether. Further washing with dichloromethane, ethyl acetate and diethyl ether gave 2.00 g of 4-chloro-2-(3-formylphenyl)thieno[2,3-b]pyridine-5-carbonitrile 22 as a tan solid, mp>250° C., MS 299.1 (M+H)⁺.

To a 0-5° C. suspension of 4-chloro-2-(3-formylphenyl)thieno[2,3-b]pyridine-5-carbonitrile 22 (1.93 g, 6.46 mmol) and 16.3 mL of 2 M dimethylamine in THF (32.6 mmol) in 80 mL of dichloromethane and 7 mL of DMF was added sodium triacetoxyborohydride (6.80 g, 32.3 mmol). After 5 minutes, 0.25 mL of acetic acid was added and the mixture was keep at 0-5° C. for 5 minutes. The cooling bath was removed and the reaction mixture was stirred at room temperature for 2 hours. Ice was added and the mixture was partitioned between cold saturated aqueous sodium bicarbonate and dichloromethane. The organic layer was washed twice with brine, dried over sodium sulfate, filtered and concentrated in vacuo. The residue was purified by column chromatography eluting with a gradient of 2 to 10% methanol in ethyl acetate to provide 569 mg of 4-chloro-2-{3-[(dimethylamino)methyl]phenyl}thieno[2,3-b]pyridine-5-carbonitrile 24 as a light yellow solid, mp 229-232° C., MS 328.1 (M+H)⁺.

A mixture of 4-chloro-2-{3-[(dimethylamino)methyl]phenyl}thieno[2,3-b]pyridine-5-carbonitrile 24 (120 mg, 0.44 mmol) and 4-methyl-5-aminoindole (78 mg, 0.53 mmol) in 3 mL of 2-ethoxyethanol was heated at 120° C. for 16 hours. An additional 50 mg of 4-methyl-5-aminoindole was added and the mixture was heated at 90° C. for 24 hours. Another additional 28 mg of 4-methyl-5-aminoindole was added and the mixture was heated at 90° C. for 24 hours. The mixture was partitioned between dichloromethane and saturated aqueous sodium bicarbonate. The aqueous phase was extracted with dichloromethane and the combined organic phases were dried over sodium sulfate, filtered and concentrated in vacuo. The residue was purified by column chromatography eluting with a gradient of 2 to 20% methanol in dichloromethane. Further purification by preparative thin layer chromatography developing with 20% methanol in ethyl acetate gave 14 mg of 2-{3-[(dimethylamino)methyl]phenyl}-4-[(4-methyl-1H-indol-5-yl)amino]thieno[2,3-b]pyridine-5-carbonitrile 187 as a tan solid, mp 227-229° C., MS 438.3 (M+H)⁺.

### EXAMPLE 18

### Preparation of 2-[4-(aminomethyl)phenyl]-4-(1H-indol-5-ylamino)thieno[2,3-b]pyridine-5-carbonitrile 188

A mixture of 4-(1H-indol-5-ylamino)-2-iodothieno[2,3-b]pyridine-5-carbonitrile hydrochlorideride 102 (200 mg, 0.44 mmol), tetrakis(triphenylphosphine)palladium (36 mg, 0.031 mmol), 4-aminomethylphenylboronic acid hydrochloride (124 mg, 0.66 mmol) in 8 mL of saturated aqueous sodium bicarbonate and 10 mL of DME was heated at reflux for 4 hours. The reaction mixture was diluted with 20 mL of water and the precipitate was collected by filtration washing with water. The solid was dried in vacuo and purified by flash column chromatography eluting with a gradient of ethyl acetate to 20% methanol in ethyl acetate to 1% concentrated aqueous ammonium hydroxide in 20% methanol in ethyl acetate. Trituration with ethyl acetate and ether followed by preparative thin layer chromatography, developing with 20% methanol in dichloromethane provided 64 mg of 2-[4-(aminomethyl)phenyl]-4-(1H-indol-5-ylamino)thieno[2,3-b]pyridine-5-carbonitrile 188 as a yellow solid, mp>260° C., MS 396.1 (M+H)⁺.

Following the procedure for the preparation of compound 188, the appropriate 2-iodo-thieno[2,3-b]pyridine-5-carbonitrile or 2-bromo-thieno[2,3-b]pyridine-5-carbonitrile was reacted with the appropriate boronic acid or boronic ester to provide the following analogs listed in Table 2. In some cases the boronic acid or boronic ester was generated in situ from the corresponding bromo or iodo analog with n-butyl lithium and an alkyl borate, such as triisopropyl borate.

TABLE 12

| Compound | | | MP |
|---|---|---|---|
| Number | Compound Name | MS | (° C.) |
| 113 | 2-{4-[(dimethylamino)methyl]phenyl}-4-[(4-methyl-1H-indol-5- | 438.2 (M + H)⁺ | 215-217 |
| | yl)amino]thieno[2,3-b]pyridine-5-carbonitrile | | |
| 156 | 2-{2-[(dimethylamino)methyl]phenyl}-4-[(4-methyl-1H-indol-5- | 438.2 (M + H)⁺ | 182 |
| | yl)amino]thieno[2,3-b]pyridine-5-carbonitrile | | |
| 189 | 4-(1H-indol-5-ylamino)-2-(4-morpholin-4-ylphenyl)thieno[2,3- | 452.1 (M + H)⁺ | >260 |
| | b]pyridine-5-carbonitrile | | |
| 190 | 2-[(1E)-4-(4-ethylpiperazin-1-yl)but-1-en-1-yl]-4-(1H-indol-5- | 457.2 (M + H)⁺ | 145-147 |
| | ylamino)thieno[2,3-b]pyridine-5-carbonitrile | | |
| 191 | 2-(5-formyl-2-thienyl)-4-(1H-indol-5-ylamino)thieno[2,3-b]pyridine- | 457.2 (M + H)⁺ | >245 |
| | 5-carbonitrile | | |
| 192 | 2-{4-[2-(dimethylamino)ethyl]phenyl}-4-(1H-indol-5- | 438.2 (M + H)⁺ | 234-235 |
| | ylamino)thieno[2,3-b]pyridine-5-carbonitrile | | |
| 193 | 2-[3-(hydroxymethyl)phenyl]-4-(1H-indol-4-ylamino)thieno[2,3- | 397.2 (M + H)⁺ | 255-258 |
| | b]pyridine-5-carbonitrile | | |
| 194 | 3-[5-cyano-4-(1H-indol-4-ylamino)thieno[2,3-b]pyridine-2-yl]-N,N- | 438.2 (M + H)⁺ | 240 |
| | dimethylbenzamide | | |
| 195 | 3-[5-cyano-4-(1H-indol-5-ylamino)thieno[2,3-b]pyridine-2-yl]-N,N- | 438.3 (M + H)⁺ | 243-248 |
| | dimethylbenzamide | | |
| 196 | 2-[3-(aminomethyl)phenyl]-4-(1H-indol-5-ylamino)thieno[2,3- | 396.1 (M + H)⁺ | >260 |
| | b]pyridine-5-carbonitrile | | |
| 197 | 2-[3-(dimethylamino)phenyl]-4-(1H-indol-5-ylamino)thieno[2,3- | 410.1 (M + H)⁺ | 246-248 |
| | b]pyridine-5-carbonitrile | | |
| 198 | 4-[5-cyano-4-(1H-indol-5-ylamino)thieno[2,3-b]pyridin-2-yl]-N- | 460.0 (M + H)⁺ | >260 |
| | methylbenzenesulfonamide | | |
| 199 | 2-(5-formyl-3-thienyl)-4-(1H-indol-5-ylamino)thieno[2,3-b]pyridine- | 401.0 (M + H)⁺ | >240 |
| | 5-carbonitrile | | |
| 200 | 2-(5-formyl-2-furyl)-4-(1H-indol-5-ylamino)thieno[2,3-b]pyridine-5- | 385.0 (M + H)⁺ | >240 |
| | carbonitrile | | |
| 201 | 2-(3-formylphenyl)-4-[(4-methyl-1H-indol-5-yl)amino] thieno[2,3 | 409.1 (M + H)⁺ | 230-231 |
| | b]pyridine-5-carbonitrile | | |
| 202 | 2-(5-formyl-2-methoxyphenyl)-4-(1H-indol-5-ylamino) thieno[2,3 | 425.1 (M + H)⁺ | 263-265 |
| | b]pyridine-5-carbonitrile | | |
| 230 | 4-{5-cyano-4-[(4-methyl-1H-indol-5-yl)amino]thieno [2,3-b]pyridin- | 474.1 (M + H)⁺ | >260 |
| | 2-yl}-N-methylbenzenesulfonamide | | |
| 231 | 4-[5-cyano-4-(1H-indol-5-ylamino)thieno[2,3-b]pyridin-2-yl]-N,N- | 474.2 (M + H)⁺ | >250 |
| | dimethylbenzenesulfonamide | | |
| 232 | 4-[5-cyano-4-(1H-indol-5-ylamino)thieno[2,3-b]pyridin-2-yl]-N-(2- | 490.2 (M + H)⁺ | 214-216 |
| | hydroxyethyl)benzenesulfonamide | | |
| 233 | 4-[5-cyano-4-(1H-indol-5-ylamino)thieno[2,3-b]pyridin-2-yl]-N- | 528.2 (M + H)⁺ | >250 |
| | cyclohexylbenzenesulfonamide | | |
| 234 | 4-(1H-indol-5-ylamino)-2-[4-(methylsulfonyl)phenyl] thieno[2,3- | 445.1 (M + H)⁺ | >245 |
| | b]pyridine-5-carbonitrile | | |
| 235 | N-{4-[5-cyano-4-(1H-indol-5-ylamino)thieno[2,3-b] pyridin-2- | 459.9 (M + H)⁺ | >245 |
| | yl]phenyl}methanesulfonamide | | |
| 236 | 4-{5-cyano-4-[(4-methyl-1H-indol-5-yl)amino]thieno [2,3-b]pyridin- | 488.2 (M + H)⁺ | >250 |
| | 2-yl}-N,N-dimethylbenzenesulfonamide | | |
| 237 | 3-{5-cyano-4-[(4-methyl-1H-indol-5-yl)amino]thieno [2,3-b]pyridin- | 488.2 (M + H)⁺ | >250 |
| | 2-yl}-N,N-dimethylbenzenesulfonamide | | |
| 238 | 2-{5-cyano-4-[(4-methyl-1H-indol-5-yl)amino]thieno [2,3-b]pyridin- | 488.3 (M + H)⁺ | >260 |
| | 2-yl}-N,N-dimethylbenzenesulfonamide | | |
| 239 | 4-(1H-indol-5-ylamino)-2-[3-(methylsulfonyl)phenyl] thieno[2,3- | 442.9 (M − H)⁻ | >245 |
| | b]pyridine-5-carbonitrile | | |
| 240 | 3-{5-cyano-4-[(4-methyl-1H-indol-5-yl)amino]thieno [2,3-b]pyridin- | 460.2 (M + H)⁺ | >260 |
| | 2-yl}benzenesulfonamide | | |
| 241 | 4-{5-cyano-4-[(4-methyl-1H-indol-5-yl)amino]thieno [2,3-b]pyridin- | 460.2 (M + H)⁺ | >260 |
| | 2-yl}benzenesulfonamide | | |
| 242 | 4-[5-cyano-4-(1H-indol-5-ylamino)thieno[2,3-b]pyridin-2- | 444.0 (M − H)⁻ | >260 |
| | yl]benzenesulfonamide | | |

### EXAMPLE 19

### Preparation of 4-(1H-indol-5-ylamino)-2-{5-[(4-methylpiperazin-1-yl)methyl]pyridine-2-yl}thieno[2,3-b]pyridine-5-carbonitrile 203

A mixture of 4-(1H-indol-5-ylamino)-2-iodothieno[2,3-b]pyridine-5-carbonitrile hydrochloride 102 (250 mg, 2.04 mmol), dichlorobis(triphenylphosphine)palladium(II) (27 mg, 0.038 mmol) and 1-methyl-4-{[6-(tributylstannyl)-3-pyridinyl]methyl}piperazine (980 mg, 2.04 mmol) in 5 mL of dioxane was heated at reflux overnight. The reaction mixture was partitioned between dichloromethane and water. The aqueous layer was extracted with dichloromethane, and the organic layers were combined, dried over magnesium sulfate, filtered and concentrated in vacuo. The residue was purified by flash column chromatography eluting with a gradient of 0 to 20% methanol in dichloromethane to 1% concentrated aqueous ammonium hydroxide in 20% methanol in dichloromethane. Trituration with hot diethyl ether provided 55 mg of 4-(1H-indol-5-ylamino)-2-{5-[(4-methylpiperazin-1-yl)methyl]pyridine-2-yl}thieno[2,3-b]pyridine-5-carbonitrile 203 as a yellow solid, mp>245° C., MS 480.1 (M+H)⁺.

Following the procedure for the preparation of compound 203, the appropriate 2-iodo-thieno[2,3-b]pyridine-5-carbonitrile or 2-bromo-thieno[2,3-b]pyridine-5-carbonitrile was reacted with the appropriate stannane to provide the following analogs listed in Table 13. In some cases, the stannane was generated in situ from the corresponding bromo- or iodo-derivative with hexamethylditin.

TABLE 13

| Compound | | | |
|---|---|---|---|
| Number | Compound Name | MS | MP (° C.) |
| 204 | 2-{5-[(dimethylamino)methyl]pyridin-2-yl}-4-(1H-indol-5- | 425.1 (M + H)⁺ | >245 |
| | ylamino)thieno[2,3-b]pyridine-5-carbonitrile | | |
| 205 | 4-(1H-indol-5-ylamino)-2-(1-methyl-1H-imidazol-5-yl)thieno[2,3- | 371.2 (M + H)⁺ | >250 |
| | b]pyridine-5-carbonitrile | | |
| 206 | 2-(2-formyl-1-methyl-1H-imidazol-5-yl)-4-(1H-indol-5- | 399.1 (M + H)⁺ | >245 |
| | ylamino)thieno[2,3-b]pyridine-5-carbonitrile | | |
| 207 | 2-[5-(1,3-dioxolan-2-yl)-2-thienyl]-4-(1H-indol-5-ylamino)thieno[2,3- | 445.2 (M + H)⁺ | >245 |
| | b]pyridine-5-carbonitrile | | |
| 208 | 2-{2-[(dimethylamino)methyl]-1,3-thiazol-4-yl}-4-(1H-indol-5- | 431.2 (M + H)⁺ | 223-227 |
| | ylamino)thieno[2,3-b]pyridine-5-carbonitrile | | |
| 209 | 6-[5-cyano-4-(1H-indol-5-ylamino)thieno[2,3-b]pyridin-2-yl]-N,N- | 437.3 (M − H)⁻ | >245 |
| | dimethylpyridine-2-carboxamide | | |

### EXAMPLE 20

### Preparation of 4-(1H-indol-5-ylamino)-2-{1-[(4-methylpiperazin-1-yl)methyl]-1H-imidazol-5-yl}thieno[2,3-b]pyridine-5-carbonitrile 210

A solution of 2-(2-formyl-1-methyl-1H-imidazol-5-yl)-4-(1H-indol-5-ylamino)thieno[2,3-b]pyridine-5-carbonitrile 206 (120 mg, 0.30 mmol) in 4 mL of dichloromethane and 0.5 mL of NMP was cooled to 0° C. and 1-methylpiperazine (0.100 mL, 0.90 mmol) was added followed by sodium triacetoxyborohydride (383 mg, 1.81 mmol). After stirring at room temperature overnight, the reaction mixture was partitioned between saturated aqueous sodium bicarbonate and ethyl acetate. The organic layer was dried over magnesium sulfate, filtered and concentrated in vacuo. The residue was purified by preparative thin layer chromatography developing with 10% methanol in dichloromethane. The solid was triturated with methanol and acetone to provide 83 mg of 4-(1H-indol-5-ylamino)-2-{1-[(4-methylpiperazin-1-yl)methyl]-1H-imidazol-5-yl}thieno[2,3-b]pyridine-5-carbonitrile 210 as a light yellow solid, mp>245° C., MS 483.2 (M+H)⁺.

Following the procedure for the preparation of compound 210, 2-(5-formyl-2-thienyl)-4-(1H-indol-5-ylamino)thieno[2,3-b]pyridine-5-carbonitrile 191 and 2-(5-formyl-3-thienyl)-4-(1H-indol-5-ylamino)thieno[2,3-b]pyridine-5-carbonitrile 199 were reacted with the appropriate amine respectively to provide the following analogs listed in Table 14.

TABLE 14

| Compound | | | MP |
|---|---|---|---|
| Number | Compound Name | MS | (° C.) |
| 211 | 2-{5- | 430.3 (M + H)⁺ | 205 |
| | [(dimethylamino)methyl]- | | |
| | 2-thienyl}-4-(1H-indol- | | |
| | 5-ylamino)thieno[2,3- | | |
| | b]pyridine-5-carbonitrile | | |
| 212 | 2-{5- | 430.0 (M + H)⁺ | 215-217 |
| | [(dimethylamino)methyl]- | | |
| | 3-thienyl}-4-(1H-indol- | | |
| | 5-ylamino)thieno[2,3- | | |
| | b]pyridine-5-carbonitrile | | |

### EXAMPLE 21

### Preparation of 4-(1H-indol-5-ylamino)-2-(pyridine-2-ylethynyl)thieno[2,3-b]pyridine-5-carbonitrile 213

A mixture of 4-(1H-indol-5-ylamino)-2-iodothieno[2,3-b]pyridine-5-carbonitrile hydrochloride 102 (150 mg, 0.36 mmol), tetrakis(triphenylphosphine)palladium (21 mg, 0.018 mmol), 2-ethynylpyridine (45 mg, 0.43 minol) and copper iodide (4 mg, 0.022) in 5 mL of dioxane and 2 mL of triethylamine was heated at 95° C. for 2 hours. The reaction mixture was cooled to room temperature and partitioned between dichloromethane and saturated aqueous sodium bicarbonate. The organic layer was dried over sodium sulfate, filtered and concentrated in vacuo. The residue was purified by flash column chromatography eluting with a gradient of 0 to 30% ethyl acetate in dichloromethane. Trituration with ethyl acetate and methanol gave 89 mg of 4(1-H-indol-5-ylamino)-2-(pyridine-2-ylethynyl)thieno[2,3-b]pyridine-5-carbonitrile 213 as a yellow solid, mp>260° C., MS 392.1 (M+H)⁺.

Following the procedure for the preparation of compound 213, a 2-bromo-thieno[2,3-b]pyridine-5-carbonitrile or 2-iodo- thieno[2,3-b]pyridine-5-carbonitrile was reacted with the appropriate ethynyl reagent to provide the following analogs listed in Table 15.

TABLE 15

| Compound | | | MP |
|---|---|---|---|
| Number | Compound Name | MS | (° C.) |
| 214 | 4-(1H-indol-5-ylamino)-2-(pyridin-3-ylethynyl)thieno[2,3- | 392.1 (M + H)⁺ | >260 |
| | b]pyridine-5-carbonitrile | | |
| 215 | 4-(1H-indol-5-ylamino)-2-(phenylethynyl)thieno[2,3-b]pyridine-5- | 391.1 (M + H)⁺ | 295-297 |
| | carbonitrile | | |
| 216 | 4-(1H-indol-5-ylamino)-2-({6-[(4-methylpiperazin-1- | 504.2 (M + H)⁺ | 230 dec |
| | yl)methyl]pyridin-2-yl}ethynyl)thieno[2,3-b]pyridine-5-carbonitrile | | |
| 217 | 2-({6-[(dimethylamino)methyl]pyridin-2-yl}ethynyl)-4-(1H-indol-5- | 449.2 (M + H)⁺ | 182-185 |
| | ylamino)thieno[2,3-b]pyridine-5-carbonitrile | | |
| 218 | 4-(1H-indol-4-ylamino)-2-(pyridin-3-ylethynyl)thieno[2,3- | 392.2 (M + H)⁺ | >250 |
| | b]pyridine-5-carbonitrile | | |
| 219 | 2-({6-[(dimethylamino)methyl]pyridin-2-yl}ethynyl)-4-(1H-indol-4- | 449.1 (M + H)⁺ | 230-233 |
| | ylamino)thieno[2,3-b]pyridine-5-carbonitrile | | |
| 220 | 2-({6-[(dimethylamino)methyl]pyridin-2-yl}ethynyl)-4-(4-methyl- | 463.2 (M + H)⁺ | 235-237 |
| | 1H-indol-5-ylamino)thieno[2,3-b]pyridine-5-carbonitrile | | |

### EXAMPLE 22

### Preparation of 4-(1H-indol-5-ylamino)-2-[4-(4-methylpiperazin-1-yl)but-1-yn-1-yl]-thieno[2,3-b]pyridine-5-carbonitrile 221

A mixture of 4-(1H-indol-5-ylamino)-2-iodothieno[2,3-b]pyridine-5-carbonitrile hydrocloride 102 (200 mg, 0.44 mmol), dichlorobis(triphenylphosphine)palladium(II) (15 mg, 0.022 mmol), 1-but-3-ynyl-4-methylpiperazine (167 mg, 1.1 mmol), copper iodide (4 mg, 0.022 mmol), potassium carbonate (304 mg, 2.2 mmol) and triphenylphosphine (23 mg, 0.088 mmol) in 3 mL of THF and 0.6 mL of methanol was heated at 60° C. for 3 hours. The reaction mixture was cooled to room temperature and partitioned between dichloromethane and water. The aqueous layer was extracted with dichloromethane, and the organic layers were combined, dried over sodium sulfate, filtered and concentrated in vacuo. The residue was purified by flash column chromatography eluting with a gradient of 0 to 20% methanol in ethyl acetate to 1% concentrated aqueous ammonium hydroxide in 20% methanol in ethyl acetate. Trituration with diethyl ether and methanol gave 73 mg of 4-(1H-indol-5-ylamino)-2-[4-(4-methylpiperazin-1-yl)but-1-yn-1-yl]thieno[2,3-b]pyridine-5-carbonitrile 221 as a yellow solid, mp 195-197° C., MS 441.2 (M+H)⁺.

Following the procedure for the preparation of compound 221, the appropriate 2-iodo-thieno[2,3-b]pyridine-5-carbonitrile or 2-bromo-thieno[2,3-b]pyridine-5-carbonitrile was reacted with the appropriate ethynyl reagent to provide the following analogs listed in Table 16.

TABLE 16

| Compound | | | MP |
|---|---|---|---|
| Number | Compound Name | MS | (° C.) |
| 222 | 4-(1H-indol-4-ylamino)-2-[4-(4-methylpiperazin-1-yl)but-1-yn- | 441.2 (M + H)⁺ | 210-212 |
| | 1-yl]thieno[2,3-b]pyridine-5-carbonitrile | | |
| 223 | 2-[3-(dimethylamino)prop-1-yn-1-yl]-4-(1H-indol-5- | 372.1 (M + H)⁺ | 233-235 |
| | ylamino)thieno[2,3-b]pyridine-5-carbonitrile | | |
| 224 | 4-(1H-indol-5-ylamino)-2-[(trimethylsilyl)ethynyl]thieno[2,3- | 387.2 (M + H)⁺ | 239-240 |
| | b]pyridine-5-carbonitrile | | |
| 225 | 2-[3-(diethylamino)prop-1-yn-1-yl]-4-(1H-indol-5- | 400.3 (M + H)⁺ | 211 |
| | ylamino)thieno[2,3-b]pyridine-5-carbonitrile | | |

### EXAMPLE 23

### Preparation of 4-(1H-indol-5-ylamino)-2-(pyridine-4-ylethynyl)thieno[2,3-b]pyridine-5-carbonitrile 226

A mixture of 4-(1H-indol-5-ylamino)-2-[(trimethylsilyl)ethynyl]thieno[2,3-b]pyridine-5-carbonitrile 224 (200 mg, 0.52 mmol), dichlorobis(triphenylphosphine) palladium(II) (18 mg, 0.026 mmol), 4-iodopyridine (139 mg, 0.68 mmol), copper iodide (5 mg, 0.026 mmol), potassium carbonate (288 mg, 2.08 mmol) and triphenylphosphine (27 mg, 0.104 mmol) in 6 mL of THF and 1.5 mL of methanol was heated at 65° C. for 2.5 hours. The reaction mixture was cooled to room temperature and partitioned between dichloromethane and water. The organic phase was dried over sodium sulfate, filtered and concentrated in vacuo. The residue was purified by flash column chromatography eluting with a gradient of 0 to 20% ethyl acetate in dichloromethane to provide 4-(1H-indol-5-ylamino)-2-(pyridine-4-ylethynyl)thieno[2,3-b]pyridine-5-carbonitrile 226 as a yellow solid, mp>250° C., MS 392.2 (M+H)⁺.

Following the procedure for the preparation of compound 226, 4-(1H-indol-5-ylamino)-2-[(trimethylsilyl)ethynyl]thieno[2,3-b]pyridine-5-carbonitrile 224 was reacted with the appropriate aromatic or heteroaryl halide to provide the following analogs listed in Table 17.

TABLE 17

| Compound | | | MP |
|---|---|---|---|
| Number | Compound Name | MS | (° C.) |
| 227 | 4-(1H-indol-5-ylamino)-2-(1H-pyrazol-4-ylethynyl)thieno[2,3- | 381.2 (M + H)+ | >260 |
| | b]pyridine-5-carbonitrile | | |
| 228 | 2-[(2-aminopyrimidin-5-yl)ethynyl]-4-(1H-indol-5- | 408.0 (M + H)+ | >260 |
| | ylamino)thieno[2,3-b]pyridine-5-carbonitrile | | |
| 229 | 2-({5-[(dimethylamino)methyl]pyridin-2-yl}ethynyl)4-(1H-indol-5- | 449.3 (M + H)+ | 205 |
| | ylamino)thienol[2,3-b]pyridine-5-carbonitrile | | |

### EXAMPLE 24

### Preparation of 4-(1H-indol-5-ylamino)-3-[4-(4-methylpiperazin-1-yl)but-1-yn-1-yl]thieno[2,3-b]pyridine-5-carbonitrile 244

Bromine (0.878 mL, 17.06 mmol) was added dropwise to a suspension of 4-chlorothieno[2,3-b]pyridine-5-carbonitrile 10 (1.66 g, 8.53 mmol) in 23 mL of acetic acid. The resulting mixture was heated at 80° C. for 24 hours. Additional bromine (0.878 mL) was added and heating at 80° C. was continued. After 24 hours, additional bromine (0.878 mL) was added and heating at 80° C. was resumed for another 24 hours. The mixture was cooled to room temperature and concentrated in vacuo. The residue was cooled to 0-5° C. and neutralized with saturated aqueous sodium bicarbonate and extracted with dichloromethane. The organic phase was washed twice with brine, dried over sodium sulfate, filtered and concentrated in vacuo. The residue was purified by column chromatography eluting with a gradient of 0 to 70% dichloromethane in hexane followed by all dichloromethane to provide 694 mg of 3,4-dibromothieno[2,3-b]pyridine-5-carbonitrile 16 as a white solid, mp 204-206° C., MS 315.8 (M−H)⁻. Additional fractions provided 831 mg of a mixture of 3,4-dibromothieno[2,3-b]pyridine-5-carbonitrile and 3-bromo-4-chlorothieno[2,3-b]pyridine-5-carbonitrile.

A mixture of 3,4-dibromothieno[2,3-b]pyridine-5-carbonitrile 16 (674 mg, 2.12 mmol) and 5-aminoindole (308 mg, 2.33 mmol) in 12 mL of ethanol was heated at reflux for 66 hours. The reaction mixture was cooled and the solid collected by filtration, followed by washing with ethanol. The solid was dried in vacuo to give 649 mg of 3-bromo-4-(1H-indol-5-ylamino)thieno[2,3-b]pyridine-5-carbonitrile hydrobromide 243 as a gray solid, mp 249-251° C., MS 369.0 (M+H)⁺.

A mixture of 3-bromo-4-(1H-indol-5-ylamino)thieno[2,3-b]pyridine-5-carbonitrile hydrobromide 243 (200 mg, 0.54 mmol), dichlorobis(triphenylphosphine)palladium(II) (19 mg, 0.03 mmol), 1-but-3-ynyl-4-methylpiperazine (205 mg, 1.35 mmol), copper iodide (5 mg, 0.03 mmol), potassium carbonate (373 mg, 2.7 mmol) and triphenylphosphine (28 mg, 0.11 mmol) in 5 mL of THF and 1 mL of methanol was heated at 60° C. for 4.5 hours. The reaction mixture was cooled to room temperature and partitioned between dichloromethane and brine. The organic layer was washed with brine, dried over sodium sulfate, filtered and concentrated in vacuo. The residue was purified by flash column chromatography eluting with a gradient of 0 to 20% methanol in ethyl acetate to 1% aqueous ammonium hydroxide in 20% methanol in ethyl acetate. Trituration with diethyl ether and ethyl acetate gave 131 mg of 4-(1H-indol-5-ylamino)-3-[4-(4-methylpiperazin-1-yl)but-1-yn-1-yl]thieno[2,3-b]pyridine-5-carbonitrile 244 as a yellow solid, mp 184-186° C., MS 441.2 (M+H)⁺.

### EXAMPLE 25

### Alternative Preparation of 4-chloro-2-iodothieno[2,3-b]pyridine-5-carbonitrile 12

Methyl 2-aminothiophene-3-carboxylate (80 g, 510 mmol) was treated with 250 mL of dimethylformamide-dimethylacetal and heated to 100° C. After heating overnight, the reaction was cooled and concentrated to give a dark oil. Tert-butanol (450 mL) was added to the residue followed by t-butyl cyanoacetate (132 g, 1020 mmol). The reaction was stirred for 4 days at room temperature. The resulting thick precipitate was filtered and washed extensively with t-butanol until the washings ran clear. The pale yellow solid was dried under vacuum to give 77 grams of methyl 2-{[(1E)-3-tert-butoxy-2-cyano-3-oxoprop-1-en-1-yl]amino}thiophene-3-carboxylate (50% yield). The mother liquor yielded an additional 10 grams of methyl 2-{[(1E)-3-tert-butoxy-2-cyano-3-oxoprop-1-en-1-yl]amino}thiophene-3-carboxylate after partial concentration and standing for several days at room temperature, mp 154-157° C.; MS (ESI) m/z 306.9 (M+H).

Diphenyl ether (250 mL) was heated to a gentle reflux using a heating mantle. Nitrogen was bubbled into the diphenyl ether as it was heating to reflux and then gently blown over the top of the solvent during the course of the reaction. Methyl 2-{[(1E)-3-tert-butoxy-2-cyano-3-oxoprop-1-en-1-yl]amino}thiophene-3-carboxylate (14 g, 45 mmol) was added in portions over a few minutes. The reaction was heated to a gentle reflux for 3 hours then cooled to room temperature. Hexane (500 mL) was added and the resultant precipitate was filtered and washed extensively with hexane. The residual diphenyl ether could be removed by stirring the solid for several hours in hexane followed by filtration giving 7.25 g of 4-hydroxythieno[2,3-b]pyridine-5-carbonitrile as a dark powder (91%), MS (ESI) m/z 174.9 (M+H).

4-Hydroxythieno[2,3-b]pyridine-5-carbonitrile (5.0 g, 28.4 mmol) was stirred as a suspension in 500 mL of CHCl₃. To the above slurry was added sequentially [bis(trifluoroacetoxy)iodo]benzene (18.3 g, 42.6 mmol) and iodine (10.8 g, 42.6 mmol). The mixture was stirred at room temperature for 24 hours then concentrated to approximately 150 mL. The resultant solid was filtered and the solid was washed extensively with hexane until the washings ran clear. The resultant brown solid (7.9 g) was treated with phosphorus oxychloride (60 mL) and DMF (0.6 mL) and heated to 70° C. overnight. The reaction was carefully poured over ice and the product was filtered and washed with water to give 8.0 g of 4-chloro-2-iodothieno[2,3-b]pyridine-5-carbonitrile 12 as a brown solid. The crude product was generally used directly in subsequent steps but could be further purified by column chromatography (EtOAc/hexane), MS (ESI) m/z 320.9 (M+H).

### EXAMPLE 26

### Preparation of Additional 4-chloro-2-iodothieno[2,3-b]pyridine-5-carbonitriles

### Preparation of 4-chloro-2-iodo-3-methylthieno2,3-pyridine-5-carbonitrile Following the Procedure Described in Example 25

Ethyl 2-{[(1E)-3-tert-butoxy-2-cyano-3-oxoprop-1-en-1-yl]amino}-4-methylthiophene-3-carboxylate was prepared from ethyl 2-amino-4-methylthiophene-3-carboxylate, mp 144° C.; MS (ESI) m/z 335; HPLC retention time=19.3 min.

3-Methyl-4-oxo-4,7-dihydrothieno[2,3-b]pyridine-5-carbonitrile was prepared from ethyl 2-{[(1E)-3-tert-butoxy-2-cyano-3-oxoprop-1-en-1-yl]amino}-4-methylthiophene-3-carboxylate, mp 285° C.; MS (ESI) m/z 188.9; HPLC retention time=6.2 min.

4-Chloro-2-iodo-3-methylthieno[2,3-b]pyridine-5-carbonitrile was prepared from 3-methyl-4-oxo-4,7-dihydrothieno[2,3-b]pyridine-5-carbonitrile, MS (APCI) m/z 333.8; HPLC retention time=18.1 min.

### Preparation of 4-chloro-2-iodo-3-isopropylthieno[2,3-b]pyridine-5-carbonitrile Following the Procedure Described in Example 25

Ethyl 2-{[(1E)-3-tert-butoxy-2-cyano-3-oxoprop-1-en-1-yl]amino}-4-isopropylthiophene-3-carboxylate was prepared from ethyl 2-amino-4-isopropylthiophene-3-carboxylate, mp 93-94° C.; MS (ESI) m/z 363.3.

3-Isopropyl-4-oxo-4,7-dihydrothieno[2,3-b]pyridine-5-carbonitrile was prepared from ethyl 2-{[(1E)-3-tert-butoxy-2-cyano-3-oxoprop-1-en-1-yl]amino}-4-isopropylthiophene-3-carboxylate, mp 285° C.; MS (ESI) m/z 188.9.

2-Iodo-3-isopropyl-4-oxo-4,7-dihydrothieno[2,3-b]pyridine-5-carbonitrile was obtained by treatment of 3-isopropyl-4-oxo-4,7-dihydrothieno[2,3-b]pyridine-5-carbonitrile with 1 M iodine monochloride in dichloromethane and NaOAc in MeOH, MS (ESI) m/z 345.1.

4-Chloro-2-iodo-3-isopropylthieno[2,3-b]pyridine-⁵-carbonitrile was prepared from 2-iodo-3-isopropyl-4-oxo-4,7-dihydrothieno[2,3-b]pyridine-5-carbonitrile, mp 177-179° C., MS (ESI) m/z 363.1.

### EXAMPLE 27

### Preparation of methyl 5-cyano-4-[(4-methyl-1H-indol-5-yl)amino]thieno[2,3-b]pyridine-2-carboxylate 245

4-Chloro-5-cyanothieno[2,3-b]pyridine 10 (3.0 g, 15.4 mmol) was stirred in 100 mL THF and cooled to −78° C. LDA (19.25 mmol, 2 M solution in THF) was added slowly and the reaction was stirred for half an hour at −78° C. Carbon dioxide (generated via dry ice) was bubbled into the reaction and the reaction was allowed to slowly warm to room temperature. The reaction was quenched with 30 mL of 1M HCl and diluted with water. The product was extracted into EtOAc and concentrated to give 3.1 g of 4-chloro-5-cyanothieno[2,3-b]pyridine-2-carboxylic acid as an orange solid that was used without further purification, MS (ESI) m/z 236.8 (M−H).

4-Chloro-5-cyanothieno[2,3-b]pyridine-2-carboxylic acid (3.1 g) was dissolved in 100 mL THF and treated with 15 mL of 2M trimethylsilyl diazomethane in THF. After half an hour, the reaction was carefully quenched with HOAc (1.2 mL), concentrated and purified by chromatography (EtOAc/hexane) to give 1.3 g of methyl 4-chloro-5-cyanothieno[2,3-b]pyridine-2-carboxylate as a yellow solid, MS (APCI) m/z 253.1; HPLC retention time=13.1 min.

Methyl 4-chloro-5-cyanothieno[2,3-b]pyridine-2-carboxylate (1.3 g, 5.1 mmol) and 4-methyl-5-aminoindole (0.98 g, 6.7 mmol) were heated to reflux in 50 mL MeOH for 1 hour. An additional 0.35 g of 4-methyl-5-amino indole was added and the heating was continued for 3 hours. The reaction was cooled to room temperature and the resultant precipitate was filtered and washed with MeOH to give 1.3 g of methyl 5-cyano-4-[(4-methyl-1H-indol-5-yl)amino]thieno[2,3-b]pyridine-2-carboxylate 245, mp 255° C., MS (ESI) m/z 363.2 (M+H), HPLC retention time=14.2 min.

Methyl 5-cyano-4-(1H-indol-5-ylamino)thieno[2,3-b]pyridine-2-carboxylate 246 was prepared via the route used to prepare compound 245 using 5-aminoindole, MS (ESI) m/z 349.2 (M+H); HPLC retention time=13.3 min.

### EXAMPLE 28

### Preparation of 5-cyano-4-[(4-methyl-1H-indol-5-yl)amino]thieno[2,3-b]pyridine-2-carboxylic acid 247

Methyl 5-cyano-4-[(4-methyl-1H-indol-5-yl)amino]thieno[2,3-b]pyridine-2-carboxylate 245 (0.6 g, 1.7 mmol) was stirred as a suspension in 15 mL MeOH and 5 mL THF. The reaction was treated with 3.3 mL of 1 M NaOH and stirred overnight. The clear solution was treated with 5 mL of 1 M HCl and 5 mL water. After stirring for 1 hour, a thick yellow precipitate formed and was filtered and dried to give 507 mg of 5-cyano-4-[(4-methyl-1H-indol-5-yl)amino]thieno[2,3-b]pyridine-2-carboxylic acid 247, mp 287° C.; HPLC retention time=11.1 min; MS (ESI) m/z 349.2 (M+H).

5-Cyano-4-(1H-indol-5-ylamino)thieno[2,3-b]pyridine-2-carboxylic acid 248 was prepared according to the route used to prepare compound 247, mp>250° C.; MS (ESI) m/z 335.2 (M+H); HPLC retention time=10.4 min.

### EXAMPLE 29

### Preparation of 4-[(4-methyl-1H-indol-5-yl)amino]-2-(pyrrolidin-1-ylcarbonyl)thieno[2,3-b]pyridine-5-carbonitrile 249

5-Cyano-4-[(4-methyl-1H-indol-5-yl)amino]thieno[2,3-b]pyridine-2-carboxylic acid 247 (75 mg, 0.21 mmol) was stirred in 5 mL dichloromethane and treated with pyrolidine (30 mg, 0.42 mmol) and EDC (N-(3-dimethylaminopropyl)-N′-ethylcarbodiimide hydrochloride) (80 mg, 0.42 mmol). After stirring overnight the reaction was evaporated onto silica gel and purified by chromatography (EtOAc/hexane) to give 4-[(4-methyl-1H-indol-5-yl)amino]-2-(pyrrolidin-1-ylcarbonyl)thieno[2,3-b]pyridine-5-carbonitrile 249, mp 256° C., HPLC retention time=17.7 min; MS (ESI) m/z 402.2 (M+H).

The following analogs shown in Table 18 were made by the procedure used to prepare 4-[(4-methyl-1H-indol-5-yl)amino]-2-(pyrrolidin-1-ylcarbonyl)thieno[2,3-b]pyridine-5-carbonitrile 249.

TABLE 18

| | | | | HPLC |
|---|---|---|---|---|
| Compound | | | | Retention |
| Number | Compound Name | MS | MP (° C.) | time (min) |
| 250 | 4-(1H-indol-5-ylamino)-2-(pyrrolidin-1- | 388.0 (M + H) | 245-265 | 12.2 |
| | ylcarbonyl)thieno[2,3-b]pyridine-5-carbonitrile | | | |
| 251 | 5-cyano-4-(1H-indol-5-ylamino)-N-pyridin-3- | 411.1 (M + H) | 185 (dec) | 8.1 |
| | ylthieno[2,3-b]pyridine-2-carboxamide | | | |
| 252 | 5-cyano-4-(1H-indol-5-ylamino)-N-pyridin-4- | 411.1 (M + H) | 180 (dec) | 7.4 |
| | ylthieno[2,3-b]pyridine-2-carboxamide | | | |
| 253 | 4-(1H-indol-5-ylamino)-2-[(4-methylpiperazin-1- | 417.2 (M + H) | 120 | 6.2 |
| | yl)carbonyl]thieno[2,3-b]pyridine-5-carbonitrile | | | |
| 254 | 5-cyano-N-(2-hydroxyethyl)-4-(1H-indol-5- | 375.8 (M + H) | 280 (dec) | 8.7 |
| | ylamino)thieno[2,3-b]pyridine-2-carboxamide | | | |
| 255 | 4-[(4-methyl-1H-indol-5-yl)amino]-2-(morpholin- | 418.3 (M + H) | 237 | 11.3 |
| | 4-ylcarbonyl)thieno[2,3-b]pyridine-5-carbonitrile | | | |
| 256 | 4-[(4-methyl-1H-indol-5-yl)amino]-2-[(4- | 431.3 (M + H) | >290 | 6.7 |
| | methylpiperazin-1-yl)carbonyl]thieno[2,3- | | | |
| | b]pyridine-5-carbonitrile | | | |
| 257 | 5-cyano-4-[(4-methyl-1H-indol-5-yl)amino]-N- | 425.2 (M + H) | >290 | 7.8 |
| | pyridin-4-ylthieno[2,3-b]pyridine-2-carboxamide | | | |
| 258 | 5-cyano-4-[(4-methyl-1H-indol-5-yl)amino]-N- | 424.3 (M + H) | N/A | 14.5 |
| | phenylthieno[2,3-b]pyridine-2-carboxamide | | | |
| 259 | N-benzyl-5-cyano-4-[(4-methyl-1H-indol-5- | 438.3 (M + H) | N/A | 13.9 |
| | yl)amino]thieno[2,3-b]pyridine-2-carboxamide | | | |
| 260 | 5-cyano-4-[(4-methyl-1H-indol-5-yl)amino]-N-(2- | 452.3 (M + H) | 155 | 14.5 |
| | phenylethyl)thieno[2,3-b]pyridine-2-carboxamide | | | |
| 261 | 5-cyano-N,N-dimethyl-4-[(4-methyl-1H-indol-5- | 376.3 (M + H) | 144 | 11.3 |
| | yl)amino]thieno[2,3-b]pyridine-2-carboxamide | | | |
| 262 | 5-cyano-N-(2-methoxyethyl)-4-[(4-methyl-1H- | 406.3 (M + H) | 260 | 8.5 |
| | indol-5-yl)amino]thieno[2,3-b]pyridine-2- | | | |
| | carboxamide | | | |
| 263 | 5-cyano-4-[(4-methyl-1H-indol-5-yl)amino]-N- | 425.3 (M + H) | 193 | 10.9 |
| | pyridin-3-ylthieno[2,3-b]pyridine-2-carboxamide | | | |
| 264 | 4-(1H-Indol-4-ylamino)-2-(pyrrolidin-1- | 388.2 (M + H) | N/A | N/A |
| | ylcarbonyl)thieno[2,3-b]pyridine-5-carbonitrile | | | |
| 265 | 4-[(4-methyl-1H-indol-5-yl)amino]-2-(piperazin-1- | 417.3 | 253 | 6.7 |
| | ylcarbonyl)thieno[2,3-b]pyridine-5-carbonitrile | | | |
| 266 | 5-cyano-4-[(4-methyl-1H-indol-5-yl)amino]-N- | 431.3 (M + H) | >260 | 6.9 |
| | piperidin-4-ylthieno[2,3-b]pyridine-2-carboxamide | | | |

The last two analogs were prepared via a Boc protected piperazine and piperidine intermediate, respectively, where the Boc group was removed by treatment with 4M HCl in dioxane.

### EXAMPLE 30

### Preparation of 4-(1H-indol-5-ylamino)-2-(pyrrolidin-1-ylmethyl)thieno[2,3-b]pyridine-5-carbonitrile 267

4-Chlorothieno[2,3-b]pyridine-5-carbonitrile 10 (400 mg, 2.05 mmol) was stirred in 20 mL dry THF and cooled to −78° C. LDA (2.9 mmol) was added dropwise as a 2M solution in THF. The reaction was stirred at −78° C. for 10 minutes then quenched with 0.6 mL of DMF. After stirring briefly, the reaction was further quenched with saturated aqueous ammonium chloride and warmed to room temperature. The crude reaction mixture was diluted with 1M HCl and the product was extracted into EtOAc giving 330 mg of 4-chloro-2-formylthieno[2,3-b]pyridine-5-carbonitrile as a dark solid. The product was generally used without further purification but an analytical sample could be obtained by silica gel chromatography (EtOAc/hexane), mp 184-185° C.; MS (ESI-FTMS) m/z 223.0.

4-Chloro-2-formylthieno[2,3-b]pyridine-5-carbonitrile (100 mg, 0.45 mmol) was stirred in 10 mL dichloroethane and treated with pyrolidine (0.63 mmol) and HOAc (0.68 mmol). After stirring for 15 minutes, sodium triacetoxy borohydride (0.90 mmol) was added and the reaction was stirred at room temperature for half an hour. The crude reaction was concentrated and purified by preparative HPLC. The purified product was refluxed in EtOH with 5-aminoindole (1.4 eq) for 9 hours. The reaction was diluted with aqueous sodium bicarbonate and the product was extracted into dichloromethane three times. 4-(1H-Indol-5-ylamino)-2-(pyrrolidin-1-ylmethyl)thieno[2,3-b]pyridine-5-carbonitrile 267 was purified by silica gel chromatography (EtOAc/hexane to remove impurities, then elution with dichloromethane/MeOH), mp 212-215° C.; MS (ESI-FTMS) m/z 374.1 (M+H); HPLC retention time=6.8 min.

The analogs in Table 19 were prepared via the procedure used to prepare compound 267.

TABLE 19

| | | | | HPLC |
|---|---|---|---|---|
| Compound | | | | Retention |
| Number | Compound Name | MS | MP (° C.) | time (min) |
| 268 | 2-(3,4-dihydroisoquinolin-2(1H)-ylmethyl)-4-(1H- | (ESI-FTMS) | 209 (dec) | 5.4 |
| | indol-5-ylamino)thieno[2,3-b]pyridine-5-carbonitrile | m/z 436.2 (M + H) | | |
| 269 | 4-(1H-indol-5-ylamino)-2-[(4-phenylpiperazin-1- | (ESI-FTMS) | 230 (dec) | 5.8 |
| | yl)methyl]thieno[2,3-b]pyridine-5-carbonitrile | m/z 465.2 (M + H) | | |

### EXAMPLE 31

### Preparation of 2-[(1E)-buta-1,3-dien-1-yl]-4-(1H-indol-5-ylamino)thieno[2,3-b]pyridine-5-carbonitrile 270 and 2-butyl-4-(1H-indol-5-ylamino)thieno[2,3-b]pyridine-5-carbonitrile 271

2-Iodo-4-(1H-indol-5-ylamino)thieno[2,3-b]pyridine-5-carbonitrile 102 (100 mg, 0.24 mmol) was dissolved in 2 mL DMF and treated with (E)-4-(3,3,4,4-tetramethylborolan-1-yl)but-3-enyl 4-methylbenzenesulfonate (127 mg, 0.36 mmol), tetrakis(triphenylphosphine) palladium(0) (15 mg) and cesium carbonate (156 mg, 0.48 mmol). The reaction mixture was heated to 110° C. for 10 minutes by microwave irradiation. The reaction was diluted with water and the product was extracted into EtOAc and purified by silica gel chromatography (EtOAc/hexane) to give 69 mg of 2-[(1E)-buta-1,3-dien-1-yl]-4-(1H-indol-5-ylamino)thieno[2,3-b]pyridine-5-carbonitrile 270, MS (ESI) m/z 343.1 (M+H).

2-[(1E)-buta-1,3-dien-1-yl]-4-(1H-indol-5-ylamino)thieno[2,3-b]pyridine-5-carbonitrile 270 (50 mg) was stirred in 10 mL EtOH and treated with 50 mg of 10% (wet) Pd/C. After stirring for half an hour under an atmosphere of hydrogen, the reaction was filtered through Celite® and concentrated to give 2-butyl-4-(1H-indol-5-ylamino)thieno[2,3-b]pyridine-5-carbonitrile 271, mp 210° C.; MS (ESI) m/z 347.1 (M+H); HPLC retention time=10.9 min.

### EXAMPLE 32

### Preparation of 4-[(4-methyl-1H-indol-5-yl)amino]-2-[(1E)-4-(4-methylpiperazin-1-yl)but-1-en-1-yl]thieno[2,3-b]pyridine-5-carbonitrile 272

2-Iodo-4-[(4-methyl-1H-indol-5-yl)amino]thieno[2,3-b]pyridine-5-carbonitrile 123 (200 mg, 0.48 mmol), (E)-4-(4,4,5,5-tetramethyl-1,3,2-dioxaborolan-2-yl)but-3-enyl 4-methylbenzenesulfonate (246 mg, 0.70 mmol), cesium carbonate (306 mg, 0.94 mmol), N-methylpiperazine (94 mg, 0.94 mmol) and tetrakis(triphenylphosphine) palladium(0) (10 mg) were stirred in 5 mL DMF and heated to 70° C. overnight. The reaction mixture was partitioned between EtOAc and water. The crude product was extracted twice into EtOAc and purified by silica gel chromatography (dichloromethane/MeOH/NH₃). The HCl salt was generated by treatment of the purified amine with excess HCl/dioxane. The hydrochloride salt of 4-[(4-methyl-1H-indol-5-yl)amino]-2-[(1E)-4-(4-methylpiperazin-1-yl)but-1-en-1-yl]thieno[2,3-b]pyridine-5-carbonitrile 272 was obtained as a while solid upon trituration with EtOH, MS (ESI) m/z 457.4 (M+H); HPLC retention time=7.1 min.

4-(1H-Indol-5-ylamino)-2-[(1E)-4-(4-methylpiperazin-1-yl)but-1-en-1-yl]thieno[2,3-b]pyridine-5-carbonitrile 273 was prepared via the route used to prepare compound 272, mp 220° C.; MS (ESI) m/z 443.3 (M+H).

### EXAMPLE 33

### Preparation of 4-(1H-indol-5-ylamino)-2-[4-(4-methylpiperazin-1-yl)butyl]thieno[2,3-b]pyridine-5-carbonitrile 274

4-(1H-Indol-5-ylamino)-2-[(1E)-4-(4-methylpiperazin-1-yl)but-1-en-1-yl]thieno[2,3-b]pyridine-5-carbonitrile 273 (120 mg) and 50 mg of Pd/C (10%, wet) in 30 mL EtOH were stirred under an atmosphere of hydrogen overnight. The reaction mixture was filtered and concentrated. 4-(1H-Indol-5-ylamino)-2-[4-(4-methylpiperazin-1-yl)butyl]thieno[2,3-b]pyridine-5-carbonitrile 274 was purified by preparative HPLC, mp 120° C. (dec.); MS (ESI) m/z 445.3 (M+H); HPLC retention time=6.6 min.

### EXAMPLE 34

### Preparation of 4-[(4-methyl-1H-indol-5-yl)amino]-2-[(1E)-3-morpholin-4-ylprop-1-en-1-yl]thieno[2,3-b]pyridine-5-carbonitrile 275

2-Iodo-4-[(4-methyl-1H-indol-5-yl)amino]thieno[2,3-b]pyridine-5-carbonitrile 123 (150 mg, 0.35 mmol), (E)-3-chloroprop-1-enylboronic acid (105 mg, 0.87 mmol), cesium carbonate (400 mg, 1.22 mmol), morpholine (76 mg, 0.87 mmol) and bis(triphenylphosphine)palladium(II)dichloride (20 mg) in 5 mL of DMF were heated to 130° C. by microwave-irradiation for 30 minutes. The reaction was cooled, filtered, and purified by preparative HPLC to give 4-[(4-methyl-1H-indol-5-yl)amino]-2-[(1E)-3-morpholin-4-ylprop-1-en-1-yl]thieno[2,3-b]pyridine-5-carbonitrile 275. The HCl salt was generated by addition of excess HCl/dioxane, mp 230° C. (dec.); HPLC retention time=7.9 min.; MS (ESI) m/z 430.1 (M+H).

The analogs in Table 20 were prepared from various 2-iodothieno[2,3-b]pyridine-5-carbonitriles via the procedure used to prepare compound 275.

TABLE 20

| | | | | HPLC |
|---|---|---|---|---|
| Compound | | | MP | Retention |
| Number | Compound Name | MS | (° C.) | time (min) |
| 276 | 4-[(4-methyl-1H-indol-5-yl)amino]-2-[(1E)-3-pyrrolidin- | 414.1 (M + H) | 141 | 7.9 |
| | 1-ylprop-1-en-1-yl]thieno[2,3-b]pyridine-5-carbonitrile | | | |
| 277 | 4-(1H-indol-5-ylamino)-2-[(1E)-3-(4-methylpiperazin-1- | 429.3 (M + H) | 230 (dec) | 6.4 |
| | yl)prop-1-en-1-yl]thieno[2,3-b]pyridine-5-carbonitrile | | | |
| 278 | 4-[(4-methyl-1H-indol-5-yl)amino]-2-[(1E)-3-(4- | 443.2 (M + H) | N/A | 6.8 |
| | methylpiperazin-1-yl)prop-1-en-1-yl]thieno[2,3- | | | |
| | b]pyridine-5-carbonitrile | | | |
| 279 | 3-methyl-4-[(4-methyl-1H-indol-5-yl)amino]-2-[(1E)-3- | 457.4 (M + H) | N/A | 6.9 |
| | (4-methylpiperazin-1-yl)prop-1-en-1-yl]thieno[2,3- | | | |
| | b]pyridine-5-carbonitrile | | | |

### EXAMPLE 35

### Preparation of (2E)-3-[5-cyano[4[(1H-indol-5-ylamino)thieno[2,3-b]pyridin-2-yl]acrylate 280 and (2E)-3-[5-cyano-4-(1H-indol-5-ylamino)thieno[2,3-b]pyridin-2-yl]acrylic acid 281

4-Chloro-2-formylthieno[2,3-b]pyridine-5-carbonitrile (530 mg, 2.4 mmol) was dissolved in 25 mL THF and treated with (carbethoxymethylene)triphenylphosphorane (3.6 mmol, 1.25 g). After 1 hour at room temperature the reaction was concentrated to dryness and purified by silica gel chromatography (dichloromethane) to give 350 mg of (E)-ethyl 3-(4-chloro-5-cyanothieno[2,3-b]pyridin-2-yl)acrylate as a white solid.

(E)-Ethyl 3-(4-chloro-5-cyanothieno[2,3-b]pyridin-2-yl)acrylate (200 mg, 0.68 mmol) was treated with 5-aminoindole (108 mg, 0.82 mmol) and 7 mL EtOH. The suspension was heated to 80° C. for 2 hours then cooled to room temperature. The precipitate was filtered and washed with EtOH to give 175 mg of ethyl (2E)-3-[5-cyano-4-(1H-indol-5-ylamino)thieno[2,3-b]pyridin-2-yl]acrylate 280 as a brown solid, mp 226° C.; MS (ESI) m/z 389.2.

Ethyl (2E)-3-[5-cyano-4-(1H-indol-5-ylamino)thieno[2,3-b]pyridin-2-yl]acrylate 280 (200 mg, 0.51 mmol) was stirred in 10 mL THF and treated with NaOH (1.03 mL of 1 M aqueous solution). After stirring overnight, an additional 0.3 mL of 1 M NaOH was added and the reaction was stirred for 4 days at room temperature. The reaction was acidified with 1 M HCl and partially concentrated. The resulted precipitate was filtered and washed with water to give 190 mg of (2E)-3-[5-cyano-4-(1H-indol-5-ylamino)thieno[2,3-b]pyridin-2-yl]acrylic acid 281, mp 230° C. (dec.); HPLC retention time=11.1 min; MS (ESI) m/z 361.1 (M+H).

Ethyl (2E)-3-{5-cyano-4-[(4-methyl-1H-indol-5-yl)amino]thieno[2,3-b]pyridin-2-yl}acrylate 282 was prepared via the route used to prepare compound 280, MS (ESI) m/z 403.2 (M+H); HPLC retention time=16.1 min.

(2E)-3-{5-cyano-4-[(4-methyl-1H-indol-5-yl)amino]thieno[2,3-b]pyridin-2-yl}acrylic acid 283 was prepared via the route used to prepare compound 281, mp>350° C.;MS (ESI) m/z 373.3; HPLC retention time=11.8 min.

### EXAMPLE 36

### Preparation of 3-[5-cyano-4-(1H-indol-5-ylamino)thieno[2,3-b]pyridin-2-yl]propanoate 284 and 3-[5-cyano-4-(1H-indol-5-ylamino)thieno[2,3-b]pyridin-2-yl]propanoic Acid 285

Ethyl (2E)-3-[5-cyano-4-(1H-indol-5-ylamino)thieno[2,3-b]pyridin-2-yl]acrylate 280 (175 mg) was dissolved in 50 mL EtOAc and treated with 50 mg of Pd/C (10%, wet). The reaction was stirred rapidly under 1 atmosphere of hydrogen for 3 days. The reaction was filtered and concentrated. The crude product was purified by silica gel chromatography (EtOAc/hexane) to give ethyl 3-[5-cyano-4-(1H-indol-5-ylamino)thieno[2,3-b]pyridin-2-yl]propanoate 284, mp 202° C.; MS (ESI) m/z 391.3 (M+H); HPLC retention time=13.7 min.

Ethyl 3-[5-cyano-4-(1H-indol-5-ylamino)thieno[2,3-b]pyridin-2-yl]propanoate 284 (25 mg) was dissolved in 1 mL THF and treated with 0.25 mL of 1 M NaOH. After stirring at room temperature overnight, the reaction was diluted with 1 M HCl and the product was extracted into dichloromethane three times. The organic layer was dried over MgSO4 and concentrated to give 3-[5-cyano-4-(1H-indol-5-ylamino)thieno[2,3-b]pyridin-2-yl]propanoic acid 285, mp 255° C.; HPLC retention time=10.1 min; MS (ESI) m/z 363.1 (M+H).

### EXAMPLE 37

### Preparation of tert-butyl (2E)-3-{5-cyano-4-[(4-methyl-1H-indol-5-yl)amino]thieno[2,3-b]pyridin-2-yl}acrylate 286 and alternative preparation of (2E)-3-{5-cyano-4-[(4-methyl-1H-indol-5-yl)amino]thieno[2,3-b]pyridin-2-yl}acrylic acid 283

2-Iodo-4-[(4-methyl-1H-indol-5-yl)amino]thieno[2,3-b]pyridine-5-carbonitrile 123 (300 mg, 0.70 mmol), t-butyl acrylate (270 mg, 2.1 mmol), trimethyl phosphite (9 mg, 0.07 mmol), palladium acetate (9 mg, 0.07 mmol), and triethylamine (101 mg, 1.0 mmol) was stirred in 3.5 mL DMF at 80° C. for 2 hours. The crude reaction was evaporated onto silica gel and tert-butyl (2E)-3-{5-cyano-4-[(4-methyl-1H-indol-5-yl)amino]thieno[2,3-b]pyridin-2-yl}acrylate 286 was purified by chromatography (EtOAc/hexane), mp 218° C.; HPLC retention time=18.4 min; MS (ESI) m/z 431.1 (M+H).

Tert-butyl (2E)-3-{5-cyano-4-[(4-methyl-1H-indol-5-yl)amino]thieno[2,3-b]pyridin-2-yl}acrylate 286 (300 mg) was dissolved in 40 mL of 5% TFA in dichloromethane. After stirring for 12 hours, the reaction was concentrated to dryness. (2E)-3-{5-Cyano-4-[(4-methyl-1H-indol-5-yl)amino]thieno[2,3-b]pyridin-2-yl}acrylic acid 283 was generally used without further purification.

(2E)-3-{5-Cyano-3-methyl-4-[(4-methyl-1H-indol-5-yl)amino]thieno[2,3-b]pyridin-2-yl}acrylic acid 287 was prepared by the route used to prepare compound 283 described immediately above, mp 329° C.; MS (ESI) m/z 389.2 (M+H); HPLC retention time=11.8 min.

### EXAMPLE 38

### General Procedure for the Synthesis of C-2 α,β-unsaturated amides

Scheme 18 below depicts an exemplary synthetic route for preparing the compounds in Table 21.

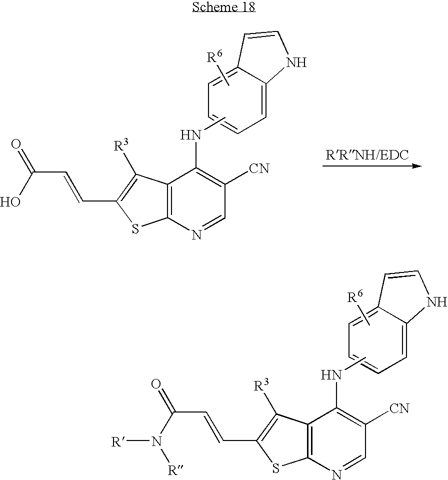

The carboxylic acid starting material (0.16 mmol) and triethyl amine (0.24 mmol) was stirred in 2 mL dichloromethane. An amine of formula R′R″NH (0.32 mmol) was added followed by EDC (0.32 mmol). DMF (1-2 mL) was added if needed to improve the solubility. After stirring overnight, the reaction mixture was evaporated onto silica gel and purified by silica gel chromatography. Alternatively, the crude reaction mixture could be dissolved in DMF and purified by preparative HPLC.

TABLE 21

| | | | | HPLC |
|---|---|---|---|---|
| Compound | | | | Retention |
| Number | Compound Name | MS | MP (° C.) | time (min) |
| 288 | 4-(1H-indol-5-ylamino)-2-[(1E)-3-oxo-3-pyrrolidin- | 414.2 (M + H) | >270 | 12.6 |
| | 1-ylprop-1-en-1-yl]thieno[2,3-b]pyridine-5- | | | |
| | carbonitrile | | | |
| 289 | (2E)-3-[5-cyano-4-(1H-indol-5-ylamino)thieno[2,3- | 402.0 (M − H) | >290 | 9.2 |
| | b]pyridin-2-yl]-N-(2-hydroxyethyl)acrylamide | | | |
| 290 | 4-(1H-indol-5-ylamino)-2-[(1E)-3-(4- | 443.3 (M + H) | 232 | 7.3 |
| | methylpiperazin-1-yl)-3-oxoprop-1-en-1- | | | |
| | yl]thieno[2,3-b]pyridine-5-carbonitrile | | | |
| 291 | 4-(1H-indol-5-ylamino)-2-[(1E)-3-(2- | 428.3 (M + H) | 214 | 13.5 |
| | methylpyrrolidin-1-yl)-3-oxoprop-1-en-1- | | | |
| | yl]thieno[2,3-b]pyridine-5-carbonitrile | | | |
| 292 | (2E)-3-[5-cyano-4-(1H-indol-5-ylamino)thieno[2,3- | 358 (M − H) | >290 | 9.5 |
| | b]pyridin-2-yl]acrylamide | | | |
| 293 | (2E)-3-[5-cyano-4-(1H-indol-5-ylamino)thieno[2,3- | 437.2 (M + H) | 175 | 8.5 |
| | b]pyridin-2-yl]-N-pyridin-3-ylacrylamide | | | |
| 294 | 4-[(4-methyl-1H-indol-5-yl)amino]-2-[(1E)-3-oxo-3- | 428.3 (M + H) | 300 (dec.) | 13.1 |
| | pyrrolidin-1-ylprop-1-en-1-yl]thieno[2,3-b]pyridine- | | | |
| | 5-carbonitrile | | | |
| 295 | 4-[(4-methyl-1H-indol-5-yl)amino]-2-[(1E)-3-(4- | 457.3 (M + H) | N/A | 7.3 |
| | methylpiperazin-1-yl)-3-oxoprop-1-en-1- | | | |
| | yl]thieno[2,3-b]pyridine-5-carbonitrile | | | |
| 296 | (2E)-3-{5-cyano-4-[(4-methyl-1H-indol-5- | 451.2 (M + H) | N/A | 8.8 |
| | yl)amino]thieno[2,3-b]pyridin-2-yl}-N-pyridin-3- | | | |
| | ylacrylamide | | | |
| 297 | (2E)-3-{5-cyano-4-[(4-methyl-1H-indol-5- | 372.1 (M − H) | N/A | 10.3 |
| | yl)amino]thieno[2,3-b]pyridin-2-yl}acrylamide | | | |
| 298 | 4-[(4-methyl-1H-indol-5-yl)amino]-2-[(1E)-3-oxo-3- | 442.2 (M + H) | 270 (dec.) | 14.5 |
| | piperidin-1-ylprop-1-en-1-yl]thieno[2,3-b]pyridine- | | | |
| | 5-carbonitrile | | | |
| 299 | 4-[(4-methyl-1H-indol-5-yl)amino]-2-{(1E)-3-oxo-3- | 511.3 (M + H) | N/A | 8.7 |
| | [(2S)-2-(pyrrolidin-1-ylmethyl)pyrrolidin-1-yl]prop- | | | |
| | 1-en-1-yl}thieno[2,3-b]pyridine-5-carbonitrile | | | |
| 300 | 2-{(1E)-3-[(3R)-3-(dimethylamino)pyrrolidin-1-yl]- | 471.1 (M + H) | 240 | 7.3 |
| | 3-oxoprop-1-en-1-yl}-4-[(4-methyl-1H-indol-5- | | | |
| | yl)amino]thieno[2,3-b]pyridine-5-carbonitrile | | | |
| 301 | 2-{(1E)-3-[(3S)-3-(dimethylamino)pyrrolidin-1-yl]- | 471.1 (M + H) | 245 | 7.3 |
| | 3-oxoprop-1-en-1-yl}-4-[(4-methyl-1H-indol-5- | | | |
| | yl)amino]thieno[2,3-b]pyridine-5-carbonitrile | | | |
| 302 | 3-methyl-4-[(4-methyl-1H-indol-5-yl)amino]-2- | 442.3 (M + H) | >270 | 13.4 |
| | [(1E)-3-oxo-3-pyrrolidin-1-ylprop-1-en-1- | | | |
| | yl]thieno[2,3-b]pyridine-5-carbonitrile | | | |
| 303 | (2E)-3-{5-cyano-4-[(4-methyl-1H-indol-5- | 450.2 (M + H) | >315 | 14.5 |
| | yl)amino]thieno[2,3-b]pyridin-2-yl}-N- | | | |
| | phenylacrylamide | | | |
| 304 | (2E)-3-{5-cyano-4-[(4-methyl-1H-indol-5- | 451.2 (M + H) | >315 | 8.7 |
| | yl)amino]thieno[2,3-b]pyridin-2-yl}-N-pyridin-4- | | | |
| | ylacrylamide | | | |
| 305 | (2E)-3-{5-cyano-4-[(4-methyl-1H-indol-5- | 402.2 (M + H) | 305 | 11.8 |
| | yl)amino]thieno[2,3-b]pyridin-2-yl}-N,N- | | | |
| | dimethylacrylamide | | | |
| 306 | (2E)-3-{5-cyano-4-[(4-methyl-1H-indol-5- | 430.3 (M + H) | 283 | 13.9 |
| | yl)amino]thieno[2,3-b]pyridin-2-yl}-N,N- | | | |
| | diethylacrylamide | | | |
| 307 | (2E)-3-{5-cyano-4-[(4-methyl-1H-indol-5- | 402.2 (M + H) | >310 | 11.7 |
| | yl)amino]thieno[2,3-b]pyridin-2-yl}-N- | | | |
| | ethylacrylamide | | | |
| 308 | (2E)-3-{5-cyano-4-[(4-methyl-1H-indol-5- | 432.2 (M + H) | 275 | 11.2 |
| | yl)amino]thieno[2,3-b]pyridin-2-yl}-N-(2- | | | |
| | methoxyethyl)acrylamide | | | |
| 309 | 4-[(4-methyl-1H-indol-5-yl)amino]-2-[(1E)-3- | 444.2 (M + H) | >310 | 11.6 |
| | morpholin-4-yl-3-oxoprop-1-en-1-yl]thieno[2,3- | | | |
| | b]pyridine-5-carbonitrile | | | |
| 310 | 2-[(1E)-3-(3-hydroxypyrrolidin-1-yl)-3-oxoprop-1- | 444.2 (M + H) | 305 | 10 |
| | en-1-yl]-4-[(4-methyl-1H-indol-5- | | | |
| | yl)amino]thieno[2,3-b]pyridine-5-carbonitrile | | | |
| 311 | 4-[(4-methyl-1H-indol-5-yl)amino]-2-[(1E)-3-oxo-3- | 443.3 (M + H) | N/A | 7.3 |
| | piperazin-1-ylprop-1-en-1-yl]thieno[2,3-b]pyridine- | | | |
| | 5-carbonitrile | | | |
| 312 | (2E)-3-{5-cyano-4-[(4-methyl-1H-indol-5- | 459.3 (M + H) | N/A | 7.7 |
| | yl)amino]thieno[2,3-b]pyridin-2-yl}-N-[2- | | | |
| | (dimethylamino)ethyl]-N-methylacrylamide | | | |
| 313 | (2E)-3-{5-cyano-4-[(4-methyl-1H-indol-5- | 417.2 (M + H) | N/A | 10.1 |
| | yl)amino]thieno[2,3-b]pyridin-2-yl}-N′,N′- | | | |
| | dimethylacrylohydrazide | | | |
| 314 | (2E)-3-{5-cyano-4-[(4-methyl-1H-indol-5- | 443.2 (M + H) | N/A | 9.7 |
| | yl)amino]thieno[2,3-b]pyridin-2-yl}-N-pyrrolidin-1- | | | |
| | ylacrylamide | | | |

### EXAMPLE 39

### Preparation of 4-[(4-methyl-1H-indol-5-yl)amino]-2-(3-oxo-3-pyrrolidin-1-ylpropyl)thieno[2,3-b]pyridine-5-carbonitrile 315

4-[(4-Methyl-1H-indol-5-yl)amino]-2-[(1E)-3-oxo-3-pyrrolidin-1-ylprop-1-en-1-yl]thieno[2,3-b]pyridine-5-carbonitrile 294 (45 mg) was dissolved in 2:1 EtOH/toluene (˜20 mL) and treated with Pd/C (10%, wet, ˜30 mg). The reaction was stirred to room temperature overnight under an atmosphere of hydrogen. Filtration and concentration gave 4-[(4-methyl-1H-indol-5-yl)amino]-2-(3-oxo-3-pyrrolidin-1-ylpropyl)thieno[2,3-b]pyridine-5-carbonitrile 315 as a solid, mp 175° C.; MS (ESI) m/z 430.3 (M+H); HPLC retention time=11.7 min.

4-(1H-Indol-5-ylamino)-2-(2-phenylethyl)thieno[2,3-b]pyridine-5-carbonitrile 316 was prepared by using a similar procedure to reduce 4-(1H-indol-5-ylamino)-2-[(E)-2-phenylvinyl]thieno[2,3-b]pyridine-5-carbonitrile 400 (infra), mp 150° C. (dec.); MS (ESI) m/z 395.3 (M+H); HPLC retention time=16.5 min

Additional Analogs Based on Example 6

Following the procedure for the preparation of compound 116 (Example 6), the appropiate 4-chlorothieno[2,3-b]pyridine-5-carbonitrile was reacted with the appropriate indole to provide the following analogs listed in Table 22. The solvent used is noted, along with in some cases the use of triethylamine.

TABLE 22

| Compound | | | | |
|---|---|---|---|---|
| Number | Compound Name | MS | MP (° C.) | Solvent |
| 317 | 4-(1H-Indol-5-ylamino)-2-iodo-3- | (ESI) m/z | 244 | ethanol |
| | methylthieno[2,3-b]pyridine-5-carbonitrile | 431.1 | | |
| 318 | 2-Iodo-3-methyl-4-[(4-methyl-1H-indol-5- | (ESI) m/z | 222 | ethanol |
| | yl)amino]thieno[2,3-b]pyridine-5-carbonitrile | 443.2 | | |
| | | (M − H) | | |
| 319 | 4-[(5-methyl-1H-indol-4-yl)amino]-2- | (ESI) m/z | >260 | ethanol |
| | phenylthieno[2,3-b]pyridine-5-carbonitrilee | 381.1 | | |
| 320 | 2-{3-[(dimethylamino)methyl]phenyl}-4-(1H- | (ESI) m/z | 267 (dec.) | ethanol |
| | pyrrolo[2,3-b]pyridin-5-ylamino)thieno[2,3- | 425.2 | | |
| | b]pyridine-5-carbonitrile | | | |
| 321 | 4-(1H-indol-5-ylamino)-2-iodo-3- | (ESI) m/z | 222-224 | 2- |
| | isopropylthieno[2,3-b]pyridine-5-carbonitrile | 459.1 | | ethoxyethanol |
| 322 | 2-phenyl-4-(1H-pyrrolo[2,3-b]pyridin-5- | (ESI) m/z | >245 | ethanol |
| | ylamino)thieno[2,3-b]pyridine-5-carbonitrile | 409.2 | | |
| 323 | 4-[(6-methyl-1H-indol-5-yl)amino]-2- | (ESI) m/z | >260 | ethanol |
| | phenylthieno[2,3-b]pyridine-5-carbonitrile | 381.2 | | |
| 324 | 2-iodo-3-isopropyl-4-[(4-methyl-1H-indol-5- | (ESI) m/z | 227-230 | ethanol, |
| | yl)amino]thieno[2,3-b]pyridine-5-carbonitrile | 473.2 | | triethylamine |
| 325 | 3-bromo-4-[(4-methyl-1H-indol-5- | (ESI) 383.1 | 225-227 | ethanol, |
| | yl)amino]thieno[2,3-b]pyridine-5-carbonitrile | | | triethylamine |

Additional Analogs Based on Example 8

Following the procedure for the preparation of compound 127 (Example 8), except using THF as the solvent instead of DMF, the appropriate 4-chlorothieno[2,3-b]pyridine-5-carbonitrile was reacted with the appropriate amine to provide the following analogs listed in Table 23.

TABLE 23

| Compound | | | |
|---|---|---|---|
| Number | Compound Name | MS | MP (° C.) |
| 326 | 2-{3-[(dimethylamino)methyl]phenyl}-4-(pyridin-3- | (ESI) m/z | 186-188 |
| | ylamino)thieno[2,3-b]pyridine-5-carbonitrile | 386.3 | |
| 327 | 2-{3-[(dimethylamino)methyl]phenyl}-4-(pyridin-4- | (ESI) m/z | 253 (dec.) |
| | ylamino)thieno[2,3-b]pyridine-5-carbonitrile | 386.3 | |

Additional Analogs Based on Example 18 (Part 1)

Following the procedure for the preparation of compound 188 (Example 18), the appropriate 2-iodo- or 2-bromothieno[2,3-b]pyridine-5-carbonitrile was reacted with the appropriate boronic acid or boronic ester to provide the following analogs listed in Table 24. In some cases the boronic acid or boronic ester was generated in situ from the corresponding bromo or iodo analog with n-butyl lithium and an alkyl borate, such as triisopropyl borate. In some cases saturated aqueous sodium carbonate was used instead of saturated aqueous sodium bicarbonate and in some cases the reaction was performed in a microwave.

TABLE 24

| Compound | | | |
|---|---|---|---|
| Number | Compound Name | MS | MP (° C.) |
| 328 | 4-(1H-indol-5-ylamino)-2-(2-naphthyl)thieno[2,3-b]pyridine-5- | (ESI) m/z | >245 |
| | carbonitrile | 415.1 | |
| 329 | 4-[(4-methyl-1H-indol-5-yl)amino]-2-(6-morpholin-4-ylpyridin-3- | (ESI) m/z | >260 |
| | yl)thieno[2,3-b]pyridine-5-carbonitrile | 467.3 | |
| 330 | 4-[(4-methyl-1H-indol-5-yl)amino]-2-(2-morpholin-4- | (ESI) m/z | >260 |
| | ylpyrimidin-5-yl)thieno[2,3-b]pyridine-5-carbonitrile | 468.2 | |
| 331 | 2-[2-(dimethylamino)pyrimidin-5-yl]-4-[(4-methyl-1H-indol-5- | (ESI) m/z | >260 |
| | yl)amino]thieno[2,3-b]pyridine-5-carbonitrile | 426.2 | |
| 332 | 2-(2-ethoxyphenyl)-4-[(4-methyl-1H-indol-5-yl)amino]thieno[2,3- | (ESI) m/z | 226-228 |
| | b]pyridine-5-carbonitrile | 425.2 | |
| 333 | methyl (4-{5-cyano-4-[(4-methyl-1H-indol-5-yl)amino]thieno[2,3- | (ESI) m/z | >245 |
| | b]pyridin-2-yl}phenyl)carbamate | 454.2 | |
| 334 | N-butyl-N′-{4-[5-cyano-4-(1H-indol-5-ylamino)thieno[2,3- | (ESI) m/z | >250 C. |
| | b]pyridin-2-yl]phenyl}urea | 481.2 | |
| 335 | 3-[5-cyano-4-(1H-indol-5-ylamino)thieno[2,3-b]pyridin-2-yl]-N- | (ESI) m/z | 219-221 |
| | [2-(dimethylamino)ethyl]benzamide | 481.2 | |
| 336 | 2-(4-formyl-3-thienyl)-4-(1H-indol-5-ylamino)thieno[2,3- | (ESI) m/z | 224-229 (dec.) |
| | b]pyridine-5-carbonitrile | 401.1 | |
| 337 | 2-(3-formyl-4-methoxyphenyl)-4-(1H-indol-5-ylamino)thieno[2,3- | (ESI) m/z | >250 |
| | b]pyridine-5-carbonitrile | 424.8 | |
| 338 | 2-(5-formyl-2-methoxyphenyl)-4-[(4-methyl-1H-indol-5- | (ESI) m/z | >250 |
| | yl)amino]thieno[2,3-b]pyridine-5-carbonitrile | 439.0 | |
| 339 | 3-{5-cyano-4-[(4-methyl-1H-indol-5-yl)amino]thieno[2,3- | (ESI) m/z | 185-187 |
| | b]pyridin-2-yl}-N-[2-(dimethylamino)ethyl]benzamide | 495.1 | |
| 340 | 2-(3-acetylphenyl)-4-[(4-methyl-1H-indol-5-yl)amino]thieno[2,3- | (ESI) m/z | >260 |
| | b]pyridine-5-carbonitrile | 423.2 | |
| 341 | 2-(5-formyl-1-benzothien-2-yl)-4-(1H-indol-5- | (ESI) m/z | >245 |
| | ylamino)thieno[2,3-b]pyridine-5-carbonitrile | 451.1 | |
| 342 | methyl {4-[5-cyano-4-(1H-indol-5-ylamino)thieno[2,3-b]pyridin- | (ESI) m/z | 240 (dec.) |
| | 2-yl]phenyl}carbamate | 440.2 | |
| 343 | 3-{5-cyano-4-[(4-methyl-1H-indol-5-yl)amino]thieno[2,3- | (ESI) m/z | >260 |
| | b]pyridin-2-yl}-N-methylbenzenesulfonamide | 474.2 | |
| 344 | 3-[5-cyano-4-(1H-indol-5-ylamino)thieno[2,3-b]pyridin-2-yl]-N- | (ESI) m/z | 254-256 |
| | methoxy-N-methylbenzamide | 454.2 | |
| 345 | 4-{5-cyano-4-[(4-methyl-1H-indol-5-yl)amino]thieno[2,3- | (ESI) m/z | 251-252 |
| | b]pyridin-2-yl}-N-[2-(dimethylamino)ethyl]benzenesulfonamide | 531.3 | |
| 346 | 4-{5-cyano-4-[(4-methyl-1H-indol-5-yl)amino]thieno[2,3- | (ESI) m/z | 176-178 |
| | b]pyridin-2-yl}-N-[2-(dimethylamino)ethyl]-N- | 545.3 | |
| | methylbenzenesulfonamide | | |
| 347 | 2-{3-[1-(dimethylamino)ethyl]phenyl}-4-[(4-methyl-1H-indol-5- | (ESI) m/z | 232-234 |
| | yl)amino]thieno[2,3-b]pyridine-5-carbonitrile | 452.3 | |
| 348 | 3-{5-cyano-4-[(4-methyl-1H-indol-5-yl)amino]thieno[2,3- | (ESI) m/z | 184-185 |
| | b]pyridin-2-yl}-N-[2-(dimethylamino)ethyl]-N-methylbenzamide | 509.3 | |
| 349 | 4-{5-cyano-4-[(4-methyl-1H-indol-5-yl)amino]thieno[2,3- | (ESI) m/z | >260 |
| | b]pyridin-2-yl}-N-[2-(dimethylamino)ethyl]benzamide | 495.3 | |
| 350 | 2-{6-[3-(dimethylamino)propoxy]pyridin-3-yl}-4-[(4-methyl-1H- | (ESI) m/z | 225-227 |
| | indol-5-yl)amino]thieno[2,3-b]pyridine-5-carbonitrile | 483.2 | |
| 351 | 2-[4-(2-chloroethoxy)phenyl]-4-[(4-methyl-1H-indol-5- | (ESI) m/z | 200-202 |
| | yl)amino]thieno[2,3-b]pyridine-5-carbonitrile | 459.2 | |
| 352 | 4-[(4-methyl-1H-indol-5-yl)amino]-2-{6-[(2-morpholin-4- | (ESI) m/z | 240-242 |
| | ylethyl)amino]pyridin-3-yl}thieno[2,3-b]pyridine-5-carbonitrile | 510.3 | |
| 353 | 3-{5-cyano-4-[(4-methyl-1H-indol-5-yl)amino]thieno[2,3- | (ESI) m/z | >260 |
| | b]pyridin-2-yl}-N,N-dmethylbenzamide | 452.3 | |
| 354 | 3-{5-cyano-4-[(4-methyl-1H-indol-5-yl)amino]thieno[2,3- | (ESI) m/z | 251-253 |
| | b]pyridin-2-yl}-N-methoxy-N-methylbenzamide | 468.3 | |
| 355 | 3-{5-cyano-4-[(4-methyl-1H-indol-5-yl)amino]thieno[2,3- | (ESI) m/z | 236-238 |
| | b]pyridin-2-yl}-N-methoxybenzamide | 468.3 | |
| 356 | 4-{5-cyano-4-[(4-methyl-1H-indol-5-yl)amino]thieno[2,3- | (ESI) m/z | >260 |
| | b]pyridin-2-yl}-N,N-dimethylbenzamide | 454.2 | |
| 357 | 4-{5-cyano-4-[(4-methyl-1H-indol-5-yl)amino]thieno[2,3- | (ESI) m/z | >260 |
| | b]pyridin-2-yl}-N-methoxy-N-methylbenzamide | 468.3 | |
| 358 | 4-{5-cyano-4-[(4-methyl-1H-indol-5-yl)amino]thieno[2,3- | (ESI) m/z | >260 |
| | b]pyridin-2-yl}-N-methoxybenzamide | 454.1 | |
| 359 | N-{3-[5-cyano-4-(1H-indol-5-ylamino)thieno[2,3-b]pyridin-2- | (ESI) m/z | >245 |
| | yl]phenyl}methanesulfonamide | 460.2 | |
| 360 | N-(3-{5-cyano-4-[(4-methyl-1H-indol-5-yl)amino]thieno[2,3- | (ESI) m/z | >245 |
| | b]pyridin-2-yl}phenyl)methanesulfonamide | 474.2 | |
| 361 | N-(4-{5-cyano-4-[(4-methyl-1H-indol-5-yl)amino]thieno[2,3- | (ESI) m/z | >245 |
| | b]pyridin-2-yl}phenyl)methanesulfonamide | 474.3 | |
| 362 | 4-[(4-methyl-1H-indol-5-yl)amino]-2-(2-naphthyl)thieno[2,3- | m/z 431.3 | 225-227 |
| | b]pyridine-5-carbonitrile | | |
| 363 | 4-[(4-methyl-1H-indol-5-yl)amino]-2-(1-naphthyl)thieno[2,3- | m/z 431.1 | 226-229 |
| | b]pyridine-5-carbonitrile | | |
| 364 | 2-(2-methoxyphenyl)-4-[(4-methyl-1H-indol-5- | (ESI) m/z | 159-163 |
| | yl)amino]thieno[2,3-b]pyridine-5-carbonitrile | 411.2 | |
| 365 | 2-(3-formyl-5-isopropoxyphenyl)-4-[(4-methyl-1H-indol-5- | (ESI) m/z | 229-232 C. |
| | yl)amino]thieno[2,3-b]pyridine-5-carbonitrile | 467.3 | |
| 366 | 2-(2-methoxy-5-methylphenyl)-4-[(4-methyl-1H-indol-5- | (ESI) m/z | >245 |
| | yl)amino]thieno[2,3-b]pyridine-5-carbonitrile | 425.3 | |
| 367 | 2-{5-[(dimethylamino)methyl]-2-ethoxyphenyl}-4-[(4-methyl-1H- | (ESI) m/z | 200 (dec.) |
| | indol-5-yl)amino]thieno[2,3-b]pyridine-5-carbonitrile | 482.4 | |
| 368 | 2-{5-[(dimethylamino)methyl]-2-methylphenyl}-4-[(4-methyl-1H- | (ESI) m/z | 154 (dec.) |
| | indol-5-yl)amino]thieno[2,3-b]pyridine-5-carbonitrile | 452.3 | |
| 369 | 3-{5-cyano-4-[(4-methyl-1H-indol-5-yl)amino]thieno[2,3- | (ESI) m/z | >260 |
| | b]pyridin-2-yl}-4-methoxy-N,N-dimethylbenzamide | 482.3 | |
| 370 | N-{2-[5-cyano-4-(1H-indol-5-ylamino)thieno[2,3-b]pyridin-2- | (ESI) m/z | >245 |
| | yl]phenyl}methanesulfonamide | 459.9 | |
| 371 | N-(2-{5-cyano-4-[(4-methyl-1H-indol-5-yl)amino]thieno[2,3- | (ESI) m/z | 243-245 |
| | b]pyridin-2-yl}phenyl)methanesulfonamide | 473.9 | |
| 372 | N-(4-{5-cyano-3-methyl-4-[(4-methyl-1H-indol-5- | (ESI) m/z | >245 |
| | yl)amino]thieno[2,3-b]pyridin-2-yl}phenyl)methanesulfonamide | 487.9 | |
| 373 | 2-(1-benzothien-2-yl)-4-[(4-methyl-1H-indol-5- | (ESI) m/z | >245 |
| | yl)amino]thieno[2,3-b]pyridine-5-carbonitrile | 437.2 | |
| 374 | 2-(5-formyl-1-benzothien-2-yl)-4-[(4-methyl-1H-indol-5- | (ESI) m/z | >245 |
| | yl)amino]thieno[2,3-b]pyridine-5-carbonitrile | 465.2 | |
| 375 | 4-[(4-methyl-1H-indol-5-yl)amino]-2-[3- | (ESI) m/z | >245 |
| | (methylsulfonyl)phenyl]thieno[2,3-b]pyridine-5-carbonitrile | 459.2 | |
| 376 | 4-[(4-methyl-1H-indol-5-yl)amino]-2-[4- | (ESI) m/z | >245 |
| | (methylsulfonyl)phenyl]thieno[2,3-b]pyridine-5-carbonitrile | 459.2 | |
| 377 | 2-(3-bromophenyl)-4-[(4-methyl-1H-indol-5-yl)amino]thieno[2,3- | (ESI) m/z | 255-257 |
| | b]pyridine-5-carbonitrile | 459.1 | |
| 378 | 2-(3-formyl-5-methylphenyl)-4-[(4-methyl-1H-indol-5- | (ESI) m/z | 229-231 |
| | yl)amino]thieno[2,3-b]pyridine-5-carbonitrile | 423.3 | |
| 379 | 2-(3-formyl-5-methyl-2-propoxyphenyl)-4-[(4-methyl-1H-indol-5- | (ESI) m/z | 242-244 |
| | yl)amino]thieno[2,3-b]pyridine-5-carbonitrile | 481.4 | |
| 380 | 4-[(4-methyl-1H-indol-5-yl)amino]-2-quinolin-3-ylthieno[2,3- | (ESI) m/z | 233-235 |
| | b]pyridine-5-carbonitrile | 432.3 | |
| 381 | 2-(2-butoxyphenyl)-4-[(4-methyl-1H-indol-5-yl)amino]thieno[2,3- | (ESI) m/z | >260 |
| | b]pyridine-5-carbonitrile | 453.5 | |
| 382 | 4-[(4-methyl-1H-indol-5-yl)amino]-2-(2- | (ESI) m/z | 248-251 |
| | propoxyphenyl)thieno[2,3-b]pyridine-5-carbonitrile | 439.5 | |
| 383 | 2-{2-[3-(dimethylamino)propoxy]phenyl}-4-[(4-methyl-1H-indol- | (ESI) m/z | 212-214 |
| | 5-yl)amino]thieno[2,3-b]pyridine-5-carbonitrile | 482.3 | |
| 384 | 2-{5-[(dimethylamino)methyl]-2-propoxyphenyl}-4-[(4-methyl- | (ESI) m/z | 170 (dec.) |
| | 1H-indol-5-yl)amino]thieno[2,3-b]pyridine-5-carbonitrile | 496.4 | |
| 385 | 2-(6-ethoxy-2-naphthyl)-4-[(4-methyl-1H-indol-5- | (ESI) m/z | >245 |
| | yl)amino]thieno[2,3-b]pyridine-5-carbonitrile | 475.4 | |
| 386 | 2-(2-formylphenyl)-4-[(4-methyl-1H-indol-5-yl)amino]thieno[2,3- | (ESI) m/z | >245 |
| | b]pyridine-5-carbonitrile | 409.2 | |
| 387 | 2-(5-formylpyridin-3-yl)-4-[(4-methyl-1H-indol-5- | (ESI) m/z | 222-224 |
| | yl)amino]thieno[2,3-b]pyridine-5-carbonitrile | 410.2 | |
| 388 | 2-(2-fluorophenyl)-4-[(4-methyl-1H-indol-5-yl)amino]thieno[2,3- | (ESI) m/z | 243-245 |
| | b]pyridine-5-carbonitrile | 399.2 | |
| 389 | 2-(2-fluoro-5-formylphenyl)-4-[(4-methyl-1H-indol-5- | (ESI) m/z | 256-257 |
| | yl)amino]thieno[2,3-b]pyridine-5-carbonitrile | 427.2 | |
| 390 | 2-(3-{[2-(dimethylamino)ethyl](methyl)amino}phenyl)-4-[(4- | (ESI) m/z | 212-214 |
| | methyl-1H-indol-5-yl)amino]thieno[2,3-b]pyridine-5-carbonitrile | 481.3 | |
| 391 | 2-[6-(dimethylamino)pyridin-3-yl]-4-[(4-methyl-1H-indol-5- | (ESI) m/z | >260 |
| | yl)amino]thieno[2,3-b]pyridine-5-carbonitrile | 425.2 | |
| 392 | 2-[2-(methoxymethyl)phenyl]-4-[(4-methyl-1H-indol-5- | (ESI) m/z | 182-185 |
| | yl)amino]thieno[2,3-b]pyridine-5-carbonitrile | 425.2 | |
| 393 | 2-{3-[(dimethylamino)methyl]phenyl}-3-isopropyl-4-[(4-methyl- | (ESI) m/z | 211-214 |
| | 1H-indol-5-yl)amino]thieno[2,3-b]pyridine-5-carbonitrile | 521.4 | |
| 394 | 2-(3-{[2-(dimethylamino)ethyl]amino}phenyl)-4-[(4-methyl-1H- | (ESI) m/z | 215-216 |
| | indol-5-yl)amino]thieno[2,3-b]pyridine-5-carbonitrile | 467.3 | |
| 395 | 4-[(4-methyl-1H-indol-5-yl)amino]-2-(2-piperidin-1-ylpyrimidin- | (ESI) m/z | >260 |
| | 5-yl)thieno[2,3-b]pyridine-5-carbonitrile | 466.3 | |
| 396 | 4-[(4-methyl-1H-indol-5-yl)amino]-2-(2-pyrrolidin-1-ylpyrimidin- | (ESI) m/z | >260 |
| | 5-yl)thieno[2,3-b]pyridine-5-carbonitrile | 452.3 | |
| 397 | 4-[(4-methyl-1H-indol-5-yl)amino]-2-(6-piperidin-1-ylpyridin-3- | (ESI) m/z | >260 |
| | yl)thieno[2,3-b]pyridine-5-carbonitrile | 465.3 | |
| 398 | 2-[2-(hydroxymethyl)phenyl]-4-[(4-methyl-1H-indol-5- | (ESI) m/z | 209-211 |
| | yl)amino]thieno[2,3-b]pyridine-5-carbonitrile | 411.2 | |
| 399 | 2-{2-[2-(dimethylamino)ethoxy]phenyl}-4-[(4-methyl-1H-indol- | (ESI) m/z | 237-238 |
| | 5-yl)amino]thieno[2,3-b]pyridine-5-carbonitrile | 468.3 | |

Additional Analogs Based on Example 18 (Part 2)

The analogs in Table 25 were prepared via one of Procedures A, B, and C described below.

Procedure A: The aryl iodide was stirred in DMF (0.1M) and treated with tetrakis(triphenylphosphine)palladium(0) (5%), the boronic acid (1.3 eq), and cesium carbonate (3 eq). The reaction was heated to 70° C. overnight. The reaction was diluted with water and the product was extracted into EtOAc and purified by silica gel chromatography. Alternatively, the crude reaction mixture could be filtered and the product purified by preparative HPLC.

Procedure B: The aryl iodide was stirred in DMF (0.1 M) and treated with palladium acetate (0.07 eq), triphenylphosphine trisulfonate (0.15 eq), the boronic acid (1.5 eq), and cesium carbonate (2 eq). The reaction was heated to 80° C. overnight then filtered. The crude reaction mixture was purified by preparative HPLC.

Procedure C: The aryl iodide was stirred in DME (0.1 M) and treated with tetrakis(triphenylphosphine)palladium(0) (5-10 mol%), the boronic acid or trialkyl stannane (1.5 eq), and aqueous sodium bicarbonate (saturated, ˜10% of DME volume). The reaction was heated to 80° C. overnight. Generally, the crude reaction mixture was evaporated onto silica gel and purified by silica gel chromatography. Alternatively, the reaction could be diluted with water and the product extracted into dichloromethane/MeOH and subsequently purified by HPLC.

TABLE 25

| | | | | | HPLC |
|---|---|---|---|---|---|
| Compound | | | | MP | Retention |
| number | Compound Name | Procedure | MS | (° C.) | time (min) |
| 400 | 4-(1H-indol-5-ylamino)-2-[(E)-2- | A | 393.3 | 273 | 17.2 |
| | phenylvinyl]thieno[2,3-b]pyridine-5- | | | | |
| | carbonitrile | | | | |
| 401 | 2-[(E)-2-(4-fluorophenyl)vinyl]-4-(1H-indol-5- | B | 411.2 | 245 | 16.3 |
| | ylamino)thieno[2,3-b]pyridine-5-carbonitrile | | | | |
| 402 | 2-[(E)-2-(3-fluorophenyl)vinyl]-4-(1H-indol-5- | B | 411.2 | >270 | 18.2 |
| | ylamino)thieno[2,3-b]pyridine-5-carbonitrile | | | | |
| 403 | 2-[(1E)-4-hydroxybut-1-en-1-yl]-4-(1H-indol- | B | 361.2 | 203 | 14 |
| | 5-ylamino)thieno[2,3-b]pyridine-5-carbonitrile | | | | |
| 404 | 4-(1H-indol-5-ylamino)-2-[(E)-2-(3- | B | 423.1 | 290 | 17.3 |
| | methoxyphenyl)vinyl]thieno[2,3-b]pyridine-5- | | | | |
| | carbonitrile | | | | |
| 405 | 4-(1H-indol-5-ylamino)-2-[(E)-2-(4- | B | 423.1 | 265 | 17.1 |
| | methoxyphenyl)vinyl]thieno[2,3-b]pyridine-5- | | | | |
| | carbonitrile | | | | |
| 406 | 4-(1H-indol-5-ylamino)-2-[(E)-2-(4- | B | 407.2 | 254 | 1 |
| | methylphenyl)vinyl]thieno[2,3-b]pyridine-5- | | | | |
| | carbonitrile | | | | |
| 407 | 2-[(E)-2-(4-chlorophenyl)vinyl]-4-(1H-indol- | B | 427 | 284 | 18.9 |
| | 5-ylamino)thieno[2,3-b]pyridine-5-carbonitrile | | | | |
| 408 | 4-(1H-indol-5-ylamino)-2-{(E)-2-[4- | B | 461 | 257 | 19.1 |
| | (trifluoromethyl)phenyl]vinyl}thieno[2,3- | | | | |
| | b]pyridine-5-carbonitrile | | | | |
| 409 | 4-(1H-indol-5-ylamino)-2-[(1E)-3-phenylprop- | B | 407.2 | N/A | 17.8 |
| | 1-en-1-yl]thieno[2,3-b]pyridine-5-carbonitrile | | | | |
| 410 | 4-(1H-indol-5-ylamino)-2-(1- | B | 393.2 | 271 | 17.5 |
| | phenylvinyl)thieno[2,3-b]pyridine-5- | | | | |
| | carbonitrile | | | | |
| 411 | 2-[(1E)-hex-1-en-1-yl]-4-(1H-indol-5- | B | 373.2 | N/A | 18.9 |
| | ylamino)thieno[2,3-b]pyridine-5-carbonitrile | | | | |
| 412 | 4-(1H-indol-5-ylamino)-2-[(1E)-3- | B | 361.1 | 222 | 14 |
| | methoxyprop-1-en-1-yl]thieno[2,3-b]pyridine- | | | | |
| | 5-carbonitrile | | | | |
| 413 | 4-{5-cyano-3-methyl-4-[(4-methyl-1H-indol- | B | 472.3 | N/A | 12.4 |
| | 5-yl)amino]thieno[2,3-b]pyridin-2- | | | | |
| | yl}benzenesulfonamide | | | | |
| 414 | 4-{5-cyano-3-methyl-4-[(4-methyl-1H-indol- | B | 488.3 | N/A | 13.8 |
| | 5-yl)amino]thieno[2,3-b]pyridin-2-yl}-N- | | | | |
| | methylbenzenesulfonamide | | | | |
| 415 | 4-[5-cyano-4-(1H-indol-5-ylamino)-3- | B | 474.3 | N/A | 13.5 |
| | methylthieno[2,3-b]pyridin-2-yl]-N- | | | | |
| | methylbenzenesulfonamide | | | | |
| 416 | 4-[(4-methyl-1H-indol-5-yl)amino]-2-[(E)-2- | B | 407.1 | 240 | 17.8 |
| | phenylvinyl]thieno[2,3-b]pyridine-5- | | | | |
| | carbonitrile | | | | |
| 417 | 4-[(4-methyl-1H-indol-5-yl)amino]-2- | C | 331.2 | 220 | 14.9 |
| | vinylthieno[2,3-b]pyridine-5-carbonitrile | | | (dec.) | |
| 418 | tert-butyl 4-{5-cyano-4-[(4-methyl-1H-indol- | C | 486.3 | 180 | 17.7 |
| | 5-yl)amino]thieno[2,3-b]pyridin-2-yl}-5,6- | | | | |
| | dihydropyridine-1(2H)-carboxylate | | | | |
| 419 | tert-butyl 4-[5-cyano-4-(1H-indol-5- | C | 472.3 | 205 | 17.3 |
| | ylamino)thieno[2,3-b]pyridin-2-yl]-3,6- | | | | |
| | dihydropyridine-1(2H)-carboxylate | | | | |
| 420 | 2-[(1E)-4-hydroxybut-1-en-1-yl]-4-[(4-methyl- | C | 375.2 | 220 | 11.7 |
| | 1H-indol-5-yl)amino]thieno[2,3-b]pyridine-5- | | | | |
| | carbonitrile | | | | |
| 421 | 2-cyclohex-1-en-1-yl-4-[(4-methyl-1H-indol- | C | 385.2 | 236 | 18.1 |
| | 5-yl)amino]thieno[2,3-b]pyridine-5- | | | | |
| | carbonitrile | | | | |
| 422 | 2-[(1E)-3-methoxyprop-1-en-1-yl]-4-[(4- | C | 375.1 | N/A | 14.4 |
| | methyl-1H-indol-5-yl)amino]thieno[2,3- | | | | |
| | b]pyridine-5-carbonitrile | | | | |
| 423 | 4-[(4-methyl-1H-indol-5-yl)amino]-2-[3- | C | 478.1 | 332 | 14.7 |
| | (pyrrolidin-1-ylcarbonyl)phenyl]thieno[2,3- | | | | |
| | b]pyridine-5-carbonitrile | | | | |
| 424 | 3-methyl-4-[(4-methyl-1H-indol-5-yl)amino]- | C | 395.2 | >280 | 17.6 |
| | 2-phenylthieno[2,3-b]pyridine-5-carbonitrile | | | | |
| 425 | 3-methyl-4-[(4-methyl-1H-indol-5-yl)amino]- | C | 421.3 | 255 | 18.6 |
| | 2-[(E)-2-phenylvinyl]thieno[2,3-b]pyridme-5- | | | | |
| | carbonitrile | | | | |
| 426 | N-(3-{5-cyano-3-methyl-4-[(4-methyl-1H- | C | 488.3 | 321 | 14 |
| | indol-5-yl)amino]thieno[2,3-b]pyridin-2- | | | | |
| | yl}phenyl)methanesulfonamide | | | | |
| 427 | 3-{5-cyano-3-methyl-4-[(4-methyl-1H-indol- | C | 474.2 | 245 | 12.8 |
| | 5-yl)amino]thieno[2,3-b]pyridin-2- | | | | |
| | yl}benzenesulfonamide | | | | |
| 428 | 3-{5-cyano-3-methyl-4-[(4-methyl-1H-indol- | C | 488.2 | >250 | 14.3 |
| | 5-yl)amino]thieno[2,3-b]pyridin-2-yl}-N- | | | | |
| | methylbenzenesulfonamide | | | | |
| 429 | 3-{5-cyano-3-methyl-4-[(4-methyl-1H-indol- | C | 509.3 | 130 | 8.8 |
| | 5-yl)amino]thieno[2,3-b]pyridin-2-yl}-N-[2- | | | | |
| | (dimethylamino)ethyl]benzamide | | | | |
| 430 | 4-{5-cyano-3-methyl-4-[(4-methyl-1H-indol- | C | 509.3 | 125 | 8.5 |
| | 5-yl)amino]thieno[2,3-b]pyridin-2-yl}-N-[2- | | | | |
| | (dimethylamino)ethyl]benzamide | | | | |
| 431 | 4-{5-cyano-3-methyl-4-[(4-methyl-1H-indol- | C | 518.3 | 308 | 12.4 |
| | 5-yl)amino]thieno[2,3-b]pyridin-2-yl}-N-(2- | | | | |
| | hydroxyethyl)benzenesulfonamide | | | | |
| 432 | 2-(1-methyl-1H-imidazol-2-yl)-4-[(4-methyl- | C | 385.2 | 125 | 7.7 |
| | 1H-indol-5-yl)amino]thieno[2,3-b]pyridine-5- | | | | |
| | carbonitrile | | | | |
| 433 | 4-(1H-indol-5-ylamino)-2-(1-methyl-1H- | C | 371.3 | >265 | 6.7 |
| | imidazol-2-yl)thieno[2,3-b]pyridine-5- | | | | |
| | carbonitrile | | | | |
| 434 | 2-(1H-indol-2-yl)-4-[(4-methyl-1H-indol-5- | C | 420.2 | 227 | 17.3 |
| | yl)amino]thieno[2,3-b]pyridine-5-carbonitrile | | | | |
| 435 | N-(2-{5-cyano-4-[(4-methyl-1H-indol-5- | C | 438.2 | 180 | 13 |
| | yl)amino]thieno[2,3-b]pyridin-2- | | | | |
| | yl}phenyl)acetamide | | | | |
| 436 | 2-(2-aminophenyl)-4-[(4-methyl-1H-indol-5- | C | 396.2 | 255 | 15.4 |
| | yl)amino]thieno[2,3-b]pyridine-5-carbonitrile | | | | |
| 437 | 2-(3-hydroxyphenyl)-4-[(4-methyl-1H-indol-5- | C | 397.2 | >280 | 14.6 |
| | yl)amino]thieno[2,3-b]pyridine-5-carbonitrile | | | | |
| 438 | 4-[(4-methyl-1H-indol-5-yl)amino]-2-pyridin- | C | 382.1 | >285 | 11.7 |
| | 3-ylthieno[2,3-b]pyridine-5-carbonitrile | | | | |
| 439 | 4-[(4-methyl-1H-indol-5-yl)amino]-2-pyridin- | C | 382.1 | >285 | 9 |
| | 4-ylthieno[2,3-b]pyridine-5-carbonitrile | | | | |
| 440 | 4-[(4-methyl-1H-indol-5-yl)amino]-2-pyridin- | C | 382.1 | >285 | 14.6 |
| | 2-ylthieno[2,3-b]pyridine-5-carbonitrile | | | | |
| 441 | 2-(4-hydroxyphenyl)-4-[(4-methyl-1H-indol-5- | C | 397.2 | 250 | 13.6 |
| | yl)amino]thieno[2,3-b]pyridine-5-carbonitrile | | | | |
| 442 | 2-(2-hydroxyphenyl)-4-[(4-methyl-1H-indol-5- | C | 397.2 | 280 | 13.4 |
| | yl)amino]thieno[2,3-b]pyridine-5-carbonitrile | | | | |

Additional Analogs Based on Examples 19, 21-23

The compounds in Table 26 were prepared following the procedure for the preparation of compounds 203, 213, 221 and 226, of Examples 19, 21-23, respectively, as noted.

TABLE 26

| Compound | | MP or | | |
|---|---|---|---|---|
| Number | Name | HPLC | MS | Preparation |
| 443 | 2-{6-[(dimethylamino)methyl]pyridin-2-yl}-4-(1H- | 198-200° C. | MS (ESI) m/z | Via route used |
| | indol-5-ylamino)thieno[2,3-b]pyridine-5-carbonitrile | | 425.1 | to prepare |
| | | | | Example 19 |
| 444 | 2-({4-[(dimethylamino)methyl]pyridin-2-yl}ethynyl)- | >250° C. | MS (ESI) m/z | Via route used |
| | 4-[(4-methyl-1H-indol-5-yl)amino]thieno[2,3- | | 463.2 | to prepare |
| | b]pyridine-5-carbonitrile | | | Example 21 |
| 445 | 2-{[6-(dimethylamino)pyridin-3-yl]ethynyl}-4-[(4- | >260° C. | MS (ESI) m/z | Via route used |
| | methyl-1H-indol-5-yl)amino]thieno[2,3-b]pyridine-5- | | 449.2 | to prepare |
| | carbonitrile | | | Example 21 |
| 446 | 4-[(4-methyl-1H-indol-5-yl)amino]-2- | 258-260° C. | MS (ESI) m/z | Via route used |
| | [(trimethylsilyl)ethynyl]thieno[2,3-b]pyridine-5- | | 401.2 | to prepare |
| | carboni | | | Example 21 |
| 447 | 4-[(4-methyl-1H-indol-5-yl)amino]-2-({5-[(4- | 216-218° C. | MS (ESI) m/z | Via route used |
| | methylpiperazin-1-yl)methyl]-3- | | 507.2 | to prepare |
| | furyl}ethynyl)thieno[2,3-b]pyridine-5-carbonitrile | | | Example 21 |
| 448 | 2-({5-[(dimethylamino)methyl]-2-thienyl}ethynyl)-4- | 185-187° C. | MS (ESI) m/z | Via route used |
| | [(4-methyl-1H-indol-5-yl)amino]thieno[2,3- | | 468.3 | to prepare |
| | b]pyridine-5-carbonitrile | | | Example 21 |
| 449 | 2-({5-[(dimethylamino)methyl]-2-furyl}ethynyl)-4- | 218-220° C. | MS (ESI) m/z | Via route used |
| | [(4-methyl-1H-indol-5-yl)amino]thieno[2,3- | | 452.1 | to prepare |
| | b]pyridine-5-carbonitrile | | | Example 21 |
| 450 | 4-[(4-methyl-1H-indol-5-yl)amino]-2-[4-(4- | HPLC rt = 5.8 min | (ESI) m/z | Via route used |
| | methylpiperazin-1-yl)but-1-yn-1-yl]thieno[2,3- | | 455.3 | to prepare |
| | b]pyridine-5-carbonitrile | | | Example 22 |
| 451 | 5-({5-cyano-4-[(4-methyl-1H-indol-5- | >260° C. | MS (ESI) m/z | Via route used |
| | yl)amino]thieno[2,3-b]pyridin-2-yl}ethynyl)-N,N- | | 477.3 | to prepare |
| | dimethylnicotinamide | | | Example 23 |
| 452 | 2-({5-[(dimethylamino)methyl]pyridin-3-yl}ethynyl)- | 130° C. (dec.) | MS (ESI) m/z | Via route used |
| | 4-[(4-methyl-1H-indol-5-yl)amino]thieno[2,3- | | 463.3 | to prepare |
| | b]pyridine-5-carbonitrile | | | Example 23 |
| 453 | 2-({6-[(dimethylamino)methyl]pyridin-3-yl}ethynyl)- | 230-232° C. | MS (ESI) m/z | Via route used |
| | 4-[(4-methyl-1H-indol-5-yl)amino]thieno[2,3- | | 463.4 | to prepare |
| | b]pyridine-5-carbonitrile | | | Example 23 |

Additional Analogs Based on Example 2

The compounds in Table 27 were prepared following the procedure for the preparation of compound 101 of Example 2.

TABLE 27

| Compound | | | |
|---|---|---|---|
| Number | Compound Name | MS | MP (° C.) |
| 454 | 2-{3-[(dimethylamino)methyl]-5-methylphenyl}-4-[(4-methyl- | (ESI) m/z | 238-241 |
| | 1H-indol-5-yl)amino]thieno[2,3-b]pyridine-5-carbonitrile | 452.4 | |
| 455 | 2-{3-[(dimethylamino)methyl]-5-methyl-2-propoxyphenyl}-4- | (ESI) m/z | 224-227 |
| | [(4-methyl-1H-indol-5-yl)amino]thieno[2,3-b]pyridine-5- | 510.4 | |
| | carbonitrile | | |
| 456 | 2-{3-[(dimethylamino)methyl]-4-methoxyphenyl}-4-(1H- | (ESI) m/z | 210 (dec.) |
| | indol-5-ylamino)thieno[2,3-b]pyridine-5-carbonitrile | 454.1 | |
| 457 | 2-{5-[(dimethylamino)methyl]-2-methoxyphenyl}-4-[(4- | (ESI) m/z | 232 (dec.) |
| | methyl-1H-indol-5-yl)amino]thieno[2,3-b]pyridine-5- | 468.3 | |
| | carbonitrile | | |
| 458 | 2-{3-[(dimethylamino)methyl]phenyl}-3-methyl-4-[(4-methyl- | (ESI) m/z | 221 |
| | 1H-indol-5-yl)amino]thieno[2,3-b]pyridine-5-carbonitrile | 452.4 | |
| 459 | 2-{5-[(dimethylamino)methyl]-2-methoxyphenyl}-3-methyl-4- | (ESI) m/z | |
| | [(4-methyl-1H-indol-5-yl)amino]thieno[2,3-b]pyridine-5- | 482.2 | |
| | carbonitrile | | |
| 460 | 2-{4-[(dimethylamino)methyl]phenyl}-3-methyl-4-[(4-methyl- | (ESI) m/z | >260 |
| | 1H-indol-5-yl)amino]thieno[2,3-b]pyridine-5-carbonitrile | 452.1 | |
| 461 | 2-{2-methoxy-5-[(4-methylpiperazin-1-yl)methyl]phenyl}-4- | (ESI) m/z | 229-231 |
| | [(4-methyl-1H-indol-5-yl)amino]thieno[2,3-b]pyridine-5- | 523.4 | |
| | carbonitrile | | |
| 462 | 2-{5-[(dimethylamino)methyl]pyridin-3-yl}-4-[(4-methyl-1H- | (ESI) m/z | >260 |
| | indol-5-yl)amino]thieno[2,3-b]pyridine-5-carbonitrile | 439.2 | |
| 463 | 2-{5-[(dimethylamino)methyl]-2-fluorophenyl}-4-[(4-methyl- | (ESI) m/z | 245-247 |
| | 1H-indol-5-yl)amino]thieno[2,3-b]pyridine-5-carbonitrile | 456.3 | |
| 464 | 2-{4-[(dimethylamino)methyl]-3-thienyl}-4-(1H-indol-5- | (ESI) m/z | 200-203 |
| | ylamino)thieno[2,3-b]pyridine-5-carbonitrile | 430.2 | |
| 465 | 2-{5-[(dimethylamino)methyl]-1-benzothien-2-yl}-4-(1H- | (ESI) m/z | >245 |
| | indol-5-ylamino)thieno[2,3-b]pyridine-5-carbonitrile | 480.3 | |
| 466 | 2-{3-[(dimethylamino)methyl]-5-isopropoxyphenyl}-4-[(4- | (ESI) m/z | 227-230 |
| | methyl-1H-indol-5-yl)amino]thieno[2,3-b]pyridine-5- | 496.4 | |
| | carbonitrile | | |
| 467 | 2-{5-[(dimethylamino)methyl]-1-benzothien-2-yl}-4-[(4- | (ESI) m/z | 202-207 |
| | methyl-1H-indol-5-yl)amino]thieno[2,3-b]pyridine-5- | 494.3 | |
| | carbonitrile | | |
| 468 | 2-{5-[(dimethylamino)methyl]-3-thienyl}-4-[(4-methyl-1H- | (ESI) m/z | 225-227 |
| | indol-5-yl)amino]thieno[2,3-b]pyridine-5-carbonitrile | 444.3 | |
| 469 | 2-(2-{[(3-hydroxypropyl)amino]methyl}phenyl)-4-[(4-methyl- | (ESI) m/z | softens at |
| | 1H-indol-5-yl)amino]thieno[2,3-b]pyridine-5-carbonitrile | 468.3 | 100° C. |
| 470 | 2-(2-{[(2-hydroxyethyl)amino]methyl}phenyl)-4-[(4-methyl- | (ESI) m/z | softens at |
| | 1H-indol-5-yl)amino]thieno[2,3-b]pyridine-5-carbonitrile | 454.3; | 140° C. |
| 471 | 2-(3-{[(2-hydroxyethyl)amino]methyl}phenyl)-4-[(4-methyl- | (ESI) m/z | softens at |
| | 1H-indol-5-yl)amino]thieno[2,3-b]pyridine-5-carbonitrile | 480.3 | 135° C. |

### EXAMPLE 40

### Preparation of 3-methyl-4-[(4-methyl-1H-indol-5-yl)amino]-2-(1,2,3,6-tetrahydropyridin-4-yl)thieno[2,3-b]pyridine-5-carbonitrile 472

2-Iodo-3-methyl-4-[(4-methyl-1H-indol-5-yl)amino]thieno[2,3-b]pyridine-5-carbonitrile 318 (250 mg, 0.56 mmol) was dissolved in 10 mL DME and treated with tert-butyl 4-(4,4,5,5-tetramethyl-1,3,2-dioxaborolan-2-yl)-5,6-dihydropyridine-1(2H)-carboxylate (260 mg, 0.84 mmol), palladium acetate (9 mg, 0.04 mmol), triphenylphosphine trisulfonate (48 mg, 0.084 mmol), and sodium bicarbonate (saturated aq., 1.0 mL). The reaction was heated to 80° C. overnight. The reaction mixture was diluted with water and the product was extracted into EtOAc and purified by silica gel chromatography (EtOAc/hexane) to give 250 mg of tert-butyl 4-(5-cyano-3-methyl-4-(4-methyl-1H-indol-5-ylamino)thieno[2,3-b]pyridin-2-yl)-5,6-dihydropyridine-1(2H)-carboxylate as an oil.

Tert-butyl 4-(5-cyano-3-methyl-4-(4-methyl-1H-indol-5-ylamino)thieno[2,3-b]pyridin-2-yl)-5,6-dihydropyridine-1(2H)-carboxylate (250 mg) was dissolved in dioxane (10 mL) and treated with 4 M HCl in dioxane (10 mL, 40 mmol). After stirring 1 hour at room temperature, the resulting solid was filtered and washed with dioxane to give 3-methyl-4-[(4-methyl-1H-indol-5-yl)amino]-2-(1,2,3,6-tetrahydropyridin-4-yl)thieno[2,3-b]pyridine-5-carbonitrile 472 as its HCl salt, mp 338° C.; MS (ESI) m/z 441.3 (M+H); HPLC retention time=7.5 min.

The analogs in Table 28 were prepared from various 2-iodothieno[2,3-b]pyridine-5-carbonitriles via the procedure used to prepare compound 472.

TABLE 28

| | | | | HPLC |
|---|---|---|---|---|
| Compound | | | | Retention |
| Number | Compound Name | MS | MP (° C.) | time (min) |
| 473 | 4-(1H-indol-4-ylamino)-2-(1,2,3,6-tetrahydropyridin- | 372.2 (M + H) | 241 (dec) | 6.7 |
| | 4-yl)thieno[2,3-b]pyridine-5-carbonitrile | | | |
| 474 | 4-[(4-methyl-1H-indol-5-yl)amino]-2-(1,2,3,6- | 386.3 (M + H) | >350 | 7.2 |
| | tetrahydropyridin-4-yl)thieno[2,3-b]pyridine-5- | | | |
| | carbonitrile | | | |

### EXAMPLE 41

### Preparation of 4-[(4-methyl-1H-indol-5-yl)amino]-2-[1-(methylsulfonyl)-1,2,3,6-tetrahydropyridin-4-yl]thieno[2,3-b]pyridine-5-carbonitrile 475

4-[(4-Methyl-1H-indol-5-yl)amino]-2-(1,2,3,6-tetrahydropyridin-4-yl)thieno[2,3-b]pyridine-5-carbonitrile 474 (110 mg, 0.29 mmol) was stirred in DMF (2 mL) and treated with triethylamine (73 mg, 0.72 mmol) and mesyl chloride (42 mg, 0.37 mmol). After stirring for half an hour, the crude reaction mixture was purified by preparative HPLC to provide 4-[(4-methyl-1H-indol-5-yl)amino]-2-[1-(methylsulfonyl)-1,2,3,6-tetrahydropyridin-4-yl]thieno[2,3-b]pyridine-5-carbonitrile 475, mp 228° C.; MS (ESI) m/z 464.3 (M+H); HPLC retention time=13.4 min.

### EXAMPLE 42

### Preparation of 2-(1-benzyl-1,2,3,6-tetrahydropyridin-4-yl)-4-[(4-methyl-1H-indol-5-yl)amino]thieno[2,3-b]pyridine-5-carbonitrile 476

4-[(4-Methyl-1H-indol-5-yl)amino]-2-(1,2,3,6-tetrahydropyridin-4-yl)thieno[2,3-b]pyridine-5-carbonitrile 474 (100 mg, 0.24 mmol) was stirred in dichloroethane (4 mL) and treated with triethylamine (19 mg, 0.19 mmol) and benzaldehyde (51 mg, 0.48 mmol). After stirring for 5 minutes, sodium triacetoxyborohydride (102 mg, 0.48 mmol) was added and the reaction was stirred for 14 hours. The crude reaction mixture was partitioned between water and dichloromethane/EtOH. The organic layer was concentrated and 2-(1-benzyl-1,2,3,6-tetrahydropyridin-4-yl)-4-[(4-methyl-1H-indol-5-yl)amino]thieno[2,3-b]pyridine-5-carbonitrile 476 was purified by preparative HPLC, MS (ESI) m/z 476.2 (M+H); HPLC retention time=9.0 min.

4-[(4-Methyl-1H-indol-5-yl)amino]-2-(1-methyl-1,2,3,6-tetrahydropyridin-4-yl)thieno[2,3-b]pyridine-5-carbonitrile 477 was prepared following the procedure for the preparation of 2-(1-benzyl-1,2,3,6-tetrahydropyridin-4-yl)-4-[(4-methyl-1H-indol-5-yl)amino]thieno[2,3-b]pyridine-5-carbonitrile 476, mp 230° C.; MS (ESI) m/z 400.2 (M+H); HPLC retention time=7.2 min.

### EXAMPLE 43

### Preparation of 4-[(4-methyl-1H-indol-5-yl)amino]-2-piperidin-4-ylthieno[2,3-b]pyridine-5-carbonitrile 478

Tert-butyl 4-{5-cyano-4-[(4-methyl-1H-indol-5-yl)amino]thieno[2,3-b]pyridin-2-yl}-5,6-dihydropyridine-1(2H)-carboxylate 418 (120 mg) and Pd/C (10%, wet, ˜20 mg) was stirred in 100 mL EtOH under an atmosphere of hydrogen for 14 hours. The reaction was filtered and concentrated to dryness. The residue was treated with 2 mL of 4 M HCl/dioxane and sonicated briefly. The reaction was allowed to stand at room temperature for 1 hour, then the resulting solid was filtered and dried. The crude solid was treated with 2 mL EtOH and 0.1 mL MeOH and heated briefly to 80° C. The resulting precipitate was filtered and washed with EtOH to give 4-[(4-methyl-1H-indol-5-yl)amino]-2-piperidin-4-ylthieno[2,3-b]pyridine-5-carbonitrile 478 as an off-white solid, MS (ESI) m/z 388.3; HPLC retention time=10.6 min.

### EXAMPLE 44

### Preparation of 2-(1-benzylpyrrolidin-3-yl)-4-[(4-methyl-1H-indol-5-yl)amino]thieno[2,3-b]pyridine-5-carbonitrile 479

4-[(4-Methyl-1H-indol-5-yl)amino]-2-vinylthieno[2,3-b]pyridine-5-carbonitrile 417 (300 mg, 0.91 mmol) was stirred as a suspension in 10 mL dichloromethane and treated with TFA (207 mg, 1.82 mmol) followed by N-benzyl-1-methoxy-N-((trimethylsilyl)methyl)methanamine (430 mg, 1.82 mmol). After stirring overnight the reaction was washed with 1M NaOH and concentrated. The crude product was purified by preparative HPLC to give the desired product. The HCl salt of 2-(1-benzylpyrrolidin-3-yl)-4-[(4-methyl-1H-indol-5-yl)amino]thieno[2,3-b]pyridine-5-carbonitrile 479 was generated by addition of HCl/dioxane, mp 185° C. (dec.); MS (ESI) m/z 464.3; HPLC retention time=9.9 min.

### EXAMPLE 45

### Preparation of 4-[(4-methyl-1H-indol-5-yl)amino]thieno[2,3-b]pyridine-5-carbonitrile 480

2-Iodo-4-[(4-methyl-1H-indol-5-yl)amino]thieno[2,3-b]pyridine-5-carbonitrile 123 (100 mg) and NaOAc (100 mg) were dissolved in EtOAc (30 mL). Pd/C (10%, wet, 30 mg) was added and the reaction was stirred for 3 hours under an atmosphere of hydrogen. The reaction was filtered, concentrated, and purified by HPLC to give 4-[(4-methyl-1H-indol-5-yl)amino]thieno[2,3-b]pyridine-5-carbonitrile 480, mp 255° C.; MS (ESI) m/z 305.1 (M+H); HPLC retention time=13.0 min.

3-Methyl-4-[(4-methyl-1H-indol-5-yl)amino]thieno[2,3-b]pyridine-5-carbonitrile 481 was prepared via the route used to prepare compound 480, mp 261° C.; MS (ESI) m/z 319.2 (M+H); HPLC retention time=14.0 min.

### EXAMPLE 46

### General Procedures for the Synthesis of C-2 Phenyl Analogs with Substituted Alkoxy Groups

Scheme 19 below illustrates an exemplary route for the preparation of C-2 substituted alkoxy analogs, such as those listed in Table 29.

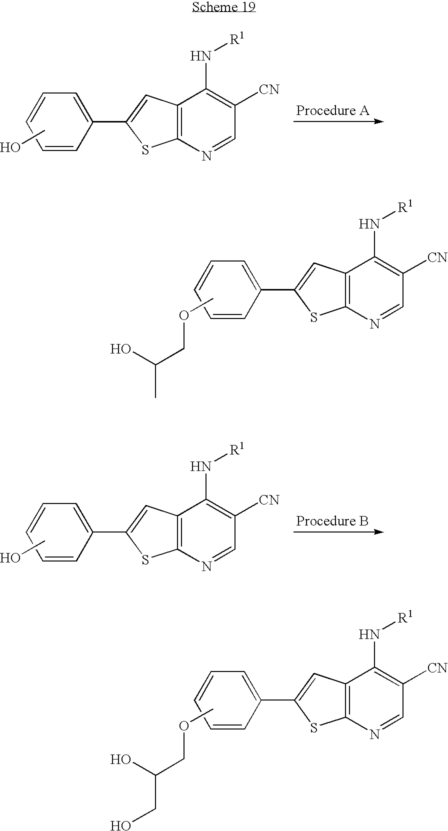

Procedure A: The phenol (0.19 mmol) was stirred as a suspension in 4 mL t-butanol and treated with the appropriate enantiomer of propylene oxide (0.95 mmol) and triethylamine (0.019 mmol). The reaction was heated to 80° C. for 24 hour then cooled to room temperature. The reaction was evaporated onto silica gel and the product was purified by silica gel chromatography.

Procedure B: The phenol (0.38 mmol), potassium carbonate (0.95 mmol), and the appropriate enantiomer of (2,2-dimethyl-1,3-dioxolan-4-yl)methyl 4-methylbenzenesulfonate (0.53 mmol) were stirred in 4 mL DMF at 80° C. overnight. The reaction was diluted with water and the crude product was extracted into EtOAc. The organic extract was washed with water twice and concentrated. The residue was dissolved in 4 mL MeOH and 1 mL water and treated with 20 mg of TsOH. The reaction was heated to 70° C. overnight then quenched with triethylamine and concentrated to dryness. The product was purified by preparative HPLC.

TABLE 29

| | | | | | HPLC |
|---|---|---|---|---|---|
| Compound | | | | | Retention |
| Number | Compound Name | Procedure | MS | MP (° C.) | time (min) |
| 482 | 2-(3-{[(2R)-2-hydroxypropyl]-oxy}phenyl)-4- | A | 455.1 | 264 | 15.8 |
| | [(4-methyl-1H-indol-5-yl)amino]thieno[2,3- | | | | |
| | b]pyridine-5-carbonitrile | | | | |
| 483 | 2-(3-{[(2S)-2-hydroxypropyl]-oxy}phenyl)-4- | A | 455.1 | 244 | 15.8 |
| | [(4-methyl-1H-indol-5-yl)amino]thieno[2,3- | | | | |
| | b]pyridine-5-carbonitrile | | | | |
| 484 | 2-(3-{[(2R)-2,3-Dihydroxypropyl]- | B | 471.3 | 241 | 12.8 |
| | oxy}phenyl)-4-[(4-methyl-1H-indol-5- | | | | |
| | yl)amino]thieno[2,3-b]pyridine-5-carbonitrile | | | | |
| 485 | 2-(3-{[(2S)-2,3-Dihydroxypropyl]- | B | 471.3 | 246 | 12.8 |
| | oxy}phenyl)-4-[(4-methyl-1H-indol-5- | | | | |
| | yl)amino]thieno[2,3-b]pyridine-5-carbonitrile | | | | |
| 486 | 2-(4-{[(2R)-2,3-dihydroxypropyl]- | B | 471.3 | 199 | 12 |
| | oxy}phenyl)-4-[(4-methyl-1H-indol-5- | | | | |
| | yl)amino]thieno[2,3-b]pyridine-5-carbonitrile | | | | |
| 487 | 2-(4-{[(2S)-2,3-dihydroxypropyl]- | B | 471.3 | 205 | 12 |
| | oxy}phenyl)-4-[(4-methyl-1H-indol-5- | | | | |
| | yl)amino]thieno[2,3-b]pyridine-5-carbonitrile | | | | |
| 488 | 2-(4-{[(2S)-2-hydroxypropyl]-oxy}phenyl)-4- | A | 455.3 | 250 | 14.5 |
| | [(4-methyl-1H-indol-5-yl)amino]thieno[2,3- | | | | |
| | b]pyridine-5-carbonitrile | | | | |
| 489 | 2-(4-{[(2R)-2-hydroxypropyl]-oxy}phenyl)-4- | A | 455.3 | 250 | 14.5 |
| | [(4-methyl-1H-indol-5-yl)amino]thieno[2,3- | | | | |
| | b]pyridine-5-carbonitrile | | | | |
| 490 | 2-(2-{[(2R)-2-hydroxypropyl]-oxy}phenyl)-4- | A | 455.3 | 250 | 14.1 |
| | [(4-methyl-1H-indol-5-yl)amino]thieno[2,3- | | | | |
| | b]pyridine-5-carbonitrile | | | | |
| 491 | 2-(2-{[(2S)-2-hydroxypropyl]-oxy}phenyl)-4- | A | 455.3 | 250 | 14.1 |
| | [(4-methyl-1H-indol-5-yl)amino]thieno[2,3- | | | | |
| | b]pyridine-5-carbonitrile | | | | |
| 492 | 2-(2-{[(2S)-2,3-dihydroxypropyl]- | B | 471.3 | 169 | 11.9 |
| | oxy}phenyl)-4-[(4-methyl-1H-indol-5- | | | | |
| | yl)amino]thieno[2,3-b]pyridine-5-carbonitrile | | | | |
| 493 | 2-(2-{[(2R)-2,3-dihydroxypropyl]- | B | 471.3 | 165 | 11.9 |
| | oxy}phenyl)-4-[(4-methyl-1H-indol-5- | | | | |
| | yl)amino]thieno[2,3-b]pyridine-5-carbonitrile | | | | |

### EXAMPLE 47

### Preparation of 2-{4-[2-(dimethylamino)ethoxy]phenyl}-4-[(4-methyl-1H-indol-5-yl)amino]thieno[2,3-b]pyridine-5-carbonitrile 494

A mixture of 2-[4-(2-chloroethoxy)phenyl]-4-[(4-methyl-1H-indol-5-yl)amino]thieno[2,3-b]pyridine-5-carbonitrile 351 (62 mg, 0.14 mmol), 2.0 M dimethylamine in THF (1.2 mL, 2.4 mmol), and sodium iodine (10 mg, 0.067 mmol) in 4 mL of DME was heated at 85° C. in a sealed tube for 20 hours. Additional 2.0 M dimethylamine in THF (0.6 mL, 1.2 mmol) was added and the mixture was heated at 85° C. in a sealed tube for an additional 24 hours then cooled to room temperature. The mixture was partitioned between dichloromethane and saturated aqueous sodium carbonate. The organic layer was dried over sodium sulfate, filtered and concentrated in vacuo. The residue was purified by preparative thin layer chromatography, developing with 15% methanol in dichloromethane, to give 30 mg of 2-{4-[2-(dimethylamino)ethoxy]phenyl}-4-[(4-methyl-1H-indol-5-yl)amino]thieno[2,3-b]pyridine-5-carbonitrile 494 as a pale yellow solid, mp 182-184° C.; MS 468.3 (M+H)+.

### EXAMPLE 48

### Preparation of 2-chloro-4-(1H-indol-5-ylamino)thieno[2,3-b]pyridine-5-carbonitrile 495

4-Chloro-5-cyanothieno[2,3-b]pyridine 10 (150 mg, 0.77 mmol) was dissolved in 7 mL THF and cooled to −78° C. LDA (1.04 mmol, in THF) was added dropwise and the reaction was stirred for 10 minutes at −78° C. Dimethylsulfamoyl chloride (166 mg, 1.15 mmol) was added and the reaction was warmed to room temperature. The reaction was diluted with water and the product was extracted into EtOAc and purified by silica gel chromatography (EtOAc/hexane) to give 75 mg of 2,4-dichlorothieno[2,3-b]pyridine-5-carbonitrile as a white solid.

2,4-Dichlorothieno[2,3-b]pyridine-5-carbonitrile (72 mg, 0.31 mmol) was treated with 5-aminoindole (0.38 mmol, 50 mg) and 3 mL EtOH. The reaction was heated to 80° C. for 2 hours then cooled to room temperature. The resulting precipitate was filtered, washed with EtOH, and purified by silica gel chromatography (EtOAc/hexane) to give 39 mg of 2-chloro-4-(1H-indol-5-ylamino)thieno[2,3-b]pyridine-5-carbonitrile 495, mp 228° C.; MS (ESI) m/z 325.1 (M+H); HPLC retention time=15.1 min.

### EXAMPLE 49

### Preparation of 3-(hydroxymethyl)-4-[(4-methyl-1H-indol-5-yl)amino]thieno[2,3-b]pyridine-5-carbonitrile 496

3-Methyl-4-oxo-4,7-dihydrothieno[2,3-b]pyridine-5-carbonitrile (8.0 g) was treated with 40 mL of POCl₃ and heated to 90° C. for 3 hours. The reaction mixture was cooled and quenched over ice. The resultant solid was filtered and washed with water to give 9.2 g of 4-chloro-3-methylthieno[2,3-b]pyridine-5-carbonitrile, MS (ESI) m/z 209.1.

4-Chloro-3-methylthieno[2,3-b]pyridine-5-carbonitrile (4.0 g, 19.2 mmol), NBS (3.4 g, 19.2 mmol), and AIBN (0.31 g, 1.92 mmol) were heated to 80° C. in 200 mL of carbon tetrachloride for 3 days. Upon cooling, 150 mL of dichloromethane, 50 mL EtOH, 100 mL of 1M NaOH, and 100 mL of water were added. After stirring for 1 hour at room temperature, the emulsion was filtered through Celite®. The organic layer was dried over MgSO₄ and concentrated to give 3.6 g of 3-(bromomethyl)-4-chlorothieno[2,3-b]pyridine-5-carbonitrile and 4-bromo-3-(bromomethyl)thieno[2,3-b]pyridine-5-carbonitrile (˜3:1 ratio).

The mixture of 3-(bromomethyl)-4-chlorothieno[2,3-b]pyridine-5-carbonitrile and 4-bromo-3-(bromomethyl)thieno[2,3-b]pyridine-5-carbonitrile (150 mg, 0.52 mmol) was treated with CaCO₃ (261 mg, 2.6 mmol) and heated to 80° C. overnight in 4 mL of 1:1 dioxane:water. The reaction was partitioned between EtOAc and dilute aqueous HCl. The organic layer was concentrated to give a yellow solid which was treated with 4-methyl-5-aminoindole (114 mg, 0.78 mmol) and 5 mL EtOH. The reaction was heated to 80° C. overnight. 3-(Hydroxymethyl)-4-[(4-methyl-1H-indol-5-yl)amino]thieno[2,3-b]pyridine-5-carbonitrile 496 was isolated after purification by preparative HPLC, mp 230° C.; MS (ESI) m/z 335.2 (M+H); HPLC retention time=11.1 min.

### EXAMPLE 50

### Preparation of 4-(4-methyl-1H-indol-5-ylamino)-3-((4-methylpiperazin-1-yl)methyl)thieno[2,3-b]pyridine-5-carbonitrile 497

3-(Hydroxymethyl)-4-[(4-methyl-1H-indol-5-yl)amino]thieno[2,3-b]pyridine-5-carbonitrile 496 (100 mg, 0.30 mmol) was stirred in 1 mL DMF and treated sequentially with triethyl amine (0.39 mmol) and mesyl chloride (0.39 mmol). After stirring overnight at room temperature, an additional quantity of triethyl amine (0.39 mmol) and mesyl chloride (0.39 mmol) were added. After stirring 3 hours at room temperature, the reaction was treated with N-methyl piperazine (0.9 mmol). After stirring for 1 hour at room temperature, the reaction was purified by preparative HPLC to give 4-(4-methyl-1H-indol-5-ylamino)-3-((4-methylpiperazin-1-yl)methyl)thieno[2,3-b]pyridine-5-carbonitrile 497, MS (ESI) m/z 417.5 (M+H), HPLC retention time=5.23 min.

### EXAMPLE 51

### Preparation of 4-(4-chloro-1H-pyrrolo[2,3-b]pyridin-5-ylamino)-2-(3-((dimethylamino)methyl)phenyl)thieno[2,3-b]pyridine-5-carbonitrile 498

4-Chloro-1H-pyrrolo[2,3-b]pyridin-5-amine (50 mg, 0.3 mmol) and 4-chloro-2-{3-[(dimethylamino)methyl]phenyl}thieno[2,3-b]pyridine-5-carbonitrile (50 mg, 0.30 mmol) were dissolved in dioxane (3 mL) and treated with potassium phosphate (127 mg), Pd(dba)₂Cl₂ (24 mg), and 2′-(dicyclohexylphosphino)-N,N-dimethylbiphenyl-2-amine (36 mg). After heating at 80° C. for 2 days, the reaction was concentrated and purified by HPLC to give 4-(4-chloro-1H-pyrrolo[2,3-b]pyridin-5-ylamino)-2-(3-((dimethylamino)methyl)phenyl)thieno[2,3-b]pyridine-5-carbonitrile 498 as a white solid, MS (ESI) m/z 459.1 (M+H); HPLC retention time=8.3 min.

### EXAMPLE 52

### Preparation of 4-(1H-indol-5-ylamino)thieno[2,3-b]pyridine-5-carbonitrile-7-oxide 499

A mixture of 4-chlorothieno[2,3-b]pyridine-5-carbonitrile 10 (250 mg, 1.28 mmol) and 77% m-CPBA (570 mg, 2.59 mmol) in 10 mL of chloroform was stirred at room temperature overnight. Additional 77% m-CPBA (300 mg) was added and the mixture was stirred at room temperature overnight. The mixture was partitioned between dichloromethane and saturated aqueous sodium bicarbonate. The organic layer was dried over magnesium sulfate, filtered and concentrated in vacuo. Trituration with diethyl ether provided a solid that was purified by flash column chromatography, eluting with a gradient of 4:1 Hexane:ethyl acetate to 100% ethyl acetate, to give 116 mg of 4-chlorothieno[2,3-b]pyridine-5-carbonitrile 7-oxide as a white solid, mp 200-203° C.; MS 211.0 (M+H)+.

A mixture of 4-chlorothieno[2,3-b]pyridine-5-carbonitrile 7-oxide (100 mg, 0.47 mmol) and 5-aminoindole (130 mg, 0.96 mmol) in 15 mL of ethanol was heated at reflux for 8 hours. The reaction mixture was cooled slightly and the off-white solid collected by filtration washing with ethanol and diethyl ether to provide 89 mg of 4-(1H-indol-5-ylamino)thieno[2,3-b]pyridine-5-carbonitrile 7-oxide 499, mp>245° C.; MS 307.1 (M+H)+.

4-(1H-indol-5-ylamino)-2-phenylthieno[2,3-b]pyridine-5-carbonitrile 7-oxide 500 was prepared following the procedure for the preparation of compound 499, 2-Phenyl-4-chlorothieno[2,3-b]pyridine-5-carbonitrile was reacted with m-CPBA to provide 2-phenyl-4-chlorothieno[2,3-b]pyridine-5-carbonitrile-7-oxide. Reaction of 2-phenyl-4-chlorothieno[2,3-b]pyridine-5-carbonitrile-7-oxide with 5-aminoindole in ethanol provided 4-(1H-indol-5-ylamino)-2-phenylthieno[2,3-b]pyridine-5-carbonitrile 7-oxide 500 as a bright yellow solid, mp>245° C.; MS 383.2 (M+H)+.

### EXAMPLE 53

### Alternate Synthesis of 2-iodo-4-[(4-methyl-1H-indol-5-yl)amino]thieno[2,3-b]pyridine5-carbonitrile 123

4-Chloro-2-iodothieno[2,3-b]pyridine-5-carbonitrile 12 (500 mg, 1.6 mmol) was treated with DMF (5 mL) and CsF (470 mg, 3.1 mmol). After heating for 2 hours at 50° C., the reaction was diluted with EtOAc was washed with water three times. The organic layer was concentrated to give the crude product which was purified by silica gel chromatography (EtOAc/hexane) to give 200 mg of 4-fluoro-2-iodothieno[2,3-b]pyridine-5-carbonitrile.

4-Fluoro-2-iodothieno[2,3-b]pyridine-5-carbonitrile (75 mg, 0.25 mmol) and 4-methyl-5-aminoindole (72 mg, 0.5 mmol) were heated to 80° C. in 1.5 mL DMF for 20 hours. Upon cooling, the crude reaction mixture was purified by HPLC to give 2-iodo-4-(4-methyl-1H-indol-5-ylamino)thieno[2,3-b]pyridine-5-carbonitrile 123 MS (ESI) m/z 451.8 (M+H), HPLC retention time=9.50 min.

4-(4-Chloro-1H-pyrrolo[2,3-b]pyridin-5-ylamino)-2-iodothieno[2,3-b]pyridine-5-carbonitrile 501 was prepared by a similar route from 4-fluoro-2-iodothieno[2,3-b]pyridine-5-carbonitrile, MS (ESI) m/z 430.9 (M+H), HPLC retention time=10.55 min.

The following compounds in Table 30 were obtained as by products.

TABLE 30

| Compound | | | |
|---|---|---|---|
| Number | Compound Name | MS | MP (° C.) |
| 502 | ethyl 5-cyano-4-(1H-indol-5- | (ESI) m/z | 240 |
| | ylamino)thieno[2,3- | 363.2 | |
| | b]pyridine-2-carboxylate | | |
| 503 | 3-isopropyl-4-[(4-methyl- | (ESI) m/z | 175 (darkens at |
| | 1H-indol-5- | 347.1 | |
| | yl)amino]thieno[2,3- | | 162° C.) |
| | b]pyridine-5-carbonitrile | | |

Additional Analogs Based on the Above Examples

The following compounds in Table 31 were prepared according to the procedure described in one or more of the examples above.

TABLE 31

| | | | | HPLC |
|---|---|---|---|---|
| Compound | | | | Retention |
| Number | Compound Name | MS | MP (° C.) | time (min) |
| 504 | 4-[(4-methyl-1H-indol-5-yl)amino]-2-pyrazin- | (ESI-FTMS) | N/A | 14.2 |
| | 2-ylthieno[2,3-b]pyridine-5-carbonitrile | 383.1[M + H]1⁺ | | |
| 505 | 2-(1H-indol-4-yl)-4-[(4-methyl-1H-indol-5- | (ESI-FTMS) | N/A | 16 |
| | yl)amino]thieno[2,3-b]pyridine-5-carbonitrile | 420.1[M + H]1⁺ | | |
| 506 | 4-[(4-methyl-1H-indol-5-yl)amino]-2- | (ESI-FTMS) | N/A | 13.1 |
| | pyrimidin-5-ylthieno[2,3-b]pyridine-5- | 383.1[M + H]1⁺ | | |
| | carbonitrile | | | |
| 507 | 4-[(4-chloro-1H-pyrrolo[2,3-b]pyridin-5- | (ESI-FTMS) | N/A | 98.5% at |
| | yl)amino]-2-iodothieno[2,3-b]pyridine-5- | 451.9[M + H]1⁺ | | 215 nm, |
| | carbonitrile | | | 15.3 |
| 508 | 3-{3-[(dimethylamino)methyl]phenyl}-4-[(4- | (ESI) m/z 438.2 | 203-205 | N/A |
| | methyl-1H-indol-5-yl)amino]thieno[2,3- | | | |
| | b]pyridine-5-carbonitrile | | | |
| 509 | N′-(3-{5-cyano-4-[(4-methyl-1H-indol-5- | (ESI) m/z 503.3 | 257 (dec.) | N/A |
| | yl)amino]thieno[2,3-b]pyridin-2-yl}phenyl)- | | | |
| | N,N-dimethylsulfamide | | | |
| 510 | 3-{5-cyano-4-[(4-methyl-1H-indol-5- | (ESI) m/z 504.3 | >260 | N/A |
| | yl)amino]thieno[2,3-b]pyridin-2-yl}-N-(2- | | | |
| | hydroxyethyl)benzenesulfonamide | | | |
| 511 | 3-{5-cyano-4-[(4-methyl-1H-indol-5- | (ESI) m/z 470.3 | >260 | N/A |
| | yl)amino]thieno[2,3-b]pyridin-2-yl}-4-fluoro- | | | |
| | N,N-dimethylbenzamide | | | |
| 512 | 3-{5-cyano-4-[(4-methyl-1H-indol-5- | (ESI) m/z 470.3 | >260 | N/A |
| | yl)amino]thieno[2,3-b]pyridin-2-yl}-5-fluoro- | | | |
| | N,N-dimethylbenzamide | | | |
| 513 | 2-[3,4-bis(2-methoxyethoxy)phenyl]-4-[(4- | (ESI) m/z 529.3 | N/A | 16.1 |
| | methyl-1H-indol-5-yl)amino]thieno[2,3- | | | |
| | b]pyridine-5-carbonitrile | | | |
| 514 | 2-(2-formyl-5-methoxyphenyl)-4-[(4-methyl- | (ESI) m/z 439.3 | >250 | N/A |
| | 1H-indol-5-yl)amino]thieno[2,3-b]pyridine-5- | | | |
| | carbonitrile | | | |
| 515 | 2-{2-[(dimethylamino)methyl]-5- | (ESI) m/z 468.3 | 180-185 | N/A |
| | methoxyphenyl}-4-[(4-methyl-1H-indol-5- | | | |
| | yl)amino]thieno[2,3-b]pyridine-5-carbonitrile | | | |
| 516 | 2-{3-[(dimethylamino)methyl]phenyl}-4-(1H- | (ESI) m/z 466.4 | 215-220 | N/A |
| | indol-5-ylamino)-3-isopropylthieno[2,3- | | | |
| | b]pyridine-5-carbonitrile | | | |
| 517 | 2-(5-{[(2-hydroxyethyl)amino]methyl}-2- | (ESI) m/z 484.3 | 213 (dec.) | N/A |
| | methoxyphenyl)-4-[(4-methyl-1H-indol-5- | | | |
| | yl)amino]thieno[2,3-b]pyridine-5-carbonitrile | | | |
| 518 | 5-{5-cyano-4-[(4-methyl-1H-indol-5- | (ESI) m/z 470.3 | >260 | N/A |
| | yl)amino]thieno[2,3-b]pyridin-2-yl}-2-fluoro- | | | |
| | N,N-dimethylbenzamide | | | |
| 519 | 2-[3-(1,1-dioxidoisothiazolidin-2-yl)phenyl]- | (ESI) m/z 500.3 | >260 | N/A |
| | 4-[(4-methyl-1H-indol-5-yl)amino]thieno[2,3- | | | |
| | b]pyridine-5-carbonitrile | | | |
| 520 | 2-(1H-indol-5-yl)-4-[(4-methyl-1H-indol-5- | (ESI) m/z 420.2 | >260 | N/A |
| | yl)amino]thieno[2,3-b]pyridine-5-carbonitrile | | | |
| 521 | 2-(1H-indol-6-yl)-4-[(4-methyl-1H-indol-5- | (ESI) m/z 420.2 | >260 | N/A |
| | yl)amino]thieno[2,3-b]pyridine-5-carbonitrile | | | |
| 522 | 4-({4-chloro-1-[(4-methylphenyl)sulfonyl]- | (ESI-FTMS) | N/A | 19 |
| | 1H-pyrrolo[2,3-b]pyridin-5-yl}amino)-2- | 605.9[M + H]1+ | | |
| | iodothieno[2,3-b]pyridine-5-carbonitrile | | | |
| 523 | 4-[(4-methyl-1H-indol-5-yl)amino]-2- | (ESI-FTMS) | N/A | 11.6 |
| | pyridazin-3-ylthieno[2,3-b]pyridine-5- | 383.1[M + H]1+ | | |
| | carbonitrile | | | |
| 524 | 4-[(4-chloro-1H-pyrrolo[2,3-b]pyridin-5- | (ESI-FTMS) | N/A | 16.2 |
| | yl)amino]-2-phenylthieno[2,3-b]pyridine-5- | 402.1[M + H]1+ | | |
| | carbonitrile | | | |
| 525 | 2-(3-formyl-2-methoxyphenyl)-4-[(4-methyl- | (ESI) m/z 439.3 | 187-190 | N/A |
| | 1H-indol-5-yl)amino]thieno[2,3-b]pyridine-5- | | | |
| | carbonitrile | | | |
| 526 | 2-{3-[(dimethylamino)methyl]-2- | (ESI) m/z 468.3 | 236 (dec.) | N/A |
| | methoxyphenyl}-4-[(4-methyl-1H-indol-5- | | | |
| | yl)amino]thieno[2,3-b]pyridine-5-carbonitrile | | | |
| 527 | 4-[(4-methyl-1H-indol-5-yl)amino]-2-(1- | (ESI) m/z 385.3 | >250 | N/A |
| | methyl-1H-pyrazol-5-yl)thieno[2,3- | | | |
| | b]pyridine-5-carbonitrile | | | |
| 528 | 2-(5-{[bis(2-hydroxyethyl)amino]methyl}-2- | (ESI) m/z 528.3 | 115 (dec.) | N/A |
| | methoxyphenyl)-4-[(4-methyl-1H-indol-5- | | | |
| | yl)amino]thieno[2,3-b]pyridine-5-carbonitrile | | | |
| 529 | 2-{3-[2-(dimethylamino)ethoxy]phenyl}-4- | (ESI) m/z 468.3 | 217-219 | N/A |
| | [(4-methyl-1H-indol-5-yl)amino]thieno[2,3- | | | |
| | b]pyridine-5-carbonitrile | | | |
| 530 | 4-[(4-chloro-1H-pyrrolo[2,3-b]pyridin-5- | (ESI) m/z 489.3 | N/A | N/A |
| | yl)amino]-2-{2-[2- | | | |
| | (dimethylamino)ethoxy]phenyl}thieno[2,3- | | | |
| | b]pyridine-5-carbonitrile | | | |

### EXAMPLE 54

### Pharmacological Testing

Evaluation of representative compounds of this invention in several standard pharmacological test procedures indicated that the compounds of this invention are effective inhibitors, of PKCθ. Based on the activity shown in the standard pharmacological test procedures, the compounds of this invention are therefore useful as anti-inflammatory agents. The test procedures used are shown below.

PKCθ Scintiplate Assay

This assay detects the phosphorylation of a biotinylated substrate by kinase utilizing radiolabeled ATP (ATP γ P33). The enzyme is either recombinant full length PKCθ (Panvera, P2996) or the purified recombinant active kinase domain of full length PKCθ (amino acids 362-706). The substrate in this assay is a biotinylated peptide with a sequence of biotin-FARKGSLRQ-CONH2. The assay buffer is composed of 100 mM Hepes, pH7.5, 2 mM MgCl₂, 20 mM β-glycerophosphate and 0.008% TritonX 100. A reaction mixture of ATP, ATP γ P33 (PerkinElmer), DTT, lipid activator, and the enzyme is prepared in the assay buffer and added to a 96 well polypropylene plate. The compound (diluted in DMSO in a separate 96-well polypropylene plate) is added to the reaction mixture and incubated at room temperature. Following the incubation, the peptide substrate is added to the reaction mixture to initiate the enzymatic reaction. The reaction is terminated with the addition of a stop solution (10 mM EDTA, 0.2% TritonX100, and 100 mM NaHPO₄) and transferred from the assay plate to a washed streptavidin-coated 96 well scintiplate (PerkinElmer). The scintiplate is incubated at room temperature, washed in PBS with 0.1% TritonX 100, and counted in the 1450 Microbeta Trilux (Wallac, Version 2.60). Counts are recorded for each well as corrected counts per minute (CCPM). The counts are considered corrected because they are adjusted according to a P33 normalization protocol, which corrects for efficiency and background differences between the instrument detectors (software version 4.40.01).

PKCθ IMAP Assay

The materials used include the following: human PKCθ full length enzyme (Panvera Catalog No. P2996); substrate peptide: 5FAM-RFARKGSLRQKNV-OH (Molecular Devices, RP7032); ATP (Sigma Cat # A2383); DTT (Pierce, 20291); 5× kinase reaction buffer (Molecular Devices, R7209); 5× binding buffer A (Molecular Devices, R7282), 5× binding buffer B (Molecular Devices, R7209); IMAP Beads (Molecular Devices, R7284); and 384-well plates (Corning Costar, 3710).

The reaction buffer was prepared by diluting the 5× stock reaction buffer and adding DTT to obtain a concentration of 3.0 mM. The binding buffer was prepared by diluting the 5× binding buffer A. A master mix solution was prepared using a 90% dilution of the reaction buffer containing 2×ATP (12 uM) and 2× peptide (200 nm). Compounds were diluted in DMSO to 20× of the maximum concentration for the IC50 measurement. 27 ul of the master mix solution for each IC50 curve was added to the first column in a 384-well plate and 3 ul of 20× compound in DMSO was added to each well. The final concentration of compound was 2× and 10% DMSO. DMSO was added to the rest of the master mix to increase the concentration to 10%. 10 ul of the master mix containing 10% DMSO was added to the rest of the wells on the plate except the 2nd column. 20 ul was transferred from the first column to the 2nd column. The compounds were serially diluted in 2:1 ratio starting from the 2nd column. A 2× (2 nM) PKCθ solution was made in the reaction buffer. 10 ul of the PKCθ solution was added to every well to achieve these final concentrations: PKCθ—1 nM; ATP—6 uM; peptide—100 nM; DMSO—5%. Samples were incubated for 25 minutes at room temperature. The binding reagent was prepared by diluting the beads in 1× binding buffer to 800:1. 50 ul of the binding reagent was added to every well and incubated for 20 minutes. FP was measured using Envision2100 (PerkinElmer Life Sciences). Wells with no ATPs and wells with no enzymes were used as controls.

The results obtained are summarized in Table 32 below. Data presented represent the average value when one or more samples were tested.

TABLE 32

| Compound # | PKCθ Scintiplate (uM) | PKCθ IMAP (uM) |
|---|---|---|
| 101 | 0.37 | 0.4 |
| 103 | 0.051 | |
| 104 | 0.28 | |
| 105 | 0.077 | |
| 106 | 0.08 | 0.14 |
| 107 | 0.079 | 0.46 |
| 109 | >150 | |
| 111 | | 0.37 |
| 112 | | 0.069 |
| 113 | | 0.014 |
| 114 | | 0.57 |
| 115 | 0.12 | |
| 116 | >3.3 | 5.6 |
| 117 | 0.028 | 0.06 |
| 118 | 2.7 | 1.9 |
| 119 | 9.1 | |
| 120 | 120 | |
| 121 | | 0.038 |
| 122 | | >55 |
| 124 | | 3.7 |
| 125 | >30 | |
| 126 | | 0.83 |
| 127 | 3 | |
| 128 | 7.3 | |
| 129 | >60 | >100 |
| 130 | | 9.3 |
| 131 | >300 | |
| 133 | 0.8 | 1.4 |
| 134 | >200 | |
| 137 | 0.082 | 0.052 |
| 138 | 0.19 | |
| 140 | 0.034 | 0.022 |
| 141 | 0.041 | 0.04 |
| 143 | 0.18 | 0.24 |
| 145 | 0.09 | |
| 146 | | 0.55 |
| 147 | 0.043 | 0.075 |
| 148 | 0.06 | 0.073 |
| 149 | 0.013 | 0.03 |
| 150 | 0.041 | 0.16 |
| 151 | 0.088 | 0.077 |
| 152 | 0.31 | |
| 153 | | 0.17 |
| 154 | | 0.18 |
| 155 | | 0.038 |
| 156 | | 0.093 |
| 157 | | 0.044 |
| 158 | | 0.057 |
| 159 | | 0.24 |
| 160 | | 0.36 |
| 161 | | 0.35 |
| 162 | | 0.27 |
| 163 | | 0.13 |
| 164 | | 0.088 |
| 165 | | 0.11 |
| 166 | | 0.16 |
| 167 | | 0.11 |
| 168 | | 0.52 |
| 169 | | 0.84 |
| 170 | | 0.43 |
| 171 | | 0.11 |
| 172 | | 0.069 |
| 173 | | 0.13 |
| 174 | | 0.24 |
| 175 | | 0.28 |
| 176 | | 0.17 |
| 177 | | 0.17 |
| 178 | | 0.3 |
| 179 | | 0.15 |
| 180 | | 0.11 |
| 181 | | 0.18 |
| 182 | | 0.13 |
| 183 | | 0.079 |
| 185 | 0.53 | |
| 186 | 0.29 | 1.6 |
| 187 | 0.0045 | 0.008 |
| 188 | 0.027 | 0.034 |
| 189 | 2.1 | |
| 190 | 0.19 | |
| 192 | 0.045 | |
| 193 | | 0.053 |
| 194 | | 0.12 |
| 195 | | 0.42 |
| 196 | | 0.038 |
| 197 | | 0.48 |
| 198 | | 0.059 |
| 201 | | 0.016 |
| 202 | | 0.1 |
| 203 | 0.11 | |
| 204 | 0.11 | 0.18 |
| 205 | 0.18 | |
| 208 | 0.05 | 0.1 |
| 209 | 0.17 | 0.15 |
| 210 | 0.58 | |
| 211 | 0.034 | 0.082 |
| 212 | | 0.037 |
| 213 | 0.075 | 0.29 |
| 214 | 0.016 | 0.19 |
| 215 | 0.5 | |
| 216 | 0.09 | 0.32 |
| 217 | 0.059 | 0.069 |
| 218 | 0.034 | 0.052 |
| 219 | | 0.084 |
| 220 | | 0.014 |
| 221 | | 0.17 |
| 222 | 0.17 | 0.15 |
| 223 | 0.088 | 1.4 |
| 225 | 0.22 | 0.44 |
| 226 | 0.076 | 0.57 |
| 227 | 0.15 | 0.29 |
| 228 | | >1.5 |
| 229 | | 0.79 |
| 230 | | 0.009 |
| 231 | | 0.21 |
| 232 | | 0.34 |
| 233 | | 40 |
| 234 | | 0.13 |
| 235 | | 1.2 |
| 236 | | 0.02 |
| 237 | | 0.05 |
| 238 | | 0.44 |
| 239 | | 1.1 |
| 240 | | 0.006 |
| 241 | | 0.013 |
| 242 | | 0.38 |
| 244 | 0.56 | 0.57 |
| 245 | | 0.068 |
| 246 | | 0.438 |
| 247 | | 0.219 |
| 248 | | 2.049 |
| 249 | | 0.048 |
| 250 | | 1.039 |
| 251 | | 1.666 |
| 252 | | 0.959 |
| 253 | | 2.282 |
| 254 | | 0.864 |
| 255 | | 0.276 |
| 256 | | 0.334 |
| 257 | | 0.025 |
| 258 | | 0.126 |
| 259 | | 0.155 |
| 260 | | 0.187 |
| 261 | | 0.222 |
| 262 | | 0.092 |
| 263 | | 0.071 |
| 264 | | 0.669 |
| 265 | | 0.232 |
| 266 | | 0.044 |
| 267 | | 3.207 |
| 268 | | 1.249 |
| 269 | | 4.029 |
| 270 | | 0.201 |
| 271 | | 1.160 |
| 272 | | 0.045 |
| 273 | | 0.447 |
| 274 | | 0.983 |
| 275 | | 0.056 |
| 276 | | 0.023 |
| 277 | | 0.304 |
| 278 | | 0.012 |
| 279 | | 0.017 |
| 280 | | 0.605 |
| 281 | | 1.962 |
| 282 | | 0.023 |
| 283 | | 0.021 |
| 284 | | 0.657 |
| 285 | | 5.023 |
| 286 | | 0.314 |
| 287 | | 0.047 |
| 288 | | 0.130 |
| 289 | | 2.068 |
| 290 | | 0.330 |
| 291 | | 0.105 |
| 292 | | 0.183 |
| 293 | | 33.556 |
| 294 | | 0.004 |
| 295 | | 0.043 |
| 296 | | 0.017 |
| 297 | | 0.012 |
| 298 | | 0.048 |
| 299 | | 0.032 |
| 300 | | 0.175 |
| 301 | | 0.171 |
| 302 | | 0.004 |
| 303 | | 0.111 |
| 304 | | 0.043 |
| 305 | | 0.002 |
| 306 | | 0.002 |
| 307 | | 0.015 |
| 308 | | 0.048 |
| 309 | | 0.053 |
| 310 | | 0.041 |
| 311 | | 0.088 |
| 312 | | 0.151 |
| 313 | | 0.104 |
| 314 | | 0.375 |
| 315 | | 0.251 |
| 316 | | 1.695 |
| 317 | | 0.718 |
| 318 | | 0.080 |
| 319 | | 1.111 |
| 320 | | 0.690 |
| 321 | | 0.345 |
| 322 | | 4.666 |
| 323 | | 0.087 |
| 324 | | 0.065 |
| 325 | | 0.015 |
| 326 | | 31.050 |
| 327 | | 16.400 |
| 328 | | 1.380 |
| 329 | | 0.160 |
| 330 | | 0.069 |
| 331 | | >25 |
| 332 | | 0.437 |
| 333 | | 0.616 |
| 334 | | >12.5 |
| 335 | | 0.124 |
| 336 | | 0.051 |
| 337 | | 0.136 |
| 338 | | 0.014 |
| 339 | | 0.009 |
| 340 | | 0.026 |
| 342 | | 0.470 |
| 343 | | 0.030 |
| 344 | | 0.818 |
| 345 | | 0.031 |
| 346 | | 0.048 |
| 347 | | 0.013 |
| 348 | | 0.018 |
| 349 | | 0.016 |
| 350 | | 0.034 |
| 351 | | 0.120 |
| 352 | | 0.023 |
| 353 | | 0.036 |
| 354 | | 0.018 |
| 355 | | 0.012 |
| 356 | | 0.050 |
| 357 | | 0.048 |
| 358 | | 0.010 |
| 359 | | 0.245 |
| 360 | | 0.023 |
| 361 | | 0.043 |
| 362 | | 0.072 |
| 363 | | 0.401 |
| 364 | | 0.022 |
| 365 | | 0.103 |
| 366 | | 0.106 |
| 367 | | 0.007 |
| 368 | | 0.100 |
| 369 | | 0.030 |
| 370 | | 1.014 |
| 371 | | 0.182 |
| 372 | | 0.627 |
| 373 | | 0.057 |
| 374 | | 0.140 |
| 375 | | 0.041 |
| 376 | | 0.026 |
| 377 | | 0.141 |
| 378 | | 0.098 |
| 379 | | 0.169 |
| 380 | | 0.016 |
| 381 | | 1.982 |
| 382 | | 0.570 |
| 383 | | 0.064 |
| 384 | | 0.093 |
| 385 | | >30 |
| 387 | | 0.002 |
| 388 | | 0.118 |
| 389 | | 0.018 |
| 390 | | 0.043 |
| 391 | | 0.086 |
| 392 | | 0.177 |
| 393 | | 0.356 |
| 394 | | 0.010 |
| 395 | | 0.281 |
| 396 | | 0.156 |
| 397 | | 0.292 |
| 398 | | 0.075 |
| 399 | | 0.001 |
| 400 | | 0.437 |
| 401 | | 2.446 |
| 402 | | 0.465 |
| 403 | | 0.394 |
| 404 | | 1.143 |
| 405 | | 0.477 |
| 406 | | 12.018 |
| 408 | | 23.549 |
| 409 | | 3.704 |
| 410 | | 1.051 |
| 411 | | 2.552 |
| 412 | | 0.413 |
| 413 | | 0.007 |
| 414 | | 0.029 |
| 415 | | 0.642 |
| 416 | | 0.034 |
| 417 | | 0.033 |
| 418 | | 0.100 |
| 419 | | 0.954 |
| 420 | | 0.014 |
| 421 | | 0.324 |
| 422 | | 0.069 |
| 423 | | 0.058 |
| 424 | | 0.194 |
| 425 | | 0.095 |
| 426 | | 0.119 |
| 427 | | 0.034 |
| 428 | | 0.060 |
| 429 | | 0.060 |
| 430 | | 0.116 |
| 431 | | 0.110 |
| 432 | | 0.049 |
| 433 | | 0.527 |
| 434 | | 0.070 |
| 435 | | 0.282 |
| 436 | | 0.215 |
| 437 | | 0.011 |
| 438 | | 0.014 |
| 439 | | 0.013 |
| 440 | | 0.016 |
| 441 | | 0.029 |
| 442 | | 0.273 |
| 443 | | 0.125 |
| 444 | | 0.176 |
| 445 | | 0.550 |
| 447 | | 0.171 |
| 448 | | 0.083 |
| 449 | | 0.025 |
| 450 | | 0.126 |
| 451 | | 0.024 |
| 452 | | 0.018 |
| 453 | | 0.060 |
| 454 | | 0.008 |
| 455 | | 0.029 |
| 456 | | 0.318 |
| 457 | | 0.005 |
| 458 | | 0.099 |
| 459 | | 0.243 |
| 460 | | 0.061 |
| 461 | | 0.006 |
| 462 | | 0.083 |
| 463 | | 0.014 |
| 464 | | 0.077 |
| 465 | | 0.049 |
| 466 | | 0.064 |
| 467 | | 0.171 |
| 468 | | 0.016 |
| 469 | | 0.151 |
| 470 | | 0.057 |
| 471 | | 0.005 |
| 472 | | 0.189 |
| 473 | | 0.143 |
| 474 | | 0.010 |
| 475 | | 0.031 |
| 476 | | 0.153 |
| 477 | | 0.041 |
| 478 | | 0.070 |
| 479 | | 1.972 |
| 480 | | 0.067 |
| 481 | | 0.046 |
| 482 | | 0.093 |
| 483 | | 0.059 |
| 484 | | 0.017 |
| 485 | | 0.040 |
| 486 | | 0.070 |
| 487 | | 0.041 |
| 488 | | 0.200 |
| 489 | | 0.175 |
| 490 | | 0.105 |
| 491 | | 0.177 |
| 492 | | 0.050 |
| 493 | | 0.045 |
| 494 | | 0.023 |
| 495 | | 0.395 |
| 496 | | 0.063 |
| 498 | | 0.003 |
| 499 | | 1.321 |
| 500 | | 16.848 |
| 502 | | 1.097 |
| 503 | | 0.011 |
| 504 | | 0.021 |
| 505 | | 0.113 |
| 506 | | 0.017 |
| 507 | | 0.069 |
| 508 | | 0.011 |
| 509 | | 0.051 |
| 510 | | 0.017 |
| 511 | | 0.066 |
| 512 | | 0.038 |
| 513 | | 1.614 |
| 514 | | 0.071 |
| 515 | | 0.016 |
| 516 | | 1.215 |
| 517 | | 0.002 |
| 518 | | 0.059 |
| 519 | | 0.049 |
| 520 | | 0.074 |
| 521 | | 0.037 |
| 522 | | 14.090 |
| 523 | | 0.015 |
| 524 | | 0.048 |
| 525 | | 0.015 |
| 526 | | 0.012 |
| 527 | | 0.035 |

Variations, modifications, and other implementations of what is described herein will occur to those of ordinary skill in the art without departing from the spirit and the essential characteristics of the invention. Accordingly, the scope of the present teachings is to be defined not by the preceding illustrative description but instead by the following claims, and all changes that come within the meaning and range of equivalency of the claims are intended to be embraced therein.

What is claimed is:

1. A compound of formula I or a pharmaceutically acceptable salt, hydrate or ester thereof: 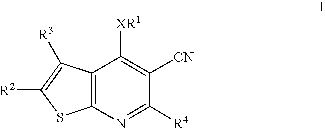
wherein:
X is a) —NR⁵—Y—, b) —O—Y—, c) —S(O)ₘ—Y—, d) —S(O)ₘNR⁵—Y—, e) —NR⁵S(O)ₘ—Y—, f)—C(O)NR⁵—Y—, g) —NR⁵C(O)—Y—, h) —C(S)NR⁵—Y—, i) —NR⁵C(S)—Y—, j) —C(O)O—Y—, k) —OC(O)—Y—, l) —C(O)—Y—, or m) a covalent bond;
Y, at each occurrence, independently is a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, or e) a covalent bond;
R¹ is a) a C₁₋₁₀ alkyl group, b) a C₃₋₁₀ cycloalkyl group, c) a 3-12 membered cycloheteroalkyl group, d) a C₆₋₁₄ aryl group, or e) a 5-13 membered heteroaryl group, wherein each of a)-e) optionally is substituted with 1-4 R⁶ groups, and provided that R¹ is not a phenyl group;
R² is a) H, b) halogen, c) —C(O)R⁸, d) —C(O)OR⁸, e) —C(O)NR⁹R¹⁰, f) —C(S)R⁸, g) —C(S)OR⁸, h) —C(S)NR⁹R¹⁰, i) a C₁₋₁₀ alkyl group, j) a C₂₋₁₀ alkenyl group, k) a C₂₋₁₀ alkynyl group, l) a C₃₋₁₀ cycloalkyl group, m) a C₆₋₁₄ aryl group, n) a 3-12 membered cycloheteroalkyl group, or o) a 5-13 membered heteroaryl group, wherein each of i)-o) optionally is substituted with 1-4 R⁶ groups;
R³ is a) H, b) halogen, c) —OR⁸, d) —NR⁹R¹⁰, e) —N(O)R⁹R¹⁰, f) S(O)ₘR⁸, g) S(O)ₘOR⁸, h) —C(O)R⁸, i) —C(O)OR⁸, j) —C(O)NR⁹R¹⁰, k) —C(S)R⁸, l) —C(S)OR⁸, m) —C(S)NR⁹R¹⁰, n) —Si(C₁₋₁₀ alkyl group)₃, o) a C₁₋₁₀ alkyl group, p) a C₂₋₁₀ alkenyl group, q) a C₂₋₁₀ alkynyl group, r) a C₃₋₁₀ cycloalkyl group, s) a C₆₋₁₄ aryl group, t) a 3-12 membered cycloheteroalkyl group, or u) a 5-13 membered heteroaryl group, wherein each of o)-u) optionally is substituted with 1-4 R⁶ groups;
R⁴ is a) H, b) halogen, c) a C₁₋₁₀ alkyl group, d) a C₂₋₁₀ alkenyl group, e) a C₂₋₁₀ alkynyl group, f) a C₁₋₁₀ haloalkyl group, g) a C₃₋₁₀ cycloalkyl group, h) a C₆₋₁₄ aryl group, i) a 3-12 membered cycloheteroalkyl group, or j) a 5-13 membered heteroaryl group, wherein each of c)-j) optionally is substituted with 1-4 R⁶ groups;
R⁵ is a) H, b) a C₁₋₁₀ alkyl group, c) a C₂₋₁₀ alkenyl group, d) a C₂₋₁₀ alkynyl group, or e) a C₁₋₁₀ haloalkyl group;
R⁶, at each occurrence, independently is a) R⁷ or b) —Y—R⁷;
R⁷, at each occurrence, independently is a) halogen, b) —CN, c) —NO₂, d) oxo, e) —OR⁸, f) —NR⁹R¹⁰, g) —N(O)R⁹R¹⁰, h) —S(O)ₘR⁸, i) —S(O)ₘOR⁸, j) —SO₂NR⁹R¹⁰, k) —C(O)R⁸, l) —C(O)OR⁸, m) —C(O)NR⁹R¹⁰, n) —C(S)R⁸, o) —C(S)OR⁸, p) —C(S)NR⁹R¹⁰, q) —Si(C₁₋₁₀ alkyl)₃, r) a C₁₋₁₀ alkyl group, s) a C₂₋₁₀ alkenyl group, t) a C₂₋₁₀ alkynyl group, u) a C₁₋₁₀ haloalkyl group, v) a C₃₋₁₀ cycloalkyl group, w) a C₆₋₁₄ aryl group, x) a 3-12 membered cycloheteroalkyl group, or y) a 5-13 membered heteroaryl group, wherein each of r)-y) optionally is substituted with 1-4 R¹¹ groups;
R⁸, at each occurrence, independently is a) H, b) —C(O)R¹⁴, c) —C(O)OR¹⁴, d) a C₁₋₁₀ alkyl group, e) a C₂₋₁₀ alkenyl group, f) a C₂₋₁₀ alkynyl group, g) a C₁₋₁₀ haloalkyl group, h) a C₃₋₁₀ cycloalkyl group, i) a C₆₋₁₄ aryl group, j) a 3-12 membered cycloheteroalkyl group, or k) a 5-13 membered heteroaryl group, wherein each of d)-k) optionally is substituted with 1-4 R¹¹ groups;
R⁹ and R¹⁰, at each occurrence, independently are a) H, b) —OR¹³, c) —NR⁴R¹⁵, d) —S(O)ₘR¹⁴, e) —S(O)ₘOR¹⁴, f) —S(O)₂NR¹⁴R¹⁵, g) —C(O)R¹⁴, h) —C(O)OR¹⁴, i) —C(O)NR¹⁴R¹⁵, j) —C(S)R¹⁴, k) —C(S)OR¹⁴, l) —C(S)NR¹⁴R¹⁵, m) a C₁₋₁₀ alkyl group, n) a C₂₋₁₀ alkenyl group, o) a C₂₋₁₀ alkynyl group, p) a C₁₋₁₀ haloalkyl group, q) a C₃₋₁₀ cycloalkyl group, r) a C₆₋₁₄ aryl group, s) a 3-12 membered cycloheteroalkyl group, or t) a 5-13 membered heteroaryl group; wherein each of m)-t) optionally is substituted with 1-4 R¹¹ groups;
R¹¹, at each occurrence, independently is a) R¹², or b) —Y—R¹²;
R¹², at each occurrence, independently is a) halogen, b) —CN, c) —NO₂, d) oxo, e) —OR¹³, f)—NR¹⁴R¹⁵, g) —N(O)R¹⁴R¹⁵, h) —S(O)ₘR¹³, i) —S(O)ₘOR¹³, j) —SO₂NR¹⁴ R¹⁵, k) —C(O)R¹³, l) —C(O)OR¹³, m) —C(O)NR¹⁴R¹⁵, n) —C(S)R¹³, o) —C(S)OR¹³, p) —(S)NR¹⁴R¹⁵, q) —Si(C₁₋₁₀ alkyl)₃, r) a C₁₋₁₀ alkyl group, s) a C₂₋₁₀ alkenyl group, t) a C₂₋₁₀ alkynyl group, u) a C₁₋₁₀ haloalkyl group, v) a C₃₋₁₀ cycloalkyl group, w) a C₆₋₁₄ aryl group, x) a 3-12 membered cycloheteroalkyl group, or y) a 5-13 membered heteroaryl group, wherein each of r)-y) optionally is substituted with 1-4 R¹⁶ groups;
R¹³ is selected from a) H, b) —C(O)R¹⁴, c) —C(O)OR¹⁴, d) a C₁₋₁₀ alkyl group, e) a C₂₋₁₀ alkenyl group, f) a C₂₋₁₀ alkynyl group, g) a C₁₋₁₀ haloalkyl group, h) a C₃₋₁₀ cycloalkyl group, i) a C₆₋₁₄ aryl group, j) a 3-12 membered cycloheteroalkyl group, or k) a 5-13 membered heteroaryl group, wherein each of d)-k) optionally is substituted with 1-4 R¹⁶ groups;
R¹⁴ and R¹⁵, at each occurrence, independently are a) H, b) a C₁₋₁₀ alkyl group, c) a C₂₋₁₀ alkenyl group, d) a C₂₋₁₀ alkynyl group, e) a C₁₋₁₀ haloalkyl group, f) a C₃₋₁₀ cycloalkyl group, g) a C₆₋₁₄ aryl group, h) a 3-12 membered cycloheteroalkyl group, or i) a 5-13 membered heteroaryl group; wherein each of b)-i) optionally is substituted with 1-4 R¹⁶ groups;
R¹⁶, at each occurrence, independently is a) halogen, b) —CN, c) —NO₂, d) —OH, e) —NH₂, f) —NH(C₁₋₁₀ alkyl), g) oxo, h) —N(C₁₋₁₀ alkyl)₂, i) —SH, j) —S(O)ₘ—C₁₋₁₀ alkyl, k) —S(O)₂OH, l) —S(O)ₘ-OC₁₋₁₀ alkyl, m) —C(O)—C₁₋₁₀ alkyl, n) —C(O)OH, o) —C(O)—OC₁₋₁₀ alkyl, p) —C(O)NH₂, q) —C(O)NH—C₁₋₁₀ alkyl, r) —C(O)N(C₁₋₁₀ alkyl)₂, s) —C(S)NH₂, t) —C(S)NH—C₁₋₁₀ alkyl, u) —C(S)N(C₁₋₁₀ alkyl)₂, v) a C₁₋₁₀ alkyl group, w) a C₂₋₁₀ alkenyl group, x) a C₂₋₁₀ alkynyl group, y) a C₁₋₁₀ alkoxy group, z) a C₁₋₁₀ alkylthio group, aa) a C₁₋₁₀ haloalkyl group, ab) a C₃₋₁₀ cycloalkyl group, ac) a C₆₋₁₄ aryl group, ad) a 3-12 membered cycloheteroalkyl group, or ae) a 5-13 membered heteroaryl group; and
m is 0, 1, or 2.

2. An N-oxide compound of formula I′: 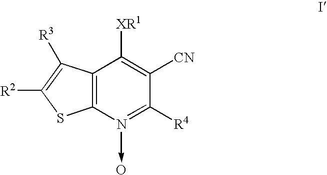
wherein R¹, R², R³, R⁴, and X are as defined in claim 1.

3. An S-oxide or S,S-dioxide compound of formula I″: 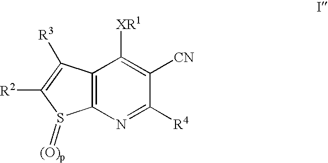
wherein p is 1 or 2, and R¹, R², R³, R⁴, and X are as defined in claim 1.

4. The compound of claim 1 or a pharmaceutically acceptable salt, hydrate or ester thereof, wherein R⁴ is H.

5. The compound of claim 1 or a pharmaceutically acceptable salt, hydrate or ester thereof, wherein X is —NR⁵—Y—, —O—, —NR⁵C(O)—, or a covalent bond, R⁵ is H or a C₁₋₆ alkyl group, and Y is a divalent C₁₋₆ alkyl group or a covalent bond.

6. The compound of claim 1 or a pharmaceutically acceptable salt, hydrate or ester thereof, wherein R¹ is a 5-13 membered heteroaryl group optionally substituted with 1-4 R⁶ groups.

7. The compound of claim 1 or a pharmaceutically acceptable salt, hydrate or ester thereof, wherein R¹ is an indolyl group, a benzimidazolyl group, a pyrrolo[2,3-b]pyridinyl group, a pyridinyl group, or an imidazolyl group, each optionally substituted with 1-4 R⁶ groups.

8. The compound of claim 1 or a pharmaceutically acceptable salt, hydrate or ester thereof, wherein R² is H, a halogen, —C(O)R⁸, —C(O)OR⁸, —C(O)NR⁹R¹⁰, a C₁₋₁₀ alkyl group, a C₂₋₁₀ alkenyl group, a C₂₋₁₀ alkynyl group, a C₃₋₁₀ cycloalkyl group, a 3-12 membered cycloheteroalkyl group, a C₆₋₁₄ aryl group, or a 5-13 membered heteroaryl group, wherein each of the C₁₋₁₀ alkyl group, the C₂₋₁₀ alkenyl group, the C₂₋₁₀ alkynyl group, the C₃₋₁₀ cycloalkyl group, the 3-12 membered cycloheteroalkyl group, the C₆₋₁₄ aryl group, and the 5-13 membered heteroaryl group is optionally substituted with 1-4 R⁶ groups.

9. The compound of claim 8 or a pharmaceutically acceptable salt, hydrate or ester thereof, wherein R⁶, at each occurrence, is independently a halogen, an oxo group, —OR⁸, —NR⁹R¹⁰, —S(O)ₘR⁸, —S(O)ₘOR⁸, —SO₂NR⁹R¹⁰, —C(O)R⁸, —C(O)OR⁸, —C(O)NR⁹R¹⁰, —Si(CH₃)₃, a —C₁₋₄ alkyl-OR⁸, a —C₁₋₄ alkyl-NR⁹R¹⁰ group, a —C₁₋₄ alkyl-C₆₋₁₄ aryl group, a —C₁₋₄ alkyl-3-12 membered cycloheteroalkyl group, a —C₁₋₄ alkyl-5-13 membered heteroaryl group, a C₁₋₁₀ alkyl group, a C₂₋₁₀ alkenyl group, a C₂₋₁₀ alkynyl group, a C₁₋₁₀ haloalkyl group, a C₃₋₁₀ cycloalkyl group, a C₆₋₁₄ aryl group, a 3-12 membered cycloheteroalkyl group, or a 5-13 membered heteroaryl group, wherein R⁸, R⁹ and R¹⁰ are as defined in claim 1 and each of the C₁₋₁₀ alkyl group, the C₂₋₁₀ alkenyl group, the C₂₋₁₀ alkynyl group, the C₃₋₁₀ cycloalkyl group, the C₆₋₁₄ aryl group, the 3-12 membered cycloheteroalkyl group, and the 5-13 membered heteroaryl group immediately above is optionally substituted with 1-4 R¹¹ groups.

10. The compound of claim 1 or a pharmaceutically acceptable salt, hydrate or ester thereof, wherein R² is a C₃₋₆ cycloalkyl group, a 3-10 membered cycloheteroalkyl group, a C₆₋₁₀ aryl group, or a 5-10 membered heteroaryl group, each of which is optionally substituted with 1-4 R⁶ groups.

11. The compound of claim 1 or a pharmaceutically acceptable salt, hydrate or ester thereof, wherein R² is a cyclohexanyl group, a cyclohexenyl group, a piperazinyl group, a piperidinyl group, a morpholinyl group, a pyrrolidinyl group, a tetrahydropyridinyl group, a dihydropyridinyl group, a phenyl group, a naphthyl group, a pyridinyl group, a pyrazolyl group, a pyridazinyl group, an indolyl group, a pyrazinyl group, a pyrimidinyl group, a thienyl group, a furyl group, a thiazolyl group, a quinolinyl group, a benzothienyl group, or an imidazolyl group, each of which is optionally substituted with 1-4 R⁶ groups.

12. The compound of claim 1 or a pharmaceutically acceptable salt, hydrate or ester thereof, wherein R² is a phenyl group optionally substituted with 1-4 R⁶ groups, wherein R⁶, at each occurrence, is independently a halogen, —OR⁸, —NR⁹R¹⁰, —S(O)ₘR⁸, —S(O)ₘOR⁸, —SO₂NR⁹R¹⁰, —C(O)R⁸, —C(O)OR⁸, —C(O)NR⁹R¹⁰, a C₁₋₆ alkyl group, a C₃₋₆ cycloalkyl group, a C₆₋₁₀ aryl group, a 3-10 membered cycloheteroalkyl group, or a 5-10 membered heteroaryl group, R⁸, R⁹ and R¹⁰ are as defined in claim 1, and each of the C₁₋₆ alkyl group, the C₃₋₆ cycloalkyl group, the C₆₋₁₀ aryl group, the 3-10 membered cycloheteroalkyl group, and the 5-10 membered heteroaryl group is optionally substituted with 1-4 R¹¹ groups.

13. The compound of claim 1 or a pharmaceutically acceptable salt, hydrate or ester thereof, wherein R² is a C₁₋₆ alkyl group, a C₂₋₆ alkenyl group, or a C₂₋₆ alkynyl group, each of which is optionally substituted with 1-4 R⁶ groups, wherein R⁶, at each occurrence, is independently a halogen, —OR⁸, —NR⁹R¹⁰, —C(O)R⁸, —C(O)OR⁸, —C(O)NR⁹R¹⁰, —Si(CH₃)₃, a phenyl group, a 5-6 membered cycloheteroalkyl group, or a 5-6 membered heteroaryl group, R⁸, R⁹ and R¹⁰ are as defined in claim 1, and each of the phenyl group, the 5-6 membered cycloheteroalkyl group, and the 5-6 membered heteroaryl group is optionally substituted with 1-4 R¹¹ groups.

14. The compound of claim 1 or a pharmaceutically acceptable salt, hydrate or ester thereof, wherein R³ is H, a halogen, a C₁₋₆ alkyl group, a C₂₋₆ alkynyl group, or a phenyl group, and each of the C₁₋₆ alkyl group, the C₂₋₆ alkynyl group, and the phenyl group is optionally substituted with 1-4 R⁶ groups.

15. A compound of claim 1 selected from the compounds listed in Table 1.

16. A pharmaceutical composition comprising the compound of claim 1 or a pharmaceutically acceptable salt, hydrate or ester thereof, and a pharmaceutically acceptable carrier or excipient.

17. A method of treating or inhibiting a pathological condition or disorder mediated by a protein kinase in a mammal, the method comprising administering to the mammal a therapeutically effective amount of the compound of claim 1 or a pharmaceutically acceptable salt, hydrate, or ester thereof.

18. The method of claim 17, wherein the protein kinase is protein kinase C.

19. The method of claim 17, wherein the pathological condition or disorder is an inflammatory disease or an autoimmune disease.

20. The method of claim 17, wherein the pathological condition or disorder is asthma, psoriasis, arthritis, rheumatoid arthritis, joint inflammation, multiple sclerosis, diabetes, or an inflammatory bowel disease.
